(19) United States
(12) Patent Application Publication
(10) Pub. No.: US 2007/0253901 A1
(43) Pub. Date: Nov. 1, 2007

(54) Atherosclerosis genes and related reagents and methods of use thereof

(76) Inventors: David Deng, Mountain View, CA (US); Anya Tsalenko, Chicago, IL (US); Amir Ben-Dor, Bellevue, WA (US); Zohar H. Yakhini, Ramat Hasharon (IL); Thomas Quertermous, Stanford, CA (US); Euan A. Ashley, Menlo Park, CA (US); Eugene Yang, Danville, CA (US); Raymond Tabibiazar, Menlo Park, CA (US); Philip Tsao, Los Altos, CA (US)

Correspondence Address: AGILENT TECHNOLOGIES, INC;ATTN: MICHAEL BECK, ESQ., 3500 DEER CREEK ROAD, MAIL STOP 26U-25, PALO ALTO, CA, 94304-1317, US

(21) Appl. No.: 11/412,437

(22) Filed: Apr. 27, 2006

(51) Int. Cl. A61K 51/00; A61K 49/10; C07K 16/46; C07K 14/54

(52) U.S. Cl. 424/1.69; 424/9.34; 530/351; 530/391.1

(57) ABSTRACT

The invention provides genes (DEA genes) that are differentially expressed in atherosclerotic lesions and polypeptides encoded by these genes. The invention provides compositions comprising a targeting agent conjugated to a functional moiety, wherein the targeting agent selectively binds to a polypeptide encoded by one a DEA gene. The functional moiety can be an imaging agent, therapeutic agent, etc. The invention further provides methods for providing diagnostic or prognostic information related to atherosclerosis involving detecting expression or activity of an expression product of one or more of the DEA genes. The invention further provides therapeutic methods comprising administering to a subject a composition comprising a targeting agent conjugated to a functional moiety that binds selectively binds to a polypeptide encoded by a DEA gene.

### BACKGROUND OF THE INVENTION

Atherosclerosis is a systemic disease in which there is a build-up of lipid-rich plaques within the walls of large arteries. Since 1900, atherosclerosis and its associated pathology, e.g., atherosclerotic coronary artery disease (CAD) and stroke, has almost invariably been the number one killer in the United States on an annual basis (see American Heart Association web site for annual statistics). In 2001, cardiovascular disease alone accounted for over a third of all deaths. The severity of the disease is not limited to the United States; the World Health Organization estimates that approximately 16.7 million people around the world die of cardiovascular disease every year (see International Cardiovascular Statistics, American Heart Association).

Atherosclerosis is a multifactorial disease stemming from many different genetic and environmental factors and is the primary disease of the coronary arteries (Poulter N. Am J Hypertens 12: 92S-95S, 1999; Ross R., N Engl J Med 340: 115-126, 1999. The role of genetics in atherosclerosis has been recognized for some time: inheritance of risk factors was first shown in classical twin studies (Evans A, et al., Twin Res 6: 432-441, 2003; Hong Y, et al., Hypertension 24: 663-670, 1994; Iliadou A, et al., J Hypertens 20: 1543-1550, 2002) and family history studies (Scheuner, M T, Genet Med. 2003 July-August; 5(4):269-85). Diabetes, hypercholesterolemia, hypertension, obesity, smoking, and physical inactivity are also known risk factors for the disease. Although atherosclerosis frequently remains clinically silent in its early stages and is often considered to be a disease associated with the later decades of life, the condition is evident at post-mortem examination even among individuals in their teens and twenties (McGill, H. C. Jr & McMahan, C. A., Am. J. Cardiol., 82, 30T-36T, 1998).

While interventional cardiology procedures such as balloon angioplasty, stenting, and atherectomy have shown some success in combating local coronary arterial disease, this has not been met by equivalent success in interrupting the underlying disease at the molecular level. Attention has focused on pharmaceutical interventions that cause a reduction in the serum levels of various lipids that are believed to contribute to disease progression. However, there is no currently approved treatment designed to target the molecular interactions of the disease process itself.

Thus it is evident that there is a need in the art for new methods for the treatment of atherosclerosis. In addition, there is a need in the art for improved methods for the diagnosis and prognosis of atherosclerosis and for evaluating response to therapy. These needs are particularly evident in view of the large number of individuals who may be at risk but have not yet manifested clinical symptoms.

### SUMMARY OF THE INVENTION

The present invention provides genes that are differentially expressed between normal blood vessel tissue and blood vessel tissue affected by atherosclerosis. These genes, and their associated polypeptides and polynucleotides, which are also provided by the invention, have been named DEA genes, DEA polynucleotides, and DEA polypeptides, where DEA stands for “differentially expressed in atherosclerosis”.

In one aspect, the invention provides genes that are differentially expressed between normal blood vessel tissue and blood vessel tissue having an athersclerotic lesion. These genes, and their associated polypeptides and polynucleotides, have been named DEA-A genes, DEA-A polynucleotides, and DEA-A polypeptides and are included among the DEA genes, DEA polynucleotides, and DEA polypeptides of the invention.

The invention also provides genes that are differentially expressed between blood vessel tissue in subjects that have diabetes and blood vessel tissue in subjects that do not have diabetes. These genes and their associated polypeptides and polynucleotides, have been named DEA-DB genes, DEA-DB polynucleotides, and DEA-DB polypeptides, respectively. These genes are included among the DEA genes, DEA polynucleotides, and DEA polypeptides of the invention. Diabetic subjects are at increased risk for atherosclerosis and frequently develop a particularly severe from of the condition. In some embodiments of the invention these genes are particularly appropriate targets for diagnosis and/or therapy in subjects having diabetes. Without wishing to be bound by any theory, genes that are overexpressed in blood vessels of diabetic subjects may be related to this increased susceptibility and increased severity. As such, these genes may be particularly suitable targets for prevention and early intervention in both diabetic and nondiabetic subjects. In addition, subjects that are not known to be diabetic but that display increased expression of these genes in their blood vessels may benefit from preventive therapy and monitoring for the development of diabetes and/or the development of atherosclerosis. Therefore these genes are appropriate for use in the diagnostic and therapeutic methods of the invention.

The invention also provides genes that are differentially expressed between non-lesion blood vessel tissue in subjects that have diabetes and non-lesion blood vessel tissue in subjects that do not have diabetes. These genes and their associated polypeptides and polynucleotides, have been named DEA-DNL genes, DEA-DNL polynucleotides, and DEA-DNL polypeptides, respectively, and are among the DEA genes, DEA polynucleotides, and DEA polypeptides of the invention. In some embodiments of the invention these genes are particularly appropriate targets for diagnosis and/or therapy in subjects having diabetes. As mentioned above, diabetic subjects are at increased risk for atherosclerosis and frequently develop a particularly severe form of the condition. Therefore, without wishing to be bound by any theory, genes that are overexpressed in blood vessels of diabetic subjects, even in blood vessel segments that do not yet exhibit evidence of atherosclerosis, may be related to this increased susceptibility and increased severity. As such, these genes may be particularly suitable targets for prevention and early intervention in both diabetic and nondiabetic subjects. In addition, subjects that are not known to be diabetic but that display increased expression of these genes may benefit from preventive therapy and monitoring for the development of diabetes and/or the development of atherosclerosis. Therefore these genes are appropriate for use in the diagnostic and therapeutic methods of the invention.

The invention also provides genes that are differentially expressed between atherosclerotic lesions in subjects that have diabetes and atherosclerotic lesions in subjects that do not have diabetes. These genes and their associated polypeptides and polynucleotides, have been named DEA-DL genes, DEA-DL polynucleotides, and DEA-DL polypeptides, respectively, and are among the DEA genes, DEA polynucleotides, and DEA polypeptides of the invention. In certain embodiments of the invention these genes are particularly appropriate targets for diagnosis and/or therapy in subjects having diabetes.

In another aspect, the invention provides cDNA and oligonucleotide arrays (e.g., microarrays) comprising probes (e.g., cDNAs or oligonucleotides) that specifically hybridize to target DEA polynucleotides. The arrays may be capable of detecting between 10% and 100% of the DEA polynucleotides. In certain embodiments of the invention at least 10%, at least 20%, at least 30%, at least 40%, at least 50%, at least 60%, at least 70%, at least 80%, at least 90%, or 100% of the probes attached to the array hybridize to a DEA polynucleotide (i.e., the probes hybridize to different DEA polynucleotides). In certain embodiments of the invention at least 10%, at least 20%, at least 30%, at least 40%, at least 50%, at least 60%, at least 70%, at least 80%, at least 90%, or 100% of the probes attached to the array hybridize to a DEA polynucleotide (i.e., the probes hybridize to different DEA polynucleotides). In some embodiments of the invention at least 80% or at least 90% of the DEA polynucleotides are DEA-A polynucleotides, DEA-DB polynucleotides, DEA-DL polynucleotides, or DEA-DNL polynucleotides.

The invention further provides protein arrays (e.g., protein microarrays) comprising binding agents (e.g., antibodies, antibody fragments, affibodies, ligands) that specifically bind to target DEA polynucleotides. The arrays may be capable of detecting between 10% and 100% of the DEA polypeptides. In certain embodiments of the invention at least 10%, at least 20%, at least 30%, at least 40%, at least 50%, at least 60%, at least 70%, at least 80%, at least 90%, or 100% of the binding agents attached to the array specifically bind to a DEA polypeptide (i.e., the binding agents specifically bind to different DEA polypeptides). In certain embodiments of the invention at least 10%, at least 20%, at least 30%, at least 40%, at least 50%, at least 60%, at least 70%, at least 80%, at least 90%, or 100% of the binding agents attached to the array specifically bind to a DEA polypeptide (i.e., the binding agents bind to different DEA polypeptides). In some embodiments of the invention at least 80% or at least 90% of the DEA polypeptides fall into the category of DEA-A polypeptides, DEA-DB polypeptides, DEA-DL polypeptides, or DEA-DNL polypeptides. It is noted that some of the DEA genes, polynucleotides, and polypeptides fall into multiple categories and are considered members of each category for purposes of determining whether these minimum percentages are met.

In additional aspects, the invention provides an RNAi agent that inhibits expression of a DEA polynucleotide, an antisense molecule that inhibits expression of a DEA polynucleotide, and a ribozyme that cleaves a DEA polynucleotide. In some embodiments of the invention the DEA polynucleotide is overexpressed in atherosclerotic lesions relative to expression in non-lesion blood vessel tissue. In some embodiments of the invention the DEA polynucleotide is a DEA-A polynucleotide. In other embodiments of the invention the DEA polynucleotide is a DEA-DB polynucleotide. In other embodiments of the invention the DEA polynucleotide is a DEA-DL polynucleotide. In other embodiments of the invention the DEA polynucleotide is a DEA-DNL polnucleotide.

In another aspect, the invention provides a binding agent, also referred to herein as a targeting agent, that specifically binds to a DEA polypeptide. The targeting agent may be, for example, an antibody, antibody fragment, affibody, or ligand. In some embodiments of the invention the DEA polynucleotide is overexpressed in atherosclerotic lesions relative to expression in non-lesion blood vessel tissue. In some embodiments of the invention the targeting agent binds to a DEA-A polypeptide. In other embodiments the targeting agent binds to a DEA-DB polypeptide. In other embodiments the targeting agent binds to a DEA-DL polypeptide. In other embodiments the targeting agent binds to a DEA-DNL polypeptide.

The invention further provides a conjugate comprising: a targeting agent linked to a functional moiety, wherein the targeting agent specifically binds to a DEA polypeptide. In various embodiments of the invention the functional moiety comprises a therapeutic agent, a radiosensitizing agent, or a diagnostic agent. The conjugate is referred to herein as a DEA-targeted conjugate. Thus the invention provides DEA-targeted diagnostic agents (e.g., DEA-targeted imaging agents), DEA-targeted radiosensitizing agents, and DEA-targeted therapeutic agents. In some embodiments of the invention the DEA polynucleotide is overexpressed in atherosclerotic lesions relative to expression in non-lesion blood vessel tissue. The therapeutic agent may be a small molecule, protein, peptide, RNAi agent, antisense molecule, ribozyme, or triplex nucleic acid. In some embodiments of the invention the targeting agent binds to a DEA-A polypeptide. In other embodiments the targeting agent binds to a DEA-DB polypeptide. In other embodiments the targeting agent binds to a DEA-DL polypeptide. In other embodiments the targeting agent binds to a DEA-DNL polypeptide.

The invention further provides a DEA-targeted delivery vehicle comprising a DEA targeting agent physically associated with a delivery vehicle. The delivery vehicle is a nanoparticle, microparticle, liposome or other lipid-based delivery vehicle, or polymer in various embodiments of the invention. In some embodiments of the invention the DEA targeting agent is covalently attached to the delivery agent. In other embodiments the DEA targeting agent is non-covalently attached to the delivery vehicle by a specific binding interaction (e.g., a streptavidin-biotin interaction or the like). In still other embodiments the DEA targeting agent is physically associated with the delivery vehicle by a non-specific physical interaction mechanism. The invention further provides a DEA-targeted delivery vehicle comprising a diagnostic or therapeutic agent. The diagnostic or therapeutic agent may be either covalently or noncovalently attached to the delivery vehicle or a component thereof, e.g., a coating layer.

The invention also provides a method of inhibiting expression of a DEA polypeptide in a cell or a subject comprising delivering an RNAi agent, a antisense oligonucleotide, ribozyme, DEA-targeted therapeutic agent, or DEA-targeted delivery vehicle comprising a therapeutic agent to the cell or subject. The subject may be an individual at risk of or suffering from atherosclerosis or at risk or suffering a condition or disease associated with atherosclerosis. The subject may have one or more risk factors for development of atherosclerosis, e.g., diabetes. In some embodiments of the invention the DEA-targeted therapeutic agent or DEA-targeted delivery vehicle specifically binds to a DEA polypeptide which is encoded by a DEA polynucleotide that is overexpressed in atherosclerotic lesions relative to its expression in non-lesion blood vessel tissue. In some embodiments of the invention the DEA polypeptide is a DEA-A polypeptide. In other embodiments the DEA polypeptide is a DEA-DB polypeptide. In other embodiments the DEA polypeptide is a DEA-DL polypeptide. In other embodiments the DEA polypeptide is a DEA-DNL polypeptide.

The invention further provides a method of treating or preventing atherosclerosis comprising steps of: (i) providing a subject in need of treatment or prevention of atherosclerosis; and (ii) administering a composition comprising a DEA-targeted therapeutic agent to the subject. The agent may be an RNAi agent, an antisense oligonucleotide, a ribozyme, or a small molecule. In some embodiments of the invention the DEA-targeted therapeutic agent comprises a DEA targeting agent that specifically binds to a DEA polypeptide encoded by a DEA polynucleotide that is overexpressed in atherosclerotic lesions relative to its expression in non-lesion blood vessel tissue.

In another aspect, the invention provides a method for detecting or quantifying atherosclerosis in a biological sample or subject comprising: determining the level of expression of a DEA polynucleotide or polypeptide in the biological sample or subject. The level of expression can be compared with known expression levels that are known to be characteristic of a particular clinical severity or histopathologic severity of atherosclerosis, and a degree of severity can be assigned to the sample or subject based on the comparison.

The invention further provides a method of targeting a molecule to an atherosclerotic lesion comprising the step of: administering a conjugate or delivery vehicle comprising the molecule to a subject having an atherosclerotic lesion, wherein the conjugate or delivery vehicle comprises a targeting agent that specifically binds to a DEA polypeptide encoded by a DEA gene, wherein the DEA gene is overexpressed in atherosclerotic lesions relative to normal blood vessel tissue.

The invention further provides a method of imaging vascular tissue in a subject comprising steps of: (i) administering a conjugate or delivery vehicle that comprises a targeting agent that specifically binds to a DEA polypeptide to the subject, wherein the conjugate or delivery vehicle comprises a functional moiety that enhances detectability of the DEA polypeptide; and (ii) subjecting the subject to an imaging procedure that detects the functional moiety.

In another aspect, the invention provides a method for identifying an agent that modulates expression or activity of a DEA polynucleotide or polypeptide comprising steps of: (i) providing a sample comprising a DEA polynucleotide or polypeptide; (ii) contacting the sample with a candidate compound; (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the expression or activity of the DEA polynucleotide or polypeptide if the level of expression or activity of the DEA polynucleotide or polypeptide is higher or lower in the presence of the compound relative to its level of expression or activity in the absence of the compound. The method may further comprise the steps of: (i) administering the compound to an animal model of atherosclerosis and (ii) determining whether the agent has a beneficial effect on the animal. The beneficial effect may be, for example, preventing atherosclerosis, delaying the onset of atheroscleroris, inhibiting the progression of atherosclerosis, decreasing the severity of atherosclerosis, increasing the life expectancy of the animal, etc. The method may further comprise the step of: identifying the agent as useful for the treatment and/or prevention of atherosclerosis.

In another aspect, the invention provides a method of providing diagnostic or prognostic information related to atherosclerosis comprising steps of: (i) providing a subject in need of diagnostic or prognostic information related to atherosclerosis; (ii) determining the level of expression or activity of a DEA polynucleotide or polypeptide, or the level of a ligand for a DEA polypeptide, in the subject or in a biological sample obtained from the subject; and (iii) utilizing the information to provide diagnostic or prognostic information.

In various embodiments of the invention the step of utilizing comprises comparing the expression level or activity of the DEA polynucleotide or polypeptide, or the level of the ligand, with predetermined ranges of values for the expression level or activity of the DEA polynucleotide or polypeptide, or predetermined ranges of values for the level of the ligand, wherein the ranges are associated with levels of risk that a subject suffers from atherosclerosis, levels of disease severity, degree of response to treatment, or another type of diagnostic or prognostic information, thereby obtaining an indication of the risk, disease severity, or degree of response to treatment.

In another aspect, the invention provides a method of providing diagnostic or prognostic information related to atherosclerosis or a condition or disease associated with atherosclerosis comprising steps of: (i) providing a subject in need of diagnostic or prognostic information related to atherosclerosis or a condition or disease associated with atherosclerosis; (ii) determining the level of expression or activity of a DEA polynucleotide or polypeptide in the subject or in a biological sample obtained from the subject; and (iii) concluding that there is an increased likelihood that the subject is at risk of or suffering from atherosclerosis or a condition or disease associated with atherosclerosis if the level of expression of DEA polynucleotide, the level or activity of the DEA polypeptide, or any combination of the foregoing, differs significantly from that in a normal subject or in a biological sample obtained from a normal subject.

In another aspect, the invention provides a method of treating or preventing atherosclerosis or a disease or condition associated with atherosclerosis comprising steps of: (i) providing a subject at risk of or suffering from atherosclerosis or a disease or condition associated with atherosclerosis; and (ii) administering a composition that modulates a DEA gene or expression product thereof to the subject.

The invention also provides a method for identifying a compound comprising steps of: (i) providing a DEA polypeptide; (ii) contacting the DEA polypeptide with the compound; and (iii) determining whether the compound specifically binds to the DEA polypeptide. The invention also provides a method for identifying a compound comprising steps of: (i) providing a DEA polypeptide having a biological activity; (ii) contacting the DEA polypeptide with the compound; and (iii) determining whether the compound increases or decreases the biological activity of the DEA polypeptide. The DEA polypeptide may be isolated from a natural source, recombinantly expressed, present on a cell surface, etc. The biological activity may be, for example, ability to bind a ligand (e.g., growth factor, cytokine, receptor, protein, lipid, etc.), kinase activity, GTPase activity, etc. In some embodiments of the invention the step of contacting the DEA polypeptide with the compound comprises contacting cells that express the DEA polypeptide with the compound.

The invention further provides a method of selecting a therapeutic regimen for a subject at risk of or suffering from atherosclerosis or a disease or condition associated with atherosclerosis comprising steps of: (i) providing a subject at risk of or suffering from atherosclerosis or a disease or condition associated with atherosclerosis (ii) determining the level of expression of a DEA polynucleotide, the level of expression or activity of a DEA polypeptide, or any combination of the foregoing, in the subject or in a biological sample obtained from the subject; and (iii) selecting a therapeutic regimen for the subject based on the determination.

In any of the inventive methods involving a determination of the expression and/or activity levels of a DEA polynucleotide and/or DEA polypeptide, the methods may comprise determining the expression and/or activity levels of a plurality of DEA polynucleotides and/or polypeptides, e.g., 2-5, 5-10, 10-25, 25-50, 50-100, 100-250, or more than 250. In embodiments in which the expression or activity level of a single DEA polynucleotide or polypeptide is determined, the DEA polynucleotide may be a DEA-A polynucleotide or DEA-A polypeptide, a DEA-DB polynucleotide or DEA-DB polypeptide, a DEA-DL polynucleotide or DEA-DL polypeptide, or a DEA-DNL polynucleotide or DEA-DNL polypeptide. Detection may be performed, for example, using a cDNA or oligonucleotide array, a protein array, etc.

This application refers to various patents, patent applications, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Harlow, E., et al., Antibodies. A Laboratory Manual, (Cold Spring Harbor Laboratory Press, 2nd ed. 1988); Kuby Immunology, 4^{th} ed., Goldsby, R. A., Kindt, T. J., and Osborne, B. (eds.); Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; Hardman, J., Limbird. E., Gilman, A. (Eds.), Braunwald, E., Zipes, D. P., and Libby, P. (eds.) Heart Disease: A Textbook of Cardiovascular Medicine. W B Saunders; 6th edition (Feb. 15, 2001); Chien, K. R., Molecular Basis of Cardiovascular Disease: A Companion to Braunwald's Heart Disease, W B Saunders; Revised edition (2003); and Goodman and Gilnian's The Pharmacological Basis of Therapeutics, 10th Ed. McGraw Hill, 2001 (referred to herein as Goodman and Gilman). In the event of a conflict or inconsistency between any of the incorporated references and the instant specification or the understanding of one or ordinary skill in the art, the specification shall control, it being understood that the determination of whether a conflict or inconsistency exists is within the discretion of the inventors and can be made at any time.

### Definitions

To facilitate understanding of the description of the invention, the following definitions are provided. It is to be understood that, in general, terms not otherwise defined are to be given their meaning or meanings as generally accepted in the art.

Antibody: In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. Preferred antibodies, antibody fragments, and/or protein domains comprising an antigen binding site may be generated and/or selected in vitro, e.g., using techniques such as phage display (Winter, G. et al. 1994. Annu. Rev. Immunol. 12:433-455, 1994), ribosome display (Hanes, J., and Pluckthun, A. Proc. Natl. Acad. Sci. USA. 94:4937-4942, 1997), etc. In various embodiments of the invention the antibody is a “humanized” antibody in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

Atherosclerotic lesion. As used herein, an “atherosclerotic lesion” is blood vessel tissue that shows evidence of atherosclerosis when assessed using an art-accepted method, e.g., examination of an appropriately processed sample of blood vessel tissue by a histopathologist skilled in the art of diagnosis of atherosclerosis. It will be understood that certain of the microarray analyses described herein were performed on samples of blood vessel tissue, e.g., blood vessel segments, that comprised atherosclerotic lesions. Such tissue samples may include portions of blood vessel tissue that do not show evidence of atherosclerosis (i.e., “normal” blood vessel tissue) though in general such portions constitute only a minor fraction of the sample (e.g., less than 25%). The terms “blood vessel tissue comprising an atherosclerotic lesion” and “atherosclerotic lesion” are used interchangeably herein.

The term “conjugate” refers to a composite entity comprised of at least two moieties attached (“conjugated”) to one another. The moieties, which may be referred to as “components” of the conjugate, are either directly linked to one another or are indirectly linked to one another through an intervening moiety or moieties, such as a bridge, spacer, or linkage moiety or moieties, which forms part of the conjugate. Preferably the moieties are covalently linked, although high affinity specific, noncovalent interactions such as antigen-antibody association, streptavidin-biotin association, or the like, which depend on specific structural features of the moieties, are also acceptable. Preferably a noncovalent association has a K_{d} of 10⁻⁶ or less, preferably 10⁻⁷ or less, more preferably 10⁻⁸ or less. The term “conjugate” encompasses fusion proteins, in which the two moieties are polypeptides. The term also encompasses entities comprising two or more polypeptides, wherein the polypeptides are joined by a non-polypeptide bond or by a non-polypeptide linking moiety. It will be appreciated that conjugation is “reciprocal”, i.e., it is equally appropriate to say with respect to first and second components of a conjugate that the first component is conjugated to the second component or that the second component is conjugated to the first component. The same principle extends to conjugates comprising more than two components.

Diagnostic agent. As used herein, a “diagnostic agent” is any compound or other entity that can be used either alone or in combination with other agents and/or suitable equipment to practice a method, process, or procedure that provides diagnostic or prognostic information. In some embodiments of the invention a diagnostic agent is administered to a subject. In other embodiments a diagnostic agent is used to perform a test on a sample obtained from a subject. Diagnostic agents include, e.g., imaging agents.

Diagnostic information: As used herein, “diagnostic information” or information for use in diagnosis is any information that is useful in determining whether a subject has or is susceptible to developing a disease or condition and/or in classifying the disease or condition into a phenotypic category or any category having significance with regards to the prognosis of or likely response to treatment of the disease or condition. The term includes prenatal diagnosis, i.e., diagnosis performed prior to the birth of the subject, including performing genetic testing on germ cells (ova and/or sperm). The term also includes determining the genotype of a subject with respect to a DEA gene for any purpose.

Diagnostic target: A gene is considered to be a “diagnostic target” if detection and/or measurement of its expression level is useful in providing diagnostic or prognostic information related to a disease or clinical condition, or for monitoring the physiological state of a cell, tissue, or organism (including monitoring the response to therapy or the progression of disease). Expression products of such genes (RNA or polypeptide) may also be referred to as diagnostic targets. Certain preferred diagnostic targets are genes that encode a polypeptide that comprises a transmembrane domain and, preferably, an extracellular portion. The prediction of protein orientation with respect to the cell membrane and the existence of transmembrane domains can be performed, for example, using the program TMpred (K. Hofmann & W. Stoffel (1993) TMbase—A database of membrane spanning proteins segments. Biol. Chem. Hoppe-Seyler 347, 166) and/or the methods described in Erik L. L. Sonnhammer, Gunnar von Heijne, and Anders Krogh: A hidden Markov model for predicting transmembrane helices in protein sequences. In Proc. of Sixth Int. Conf. on Intelligent Systems for Molecular Biology, p 175-182 Ed J. Glasgow, T. Littlejohn, F. Major, R. Lathrop, D. Sankoff, and C. Sensen. Menlo Park, Calif.: AAAI Press, 1998.

Certain preferred diagnostic targets are genes that encode secreted polypeptides, e.g., polypeptides that are secreted into the extracellular space and/or bloodstream. Detection of such polypeptides can typically be conveniently performed on a body fluid sample, e.g., a blood sample. A secreted polypeptide can be identified by the presence of a signal peptide. As is known in the art, a signal peptide is a short (e.g., ˜15-60 amino acids long) peptide chain that directs the cotranslational or post-translational transport of a polypeptide that includes the signal peptide across a membrane, e.g., into the endoplasmic reticulum. Such transport typically leads to the eventual secretion of the polypeptide by the cell. Some signal peptides are cleaved from the polypeptide after the polypeptide is transported across a membrane. Signal peptides may also be called targeting signals or signal sequences. A gene or polynucleotide that encodes a secreted polypeptide can be identified by the presence of a portion that encodes a signal peptide.

Differential expression: A gene or cDNA clone exhibits “differential expression” at the RNA level if its RNA transcript varies in abundance between different cell types, tissues, samples, etc., at different times, or under different conditions. A gene exhibits differential expression at the protein level if a polypeptide encoded by the gene or cDNA clone varies in abundance between different cell types, tissues, samples, etc., or at different times. In the context of a microarray experiment, differential expression generally refers to differential expression at the RNA level. Differential expression, as used herein, may refer to both quantitative as well as qualitative differences in the temporal and/or tissue expression patterns. In general, differentially expressed genes may be used to identify or detect particular cell types, tissues, physiological states, etc., to distinguish between different cell types, tissues, or physiological states. Differentially expressed genes and their expression products may be diagnostic and/or therapeutic targets or may interact with such targets. Differentially expressed genes may also be referred to as “upregulated” or “overexpressed” if they are expressed at a higher level in a first cell type, tissue, sample, condition, or state of interest etc. than in a second cell type, tissue, sample, condition, or state. Differentially expressed genes may also be referred to as “downregulated” or “underexpressed” if they are expressed at a lower level in a first cell type, tissue, sample, condition, or state of interest etc. than in a second cell type, tissue, sample, condition, or state.

Effective amount: In general, an “effective amount” of an active agent refers to an amount necessary to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, in the case of an agent for the treatment of atherosclerosis or a condition associated with atherosclerosis, an effective amount may be an amount sufficient to result in clinical improvement of the individual, e.g., increased exercise tolerance/capacity, subjective improvement of other symptoms such as pain on exertion, etc., and/or improved results on a quantitative test of cardiac functioning, e.g., ejection fraction, exercise capacity (e.g., time to exhaustion), etc. According to certain embodiments of the invention an effective amount results in an improvement in a quantitative measure or index that reflects the extent and/or severity of atherosclerosis, e.g., an imaging procedure that evaluates the degree of narrowing of an artery, etc.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as tRNAs. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification). A compound or agent is said to increase gene expression if application of the compound or agent to a cell or subject results in an increase in either an RNA or polypeptide expression product or both. A compound or agent is said to decrease gene expression if application of the compound or agent to a cell or subject results in a decrease in either an RNA or polypeptide expression product or both.

Hybridize. The term “hybridize”, as used herein, refers to the interaction between two complementary nucleic acid sequences. The degree and specificity of hybridization is affected by the stringency of the conditions under which the nucleic acid molecules are exposed to each other. Factors such as temperature, ionic strength of the solution, pH, presence of destabilizing agents such as formamide or stabilizing agents may all influence the degree and specificity of hybridization. Hybridization conditions are generally referred to as high, medium, or low stringency. The phrase “hybridizes under high stringency conditions” describes an interaction that is sufficiently stable that it is maintained under art-recognized high stringency conditions. Hybridization under high stringency conditions only occurs between sequences with a very high degree of complementarity. One of ordinary skill in the art will be able to select appropriate hybridization conditions or systematically vary such conditions to perform the various assays described herein. In general, high stringency conditions are selected to be approximately 5-10° C. lower than the thermal melting point (Tₘ) for the specific double-stranded sequence at a particular pH and ionic strength, where the Tₘ is the temperature at which 50% of the probes complementary to the target hybridize to the target at equilibrium, assuming targets are present in excess. One of ordinary skill in the art will recognize that the parameters for different degrees of stringency will generally differ based various factors such as the length of the hybridizing sequences, whether they contain RNA or DNA, etc. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.); high stringency (e.g., 6×SSC at about 45° C., followed by one or more washes in 0.2×SSC, 0.1% SDS at 65° C.); and very high stringency (e.g., 0.5M sodium phosphate, 0.1% SDS at 65° C., followed by one or more washes at 0.2×SSC, 1% SDS at 65° C.) Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001.

Isolated: As used herein, “isolated” means 1) separated from at least some of the components with which it is usually associated in nature; 2) prepared or purified by a process that involves the hand of man; and/or 3) not occurring in nature.

Ligand. As used herein, “ligand” means a molecule that specifically binds to a target such as a polypeptide through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity, the term as used herein applies more generally.

Marker: A “marker” may be any gene or gene product (e.g., protein, peptide, mRNA) that indicates or identifies a particular diseased or physiological state (e.g., carcinoma, normal, dysplasia) or indicates or identifies a particular cell type, tissue type, or origin. The expression or lack of expression of a marker gene may indicate a particular physiological or diseased state of a individual, organ, tissue, or cell. Preferably, the expression or lack of expression may be determined using standard techniques such as Northern blotting, in situ hybridization, RT-PCR, real-time RT-PCR, sequencing, immunochemistry, immunoblotting, oligonucleotide or cDNA microarray or membrane array, protein microarray analysis, mass spectrometry, etc. In certain embodiments of the invention, the level of expression of a marker gene is quantifiable.

Non-lesion blood vessel tissue. “Non-lesion blood vessel tissue” is blood vessel tissue, e.g., arterial wall tissue, that has been determined to be essentially free of evidence of atherosclerosis using an art-accepted method, e.g., examination of an appropriately processed sample of blood vessel tissue by a histopathologist skilled in the art of diagnosis of atherosclerosis. Such tissue is also referred to herein as “normal”. Use of the term “normal” is intended to refer to the appearance of the tissue upon histopathological examination using art-accepted methods and is not intended to exclude tissue that may have an underlying genetic and/or biochemical alteration or characteristic that increases the likelihood that atherosclerosis will develop in the blood vessel relative to the likelihood that atherosclerosis would develop in a subject not having the alteration or characteristic.

Operably linked. As used herein, “operably linked” refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Peptide, polypeptide, or protein: According to the present invention, a “peptide”, “polypeptide”, or “protein” comprises a string of at least three amino acids linked together by peptide bonds. The terms may be used interchangeably although a peptide generally represents a string of between approximately 8 and 30 amino acids. Peptide may refer to an individual peptide or a collection of peptides. Peptides preferably contain only natural amino acids, although non-natural amino acids (i.e., compounds that do not occur in nature but that can be incorporated into a polypeptide chain; see, for example, the web site having URL www.cco.caltech.edu/˜dadgrp/Unnatstruct.gif) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide, but such modifications may confer desirable properties, e.g., enhanced biological activity, on the peptide.

A compound or agent is said to increase expression of a polypeptide if application of the compound or agent to a cell or subject results in an increase in the amount of the polypeptide synthesized by the cell. Preferably the increased synthesis results in an increased steady state level of the polypeptide in the cell, extracellular matrix, and/or blood. A compound or agent is said to decrease expression of a polypeptide if application of the compound or agent to a cell or subject results in a decrease in the amount of the polypeptide synthesized by the cell. Preferably the decreased synthesis results in a decreased steady state level of the polypeptide in the cell, extracellular matrix, and/or blood.

Polynucleotide or oligonucleotide: “Polynucleotide” or “oligonucleotide” refers to a polymer of nucleotides. Typically, a polynucleotide comprises at least three nucleotides. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

A compound or agent is said to increase expression of a polynucleotide if application of the compound or agent to a cell or subject results in an increase in the amount of the polynucleotide synthesized by the cell or results in an increase in the amount of a translation product of the polynucleotide synthesized by the cell, or both. Preferably the increased synthesis results in an increased steady state level of the polynucleotide in the cell and/or an increased level of the polypeptide in the cell, extracellular matrix, and/or blood. A compound or agent is said to decrease expression of a polynucleotide if application of the compound or agent to a cell or subject results in a decrease in the amount of the polynucleotide synthesized by the cell or results in a decrease in the amount of a translation product of the polynucleotide synthesized by the cell, or both. Preferably the decreased synthesis results in a decreased steady state level of the polynucleotide in the cell and/or a decreased level of the polypeptide in the cell, extracellular matrix, and/or blood.

Prognostic information and predictive information: As used herein the terms “prognostic information” and “predictive information” are used interchangeably to refer to any information that may be used to foretell any aspect of the course of a disease or condition either in the absence or presence of treatment. Such information may include, but is not limited to, the average life expectancy of a individual, the likelihood that a individual will survive for a given amount of time (e.g., 6 months, 1 year, 5 years, etc.), the likelihood that a individual will be cured of a disease, the likelihood that a individual's disease will respond to a particular therapy (wherein response may be defined in any of a variety of ways). Prognostic and predictive information are included within the broad category of diagnostic information.

Purified: As used herein, “purified” means separated from one or more compounds or entities, e.g., one or more compounds or entities with which it is naturally found. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure. In the context of a preparation of a single nucleic acid molecule, a preparation may be considered substantially pure if the nucleic acid represents a majority of all nucleic acid molecules in the preparation, preferably at least 75%, yet more preferably at least 90%, or greater, as listed above.

Regulatory sequence: The term “regulatory sequence” is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc.

Sample. As used herein, a “sample” obtained from a subject may include, but is not limited to, any or all of the following: a cell or cells, a portion of tissue, blood, serum, ascites, urine, saliva, amniotic fluid, cerebrospinal fluid, and other body fluids, secretions, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling. The term “sample” may also refer to any material derived by isolating, purifying, and/or processing a sample obtained directly from a subject. Derived samples may include nucleic acids or proteins extracted from the sample or obtained by subjecting the sample to techniques such as amplification or reverse transcription of mRNA, etc. A derived sample may be, for example, a homogenate, lysate, or extract prepared from a tissue, cells, or other constituent of an organism (e.g., a body fluid).

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

Specific binding: As used herein, the term “specific binding” refers to an interaction between a target molecule (typically a target polypeptide) and a binding molecule such as an antibody or ligand. The interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute but generally refers to the context in which the binding is performed. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodiagnostic purposes. Once the specificity of a binding molecule is established in one or more contexts, it may be employed in other, preferably similar, contexts without necessarily re-evaluating its specificity. In the context of an interaction between an antibody or ligand and a polypeptide, according to certain embodiments of the invention a molecule exhibits specific binding if it binds to the polypeptide at least 5 times as strongly as to other polypeptides present in a cell lysate, e.g., a myocardial cell lysate. According to certain embodiments of the invention a molecule exhibits specific binding if it binds to the polypeptide at least 10 times as strongly as to other polypeptides present in a cell lysate. According to certain embodiments of the invention a molecule exhibits specific binding if it binds to the polypeptide at least 50 times as strongly as to other polypeptides present in a cell lysate. According to certain embodiments of the invention a molecule exhibits specific binding if it binds to the polypeptide at least 100 times as strongly as to other polypeptides present in a cell lysate.

Subject: The term “subject”, as used herein, refers to an individual to whom an agent is to be delivered, e.g., for experimental, diagnostic, and/or therapeutic purposes. Preferred subjects are mammals, including humans. Other preferred mammalian subjects include rats, mice, other rodents, non-human primates, rabbits, sheep, cows, dogs, cats, and other domesticated animals and/or animals of agricultural interest.

Therapeutic agent: The term “therapeutic agent” is used consistently with its meaning in the art to refer to an agent that is administered to a subject to treat a disease, disorder, or other clinically recognized condition that is harmful to the subject, or for prophylactic purposes.

Therapeutic target: Certain genes that are differentially expressed in cells, tissues, etc., represent “therapeutic targets”, in that modulating expression of such a gene (e.g., increasing expression, decreasing expression, or altering temporal properties of expression) and/or modulating the activity or level of an expression product of the gene may alter the biochemical or physiological properties of the cell or tissue so as to treat or prevent a disease or clinical condition. For example, in the context of the present invention, modulation of the expression of certain of the differentially expressed genes described herein may treat or prevent atherosclerosis. Modulating the activity of an expression product, e.g., by administering a compound such as a small molecule or antibody that affects the activity, by altering phosphorylation or glycosylation state, may treat or prevent atherosclerosis. Expression products (RNA or polypeptide) of the therapeutic target genes may also be referred to as therapeutic targets.

Certain preferred therapeutic targets include, but are not limited to, genes that encode a polypeptide that comprises a transmembrane domain and, preferably, an extracellular portion. The prediction of protein orientation with respect to the cell membrane and the existence of transmembrane domains can be performed as described above. Certain preferred therapeutic targets are genes that encode polypeptides having a have a recognized biochemical activity. For example, and without limitation, genes that encode receptors such as G protein coupled receptors, receptors comprising a kinase domain, etc., are of particular interest. A determination that a gene encodes a polypeptide having a recognized biochemical activity can be made based either on a direct experimental assessment of the activity of the polypeptide or based on homology of the polypeptide to polypeptides recognized in the art as possessing the activity.

Treating: As used herein, “treating” refers to administering an agent to a subject following the development of one or more symptoms indicative of atherosclerosis or following the development of a disease or condition associated with atherosclerosis, or following the development of one or more symptoms of a disease or condition in which atheroscleroris commonly occurs (i.e., in which at least 5% of subjects diagnosed with the disease eventually experience atherosclerosis), e.g., in order to reverse, alleviate, reduce the severity of, eliminate, and/or inhibit the progression of atherosclerosis. A DEA-targeted therapeutic agent can also be administered prophylactically, i.e., before development of any symptom indicative of atheroscleroris or a disease or condition associated with atheroscleroris or before development of one or more symptoms of a disease or condition in which atherosclerosis commonly occurs, for the purpose of preventing or delaying development of atherosclerosis.

Vascular tissue: The terms “vascular tissue” and “blood vessel tissue” are used interchangeably herein to refers to those tissues that are found in and/or make up the wall of blood vessels. Cells typically found in such tissues (referred to herein as “vascular system cells” or “blood vessel cells” include, but are not limited to, endothelial cells (which form a layer of squamous epithelium that lines the cavities of the heart, blood vessels (including capillaries), and lymph vessels), smooth muscle cells, fibroblasts, and macrophages.

Vector: The term “vector” is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (which may comprise sequences derived from viruses), cosmids, and virus vectors. Virus vectors include, e.g., replication defective retroviruses, adenoviruses, adeno-associated viruses, and lentiviruses. As will be evident to one of ordinary skill in the art, virus vectors may include various viral components in addition to nucleic acid(s) that mediate entry of the transferred nucleic acid.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1 presents heat maps showing differential gene expression in the vascular wall of diabetic and non-diabetic individuals. (a) Expression profiles of the 103 diseased and non-diseased vascular segments were compared between diabetic (34/103) and non-diabetic patients (69/103) using SAM. A total of 342 probes were identified as differentially regulated (FDR=0.005). The heat map reflects normalized gene expression ratios and is organized with individual hybridizations arranged along the x-axis. These ratios are depicted by color intensity such that highest expressions correspond to bright red and bright green, respectively. A collapsed list of unique genes accompanies the heat map. Magenta text denotes genes that encode inflammatory mediators, and blue text signifies genes that were identified as cytokine-responsive by array hybridization experiments using RNA from (TNF-α)-stimulated primary human endothelial and smooth muscle cells. (b) Expression profiles of thirty-six normal vascular segments were compared between diabetics (11/36) and non-diabetics (25/36) using SAM. 63 genes were identified as differentially regulated (FDR=0.06)

FIG. 2 presents heat maps showing decreased expression of inflammatory markers in coronary arteries of statin-treated patients. (a) Expression profiles of 100 vascular segments were compared in the context of statin treatment using SAM. 117 probes were identified as differentially regulated (FDR of 0.05). The heatmap reflects normalized expression ratios and is organized as in FIG. 1, with the collapsed gene list showing a portion of those genes expressed at statistically lower levels in statin-treated tissues. Magenta and blue texts denote genes that encode inflammatory mediators and cytokine-responsive genes, respectively. (b) Expression profiles of 34 diabetic vascular samples were compared between statin-treated (9/34) and untreated (25/34) patients using SAM. 318 of the most differentially regulated genes are shown (FDR=0.0016).

### DETAILED DESCRIPTION OF CERTAIN EMBODIMENTS OF THE INVENTION

I. DEA Genes, Polynucleotides, and Polypeptides

The recent availability of human genetic information and reagents has allowed the development of high throughput genomics platforms such as microarrays. The study of large-scale expression data with sophisticated statistical algorithms has provided significant molecular insights into complex human diseases within the context of clinical variables. While this strategy has been widely used in cancer studies, adoption of this paradigm in cardiovascular system diseases has been limited. No studies to date have explored how vascular wall gene expression is modulated by risk factors such as atherosclerosis or by therapeutic agents.

In the present invention gene expression profiles in vascular disease were examined by performing transcriptional profiling experiments with human coronary artery samples using a custom vascular wall microarray. The samples were obtained from explanted hearts of individuals undergoing orthotopic heart transplant, thus providing a unique sample set which included subjects having various risk factors and subjects who were undergoing treatment with various commonly used pharmaceutical agents. Differences in gene expression between normal and diseased blood vessel segments were identified. In addition, differences in gene expression between normal blood vessel segments in individuals with diabetes and individuals without diabetes were identified. Also, differences in gene expression between atherosclerotic lesions in individuals with diabetes and individuals without diabetes were identified. Microarray analysis of mRNA expression was performed on the samples as described in more detail in Example 1. Data analysis involved use of two different statistical tests to identify genes that were significantly overexpressed or underexpressed in different sample sets.

Microarray analysis resulted in the identification of a number of genes that are overexpressed (upregulated) in atherosclerotic lesions and a number of genes that are underexpressed (downregulated) in atherosclerotic lesions. Microarray analysis also resulted in the identification of a number of genes that are overexpressed (upregulated) in non-lesion vascular tissue and a number of genes that are underexpressed (down-regulated) in non-lesion vascular tissue. Accession numbers that correspond to genes that are upregulated in lesion samples and downregulated in non-lesion samples, i.e., they are overexpressed in atherosclerotic lesions relative to their expression in normal blood vessel tissue, are listed in the upper portion of Table 1 (no lesion<lesion). Accession numbers that correspond to genes that are downregulated in lesion samples and upregulated in non-lesion samples, i.e., they are underexpressed in atherosclerotic lesions relative to their expression in normal blood vessel tissue, are listed in the lower portion of Table 1 (lesion<no lesion). It should be noted that the tables are nonlimiting and other genes mentioned herein are also within the scope of the invention.

As used herein, an accession number is said to “correspond to” a gene, polynucleotide, or polypeptide, if the accession number provides sufficient information to allow one or ordinary skill in the art to identify the gene, polynucleotide, or polypeptide using publicly available databases such as Genbank. In the case of the instant invention, the accession numbers provided herein identify cDNA sequences (or, equivalently, mRNA sequences). One of ordinary skill in the art would access the database, enter the accession number, and perform a search. The search would retrieve information about the cDNA including, but not limited to, its sequence. One of ordinary skill in the art would recognize that the mRNA is transcribed from a particular gene; thus the accession number also identifies and corresponds to that particular gene and polypeptide. One of ordinary skill in the art would recognize that the mRNA encodes a particular polypeptide; thus the accession number also identifies and corresponds to that particular polypeptide. One of ordinary skill in the art would also recognize that a number of different mRNA species could be transcribed from a particular gene or could result from alternative splicing of a primary transcript and that a single gene could thus correspond to a variety of different polynucleotides (e.g., mRNAs, cDNAs, etc.) and/or polypeptides. In certain instances a cDNA or mRNA may be less than “full length”. One of ordinary skill in the art would readily be able to identify a full length cDNA by any of a variety of methods. For example, one of ordinary skill in the art could use the cDNA to probe a cDNA library. The cDNA sequence could also be used to search for additional sequences that comprise or overlap with the sequence.

Genes that correspond to the accession numbers listed in Table 1 are referred to herein as DEA-A genes. A large number of genes were identified for the first time in association with CAD, including a novel matrix metalloproteinase, MMP-10, and a number of other genes. Additional genes of particular use in the compositions and methods of the invention include, but are not limited to, myristoylated alanine-rich protein kinase C substrate (MARCKS), secreted phosphoprotein 1 (also known as osteopontin, bone sialoprotein 1, early T-lymphocyte activation 1), oxidised low density lipoprotein (lectin-like) receptor 1, integral membrane protein 2A, and integral membrane protein 2B. In certain embodiments of the invention the gene that encodes an inflammatory mediator or cytokine-responsive gene. Certain DEA-A genes of particular interest in the practice of the present invention are described in the Examples.

Genes that are upregulated in blood vessel samples from diabetic individuals and downregulated in blood vessel samples from nondiabetic individuals were also identified. These genes are overexpressed in blood vessel tissue of diabetic individuals relative to their expression in blood vessel tissue of nondiabetic individuals (tissue not categorized as lesion or non-lesion). Accession numbers that correspond to these genes are listed in Table 2. A number of novel cytokine genes and genes encoding immune response factors were more highly expressed in samples from diabetics. Granulocyte chemotactic protein 2 (CXCL6), a factor known to mediate granulocyte migration by binding to the IL-8 receptor but not previously associated with CAD, was expressed at much higher levels in diabetic arteries. The invention provides a method of treating or inhibiting progression of atherosclerosis comprising administering an antagonist of the IL-8 receptor to a subject. In certain embodiments of the invention the subject is a diabetic. Any of a variety of agents can be used to inhibit the IL-8 receptor. For example, isoxazoles and oxadiazoles of use in the method as IL-8 receptor antagonists are disclosed in U.S. Pub. No. 20030216386. Pyrimidine derivatives of use in the method as IL-8 receptor antagonists are described in U.S. Pub. No. 20040087601. Other IL-8 receptor antagonists of use in the method are described in U.S. Pat. Nos. 5,886,044, 5,780,483, 6,005,008, 5,929,250, 6,015,908; or 5,919,776, WO99/65310, WO 0012489, WO 0009511, WO 9942464, WO 9942463, WO 9942461, WO00/05216, WO99/36069, WO99/36070, WO00/06557, PCT/US99/23776, and/or PCT/US99/29940. Other genes that were overexpressed in diabetic arteries include the cytokines IL-6 and IL-1a, chemokines IL-8, RANTES, macrophage chemoattractant protein (MCP-1), and lymphokine macrophage migration inhibitory factor. Genes that are downregulated in blood vessel samples from diabetic individuals and upregulated in blood vessel samples from nondiabetic individuals were also identified. These genes are underexpressed in blood vessel tissue of diabetic individuals relative to their expression in nondiabetic individuals. Accession numbers that correspond to these genes are also listed in Table 2. The genes corresponding to accession numbers listed in Table 2 are collectively referred to as DEA-DB genes herein.

Genes that are upregulated in atherosclerotic lesions from nondiabetic individuals and downregulated in atherosclerotic lesions from diabetic individuals were also identified. These genes are underexpressed in atherosclerotic lesions from diabetic individuals relative to their expression in nondiabetic individuals. Accession numbers that correspond to these genes are listed in Table 3. In addition, genes that are downregulated in atherosclerotic lesions from nondiabetic individuals and upregulated in atherosclerotic lesions from diabetic individuals were identified. These genes are overexpressed in atherosclerotic lesions of diabetic individuals relative to their expression in atherosclerotic lesions of nondiabetic individuals. Accession numbers that correspond to these genes are also listed in Table 3. The genes corresponding to accession numbers listed in Tables 3A and 3B are collectively referred to as DEA-DL genes herein.

Genes that are upregulated in non-lesion vascular tissue from diabetic individuals and downregulated in non-lesion vascular tissue from nondiabetic individuals were also identified. Accession numbers that correspond to these genes are listed in Table 4. Genes that are downregulated in non-lesion vascular tissue from diabetic individuals and upregulated in non-lesion vascular tissue from nondiabetic individuals were also identified. Accession numbers that correspond to these genes are listed in Table 4. The genes corresponding to accession numbers listed in Table 4 are collectively referred to as DEA-DNL genes herein.

A common approach employed in efforts to prevent and/or treat atherosclerosis and CAD is the administration of pharmaceutic agents that lower blood cholesterol levels. One important class of such agents consists of HMG-CoA reductase inhibitors, which include compounds known as “statins”. Examples include simvastatin, atorvastatin, fluvastatin, lovastatin, and pravastatin. The present invention encompasses the recognition that genes that are differentially regulated in blood vessel tissue of subjects who either have or have not been treated with a lipid lowering agent such as a statin are important targets for diagnosis and therapy of atherosclerosis. Furthermore, identification of differences in the expression profiles of treated vs. untreated tissue is of use to identify additional compounds that would be expected to have a similarly beneficial effect in inhibiting atherosclerosis as that of the statins. Genes that are differentially regulated in blood vessel tissue of subjects either treated or not treated with a statin are shown in Table 8. The invention provides a method of identifying a compound comprising steps of: determining the expression level of a multiplicity of genes listed in Table 8 in a subject to whom the compound has been administered with the expression level of those genes in a subject to whom the compound has not been administered; and determining whether administration of the compound alters the level of expression of the genes to more closely resemble the profile of a subject treated with a statin. The method can include obtaining a sample from a subject to whom the compound has been administered. The sample is typically a blood vessel sample. The subject can be an animal that serves as an animal model for atherosclerosis, diabetes, dyslipidemia, etc. The method can include a step of comparing the expression of one or more genes listed in Table 8 with the level of expression of those genes in a subject treated with a statin. The method can include a step of screening a multiplicity of compounds to identify one or more compounds that cause a significant number of genes (e.g., at least 5, 10, 25, 50, etc.) to switch from an expression pattern characteristic of a subject not treated with a statin to an expression pattern characteristic of a subject treated with a statin. The subject may or may not have atherosclerosis or CAD. The compounds can be members of compound libraries, e.g., natural product libraries or combinatorially synthesized libraries, as described elsewhere herein. The invention further includes compounds identified according to any of these methods.

Identification of the genes listed in Tables 1-4 and/or 8 provides a wide variety reagents and methods, as described below. For example, these genes and their expression products, e.g., mRNA and encoded polypeptides, are pharmacological targets for therapies aimed at preventing or treating atherosclerosis or any of its symptoms or manifestations. In addition, identification of genes that are upregulated in atherosclerotic lesions permits the targeting of molecules, including imaging agents and therapeutic agents, e.g., to atherosclerotic lesions, e.g., for purposes including, but not limited to, diagnosis, prognosis, treatment, imaging, or assessment of treatments for conditions associated with atherosclerosis. Measurement of the expression level of the genes newly identified as upregulated or downregulated in atherosclerosis improves diagnosis and prognosis of atherosclerosis and/or a disease or condition associated with atherosclerosis. Thus the invention provides diagnostic methods, reagents, and methods for the treatment of athersoscierosis and/or a disease or condition associated with atherosclerosis as described further below. In any of the aspects and embodiments of the invention described herein the DEA gene can be selected from genes corresponding to accession numbers listed in Table 1-4 and 8. In any of the aspects and embodiments of the invention described herein that involve a DEA gene, the DEA gene can be selected from DEA-A genes, DEA-DB genes, DEA-DL genes, DEA-DNL, and/or DEA-S genes.

It is noted that although the genes identified herein are human genes, the corresponding genes in other mammalian species are also of use in the present invention. In particular, the invention encompasses diagnostic and therapeutic methods for use in non-human mammalian species based on the corresponding genes in such species.

Polypeptide expression products of the genes identified in Tables 1-4 and 8 are referred to herein as DEA polypeptides. In certain embodiments of the invention a DEA polypeptide comprises the complete amino acid sequence encoded by a mRNA transcribed from the corresponding DEA gene. In addition, in certain embodiments of the invention DEA polypeptides comprise less than the complete amino acid sequence encoded by the corresponding DEA gene. For example alternate splicing or post-translational processing may give rise to shorter polypeptides that comprise less than the entire amino acid sequence encoded by the corresponding DEA gene. In general, such DEA polypeptides will comprise at least 10 continuous amino acid residues encoded by the corresponding DEA gene, at least 20 continuous amino acid residues encoded by the corresponding DEA gene, at least 30 continuous amino acid residues encoded by the corresponding DEA gene, at least 40 continuous amino acid residues encoded by the corresponding DEA gene, at least 50 continuous amino acid residues encoded by the corresponding DEA gene, etc. In various embodiments of the invention a DEA polypeptide comprises a polypeptide whose sequence comprises at least 10% of the amino acid sequence encoded by the corresponding DEA gene. In other embodiments of the invention a DEA polypeptide comprises a polypeptide whose sequence comprises at least 20%, at least 30%, at least 40%, at least 50%, at least 60%, at least 70%, at least 80%, at least 90%, at least 95%, or 100% of the amino acid sequence encoded by the corresponding DEA gene. In certain embodiments of the invention a DEA polypeptide consists of the complete polypeptide encoded by the corresponding DEA gene.

As noted above, certain of the DEA polypeptides are encoded by genes that are overexpressed or underexpressed in atherosclerotic lesions, overexpressed or underexpressed in diabetic blood vessels, overexpressed or underexpressed in atherosclerotic lesions from diabetic individuals, overexpresed or underexpressed in nonlesion vascular tissue from diabetic individuals, or differentially expressed in samples from patients who had or had not been treated with a statin. A DEA polypeptide may, but need not, display a similar pattern of overexpression or underexpression as the gene that encodes it. In other words, while in many cases the pattern of overexpression or underexpression of a protein parallels that of the gene that encodes it, one of ordinary skill in the art will appreciate that this is not invariably the case. If desired, one of ordinary skill in the art can readily determine whether any particular DEA polypeptide is overexpressed or underexpressed.

In any of the aspects and embodiments of the invention described herein that involve a DEA polypeptide, the DEA polypeptide can be selected from the group of: polypeptides encoded by genes corresponding to accession numbers listed in Table 1-4 and 8. In any of the aspects and embodiments of the invention described herein that involve a DEA polypeptide, the DEA polypeptide can be selected from the group of: polypeptides encoded by DEA-A genes, polypeptides encoded by DEA-DB genes, polypeptides encoded by DEA-DL genes, and polypeptides encoded by DEA-DNL genes.

II. Antibodies that Bind to DEA Polypeptides

The invention provides a variety of different antibodies that bind to the polypeptides encoded by the DEA genes identified herein. An antibody that binds to a DEA polypeptide may be referred to herein as a “DEA antibody”. The invention provides an antibody or other agent that specifically binds to a DEA polypeptide encoded by a polynucleotide whose sequence comprises the sequence of a polynucleotide whose Genbank accession number is selected from the group of Genbank accession numbers listed in any of Tables 1-4 or 8. In particular, the invention provides an antibody or other specific binding agent that specifically binds to a DEA polypeptide encoded by a gene selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

According to certain embodiments of the invention the antibody is a polyclonal antibody, while in other embodiments the antibody is monoclonal. Generally applicable methods for producing antibodies are well known in the art. It is noted that antibodies can be generated by immunizing animals (or humans) either with a full length polypeptide, a partial polypeptide, fusion protein, or peptide (which may be conjugated with another moiety to enhance immunogenicity). The exact specificity of the antibody will vary depending upon the particular preparation used to immunize the animal and on whether the antibody is polyclonal or monoclonal. For example, if a peptide is used the resulting antibody will bind only to the antigenic determinant represented by that peptide. Polyclonal or monoclonal antibodies that bind to a DEA polypeptide can be produced using standard methods. See, e.g., Harlow, supra. In a nonlimiting embodiment a DEA antibody is generated by the hybridoma technique, which involves immunizing a mammal with at least a portion of a DEA polypeptide, e.g., a portion of the extracellular domain of a DEA polypeptide in the case of DEA polypeptides that comprise an extracellular domain, isolating immune system cells (e.g., splenocytes, B cells, T cells) from the immunized mammal, fusing the immune system cells with myeloma cells, and identifying a clone from a hybridoma generated from the fusion, wherein the clone produces an antibody capable of binding to a DEA polypeptide. cDNA encoding the antibody can be cloned from the hybridoma, e.g., optionally using an amplification technique such as PCR. The coding sequence can then be used, e.g., to express the antibody in a recombinant host cell or transgenic organism. The sequences can be subjected to alteration such as random mutagenesis, chain or DNA shuffling methods, etc. The sequence can be modified, e.g., to humanize the antibody, combined with other antibody sequences, etc.

Phage display, in which antibody fragments are displayed on the surface of phage as fusions with a phage coat protein, can also be used to identify an antibody that binds to a DEA polypeptide. After displaying an antibody fragment on the surface of the phage, antigen specific phage are selected and enriched by multiple rounds of affinity panning. See, e.g., U.S. Pat. Nos. 5,855,885; 5,817,215; 6,172,197; 6,806,079. Libraries of antibody genes can be prepared from variable genes isolated from immunized animals, non-immunized animals, or synthetic libraries of genes can be used.

In some embodiments the antibody is a single chain antibody. Examples of techniques which can be used to produce single-chain Fvs and antibodies include those described in U.S. Pat. Nos. 4,946,778 and 5,258,498; Huston et al., Methods in Enzymology 203:46-88, 1991; Shu et al., PNAS 90:7995-7999, 1993; and Skerra et al., Science 240:1038-1040, 1988. Single chain antibodies are formed by linking the heavy and light chain fragments of the Fv region of an antibody via a linker such as a peptide bridge, resulting in a single chain polypeptide. The fragments can be synthesized separately and linked in vitro. However, in a preferred embodiment a recombinant nucleic acid that encodes the fragments, optionally separated by a peptide spacer, is expressed, e.g., in cells or in a transgenic plant, is used to produce the single chain antibody.

Both monospecific and multispecific (e.g., bispecific) antibodies are within the scope of the invention. Monovalent antibodies, bivalent antibodies, and antibodies having higher degrees of valency are also within the scope of the invention. A bispecific antibody has two distinct antigen binding sites that bind to different antigens. Antibody valency refers to the number of antigen binding sites. Bispecific or trispecific antibodies can be prepared, for example, by linking Fab′ fragments obtained from antibodies that bind to different antigens (Somasundaram C, et al., Hum Antibodies, 9(1):47-54, 1999). Single chain antibodies can be mono- or bispecific, and can be bivalent, trivalent, or tetravalent. A bispecific antibody has two distinct antigen binding sites that bind to different antigens. Antibody valency refers to the number of antigen binding sites. Construction of tetravalent, bispecific single chain antibodies is taught, for example, in Coloma and Morrison, Nat. Biotechnol. 15:159-163, 1997. Construction of bivalent, bispecific single chain antibodies is taught in Mallendar and Voss, J. Biol. Chem. 269:199-216, 1994. See also Cao Y and Suresh M R., Bioconjug Chem., 9(6):635-44, 1998. Bi- and tri-specific multimers can be formed by association of different scFv molecules. Varying the spacer length can determine whether See Joosten, V., et al., Microb Cell Fact., 2(1):1, 2003, for discussion of antibody fragments and antibody fusion proteins, with an emphasis on their production in yeasts and filamentous fungi.

In addition to antibodies such as those described above, antibody fragments that retain capability to bind to a DEA polypeptide can be used. For example, single domain binding proteins based upon immunoglobulin VH and VH-like domains can be used (Nuttall S D, et al, Curr Pharm Biotechnol., 1(3):253-63, 2000).

One of ordinary skill in the art will recognize that once an antibody that binds to a DEA polypeptide has been identified, changes can be made in the sequence without significantly altering the structure, e.g., without significantly reducing the ability of the antibody to bind the DEA polypeptide. Therefore, additional DEA antibodies and DEA antibody fragments can be generated by making additions, substitutions, and/or deletions to known antibody sequences, e.g., by performing site-directed mutagenesis of a polynucleotide that encodes an antibody chain or by chemical synthesis. Such variant antibodies or antibody fragments that bind to a DEA polypeptide could also be used, provided that they retain ability to bind to a DEA polypeptide. In certain embodiments of the invention a variant has substantial sequence identity or substantial sequence homology to a DEA antibody generated by a human or other animal or by phage display. For example, in a nonlimiting embodiment, a DEA antibody is at least 80% identical to a DEA antibody generated by a human or other animal or by phage display.

As mentioned above, it may be desirable to develop and/or select antibodies that specifically bind to particular regions of a DEA polypeptide, e.g., an extracellular domain. Such specificity may be achieved by immunizing the animal with peptides or polypeptide fragments that correspond to that region. Alternately, a panel of monoclonal antibodies can be screened to identify those that specifically bind to the desired region. The invention therefore provides, for each of the DEA polypeptides, a panel of antibodies wherein each member of the panel specifically recognizes a different antigenic determinant present in the DEA polypeptide.

In general, certain preferred antibodies possess high affinity, e.g., a K_{d} of <200 nM, and preferably, of <100 nM for their target. According to certain embodiments of the invention preferred antibodies do not show significant reactivity with tissues other than vascular tissues e.g., tissues of key importance such as kidney, brain, liver, bone marrow, colon, breast, prostate, thyroid, gall bladder, lung, adrenals, muscle, nerve fibers, pancreas, skin, etc. (Of course the antibodies may show significant reactivity with vascular structures within those tissues.) In the context of reactivity with tissues, the term “significant reactivity”, as used herein, refers to an antibody or antibody fragment, which, when applied to a tissue of interest under conditions suitable for immunohistochemistry, will elicit either no staining or negligible staining, e.g., only a few positive cells scattered among a field of mostly negative cells.

The invention provides various methods of using the antibodies described above. For example, the antibodies may be used to perform immunohistochemical analysis, immunoblotting, ELISA assays, etc., in order to detect the polypeptide to which the antibody specifically binds. In the case of DEA polypeptides that are released into the bloodstream, detection of the DEA polypeptide in a blood sample can provide a diagnostic test for atherosclerosis, as described further below. The antibodies may be used as components of antibody arrays. The antibodies may also be used for imaging studies, as described further below. In addition, the antibodies are useful for delivering attached moieties such as diagnostic or therapeutic agents to an atherosclerotic lesion or to a site within a blood vessel that is at risk of developing an atherosclerotic lesion. The antibodies are also useful as a targeting component of a targeted delivery vehicle (e.g., a microparticle, nanoparticle, liposome, etc.), and as therapeutic agents. In some embodiments an antibody that binds to a DEA polypeptide that is a receptor for an endogenous ligand, e.g., a cytokine or chemokine receptor is used as a therapeutic agent for treatment or prophylaxis of atherosclerosis. In an embodiment of particular interest, the receptor is one that is overexpressed in atherosclerotic lesions.

III. DEA Ligands and Methods for their Identification

In another aspect, the invention provides ligands that specifically bind to a DEA polypeptide. Such a ligand may be referred to herein as a “DEA ligand”. The term “ligand” is intended to encompass any type of molecule capable of specific binding, other than antibodies as described above. Ligands may be, for example, peptides, non-immunoglobulin polypeptides, nucleic acids, protein nucleic acids (PNAs), aptamers, small molecules, etc. Ligands that specifically bind to any of the DEA polypeptides described herein may be identified using any of a variety of approaches. For example, ligands may be identified by screening libraries, e.g., small molecule libraries. Naturally occurring or artificial (non-naturally occurring) ligands, particularly peptides or polypeptides, may be identified using a variety of approaches including, but not limited to, those known generically as two- or three-hybrid screens, the first version of which was described in Fields S. and Song O., Nature 1989 Jul. 20; 340(6230):245-6. Nucleic acid or modified nucleic acid ligands may be identified using, e.g., systematic evolution of ligands by exponential enrichment (SELEX) (Tuerk, C. and Gold., L, Science 249(4968): 505-10, 1990), or any of a variety of directed evolution techniques that are known in the art. For example, an aptamer is an oligonucleotide (e.g., DNA, RNA, which can include various modified nucleotides, e.g., 2′-O-methyl modified nucleotides) that binds to a particular protein. See, e.g., Brody E N, Gold L. J. Biotechnol., 74(1):5-13, 2000. In certain embodiments of the invention the ligand is an aptamer that binds to a DEA polypeptide. See also Jellinek, D., et al., Biochemistry, 34(36): 11363-72, 1995, describing identification of high-affinity 2′-aminopyrimidine RNA ligands to basic fibroblast growth factor (bFGF). Screens using nucleic acids, peptides, or polypeptides as candidate ligands may utilize nucleic acids, peptides, or polypeptides that incorporate any of a variety of nucleotide analogs, amino acid analogs, etc. Various nucleotide analogs are known in the art, and other modifications of a nucleic acid chain, e.g., in the backbone, can also be used, as described elsewhere herein.

A variety of engineered ligand-binding proteins with antibody-like properties are known in the art. For example, anticalins offer an alternative type of ligand-binding protein, which is constructed on the basis of lipocalins as a scaffold (Skerra, J., J. Biotechnol., 74(4):257-75, 2001). Affibodies, which are binding proteins generated by phage display from combinatorial libraries constructed using the protein A-derived Z domain as a scaffold, can also be used. See, e.g., Nord K, Eur J Biochem., 268(15):4269-77, 2001. Thus the invention provides an affibody or anticalin that specifically binds to a DEA polypeptide.

Peptides or polypeptides may incorporate one or more unnatural amino acids (e.g., amino acids that are not naturally found in mammals, or amino acids that are not naturally found in any organism). Such amino acids include, but are not limited to, cyclic amino acids, diamino acids, β-amino acids, homo amino acids, alanine derivatives, phenylalanine boronic acids, proline and pyroglutamine derivatives, etc. Alterations and modifications may include the replacement of an L-amino acid with a D-amino acid, or various modifications including, but not limited to, phosphorylation, carboxylation, alkylation, methylation, etc.

Polypeptides incorporating unnatural amino acids may be produced either entirely artificially or through biological processes, e.g., in living organisms. Use of unnatural amino acids may have a number of advantages. For example, unnatural amino acids may be utilized as building blocks, conformational constraints, molecular scaffolds, or pharmacologically active products. They represent a broad array of diverse structural elements that may be utilized, e.g., for the development of new leads in peptidic and non-peptidic compounds. They may confer desirable features such as enhanced biological activity, proteolytic resistance, etc. See, e.g., Bunin, B. A. et al., Annu. Rep. Med. Chem. 1999, 34, 267; Floyd, C. D. et al., Prog. Med. Chem. 1999, 36, 91; Borman, S. Chem. Eng. News 1999, 77, 33; Brown, R. K. Modern Drug Discovery 1999, 2, 63; and Borman, S. Chem. Eng. News 2000, 78, 53, describing various applications of unnatural amino acids. Once a ligand is identified, modifications such as those described above may be made.

In general, a screen for a ligand that specifically binds to any particular DEA polypeptide may comprise steps of contacting DEA polypeptide with a candidate ligand under conditions in which binding can take place; and determining whether binding has occurred. Any appropriate method for detecting binding, many of which are well known in the art, may be used. One of ordinary skill in the art will be able to select an appropriate method taking into consideration, for example, whether the candidate ligand is a small molecule, peptide, nucleic acid, etc. For example, the candidate ligand may be tagged, e.g., with a radioactive molecule. The DEA polypeptide can then be isolated, e.g., immunoprecipitated from the container in which the contacting has taken place, and assayed to determine whether radiolabel has been bound. This approach may be particularly appropriate for small molecules. Binding can be confirmed by any of a number of methods, e.g., radiolabel assays, plasmon resonance assays, etc. Phage display represents another method for the identification of ligands that specifically bind to DEA polypeptides. In addition, determination of the partial or complete three-dimensional structure of a DEA polypeptide (e.g., using nuclear magnetic resonance, X-ray crystallography, etc.) may facilitate the design of appropriate ligands.

Functional assays may also be used to identify ligands, particularly ligands that behave as agonists or antagonists, activators, or inhibitors of particular DEA polypeptides. For such assays it is necessary that the polypeptide of interest possesses a measurable or detectable functional activity and that such functional activity is increased or decreased upon binding of the ligand. Examples of functional activities of a polypeptide include, e.g., ability to catalyze a chemical reaction either in vitro or in a cell, ability to induce a change of any sort in a biological system, e.g., a change in cellular phenotype, a change in gene transcription, a change in membrane current, a change in intracellular or extracellular pH, a change in the intracellular or extracellular concentration of an ion, etc. when present within a cell or when applied to a cell.

Ligands that bind to DEA polypeptides have a variety of uses, some of which are described below. For example, they may serve as components of targeted conjugates and/or delivery vehicles. Ligands that modulate the expression and/or activity of a DEA polypeptide can also be used for therapeutic purposes.

Certain of the methods for identifying ligands may be performed in vitro, e.g., using a DEA polypeptide or a significantly similar polypeptide or fragment thereof produced using recombinant DNA technology. Certain of the methods may be performed by applying the test compound to a cell that expresses the polypeptide and measuring the expression or activity of the polypeptide, which may involve isolating the polypeptide from the cell and subsequently measuring its amount and/or activity. In certain of the methods the polypeptide may be a variant that includes a tag (e.g., an HA tag, 6×His tag, Flag tag, etc.) which may be used, for example, to facilitate isolation or the variant may be a fusion protein.

In general, an appropriate method for measuring activity of a polypeptide will vary depending on the polypeptide. For example, if the polypeptide has a known biological or enzymatic activity, or is homologous to a polypeptide with a known biological or enzymatic activity, that activity will be measured using any appropriate method known in the art. Thus if the polypeptide is a kinase a kinase assay will be performed. If the molecule is a cytokine, biological assays such as the ability to activate and/or trigger migration of other cell types can be assessed. If the molecule is a growth factor or growth factor receptor, the ability of the polypeptide to cause cell proliferation can be assessed.

Compounds suitable for screening according to the above methods include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem. Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments of the invention the methods are performed in a high-throughput format using techniques that are well known in the art, e.g., in multiwell plates, using robotics for sample preparation and dispensing, etc. Representative examples of various screening methods may be found, for example, in U.S. Pat. No. 5,985,829, U.S. Pat. No. 5,726,025, U.S. Pat. No. 5,972,621, and U.S. Pat. No. 6,015,692. The skilled practitioner will readily be able to modify and adapt these methods as appropriate.

Molecular modeling can be used to identify a pharmacophore for a particular target, i.e., the minimum functionality that a molecule must have to possess activity at that target. Such modeling can be based, for example, on a predicted structure for the target (e.g., a two-dimensional or three-dimensional structure). Software programs for identifying such potential lead compounds are known in the art, and once a compound exhibiting activity is identified, standard methods may be employed to refine the structure and thereby identify more effective compounds. For example computer-based screening can be used to identify small organic compounds that bind to concave surfaces (pockets) of proteins, can identify small molecule ligands for numerous proteins of interest (Huang, Z., Pharm. & Ther. 86: 201-215, 2000). In silico discovery of small molecules that bind to a protein of interest will typically involve, for example pharmacophore-aided database searches, virtual protein-ligand docking, and/or structure-activity modeling. For example, the computer program DOCK and variants thereof is widely used (Lorber, D. and Shoichet, B., Protein Science, 7:938-950, 1998). Other examples of suitable programs include Autodock and Flexx. It is noted that these programs and the hardware used to run them have undergone significant improvement since their introduction. Databases providing compound structures suitable for virtual screening are available in the art. For example, ZINC is a database that provides a library of 727, 842 molecules, each with 3D structure, which was prepared using catalogs of compounds that are commercially available (Irwin J J and Shoichet B K. J Chem Inf Model., 45(1):177-82, 2005). Each molecule in the library contains vendor and purchasing information and is ready for docking using a number of popular docking programs. In one embodiment the structure of a DEA polypeptide is screened against a database using a computer-based method to identify small molecules that bind to the DEA polypeptide. Assays to identify and/or to confirm molecules that bind to a DEA polypeptide could include functional assays, e.g., assessing the ability of a compound to prevent blood coagulation. Radioligand binding assays, competition assays, immunologically based assays, etc., could also be used.

According to certain of the inventive screening methods for identifying activators or inhibitors of a DEA polypeptide the DEA polypeptide is expressed in cells. In general, a wide variety of cells can be used, e.g., Xenopus oocytes, yeast cells, mammalian cells, etc. Numerous different types of mammalian cell lines are suitable, e.g., CHO cells, HEK293 cells, L cells, BHK cells, etc. Primary cells, e.g., vascular endothelial cells, vascular smooth muscle cells, etc., can also be used. In certain embodiments of the invention the screening assay involves detecting an alteration in a cellular phenotype. The phenotype can be any detectable morphological or biochemical characteristic of the cell that is affected by or dependent on the level of expression of the DEA polypeptide.

Thus the invention provides a method for screening for a ligand for a DEA polypeptide comprising steps of: (i) providing a sample comprising a DEA polypeptide; (ii) contacting the sample with a candidate compound; (iii) determining whether the level of activity of the polypeptide in the presence of the compound is increased or decreased relative to the level of activity of the DEA polypeptide in the absence of the compound; and (iv) identifying the compound as a ligand of the DEA polypeptide if the level of activity of the DEA polypeptide is higher or lower in the presence of the compound relative to its level of activity in the absence of the compound. In certain embodiments of the method the sample comprises cells that express the DEA polypeptide.

Identified compounds can be further tested in vitro or in vivo. For example, it may be desirable to include an additional step of (v) administering the compound to an animal suffering from or at risk of developing atherosclerosis or a disease or condition associated with atherosclerosis and evaluating the response. Response can be evaluated in any of a variety of ways, e.g., by assessing clinical features, laboratory data, blood vessel images, etc. A comparison may be performed with similar animals who did not receive the compound or who received a lower or higher amount of the compound. A number of animal models (e.g., mouse, rat, rabbit, pig, etc.) for atherosclerosis and for diseases associated with atherosclerosis, such as diabetes, are known in the art. Such models may involve genetic alterations, administration of drugs, etc., to include the development of atherosclerosis or a disease associated with atherosclerosis. See, e.g., Jawein, J., et al., J Physiol Pharmacol., 55(3):503-17, 2004, for a discussion of mouse models of atherosclerosis. See, e.g., Yanni, A., Lab Anim. 38(3):246-56, 2004, for a discussion of rabbit models of atherosclerosis. See, e.g., Rees, D A and Alcolado, J. C., Diabet Med., 22(4):359-70, 2005, for a discussion of animal models of diabetes.

The invention includes compounds identified using the above methods, e.g., compounds that increase or decrease one or more activities of a DEA polypeptide.

In general, a wide variety of different compounds can be screened. Numerous libraries of natural products, synthetic molecules, combinatorial libraries, etc., are known in the art, and any of these can be used, as mentioned above. In addition, the assays can be used to test variants of known ligands of a receptor that is identified as a DEA polypeptide herein, e.g., a cytokine or chemokine receptor.

IV. Targeting Agents, Targeted Conjugates, and Targeted Delivery Vehicles

The invention provides a variety of different targeting agents that bind to the polypeptides encoded by the DEA genes identified herein. Such targeting agents are useful for a variety of purposes including diagnostic, therapeutic, as targeted delivery vehicles or components of such vehicles, for research purposes, etc. The invention provides a targeting agent that specifically binds to a DEA polypeptide encoded by a polynucleotide whose sequence comprises the sequence of a polynucleotide whose Genbank accession number is selected from the group of Genbank accession numbers listed in any of Tables 1-4 or 8. In particular, the invention provides a targeting agent that specifically binds to a DEA polypeptide encoded by a gene selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy 1, COX-2, and ADAMTS1. The invent The targeting agent can be an antibody or ligand that specifically binds to a DEA polypeptide. Such antibodies and ligands are described above.

In another aspect, the invention provides a conjugate comprising a targeting agent linked with a functional moiety, wherein the targeting agent specifically binds to a DEA polypeptide. Targeting agents may be any agent that specifically binds to a DEA polypeptide. In particular, targeting agents can be antibodies or ligands that specifically bind to a DEA polypeptide, as described above.

In general, these conjugates possess at least two functions, one of which is specifically binding to a DEA polypeptide. By “functional moiety” is meant any compound, agent, molecule, etc., that possesses an activity or property that alters, enhances, or otherwise changes the ability of the targeting agent to fulfill any particular purpose or that enables the targeting agent to fulfill a new purpose. Such purposes include, but are not limited to, providing diagnostic and/or prognostic information and/or treatment of diseases or conditions associated with atherosclerosis, or imaging vascular tissue, e.g., imaging atherosclerotic lesions in blood vessel walls.

By “linked” is generally meant covalently bound or, if noncovalently bound, physically associated via intermolecular forces approximately equal in strength to that of covalent bonds and exhibiting specific binding. Thus a noncovalent interaction between two molecules that has very slow dissociation kinetics can function as a link. For example, an antibody associated with its cognate antigen is generally considered linked. As another example, reactive derivatives of phospholipids can be used to link the liposomes or cell membranes in which they are incorporated to antibodies or enzymes. Targeting agents, e.g., antibodies or ligands linked to a functional moiety will be referred to herein as conjugates or heteroconjugates. According to certain embodiments of the invention the functional moiety is a compound (e.g., a polymer such as polyethylene glycol) that stabilizes the targeting agent and/or increases its resistance to degradation. According to certain embodiments of the invention the functional moiety is a diagnostic agent or a therapeutic agent. Suitable diagnostic and therapeutic agents are discussed below. It will be appreciated that a conjugate can comprise multiple either identical or different DEA targeting agents and can comprise multiple either identical or different functional moieties.

According to certain embodiments of the invention the targeting agent is synthesized using precursors, e.g., amino acids, that contain the functional moiety. For example, an antibody or a polypeptide ligand can be synthesized using amino acid precursors that contain flourine-19 instead of hydrogen at one or more positions, or that contain nitrogen-15 or oxygen-17 instead of the more abundant isotope at one or more positions. As a second example, where the functional moiety is a polypeptide, the composition may be produced as a fusion protein, as described above, wherein one portion of the fusion protein (the antibody or ligand) specifically binds to the DEA polypeptide and a second portion of the fusion protein consists of or comprises a functional moiety. Alternately, polypeptides may be modified to incorporate a functional moiety. For example, the methods described in Haruta, Y., and Seon, B. K., Proc. Nat. Acad. Sci., 83, 7898-7902 (1986) may be used to iodinate antibodies and other polypeptides. See also Tabata, M., et al., Int. J. Cancer, Vol. 82, Issue 5: 737-742, 1999. Functional moieties incorporated into a targeting agent of the invention during synthesis or added to the antibody or ligand subsequently are considered “linked” to the targeting agent.

Functional moieties may be linked to targeting agents such as antibodies by any of a number of methods that are well known in the art. Examples include, but are not limited to, the glutaraldehyde method which couples primarily through the α-amino group and ε-amino group, maleimide-sulfhydryl coupling chemistries (e.g., the maleimidobenzoyl-N-hydroxysuccinimide ester (MBS) method), and periodate oxidation methods, which specifically direct the coupling location to the Fc portion of the antibody molecule. In addition, numerous cross-linking agents are known, which may be used to link the targeting agent to the functional moiety.

A wide variety of methods (selected as appropriate taking into consideration the properties and structure of the ligand and functional moiety) may likewise be used to produce the DEA-targeted conjugates of the invention. Suitable cross-linking agents include, e.g., carboiimides, N-Hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc. According to certain embodiments of the invention the functional moiety is a compound (e.g., polyethylene glycol) that stabilizes the ligand and/or increases its resistance to degradation.

For additional information on conjugation methods and crosslinkers see generally the journal Bioconjugate Chemistry, published by the American Chemical Society, Columbus Ohio, PO Box 3337, Columbus, Ohio, 43210. This journal reports on advances concerning the covalent attachment of active molecules to biopolymers, surfaces, and other materials. Coverage spans conjugation of antibodies and their fragments, nucleic acids and their analogs, liposomal components, and other biologically active molecules with each other or with any molecular groups that add useful properties. Such molecular groups include small molecules, radioactive elements or compounds, polypeptides, etc. See also “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991. The following section presents a number of examples of specific conjugation approaches and cross-linking reagents. However, it is to be understood that the invention is not limited to these methods, and that selection of an appropriate method may require attention to the properties of the particular functional moiety, substrate, or other entity to be linked to the targeting agent.

According to certain embodiments of the invention a bifunctional crosslinking reagent is used to couple a functional moiety with a targeting agent of the invention. In general, bifunctional crosslinking reagents contain two reactive groups, thereby providing a means of covalently linking two target groups. The reactive groups in a chemical crosslinking reagent typically belong to various classes of functional groups such as succinimidyl esters, maleimides, and iodoacetamides. Bifunctional chelating agents may also be used. For example, a targeting agent of the invention may be coupled with a chelating agent, which may be used to chelate a functional moiety such as a metal. Bifunctional chelating agents may be used to couple more than one functional moiety to a targeting agent of the invention. For example, according to certain embodiments of the invention one or more of the functional moieties is useful for imaging and/or one or more of the functional moieties is useful for therapy. Appropriate chelating agents for use with the antibodies or ligands of the invention include polyaminocarboxylates, e.g., DTPA, macrocyclic polyaminocarboxylates such as 1, 4, 7, 10-tetraazacyclododecane N,N′,N″,N′″-tetraacetic acid (DOTA), etc. See Lever, S., J. Cell. Biochem. Suppl., 39:60-64, 2002, and references therein.

The most common schemes for forming a heteroconjugate involve the indirect coupling of an amine group on one biomolecule to a thiol group on a second biomolecule, usually by a two- or three-step reaction sequence. The high reactivity of thiols and their relative rarity in most biomolecules make thiol groups good targets for controlled chemical crosslinking. If neither molecule contains a thiol group, then one or more can be introduced using one of several thiolation methods. The thiol-containing biomolecule may then be reacted with an amine-containing biomolecule using a heterobifunctional crosslinking reagent, e.g., a reagent containing both a succinimidyl ester and either a maleimide or an iodoacetamide. Amine-carboxylic acid and thiol-carboxylic acid crosslinking may also be used. For example, 1-Ethyl-3-(3-dimethylaminopropyl)carbodiimide (EDAC) can react with biomolecules to form “zero-length” crosslinks, usually within a molecule or between subunits of a protein complex. In this chemistry, the crosslinking reagent is not incorporated into the final product. The water-soluble carbodiimide EDAC crosslinks a specific amine and carboxylic acid between subunits of allophycocyanin, thereby stabilizing its assembly. See, e.g., Yeh S W, et al., “Fluorescence properties of allophycocyanin and a crosslinked allophycocyanin trimer.”, Cytometry 8, 91-95 (1987).

Several methods are available for introducing thiols into biomolecules, including the reduction of intrinsic disulfides, as well as the conversion of amine, aldehyde or carboxylic acid groups to thiol groups. Disulfide crosslinks of cystines in proteins can be reduced to cysteine residues by dithiothreitol (DTT), tris-(2-carboxyethyl)phosphine (TCEP), or tris-(2-cyanoethyl)phosphine. Amines can be indirectly thiolated by reaction with succinimidyl 3-(2-pyridyldithio)propionate (SPDP) followed by reduction of the 3-(2-pyridyldithio)propionyl conjugate with DTT or TCEP. Amines can be indirectly thiolated by reaction with succinimidyl acetylthioacetate followed by removal of the acetyl group with 50 mM hydroxylamine or hydrazine at near-neutral pH. Tryptophan residues in thiol-free proteins can be oxidized to mercaptotryptophan residues, which can then be modified by iodoacetamides or maleimides

For purpose of covalently linking active molecules (e.g., therapeutic agents) to targeting agents, it may be preferred to select methods that result in a conjugate wherein the targeting agent is separable from the therapeutic agent to allow the agent to enter the cell. Thiol-cleavable, disulfide-containing conjugates may be employed for this purpose. Cells are able to break the disulfide bond in the cross-linker, which permits release of the agent within the target cell. Examples of suitable cross-linkers include 2-iminothiolane (Traut's reagent), N-succinimidyl 3-(2-pyridyldithio)propionate (SPDP), etc. In addition, it is generally preferable to select methods that do not significantly impair the ability of the targeting agent to specifically bind to its target and do not significantly impair the ability of the functional moiety to perform its intended function. One of ordinary skill in the art will be able to test the conjugate to determine whether the targeting agent retains binding ability and/or whether the functional moiety retains its function.

According to certain embodiments of the invention the functional moiety is released from the targeting agent upon uptake into the cell. For example, the functional moiety may be attached to the targeting agent via a linker or spacer that is cleaved by an intracellular enzyme such as a protease. In other embodiments of the invention the functional moiety is released from the targeting agent upon arrival in the vicinity of an atherosclerotic lesion. In such embodiments the functional moiety may be attached to the targeting agent via a linker or spacer that is cleaved by an enzyme that is present on or in a blood vessel wall in the vicinity of an atherosclerotic lesion. For example, the enzyme may be overexpressed in atherosclerotic lesions. As noted above the present invention provides the discovery that certain MMPs are overexpressed in atherosclerotic lesions. A functional moiety can be attached to a targeting agent by a peptide linker that comprises a cleavage sites for such enzymes. According to certain embodiments of the invention the functional moiety is an antisense molecule, ribozyme, siRNA, or shRNA which may be targeted to any transcript present in blood vessel cell. In general, the antibodies and ligands of the invention that specifically bind to DEA polypeptides may be used as described in Allen, T., Nature Reviews Cancer, Vol. 2, pp. 750-765, 2002, and references therein.

According to certain embodiments of the invention the functional moiety is one that causes, either directly or indirectly, a change in the physiological (i.e., functional) and/or biochemical state of a cell with which it comes into contact. In general, a change in the physiological state of a cell will involve multiple biochemical changes. By “directly causing” is meant that the functional moiety either causes the change itself or by interacting with one or more cellular or extracellular constituents (e.g., nucleic acid, protein, lipid, carbohydrate, etc.) not introduced or induced by the hand of man. The category of direct causation includes instances in which the functional moiety initiates a “pathway”, e.g., in which the functional moiety interacts with one or more constituents, which causes a change in the interaction(s) of this constituent with other constituents, ultimately leading to the alteration in physiological or biochemical state of the cell. By “indirectly causing” is meant either (i) that the functional moiety itself does not cause the change but must be converted into an active form (e.g., by a cellular enzyme) in order to cause the change; or (ii) that the functional moiety itself does not cause the change but instead acts on a second agent that causes the change, which second agent is also introduced to or induced in the cell, its surface, or vicinity by the hand of man.

Various examples of changes in physiological or biological state include, but are not limited to, increases or decreases in gene expression (e.g., increases or decreases in transcription, translation, and/or mRNA or protein turnover), alterations in subcellular localization or secretion of a cellular constituent, alteration in cell viability or growth rate, alteration in differentiation state, etc. According to certain embodiments of the invention the functional moiety is a growth stimulatory or inhibitory agent. For example, the functional moiety may comprise or encode a growth factor, a growth factor receptor, or an agonist or antagonist of a growth factor receptor, wherein the growth factor, growth factor receptor, growth factor receptor agonist, or growth factor receptor antagonist stimulates or inhibits growth or division of blood vessel cells.

According to certain embodiments of the invention the functional moiety is a nucleic acid, which may serve as a template for a transcript to be expressed in the cell. The transcript may encode a polypeptide to be expressed within the cell or may act as a ribozyme, antisense molecule, siRNA, shRNA, any of which may reduce or inhibit expression of a target transcript, e.g., by cleaving the transcript (in the case of ribozymes), causing degradation of the transcript, and/or inhibiting its translation. It will be appreciated that the effect of a ribozyme, antisense molecule, siRNA, or shRNA will depend, in general, upon the particular target transcript.

The invention further provides a variety of delivery vehicles targeted to vascular tissue. The delivery vehicles comprise a targeting agent, e.g., a DEA antibody or DEA ligand, that specifically binds to a DEA polypeptide. In certain embodiments of the invention the targeting agent specifically binds to a DEA polypeptide that is overexpressed in atherosclerotic lesions. In general, delivery vehicles are employed to improve the ability of a functional moiety, e.g., a diagnostic or therapeutic agent, to achieve its desired effect at or on a cell, tissue, organ, subject, etc., e.g., by increasing the likelihood that the agent will reach its intended site of activity. By “delivery vehicle” is meant a natural or artificial substance that is physically associated with an agent such as a diagnostic or therapeutic agent and provides one or more of the following functions among others: (1) conveys the agent within the body; (2) facilitates the binding to and/or uptake of the agent by cells, tissues, organs, etc.; (3) increases stability of the agent, e.g., increases half-life of the agent in the body; (4) changes other pharmacokinetic properties of the agent from what they would have been in the absence of the delivery vehicle.

The agent may be associated with the delivery vehicle in any of a number of ways. For example, the agent may be bonded to the delivery vehicle (e.g., via covalent or noncovalent bonds). In certain embodiments of the invention the agent is physically associated with a delivery vehicle by a nonspecific interaction mechanism. A “nonspecific interaction mechanism” is a physical interaction in which one or more entities is entrapped, embedded, enclosed, or encapsulated within another entity, or entangled with another entity, or dissolved in another entity, or dispersed in another entity, or impregnated with another entity, or adsorbed to another entity, so as to maintain a physical association therebetween. By “dispersed within” is meant that individual molecules of the agent are intermingled with molecules comprising the material from which the delivery vehicle is made as opposed to existing in discrete clusters. Discrete clusters of the agent may be dispersed within the delivery vehicle.

According to the invention a DEA targeting agent is incorporated in and/or linked to the delivery vehicle for targeting to an atherosclerotic lesion or blood vessel site that is at risk of developing an atherosclerotic lesion. Typically at least the portion of the targeting agent that binds to the DEA polypeptide is present at the surface of the delivery vehicle so that it can interact with the DEA polypeptide, while the molecule to be delivered is typically inside. Such targeted delivery vehicles may be used for the delivery of a wide variety of agents to atherosclerotic lesions or blood vessel sites at risk of developing an atherosclerotic lesion.

In certain embodiments of the invention a targeting agent of the invention is conjugated to a microparticle, a nanoparticle, liposome, or other lipid-containing agent that can serve as a carrier. In other embodiments the targeting agent is physically associated with a microparticle, nanoparticle, liposome, or other lipid-containing agent by a nonspecific interaction mechanism. The microparticles, nanoparticles, liposomes, or other lipid-containing agents can incorporate functional moieties such as therapeutic agents or diagnostic agents (e.g., agents useful for imaging) and are used as delivery vehicles for such moieties. The term “microparticle” as used herein is intended to encompass any particulate bead, sphere, particle, capsule, or carrier, which can be biodegradable or nonbiodegradable, comprised of naturally-occurring or synthetic, organic or inorganic materials, that is substantially nontoxic when administered to a subject. The microparticle optionally comprises a coating layer, which is optionally biodegradable. In some embodiments of the invention the microparticle is impregnated with or encapsulates a therapeutic agent. Alternately, a therapeutic agent is coated on the surface of the microparticle, or a coating of the microparticle is impregnated with a therapeutic agent. In some embodiments a therapeutic agent is attached to the microparticle either directly or by a linker. The therapeutic agent diffuses out of the microparticle or coating layer and/or is released as the microparticle, coating layer, or both, degrades in the body and/or is released by cleavage of the linking moiety.

The targeted microparticles of the invention can be any particulate bead, sphere, particle, capsule, or carrier having a diameter of about 10 nm to about 500 microns in the case of particles that are approximately spherical. Generally, a microparticle has a diameter of 500 microns or less, e.g., between 50 and 500 microns, between 20 and 50 microns, between 1 and 20 microns, between 1 and 10 microns, and a nanoparticle will have a diameter of less than 1 micron. A microparticle having a diameter less than approximately 1000 nm is considered to be a nanoparticle. In certain embodiments the microparticles are nanoparticles having a diameter of less than approximately 500 nm, e.g. between approximately 100-200 nm, approximately 100 nm, etc. One of ordinary skill in the art will appreciate that the microparticle need not be spherical but can assume any of a number of regular or irregular shapes, in which case the relevant dimension will be the longest dimension of any cross-section of the particle.

The targeted microparticles of the invention can comprise, for example, polystyrene, cellulose, silica, and various polysaccharides including dextran, agarose, cellulose and modified, crosslinked and derivatized embodiments thereof. Alternately, microparticles of the invention can be formed from a wide variety of additional polymers including, but not limited to, polymers mentioned above. Specific biocompatible, biodegradable polymers include, for example, poly(lactides), poly(glycolides), poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetals, polycyanoacrylates, polyetheresters, poly(dioxanone)s, poly(alkylene alkylates), copolymers of polyethylene glycol and polyorthoesters, biodegradable polyurethanes, blends and copolymers of the foregoing polymers. A specific example is an N-(2-hydroxypropyl)methacrylamide copolymer (HPMA). Natural polymers such as albumin, gelatin, chitosan, alginate, collagen or mixtures thereof can also be used. In a preferred embodiment the nanoparticles comprise chitosan or a poly(lactide-co-glycolide (PLGA). Derivatized microparticles are available commercially and include microparticles derivatized with carboxyalkyl groups such as carboxymethyl, phosphoryl and substituted phosphoryl groups, sulfate, sulfhydryl and sulfonyl groups, and amino and substituted amino groups. Methods for making microparticles and nanoparticles, and for encapsulating therapeutic agents therein, or otherwise physically associating an agent with a microparticle, are known in the art and include spray drying, spray-freeze drying, phase separation, single or double emulsion solvent evaporation, solvent extraction, and simple and complex coacervation. Diagnostic or therapeutic agents can be loaded into microparticles during their formation or afterwards. In general, the methods described above for producing a conjugate comprising a targeting agent and a functional moiety are also of use for attaching a targeting agent to a delivery agent.

Liposomes employed in the present invention can be prepared using any one of a variety of conventional liposome preparatory techniques. As will be readily apparent to those skilled in the art, such conventional techniques include sonication, chelate dialysis, homogenization, solvent infusion coupled with extrusion, freeze-thaw extrusion, microemulsification, as well as others. These techniques, as well as others, are discussed, for example, in U.S. Pat. No. 4,728,578, U.K. Patent Application G.B. 2193095 A, U.S. Pat. Nos. 4,533,254; 4,728,575; 4,737,323; 4,753,788 and 4,935,171. See also Gregoriades, G. (ed.), Liposome Technology, vol. 1-3, CRC, Boca Raton, 1984; Gregoriades, G. (ed.), Liposomes as Drug Carriers, John Wiley & Sons, Chichester, 1988, 1984; Lasic, D. D., Liposomes: From Physics to Applications, Elsevier, Amsterdam, 1993; Martin, F. & Lasic, D. (eds.) Stealth Liposomes, CRC, Boca Raton, 1995; Woodle, M. C & Storm, G. (eds.), Long Circulating Liposomes. Old Drugs, New Therapeutics, Springer, Berlin, 1997; Torchilin, V. P. & Weissig, V. (eds.), Liposomes. Practical Approach, Oxford University Press, Oxford, 2003. In certain embodiments of the invention a reagent used to crosslink a liposome or other lipid-containing agent to a biomolecule such as a DEA antibody or a small molecule comprises a phospholipid derivative to anchor one end of the crosslink in the lipid layer and a reactive group at the other end to provide a point of attachment to the target biomolecule. In certain embodiments of the invention a polymerized liposome is used. In certain embodiments the liposome is coated with a polymer. For example, the liposome may have polyethylene glycol (PEG) or a similar polypeptide attached to or coated on its surface. Such polymers may stabilize the liposome, reduce its clearance from the body, and/or reduce its immunogenicity. The liposome may be loaded with a functional moiety such as a diagnostic or therapeutic agent either during or after its formation. The agent may be contained in an aqueuous core of the liposome or can be incorporated into or attached to its surrounding membrane.

It will be appreciated that a delivery vehicle of the invention can comprise multiple either identical or different DEA targeting agents and can comprise multiple either identical or different functional moieties.

The invention further provides a targeting agent, e.g., an antibody or ligand that specifically binds to a DEA polypeptide, conjugated to a support. The support can be, for example, a nanosphere, microsphere, or bead such as those described above but could alternatively be a nonparticulate support. The support can be made out of any of a variety of materials including, but not limited to, agarose, polyacrylamide, nylon, dextran, polyethylene glycol, polysaccharides such as PLA, PLGA or chitosan, other polymers, etc. A support comprising an agent that specifically binds to a DEA polypeptide can be used, e.g., for detecting the DEA polypeptide either in vitro (e.g., in isolated cells, in a cell lysate, etc.) or in vivo. Such supports can also be used for isolating, and/or purifying a DEA polypeptide.

V. Reagents and Methods for Detection and Imaging of Vascular Tissue

As described above, the invention provides a conjugate comprising a targeting agent linked to a functional moiety, wherein the targeting agent specifically binds to a DEA polypeptide. The invention further provides a delivery vehicle comprising a functional moiety and a targeting agent that specifically binds to a DEA polypeptide. According to certain embodiments of the invention the functional moiety is a readily detectable moiety. In general, a readily detectable moiety has a property such as fluorescence, chemiluminescence, radioactivity, color, magnetic or paramagnetic properties, etc., which property renders it detectable by instruments that detect fluorescence, chemiluminescence, radioactivity, color, or magnetic resonance, etc. Alternately, a readily detectable moiety may comprise or encode an enzyme that acts on a substrate to produce a readily detectable compound. According to certain embodiments of the invention the readily detectable moiety is one that, when present at a target site subsequent to administration of the inventive composition to a subject, can be detected from outside the body. In certain preferred embodiments of the invention the readily detectable moiety can be detected non-invasively.

A variety of different detectable moieties suitable for imaging (e.g., moieties suitable for detection by X-ray, fluoroscopy, computed tomography, magnetic resonance imaging, positron emission tomography, gamma tomography, electron spin resonance imaging, optical or fluorescence microscopy, etc.) can be used. Such agents are referred to herein as “imaging agents”. Imaging agents include, but are not limited to, radioactive, paramagnetic, or supraparamagnetic atoms (or molecules containing them). Suitable radioactive atoms include technetium-99m, thallium-211, iodine-133; atoms with magnetic moments such as iodine-123, iodine-131, indium-111, fluorine-19, carbon-13, nitrogen-15, oxygen-17, gadolinium, manganese, or iron. Other suitable atoms include rhenium-186 and rhenium-188. Useful paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), europium, and erbium (III), with gadolinium being particularly preferred. Gd-chelates, e.g., DTPA chelates, may be used. For example, the water soluble Gd(DTPA)²-chelate, is one of the most widely used contrast enhancement agents in experimental and clinical imaging research. The DTPA chelating ligand may be modified, e.g., by appending one or more functional groups preferably to the ethylene diamine backbone. Another agent of use is Gadlfluorine M (Schering AG), which is a lipophilic, macrocyclic water-soluble gadolinium chelate complex (Aguinaldo, J. G. S., et al, Mol. Imaging, 2: 282, 2003). Ions useful in other contexts, such as X-ray imaging, include but are not limited to lanthanum (III), gold (III), lead (II), and bismuth (III). Additional moieties useful for imaging include gallium-67, copper-67, yttrium-90, and astatine-211. Moieties useful for optical or fluorescent detection include fluorescein and rhodamine and their derivatives. Agents that induce both optical contrast and photosensitivity include derivatives of the phorphyrins, anthraquinones, anthrapyrazoles, perylenequinones, xanthenes, cyanines, acridines, phenoxazines and phenothiazines (Diwu, Z. J. and Lown, J. W., Pharmacology and Theraeutics 63: 1-35, 1994; Grossweiner, L. I., American Chemical Society Symposium Series 559: 255-265, 1994).

Appropriate imaging procedures include, but are not limited to, X-ray, fluoroscopy, computed tomography, magnetic resonance imaging, positron emission tomography and variants thereof such as SPECT or CT-PET, gamma tomography, electron spin resonance imaging, ultrasound imaging, optical or fluorescence microscopy, etc. Further information regarding methods and applications of molecular imaging in contexts including basic research, diagnosis, therapeutic monitoring, drug development, etc., may be found in articles appearing in the Journal of Cellular Biochemistry, Volume 87, Issue S39 (Supplement), 2002. See also Choudhury, R. P., et al., Nature Reviews Drug Discovery, 3: 913-925, 2004, for a review. See also the references listed in that article, all of which are incorporated herein by reference.

The readily detectable moiety may be linked to the DEA targeting agent using various methods as described above or may be associated with a DEA-targeted delivery vehicle. See, e.g., U.S. Pat. Nos. 5,021,236 and 4,472,509, for various diagnostic agents known in the art to be useful for imaging purposes and methods for their attachment to antibodies. See also discussion above describing coupling of antibodies and ligands of the invention with functional moieties. It is noted that many of the detectable moieties mentioned herein may also be useful for therapeutic applications.

Accordingly, the invention provides a method of imaging vascular tissue in a sample or subject, comprising steps of: (i) administering to the sample or subject an effective amount of a targeting agent that specifically binds to a DEA polypeptide, wherein the targeting agent is linked to a functional moiety that enhances detectability of vascular system cells by an imaging procedure; and (ii) subjecting the sample or subject to the imaging procedure. The targeting agent may be, for example, an antibody or ligand that specifically binds to the DEA polypeptide. The invention also provides a method of imaging vascular tissue in a sample or subject, comprising steps of: (i) administering to the sample or subject an effective amount of a delivery vehicle comprising a targeting agent that specifically binds to a DEA polypeptide and also comprising a functional moiety that enhances detectability of vascular system cells by an imaging procedure; and (ii) subjecting the sample or subject to the imaging procedure. The targeting agent may be, for example, an antibody or ligand that specifically binds to the DEA polypeptide. Exemplary delivery vehicles include liposomes with amphipathic chelates embedded in the outer membrane (Sipkins, D A, et al., Nature Med., 623-626, 1998), perfluorocarbon emulsions (Yu, et al, Magn. Reson. Med, 44: 867-872, 2000), etc.

The methods are useful for imaging vascular tissue for any of a wide variety of purposes. In general, the level of expression of the DEA polypeptide will be reflected in a characteristic of the image such as intensity. The level of expression can be useful in diagnosing disease (e.g., atherosclerosis and related conditions), assessing disease severity, and/or monitoring the course of the disease or response to treatment. Thus in certain embodiments the method is a method of detecting an atherosclerotic lesion. In certain embodiments the method is a method of providing diagnostic or prognostic information related to atherosclerosis or a disease or condition associated with atherosclerosis.

In the case of certain of the DEA genes identified herein, this work provides the first evidence that these genes are expressed in atheroscelerotic lesions. Imaging the expression of these genes will be useful for purposes unrelated to assessing risk or severity of atherosclerosis, response to treatment for atherosclerosis, etc. For example, the fact that certain of these genes are expressed, e.g., overexpressed, in atherosclerotic lesions indicates that detecting their expression, e.g., by means of imaging, will allow visualization of atherosclerotic lesions for purposes such as assessing the severity or extent of atherosclerosis, evaluating the response to therapy, determining when an intervention such as angioplasty, stent placement, atherectomy, or cardiac revascularization is warranted, etc.

It is noted that the invention includes embodiments in which the DEA polypeptide whose expression is detected is overexpressed in atherosclerotic lesions relative to its expression in nonlesion vascular tissue and also includes embodiments in which the DEA polypeptide whose expression is detected is underexpressed in atherosclerotic lesions relative to its expression in nonlesion vascular tissue. In the former case, detection of the polypeptide, particularly at high levels, is indicative of and/or correlates positively with, the extent and/or severity of an atherosclerotic lesion, while absence of or low level expression of the polypeptide is indicative of and/or correlates positively with the lack of an atherosclerotic lesion, i.e., the presence of normal vascular tissue. In the latter case, detection of the polypeptide is indicative of and/or correlates positively with the presence of normal vascular tissue, while absence of or low level expression of the polypeptide correlates with, i.e., is indicative of and/or correlates positively with the presence of an atherosclerotic lesion. In one embodiment the DEA polypeptide that is detected is encoded by the oxidized LDL receptor 1 gene (corresponding to accession number AA682386).

VI. Reagents and Methods for Modulating Expression and/or Activity of DEA Polynucleotides and Polypeptides

Since the DEA genes are potential therapeutic targets for atherosclerosis and/or diseases or conditions associated with atherosclerosis, it is desirable to be able to modulate their expression and/or activity, both for therapeutic and other purposes. The invention therefore provides a variety of methods for altering expression and/or functional activity of a DEA gene, which are further described below. The invention encompasses methods for screening compounds for preventing or treating atherosclerosis or a disease or clinical condition associated with atherosclerosis by assaying the ability of the compounds to modulate the expression of the DEA genes disclosed herein or activity of the protein products of these genes. Appropriate screening methods include, but are not limited to, assays for identifying compounds and other substances that interact with (e.g., bind to) the target gene protein products.

A. Methods for Reducing Gene Expression

1. Antisense Nucleic Acids and Methods of Use

Antisense nucleic acids are generally single-stranded nucleic acids (DNA, RNA, modified DNA, modified RNA, or peptide nucleic acids) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a DEA polypeptide may be achieved by the administration of an antisense nucleic acid or peptide nucleic acid (PNA) comprising sequences complementary to those of the mRNA that encodes the polypeptide. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

Peptide nucleic acids (PNA) are analogs of DNA in which the backbone is a pseudopeptide rather than a sugar. PNAs mimic the behavior of DNA and bind to complementary nucleic acid strands. The neutral backbone of a PNA can result in stronger binding and greater specificity than normally achieved using DNA or RNA. Binding typically reduces or inhibits the function of the target nucleic acid. Peptide nucleic acids and their use are described in Nielsen, P. E. and Egholm, M., (eds.) “Peptide Nucleic Acids: Protocols and Applications” (First Edition), Horizon Scientific Press, 1999.

According to various embodiments of the invention the antisense oligonucleotides have a variety of lengths. For example, they may comprise between 8 and 60 contiguous nucleotides complementary to a DEA mRNA, between 10 and 60 contiguous nucleotides complementary to a DEA mRNA, or between 12 and 60 contiguous nucleotides complementary to a DEA mRNA. According to certain embodiments of the invention a DEA antisense olignucleotide need not be perfectly complementary to the corresponding mRNA but may have up to 1 or 2 mismatches per 10 nucleotides when hybridized to the corresponding mRNA.

The invention further encompasses a method of inhibiting expression of a DEA polypeptide in a cell or a subject comprising delivering a DEA antisense oligonucleotide to the cell or subject or expressing such an antisense oligonucleotide within a cell or cells of the subject. In addition, the invention provides a method of treating a condition associated with atherosclerosis comprising steps of (i) providing a subject in need of treatment for atherosclerosis or a disease or condition associated with atherosclerosis; and (ii) administering a pharmaceutical composition comprising an effective amount of a DEA antisense oligonucleotide to the subject, thereby alleviating one or more symptoms of atherosclerosis in the subject.

2. DEA Ribozymes and Methods of Use

Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. Extensive discussion of ribozyme technology and its uses is found in Rossi, J. J., and Duarte, L. C., Intracellular Ribozyme Applications Principles and Protocols, Horizon Scientific Press, 1999.

The invention provides a ribozyme designed to cleave a DEA mRNA. The invention further encompasses a method of inhibiting expression of a DEA polypeptide in a cell or subject comprising delivering a ribozyme designed to cleave a DEA mRNA to the cell or subject or expressing such a ribozyme within a cell or cells of the subject. In addition, the invention provides a method of treating a condition associated with atherosclerosis comprising steps of (i) providing a subject in need of treatment for a condition associated with atherosclerosis; and (ii) administering a pharmaceutical composition comprising an effective amount of a ribozyme designed to cleave DEA mRNA to the subject, thereby alleviating the condition.

3. Reagents for Reducing Expression by RNA Interference and Methods of Use

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA that contain regions complementary to one strand (the antisense strand) of the dsRNA in cells of various types after first undergoing processing by an RNase III-like enzyme called DICER (Bernstein et al., Nature 409:363, 2001) into smaller dsRNA molecules. These molecules comprise two 21 nt strands, each of which has a 5′ phosphate group and a 3′ hydroxyl, and includes a 19 nt region precisely complementary with the other strand, so that there is a 19 nt duplex region flanked by 2 nt-3′ overhangs and are known as short interfering RNA (siRNA). An siRNA typically comprises a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

RNAi can also be achieved using molecules referred to as short hairpin RNAs (shRNA), which are single RNA molecules comprising at least two complementary portions capable of self-hybridizing to form a duplex structure sufficiently long to mediate RNAi (typically at least 19 base pairs in length), and a loop, typically between approximately 1 and 10 nucleotides in length and more commonly between 4 and 8 nucleotides in length that connects the two nucleotides that form the last nucleotide pair at one end of the duplex structure. shRNAs are thought to be processed into siRNAs by the conserved cellular RNAi machinery. Thus shRNAs are precursors of siRNAs and are similarly capable of inhibiting expression of a target transcript.

siRNAs and shRNAs have been shown to downregulate gene expression when transferred into mammalian cells by such methods as transfection, electroporation, or microinjection, or when expressed in cells via any of a variety of plasmid-based approaches. RNA interference using siRNA and/or shRNA is reviewed in, e.g., Tuschl, T., Nat. Biotechnol., 20: 446-448, May 2002. See also Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002). A number of variations in structure, length, number of mismatches, size of loop, identity of nucleotides in overhangs, etc., are consistent with effective RNAi-mediated gene silencing. For example, one or more mismatches between the target mRNA and the complementary portion of the siRNA or shRNA may still be compatible with effective silencing.

It is thought that intracellular processing (e.g., by DICER) of a variety of different precursors results in production of RNAs of various kinds that are capable of effectively mediating gene silencing. For example, in addition to the siRNA and shRNA structures described above, DICER can process ˜70 nucleotide hairpin precursors with imperfect duplex structures, i.e., duplexes that are interrupted by one or more mismatches, bulges, or inner loops within the stem of the hairpin into single-stranded RNAs called microRNAs (miRNA) that are believed to hybridize within the 3′ UTR of a target mRNA and repress translation. See, e.g., Lagos-Quintana, M. et al., Science, 294, 853-858, 2001; Pasquinelli, A., Trends in Genetics, 18(4), 171-173, 2002, and references in the foregoing two articles for discussion of miRNAs and their mechanisms of silencing.

Accordingly, the invention provides siRNA and shRNA that inhibit expression of an mRNA encoding any of the DEA polypeptides. The term “DEA RNAi agent” includes any siRNA or shRNA (or precursors thereof) that inhibits expression of a DEA mRNA transcript. An RNAi agent is considered to inhibit expression of a target transcript if the stability or translation of the target transcript is reduced in the presence of the siRNA as compared with its absence. Typically the antisense portion of an RNAi agent shows at least about 80%, preferably at least about 90%, more preferably at least about 91%, 92%, 93%, 94%, 95%, 96%, 97%, 98%, 99%, or 100% precise sequence complementarity with the target transcript for a stretch of at least about 17, more preferably at least about 18 or 19 to about 21-23 nucleotides.

The invention encompasses a method of inhibiting expression of a DEA gene in a cell or subject comprising delivering an siRNA or shRNA targeted to DEA mRNA to the cell or subject. In addition, the invention provides a method of treating a condition associated with atherosclerosis comprising steps of (i) providing a subject in need of treatment for atherosclerosis or a disease or condition associated with atherosclerosis; and (ii) administering a pharmaceutical composition comprising an effective amount of an siRNA or shRNA targeted to DEA mRNA to the subject, thereby alleviating the condition.

As mentioned above, siRNAs and shRNAs have been shown to effectively reduce gene expression when expressed intracellularly, e.g., by delivering vectors such as plasmids, viral vectors such as adenoviral, retroviral or lentiviral vectors, or viruses to cells. Such vectors, referred to herein as RNAi-inducing vectors, are vectors whose presence within a cell results in transcription of one or more RNAs that self-hybridize or hybridize to each other to form an shRNA or siRNA. In general, the vector comprises a nucleic acid operably linked to expression signal(s) so that one or more RNA molecules that hybridize or self-hybridize to form an siRNA or shRNA are transcribed when the vector is present within a cell. Thus the vector provides a template for intracellular synthesis of the RNA or RNAs or precursors thereof. The vector will thus contain a sequence or sequences whose transcription results in synthesis of two complementary RNA strands having the properties of siRNA strands described above, or a sequence whose transcription results in synthesis of a single RNA molecule containing two complementary portions separated by an intervening portion that forms a loop when the two complementary portions hybridize to one another.

Selection of appropriate siRNA and shRNA sequences can be performed according to guidelines well known in the art, e.g., taking factors such as desirable GC content into consideration. See, e.g., Ambion Technical Bulletion #506, available at the web site having URL www.ambion.com/techlib/tb/tb_{—}506.html. Following these guidelines approximately half of the selected siRNAs effectively silence the corresponding gene, indicating that by selecting about 5 siRNAs it will almost always be possible to identify an effective sequence. A number of computer programs that aid in the selection of effective siRNA/shRNA sequences are known in the art, which yield even higher percentages of effective siRNAs. See, e.g., Cui, W., et al., “OptiRNai, a Web-based Program to Select siRNA Sequences”, Proceedings of the IEEE Computer Society Conference on Bioinformatics, p. 433, 2003. Pre-designed siRNAs targeting over 95% of the mouse or human genome are commercially available, e.g, from Ambion and/or Cenix Biosciences. See web site having URL www.ambion.com/techlib/tn/104/5.html. As is known in the art, siRNAs and shRNAs can be delivered using a variety of delivery agents that increase their potency.

4. Synthesis, Delivery Methods and Modifications

Antisense nucleic acids, ribozymes, siRNAs, or shRNAs can be delivered to cells by standard techniques such as microinjection, electroporation, or transfection. Antisense nucleic acids, ribozymes, siRNAs, or shRNAs can be formulated as pharmaceutical compositions and delivered to a subject using a variety of approaches, as described further below. According to certain embodiments of the invention the delivery of antisense, ribozyme, siRNA, or shRNA molecules is accomplished via a gene therapy approach in which vectors (e.g., viral vectors such as retroviral, lentiviral, or adenoviral vectors, etc.) are delivered to a cell or subject, or cells directing expression of the molecules (e.g., cells into which a vector directing expression of the molecule has been introduced) are administered to the subject. Delivery methods are discussed further below.

It may advantageous to employ various nucleotide modifications and analogs to confer desirable properties on the antisense nucleic acid, ribozyme, siRNA, or shRNA. Numerous nucleotide analogs, nucleotide modifications, and modifications elsewhere in a nucleic acid chain are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.), referenced above, and references therein. As will be appreciated by one of ordinary skill in the art, analogs and modifications may be tested using, e.g., the assays described herein or other appropriate assays, in order to select those that effectively reduce expression of the target nucleic acid. The analog or modification preferably results in a nucleic acid with increased absorbability (e.g., increased absorbability across a mucus layer, increased oral absorption, etc.), increased stability in the blood stream or within cells, increased ability to cross cell membranes, etc.

Antisense RNAs, ribozymes, siRNAs or shRNAs may be prepared by any method known in the art for the synthesis of nucleic acid molecules. These include techniques for chemical synthesis such as solid phase phosphoramidite chemical synthesis. In the case of siRNAs, the structure may be stabilized, for example by including nucleotide analogs at one or more free strand ends in order to reduce digestion, e.g., by exonucleases. This may also be accomplished by the use of deoxy residues at the ends, e.g., by employing dTdT overhangs at each 3′ end. Alternatively, antisense, ribozyme, siRNA or shRNA molecules may be generated by in vitro transcription of DNA sequences encoding the relevant molecule. Such DNA sequences may be incorporated into a wide variety of vectors with suitable RNA polymerase promoters such as T7, T3, or SP6.

Antisense, ribozyme, siRNA or shRNA molecules may be generated by intracellular synthesis of small RNA molecules, as described above, which may be followed by intracellular processing events. For example, intracellular transcription may be achieved by cloning templates into RNA polymerase III transcription units, e.g., under control of a U6 or H1 promoter. In one approach for intracellular synthesis of siRNA, sense and antisense strands are transcribed from individual promoters, which may be on the same construct. The promoters may be in opposite orientation so that they drive transcription from a single template, or they may direct synthesis from different templates. However, it may be preferable to express a single RNA molecule that self-hybridizes to form a hairpin RNA that is then cleaved by DICER within the cell.

The antisense, ribozyme, siRNA, or shRNA molecules of the invention may be introduced into cells by any of a variety of methods. For instance, antisense, ribozyme, siRNA, or shRNA molecules or vectors encoding them can be introduced into cells via conventional transformation or transfection techniques. As used herein, the terms “transformation” and “transfection” are intended to refer to a variety of art-recognized techniques for introducing foreign nucleic acid (e.g., DNA or RNA) into a host cell, including calcium phosphate or calcium chloride co-precipitation, DEAE-dextran-mediated transfection, lipofection, injection, or electroporation.

Vectors that direct in vivo synthesis of antisense, ribozyme, siRNA, or shRNA molecules constitutively or inducibly can be introduced into cell lines, cells, or tissues. In certain preferred embodiments of the invention, inventive vectors are gene therapy vectors (e.g., adenoviral vectors, adeno-associated viral vectors, retroviral or lentiviral vectors, or various nonviral gene therapy vectors) appropriate for the delivery of a construct directing transcription of an siRNA to mammalian cells, most preferably human cells.

Preferred siRNA, shRNA, antisense, or ribozyme compositions reduce the level of a target transcript and its encoded protein by at least 2-fold, preferably at least 4-fold, more preferably at least 10-fold or more. The ability of a candidate siRNA to reduce expression of the target transcript and/or its encoded protein may readily be tested using methods well known in the art including, but not limited to, Northern blots, RT-PCR, microarray analysis in the case of the transcript, and various immunological methods such as Western blot, ELISA, immunofluorescence, etc., in the case of the encoded protein. In addition, the potential of any siRNA, shRNA, antisense, or ribozyme composition for treatment of a particular condition or disease associated with atherosclerosis may also be tested in appropriate animal models or in human subjects, as is the case for all methods of treatment described herein. Appropriate animal models include mice, rats, rabbits, sheep, dogs, etc., with experimentally induced atherosclerosis.

5. Delivery of Nucleic Acids to a Subject

The various nucleic acids described above (e.g., nucleic acids encoding DEA polypeptides, fragments, and variants; antisense oligonucleotides complementary to DEA mRNA, ribozymes designed to cleave DEA mRNA, siRNA or shRNA targeted to DEA mRNA may be delivered to a subject using any of a variety of approaches, including those applicable to non-nucleic acid agents such as IV, intranasal, oral, etc. However, according to certain embodiments of the invention the nucleic acids are delivered via a gene therapy approach, in which a construct capable of directing expression of one or more of the inventive nucleic acids is delivered to cells or to the subject (ultimately to enter cells, where transcription may occur). Thus according to certain embodiments of the invention the vectors described above include gene therapy vectors appropriate for the delivery of a construct that directs expression of a DEA polypeptide, variant, fragment, etc., or a construct directing transcription of an antisense oligonucleotide complementary to a DEA mRNA, or a ribozyme designed to cleave DEA mRNA, or an siRNA or shRNA targeted to a DEA mRNA to mammalian cells, more preferably cells of a domestricated mammal, and most preferably human cells. A variety of gene therapy vectors are known in the art. Suitable gene therapy vectors include viral vectors such as adenoviral or adeno-associated viral vectors, retroviral vectors and lentiviral vectors. In certain instances lentiviruses may be preferred due, e.g., to their ability to infect nondividing cells. See, e.g., Mautino and Morgan, AIDS Individual Care STDS 2002 January; 16(1):11-26. See also Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

A number of nonviral vectors and gene delivery systems exist, any of which may be used in the practice of the invention. For example, extrachromosomal DNA (e.g., plasmids) may be used as a gene therapy vector. See, e.g., Stoll, S. and Calor, M, “Extrachromosomal plasmid vectors for gene therapy”, Curr Opin Mol Ther, 4(4):299-305, 2002. According to one approach, the inclusion of appropriate genetic elements from various papovaviruses allows plasmids to be maintained as episomes within mammalian cells. Such plasmids are faithfully distributed to daughter cells. In particular, viral elements of various polyomaviruses and papillomaviruses such as BK virus (BKV), bovine papilloma virus 1 (BPV-1) and Epstein-Barr virus (EBV), among others, are useful in this regard. The invention therefore provides plasmids that direct expression of a DEA polypeptide, variant, fragment, etc., or a construct directing transcription of an antisense oligonucleotide complementary to a DEA mRNA, or a ribozyme designed to cleave DEA mRNA, or an siRNA targeted to a DEA mRNA to mammalian cells, preferably domesticated mammal cells, and most preferably human cells. According to certain embodiments of the invention the plasmids comprise a viral element sufficient for stable maintenance of the transfer plasmid as an episome within mammalian cells. Appropriate genetic elements and their use are described, for example, in Van Craenenbroeck, et al., Eur. J. Biochem. 267, 5665-5678 (2000) and references therein, all of which are incorporated herein by reference. Plasmids can be delivered as “naked DNA” or in conjunction with a variety of delivery vehicles.

Protein/DNA polyplexes represent an approach useful for delivery of nucleic acids to cells and subjects. These vectors may be used to deliver constructs directing transcription of the inventive nucleic acids (constructs that direct transcription of DEA polypeptides, fragments, or variants, antisense molecules, ribozymes, or siRNAs) or may be used to deliver the nucleic acids themselves. Thus their use is not limited to gene therapy. See, e.g., Cristiano, R., Surg. Oncol. Clin. N. Am., II (3), 697-715, 2002. Cationic polymers and liposomes may also be used for these purposes. See, e.g., Merdan, T., et al., “Prospects for cationic polymers in gene and oligonucleotide therapy against cancer”, Adv Drug Deliv Res, 54(5), 715-58, 2002; Liu, F. and Huang, L., “Development of non-viral vectors for systemic gene delivery”, J. Control. Release, 78(1-3):259-66, 2002; Maurer, N., et al., “Developments in liposomal drug delivery systems”, Expert Opin Biol Ther, 1(2), 201-26, 2001; and Li, S. and Ma, Z., “Nonviral gene therapy”, Curr Gene Ther, 1(2), 201-26, 2001. See Rasmussen, H., Curr Opin Mol. Ther, 4(5), 476-81, 2002 for a review of angiogenic gene therapy strategies for the treatment of cardiovascular disease. Numerous reagents and methods for gene therapy are described in Philips, I., (ed.), Methods in Enzymology, Vol. 346: Gene Therapy Methods, Academic Press, 2002.

Any of the nucleic acid delivery vehicles (or nucleic acids themselves) can be targeted for delivery to specific cells, tissues, etc. In particular, they can be targeted to cardiac cells using antibodies or ligands that specifically bind to a DEA polypeptide as discussed further below. Nucleic acids can be directly conjugated to such antibodies or ligands, which then deliver the nucleic acids to cardiac cells.

Gene therapy protocols may involve administering an effective amount of a gene therapy vector comprising a nucleic acid capable of directing expression of a DEA polynucleotide, variant, or fragment, DEA antisense nucleic acid, or a ribozyme or siRNA targeted to a DEA mRNA to a subject. Another approach that may be used alternatively or in combination with the foregoing is to isolate a population of cells, e.g., stem cells or immune system cells from a subject, optionally expand the cells in tissue culture, and administer a gene therapy vector to the cells in vitro. The cells may then be returned to the subject. Optionally, cells expressing the desired polynucleotide, siRNA, etc., can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in individuals undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

B. Methods for Increasing Gene Expression

Additional methods for identifying compounds capable of modulating gene expression are described, for example, in U.S. Pat. No. 5,976,793. These methods may be either to identify compounds that increase gene expression or to identify compounds that decrease gene expression. The screening methods described therein are particularly appropriate for identifying compounds that do not naturally occur within cells and that modulate the expression of genes of interest whose expression is associated with a defined physiological or pathological effect within a multicellular organism. Additional methods for identifying agents that increase expression of genes are found in Ho, S., et al., Nature, 382, pp. 822-826, 1996, which describes homodimeric and heterodimeric synthetic ligands that allow ligand-dependent association and disassociation of a transcriptional activation domain with a target promoter to increase expression of an operatively linked gene.

Expression can also be increased by introducing additional copies of a coding sequence into a cell of interest, i.e., by introducing a nucleic acid comprising the coding sequence into the cell. Preferably the coding sequence is operably linked to regulatory signals such as promoters, enhancers, etc., that direct expression of the coding sequence in the cell. The nucleic acid may comprise a complete DEA gene, or a portion thereof, preferably containing the coding region of the gene. The nucleic acid may be introduced into cells grown in culture or cells in a subject using any suitable method, e.g., any of those described above.

C. Identifying Agents that Modulate Expression of a DEA Gene

Agents such as antisense molecules, siRNAs, shRNAs, ribozymes, other nucleic acids, peptides or polypeptides, small molecules, etc., can be tested to determine whether they modulate the expression of a DEA gene. The invention provides a method for identifying an agent that modulates expression of a DEA polynucleotide or polypeptide comprising steps of: (i) providing a sample comprising cells that express a DEA polynucleotide or polypeptide; (ii) contacting the cells with a candidate agent; (iii) determining whether the level of expression of the polynucleotide or polypeptide in the presence of the compound is increased or decreased relative to the level of expression or activity of the polynucleotide or polypeptide in the absence of the compound; and (iv) identifying the compound as a modulator of the DEA polynucleotide or polypeptide if the level of expression or activity of the DEA polynucleotide or polypeptide is higher or lower in the presence of the compound relative to its level of expression or activity in the absence of the compound.

Expression of a DEA polynucleotide or polypeptide can be measured using a variety of methods well known in the art in order to determine whether any candidate agent increases or decreases expression (or for other purposes). In general, any measurement technique capable of determining RNA or protein presence or abundance may be used for these purposes. For RNA such techniques include, but are not limited to, microarray analysis (For information relating to microarrays and also RNA amplification and labeling techniques, which may also be used in conjunction with other methods for RNA detection, see, e.g., Lipshutz, R., et al., Nat Genet., 21(1 Suppl):20-4, 1999; Kricka L., Ann. Clin. Biochem., 39(2), pp. 114-129; Schweitzer, B. and Kingsmore, S., Curr Opin Biotechnol 2001 February; 12(1):21-7; Vineet, G., et al., Nucleic Acids Research, 2003, Vol. 31, No. 4; Cheung, V., et al., Nature Genetics Supplement, 21:15-19, 1999; Methods Enzymol, 303:179-205, 1999; Methods Enzymol, 306: 3-18, 1999; M. Schena (ed.), DNA Microarrays: A Practical Approach, Oxford University Press, Oxford, UK, 1999. See als U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; 6,235,483; WO 93/17126; WO 95/11995; WO 95/35505; EP 742 287; EP 799 897; 5,514,545; 5,545,522; 5,716,785; 5,932,451; 6,132,997; 6,235,483; US Patent Application Publication 20020110827).

Other methods for detecting expression of DEA polynucleotides include Northern blots, RNAse protection assays, reverse transcription (RT)-PCR assays, real time RT-PCR (e.g., Taqman™ assay, Applied Biosystems), SAGE (Velculescu et al. Science, vol. 270, pp. 484-487, October 1995), Invader® technology (Third Wave Technologies), etc. See, e.g., E is, P. S. et al., Nat. Biotechnol. 19:673 (2001); Berggren, W. T. et al., Anal. Chem. 74:1745 (2002), etc. Methods for detecting DEA polypeptides include, but are not limited to, immunoblots (Western blots), immunofluorescence, flow cytometry (e.g., using appropriate antibodies), mass spectrometry, and protein microarrays (Elia, G., Trends Biotechnol, 20(12 Suppl):S19-22, 2002, and reference therein).

D. Reagents and Methods for Modulating Functional Expression or Activity of a DEA Polypeptide

As discussed above, the invention provides methods for identifying ligands that modulate (e.g., increase or decrease) activity of a DEA polypeptide and methods for identifying agents that modulate expression of a DEA polynucleotide or polypeptide. More generally, the invention also provides a method for identifying an agent that modulates expression or activity of a DEA polynucleotide or polypeptide comprising steps of: (i) providing a sample comprising a DEA polynucleotide or polypeptide; (ii) contacting the sample with a candidate compound; (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the expression or activity of the DEA polynucleotide or polypeptide if the level of expression or activity of the DEA polynucleotide or polypeptide is higher or lower in the presence of the compound relative to its level of expression or activity in the absence of the compound. In certain embodiments of the method the sample comprises cells that express the DEA polypeptide. The agents to be screened include any of those discussed above. Agents identified according to the above methods may be further tested in subjects, e.g., humans or other animals. The subject may be normal or may be suffering from or at risk of atherosclerosis of a condition or disease associated with atherosclerosis. The test may involve determining whether administration of the agent reduces or alleviates one or more symptoms or signs of atherosclerosis or improves a prognostic variable such as exercise capacity.

The invention further provides a method for identifying an agent that modulates expression or activity of a DEA polynucleotide or polypeptide comprising steps of: (i) providing a sample comprising a DEA polynucleotide or polypeptide; (ii) contacting the sample with a candidate compound; (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the expression or activity of the DEA polynucleotide or polypeptide if the level of expression or activity of the DEA polynucleotide or polypeptide is higher or lower in the presence of the compound relative to its level of expression or activity in the absence of the compound. The method may further include the step of identifying the agent as being useful for treatment and/or prevention of atherosclerosis.

The invention also provides a method for identifying a therapeutic agent for the treatment and/or prevention of atherosclerosis or a disease or condition associated with atherosclerosis comprising the step of: identifying an agonist or antagonist of a polynucleotide or polypeptide encoded by a DEA gene. The agonist or antagonist is identified according to any appropriate screening assay. One of ordinary skill in the art will be able to select an appropriate screening assay taking into consideration any available information about the biochemical and/or functional activity of the product encoded by the DEA gene.

VII. Diagnostic Applications

Genes identified as upregulated or downregulated in atherosclerosis serve as diagnostic targets. The invention therefore provides a method for providing diagnostic or prognostic information related to atherosclerosis or to a disease or condition associated with atherosclerosis comprising steps of: (i) providing a subject in need of diagnostic or prognostic information related to atherosclerosis or to a disease or condition associated with atherosclerosis; and (ii) determining the level of expression or activity of a DEA polynucleotide or polypeptide in the subject or in a biological sample obtained from the subject. The method may further comprise the step of (iii) comparing the determined level of expression or activity with known level(s) determined previously in the subject or in normal subjects or in subjects with atherosclerosis, or in a biological sample obtained from the subject or from normal subjects or from subjects with atherosclerosis. The determined level of expression or activity can be correlated with values that have been associated with particular diagnostic categories (e.g., American Heart Association Classification of atherosclerosis), disease outcomes, likelihood of responding positively to particular treatments, time to progression to a more severe state, etc. The information can be provided to the subject and/or used to guide therapeutic decisions, e.g., the advisability of initiating or terminating various therapies, etc. By “normal subject” is meant a subject not suffering from atherosclerosis or from a disease or clinical condition associated with atherosclerosis as determined using a classification method accepted in the art. The classification method may be based on clinical criteria, laboratory criteria, qualitative and/or quantitative tests including imaging tests, etc.

According to certain embodiments of the invention, a level of expression or activity of a DEA polynucleotide or polypeptide that is higher than would be expected in a normal subject or in a biological sample obtained from a normal subject, indicates an increased likelihood that the subject is at risk of or suffering from atherosclerosis or a disease or condition associated with atherosclerosis. A level of expression or activity of a DEA polynucleotide or polypeptide that is higher in the subject or in a biological sample obtained from the subject than the level determined previously for that subject indicates that the subject's disease has become more severe and/or that the subject has not responded to therapy. According to certain embodiments of the invention the level of expression of a DEA polynucleotide or polypeptide is an indicator of the severity of atherosclerosis or of a disease or condition associated with atherosclerosis, with a higher level, e.g., relative to normal being indicative of greater severity.

According to certain embodiments of the invention, a level of expression or activity of a DEA polynucleotide or polypeptide that is lower than would be expected in a subject with atherosclerosis or in a biological sample obtained from a subject with atherosclerosis, indicates a decreased likelihood that the subject is at risk of or suffering from atherosclerosis or a disease or condition associated with atherosclerosis. A level of expression or activity of a DEA polynucleotide or polypeptide that is lower in the subject or in a biological sample obtained from the subject than the level determined previously for that subject indicates that the subject's disease has become less severe and/or that the subject has responded to therapy. According to certain embodiments of the invention the level of expression of a DEA polynucleotide or polypeptide is an indicator of the severity of atherosclerosis or of a disease or condition associated with atherosclerosis, with a lower level, e.g., relative to that typically found in atherosclerosis, being indicative of lower severity.

According to certain embodiments of the invention, a level of expression or activity of a DEA polynucleotide or polypeptide that is lower than would be expected in a normal subject or in a biological sample obtained from a normal subject, indicates an increased likelihood that the subject is at risk of or suffering from atherosclerosis or a disease or condition associated with atherosclerosis. A level of expression or activity of a DEA polynucleotide or polypeptide that is lower in the subject or in a biological sample obtained from the subject than the level determined previously for that subject indicates that the subject's disease has become more severe and/or that the subject has not responded to therapy. According to certain embodiments of the invention the level of expression of a DEA polynucleotide or polypeptide is an indicator of the severity of atherosclerosis or of a disease or condition associated with atherosclerosis, with a lower level, e.g., relative to normal being indicative of greater severity.

According to certain embodiments of the invention, a level of expression or activity of a DEA polynucleotide or polypeptide that is higher than would be expected in a subject with atherosclerosis or in a biological sample obtained from a subject with atherosclerosis, indicates a decreased likelihood that the subject is at risk of or suffering from atherosclerosis or a disease or condition associated with atherosclerosis. A level of expression or activity of a DEA polynucleotide or polypeptide that is higher in the subject or in a biological sample obtained from the subject than the level determined previously for that subject indicates that the subject's disease has become less severe and/or that the subject has responded to therapy. According to certain embodiments of the invention the level of expression of a DEA polynucleotide or polypeptide is an indicator of the severity of atherosclerosis or of a disease or condition associated with atherosclerosis, with a higher level, e.g., relative to that found in subjects with atherosclerosis, being indicative of lesser severity.

In any of the foregoing methods the level of expression of an expression product (e.g., an RNA transcribed from a gene or a polypeptide encoded by such an RNA) can be determined according to standard methods, some of which are described elsewhere herein. For example, a sample of cardiac tissue (cardiac biopsy) can be obtained. Such biopsies are routinely performed, e.g., to assess rejection following cardiac transplant. Endocardial or myocardial biopsies can be done using a catheter inserted into the heart via the jugular vein. RNA can be detected using in situ hybridization or extracted and measured, optionally being amplified prior to measurement. RT-PCR can be used. Protein expression can be measured using various immunological techniques including immunohistochemistry, immunoblot, immunoassays such as ELISA assays, etc.

Rather than determining the level of expression of a polynucleotide or polypeptide, in certain embodiments of the invention the functional activity of the polypeptide is measured. For example, in the case of a kinase, kinase activity can be measured. Methods for doing so are well known in the art and can utilize either endogenous substrates or synthetic substrates, e.g., substrates containing consensus sequences for phosphorylation for either serine/threonine or tyrosine kinases. Activity of other polypeptides having known biological and/or enzymatic activities can be measured using any of a variety of methods known in the art, as appropriate for the particular activity.

Instead of determining the expression level or activity of a polynucleotide or polypeptide in a sample obtained from a subject, the expression level can be measured using imaging as described above. Activity can also be measured using imaging techniques, e.g., by targeting a substrate for an enzymatic reaction catalyzed by the polypeptide to cardiac cells and monitoring conversion of the substrate into product by performing sequential imaging. Labeled substrates can be used to facilitate such monitoring. Methods for performing functional imaging, either invasively or noninvasively, are known in the art.

In the case of certain diagnostic targets, the polypeptide encoded by the gene is secreted from cells and circulates in the bloodstream. In such cases the level of expression or activity of the gene product can be measured in a blood or serum sample obtained from the subject. Polypeptides that are secreted by cells typically include a signal sequence that directs their secretion. In addition, certain of the gene products encode receptors. The invention also provides diagnostic methods based on the measurement of levels of endogenous ligands for these receptors. According to certain embodiments of the invention the level of an endogenous ligand for a DEA polypeptide is measured instead of or in addition to the level of expression or activity of the corresponding DEA polypeptide. wherein the level of the ligand correlates with disease severity in atherosclerosis. The level of the ligand can be measured using any suitable method, e.g., radioimmunoassay, ELISA, functional assays, etc.

Thus the invention provides a method for providing diagnostic or prognostic information related to atherosclerosis or to a disease or condition associated with atherosclerosis comprising steps of: (i) providing a subject in need of diagnostic or prognostic information related to atherosclerosis or to a disease or condition associated with atherosclerosis; and (ii) determining the level of a ligand for a DEA polypeptide in the subject or in a biological sample obtained from the subject. The method may further comprise the step of (iii) comparing the determined level with known level(s) determined previously in the subject or in normal subjects or in subjects with atherosclerosis, or in a biological sample obtained from the subject or from normal subjects or from subjects with atherosclerosis. The determined level of the ligand can be correlated with values that have been associated with particular diagnostic categories (e.g., in accordance with American Heart Association histological classification of atherosclerosis lesions as Grade I-V), disease outcomes, likelihood of responding positively to particular treatments, time to progression to a more severe state, etc. The information can be provided to the subject and/or used to guide therapeutic decisions, e.g., the advisability of initiating or terminating various therapies, etc.

According to certain embodiments of the invention, a level of expression or activity of a ligand for a DEA polypeptide that is higher than would be expected in a normal subject or in a biological sample obtained from a normal subject, indicates an increased likelihood that the subject is at risk of or suffering from atherosclerosis or a disease or condition associated with atherosclerosis. A level of ligand for a DEA polynucleotide or polypeptide that is higher in the subject or in a biological sample obtained from the subject than the level determined previously for that subject indicates that the subject's disease has become more severe and/or that the subject has not responded to therapy. According to certain embodiments of the invention the level of a ligand for a DEA polypeptide is an indicator of the severity of atherosclerosis or of a disease or condition associated with atherosclerosis, with a higher level, e.g., relative to normal being indicative of greater severity.

According to certain embodiments of the invention, a level of a ligand for a DEA polypeptide that is lower than would be expected in a subject with atherosclerosis or in a biological sample obtained from a subject with atherosclerosis, indicates a decreased likelihood that the subject is at risk of or suffering from atherosclerosis or a disease or condition associated with atherosclerosis. A level of a ligand for a DEA polypeptide that is lower in the subject or in a biological sample obtained from the subject than the level determined previously for that subject indicates that the subject's disease has become less severe and/or that the subject has responded to therapy. According to certain embodiments of the invention the level of a ligand for a DEA polypeptide is an indicator of the severity of atherosclerosis or of a disease or condition associated with atherosclerosis, with a lower level, e.g., relative to that typically found in atherosclerosis, being indicative of lower severity.

According to certain embodiments of the invention, a level of a ligand for a DEA polypeptide that is lower than would be expected in a normal subject or in a biological sample obtained from a normal subject, indicates an increased likelihood that the subject is at risk of or suffering from atherosclerosis or a disease or condition associated with atherosclerosis. A level of a ligand for a DEA polypeptide that is lower in the subject or in a biological sample obtained from the subject than the level determined previously for that subject indicates that the subject's disease has become more severe and/or that the subject has not responded to therapy. According to certain embodiments of the invention the level of a ligand for a DEA polypeptide is an indicator of the severity of atherosclerosis or of a disease or condition associated with atherosclerosis, with a lower level, e.g., relative to normal being indicative of greater severity.

According to certain embodiments of the invention, a level of a ligand for a DEA polypeptide that is higher than would be expected in a subject with atherosclerosis or in a biological sample obtained from a subject with atherosclerosis, indicates a decreased likelihood that the subject is at risk of or suffering from atherosclerosis or a disease or condition associated with atherosclerosis. A level of a ligand for a DEA polypeptide that is higher in the subject or in a biological sample obtained from the subject than the level determined previously for that subject indicates that the subject's disease has become less severe and/or that the subject has responded to therapy. According to certain embodiments of the invention the level of a ligand for a DEA polypeptide is an indicator of the severity of atherosclerosis or of a disease or condition associated with atherosclerosis, with a higher level, e.g., relative to that found in subjects with atherosclerosis, being indicative of lesser severity.

As a particular example, the invention provides a method of providing diagnostic or prognostic information related to atherosclerosis or to a disease or condition associated with atherosclerosis comprising steps of: (i) providing a subject in need of diagnostic or prognostic information related to atherosclerosis or to a disease or condition associated with atherosclerosis; and (ii) determining the level of a DEA polypeptide in the subject or in a biological sample obtained from the subject. The method may further comprise the step of (iii) comparing the determined level with known level(s) determined previously in the subject or in normal subjects or in subjects with atherosclerosis, or in a biological sample obtained from the subject or from normal subjects or from subjects with atherosclerosis. The sample, can be, e.g., a blood, plasma, or serum sample in certain embodiments of the invention. The measurement can be performed, using for example, a radioimmunoassay or ELISA, etc. In certain embodiments of the invention the DEA polypeptide is selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

VIII. Therapeutic Applications

As discussed above, the discovery that expression of DEA genes is upregulated or downregulated in atherosclerosis suggests that these genes and their expression products are appropriate targets for treatment or prevention of atherosclerosis and diseases and clinical conditions associated with atherosclerosis (including, but are not limited to hypertension, restenosis, ischemic cardiovascular diseases, ischemic cerebrovascular disease, diabetes, peripheral arterial disease, etc.). Thus the invention provides a method for treating atherosclerosis or a disease or clinical condition associated with atherosclerosis comprising: (i) providing a subject at risk of or suffering from a disease or clinical condition associated with atherosclerosis; and (ii) administering a compound that modulates expression or activity of a DEA polynucleotide or polypeptide to the subject. The compounds can be administered prophylactically. In certain embodiments of the invention the DEA polypeptide is encoded by a gene selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

The invention further provides a method for treating atherosclerosis or a disease or clinical condition associated with atherosclerosis comprising: (i) providing a subject at risk of or suffering from a disease or clinical condition associated with atherosclerosis; and (ii) administering a compound that modulates an endogenous ligand for a DEA polypeptide to the subject. By “modulate” is meant to enhance or reduce the level or activity of a molecule or to alter the temporal or spatial pattern of its expression or activity, in various embodiments of the invention. For example an agent that acts as an agonist or antagonist at a particular receptor is considered to modulate the receptor. The compounds can be administered prophylactically. In certain embodiments of the invention the DEA polypeptide is encoded by a gene selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

A variety of methods of modulating the expression or activity of DEA gene expression products and/or ligands are provided above. Any of the agents identified according to such methods may be used to modulate expression or activity of the DEA gene expression products and/or ligands for therapeutic or other purposes.

The invention provides a method for treating atherosclerosis or a disease or clinical condition associated with atherosclerosis comprising: (i) providing a subject at risk of or suffering from a disease or clinical condition associated with atherosclerosis; and (ii) administering a conjugate comprising a DEA targeting agent and a therapeutic agent to the subject. The invention also provides a method for treating atherosclerosis or a disease or clinical condition associated with atherosclerosis comprising: (i) providing a subject at risk of or suffering from a disease or clinical condition associated with atherosclerosis; and (ii) administering a delivery vehicle comprising a DEA targeting agent and a therapeutic agent to the subject. Any of the conjugates or delivery vehicles described above can be used.

A variety of different therapeutic agents can be used in the conjugates or delivery vehicles of the invention. In certain embodiments the therapeutic agent is an anti-inflammatory agent. Nonlimiting examples of anti-inflammatory agents of use in the invention include aspirin, non-steroidal anti-inflammatory agents (e.g, COX-1 and/or COX-2 inhibitors), corticosteroids, an antibody that binds to TNF-α (e.g., infliximab, Remicade®), a polypeptide that is a soluble TNF-α receptor (e.g., etanercept; Enbrel®), anti-cytokine antibodies, cytokine antagonists, anti-inflammatory cytokines, gold; penicillamine; chloroquine; hydroxychloroquine; chlorambucil; cyclophosphamide; cyclosporine, etc.

The invention further provides a method for treating atherosclerosis or a disease or clinical condition associated with atherosclerosis comprising: (i) providing a subject at risk of or suffering from a disease or clinical condition associated with atherosclerosis; and (ii) administering an agonist or antagonist of a DEA polypeptide to the subject.

IX. Pharmaceutical Compositions and Kits

The invention provides a variety of compositions, e.g., pharmaceutical compositions. For example, the invention provides compositions, e.g., pharmaceutical compositions, containing DEA antisense nucleic acids, DEA RNAi agents, DEA ribozymes, or vectors for endogenous expression of one or more of these nucleic acids. The invention further provides a composition comprising an effective amount of an antibody that specifically binds to a DEA polypeptide and a pharmaceutically acceptable carrier. The invention further provides a composition comprising an effective amount of a ligand that specifically binds to a DEA polypeptide, and a pharmaceutically acceptable carrier. The antibodies and ligands may be conjugated with any of the therapeutic agents discussed above. The invention further provides a composition comprising a conjugate comprising a DEA targeting agent and a therapeutic agent. The invention further provides a composition comprising a delivery vehicle comprising a DEA targeting agent and a therapeutic agent.

Compositions containing antibodies, ligands, conjugates, antisense nucleic acids, siRNA, shRNA, ribozymes, vectors for endogenous expression of nucleic acids such as siRNAs, shRNAs, ribozymes, antisense molecules, peptides, and/or small molecules or other therapeutic agents as described herein may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include parenteral, transmucosal, rectal, and vaginal. Inventive pharmaceutical compositions typically include one or more therapeutic agents, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Compositions can also be delivered directly to a site of tissue injury or surgery. They may be administered by catheter or using diagnostic/therapeutic equipment such as bronchoscopes, colonoscopes, endoscopes, laparoscopes, etc. Inventive compositions may also be delivered as implants or components of implantable devices. For example, inventive compositions may be used to coat stents and/or vascular grafts. In certain embodiments of the invention the composition is used to coat a drug-eluting stent or other implantable or indwelling device such as a catheter, PIC line, shunt, pacemaker, defibrillator, artificial valve, etc. See, e.g., U.S. Pat. Nos. 6,517,889; 6,273,913; 6,258,121; 6,251,136; 6,248,127; 6,231,600; 6,203,551; 6,153,252; 6,071,305; 5,891,507; 5,837,313 and published U.S. patent application No.: US2001/0027340 for descriptions of stents and various implantable devices that can be coated with the compositions of the invention. Such coated devices and methods of using them to treat a subject are an additional aspect of the invention.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive therapeutic agents are preferably delivered in the form of an aerosol spray from pressured container or dispenser which contains a suitable propellant, e.g., a gas such as carbon dioxide, or a nebulizer. It is noted that the lungs provide a large surface area for systemic delivery of therapeutic agents. The agents may be encapsulated, e.g., in polymeric microparticles such as those described in U.S. publication 20040096403, or in association with any of a wide variety of other drug delivery vehicles that are known in the art. In other embodiments of the invention the agents are delivered in association with a charged lipid as described, for example, in U.S. publication 20040062718. It is noted that the latter system has been used for administration of a therapeutic polypeptide, insulin, demonstrating the utility of this system for administration of peptide agents.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, mass spectrometry, etc.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with a therapeutic agent as described herein, can include a single treatment or, in many cases, can include a series of treatments.

Exemplary doses include milligram or microgram amounts of the inventive therapeutic agent per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses of a therapeutic agent depend upon the potency of the agent, and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

Inventive pharmaceutical compositions can be included in a container, pack, or dispenser together with instructions for administration.

Also provided are kits containing any one or more of the polynucleotides, polypeptides, specific binding agents such as antibodies, etc., described herein. The kit may further include instructions for use and/or any of a variety of other reagents including, e.g., a control sample, a control antibody, a buffer, a wash solution, substrate, etc. The reagents may be provided in one or more vessels or containers, optionally enclosed within a larger container for convenient commercial sale.

X. Computer-Readable Medium

The invention includes a computer-readable medium (e.g., a hard disk, floppy disk, compact disk, zip disk, flash memory, magnetic memory, etc.) that stores information related to any of the genes, polypeptides, and/or methods described above. The information may be organized in the form of a database, i.e., a collection of data that is organized so that its contents can easily be accessed, managed and updated. The information may identify one or more genes that are listed in Table 1-4 or 8 or mentioned herein. The information may indicate the nature of the conditions or samples in which differential expression was observed, may identify genes whose expression is altered by administration of an agent such as a statin, aspirin, or other therapeutic agent or candidate therapeutic agent, etc. The genes may be listed in order or ranked, e.g., according to the significance of their differential regulation. The computer-readable medium may store information identifying genes that are not differentially regulated, provided that it also includes information pertaining to genes that are differentially regulated and identifies those genes as being relevant to CAD, diabetes, atherosclerosis, etc. Additional information related to the gene(s) and/or to their role in CAD, diabetes, atherosclerosis or the diagnosis, treatment or prevention thereof can be included, e.g., (i) quantitative information related to the extent to which the gene(s) is/are differentially regulated and/or its significance; (ii) information identifying a biological pathway or process enriched in one or more of the genes; (iii) results obtained by administering an agent that modulates expression or activity of one or more of the genes to a subject, etc. The invention also includes a method comprising the step of electronically sending or receiving any of the afore-mentioned information and, optionally, storing at least part of the information and/or creating a new computer-readable medium or copy containing at least part of the information.

### EXEMPLIFICATION

### Example 1

### Identification of Genes that are Differentially Expressed in Atherosclerosis

Materials and Methods

The following materials and methods were employed in all the examples described below.

Development of the Custom Vascular Wall Microarray

Human aortic smooth muscle cells (HASMC) and human aortic endothelial cells (HAEC) (Clonetics, San Diego, Calif.) were serum starved and stimulated separately with 10 ng/cc TNF-α (R&D Systems, Minneapolis, Minn.). HASMC were also stimulated with 3 ng/cc TGF-β (R&D Systems) and 20 ng/cc PDGF-BB (R&D Systems). Cells collected at 30 minute, 3 hour, and 24 hour time points were pooled, and poly(A)⁺ RNA isolated, and suppression subtraction performed in both directions as described (Ho, M., et al., Physiol Genomics, 13: 249-262, 2003). A total of 6954 cDNAs were cloned into plasmid, miniprepped, sequenced, and matched to Genbank accession numbers which were collapsed into Unigene clusters and RefSeq annotation applied where possible. In addition, a set of 384 endothelial cell-restricted genes were identified by searching publicly available gene expression databases, and 138 monocyte/macrophage, T cell, and B cell genes were selected on the basis of their role in inflammation or immune function (Ho, M., et al., supra). IMAGE clones for these genes were purchased (Research Genetics, Carlsbad, Calif.) and sequence verified. All cDNA clones were amplified by polymerase chain reaction (PCR) and then printed on glass slides (Agilent Technologies, Inc., Palo Alto, Calif.).

Human Tissue Sample Collection

Major epicardial coronary arteries were removed from explanted hearts of patients undergoing orthotopic heart transplantation. The vessels were dissected longitudinally to expose the endoluminal surface and lesions identified and scored by inspection through a dissecting microscope. Arteries were divided into 1.0-2.0 cm normal (disease-free) or diseased segments. RNA was isolated from tissue samples and tissue-cultured cells and labeled as per established methodology (Ho, M., et al., supra). Reference RNA was composed of a mixture of 5 μg total human umbilical vein endothelial cell RNA and 5 μg total HeLa cell RNA. This study was approved by the Institutional Review Board of Stanford University.

RNA Isolation and Array Hybridization

RNA was isolated from tissue samples and tissue-cultured cells as per established methodology ((Ho, M., et al., supra). RNA quality was assessed by using the RNA 6000 Nano Chip and Bioanalyzer (Agilent Technologies, Palo Alto, Calif.). Reference RNA, composed of a mixture of 5 μg total HUVEC (Clonetics, San Diego, Calif.) RNA and 5 μg total HeLa (American Type Culture Collection, Manassas, Va.) RNA, was primed and labeled with Cy3-dCTP during reverse transcription. 10 μg of total sample RNA was primed and labeled with Cy5-dCTP. Labeled cRNAs were purified, and employed in array hybridization as described previously (Ho, M., et al., supra).

Data Analysis

Microarrays were scanned on an Agilent G2565AA Microarray Scanner System and images were quantified using Agilent Feature Extraction Software (Version A.6.1.1). Local background subtraction was performed and a LOWESS algorithm used for data normalization. Significance Analysis of Microarrays (SAM) software was used for data analysis (available at the web site having URL www-stat.stanford.edu/˜tibs/SAM/) (Tusher, V. G., et al., Proc Natl Acad Sci USA 98, 5116-21, 2001). Microarray data was also analyzed with the Threshold Number of Misclassifications (TNoM), a non-parametric score representing how well a gene separates two sample classes (Ho, M., et al., supra; Ben-Dor, A., et al., in Proceedings of the Fifth International Conference on Computational Biology, pp. 31-38, 2001). To simplify presentation, gene lists were collapsed at the level of accession number by listing only once, in order of first appearance. In an alternative strategy, accession numbers were collapsed by calculating a mean value across multiple probes for each accession number, and data analysis conducted on the collapsed data. Both strategies generated similar results.

The lists of informative genes were further analyzed using gene ontology (GO) annotation (available at the web site having URL www.geneonltology.org), to identify molecular functions and processes that were over- or under-represented among the most significant genes. Molecular function, cellular component and biological process descriptions of the genes were obtained using the Biomolecule Naming Service (BNS), which links to publicly available functional annotation. BNS was developed at Agilent Laboratories and is available at the web site having URL openbns.sourceforge.net. The analysis was performed separately for several GO terms including inflammatory response, immune response, interleukin, cytokine, chemotaxis, growth factor, etc. Lists of genes for these analyses were determined by the TNoM score and an FDR cutoff of 0.05. For each GO term t of interest, we counted the number of genes in the list annotated by t and compared this number to the overall representation of t. The statistical significance of the observed difference is reported as the associated p-value.

Results

A total of 103 human coronary artery samples were collected, along with clinical information, from 17 patients at the time of orthotopic heart transplantation (Table 5). Total RNA isolated from these samples was used for hybridization to the custom cDNA microarray. Differences in gene expression between normal (36/103 samples) and diseased (67/103) blood vessel segments were studied by performing an unpaired, two-class analysis with SAM and by determining the TNoM score (Ho, M., et al., supra). When a false detection rate (FDR) of <0.05 was used as a cutoff, SAM identified 443 probes while TNoM generated an overlapping list of 787 probes that were differentially regulated between diseased and non-diseased vascular samples (see Table 1).

As noted above, a large number of genes were identified for the first time in association with CAD, including a novel matrix metalloproteinase, MMP-10, and a number of other genes. Other genes that were identified as being upregulated in atherosclerosis included matrix metalloproteinases MMP-1, MMP-2, MMP-3, macrophage scavenger receptor-1, and tissue type plasminogen activator. Certain of these genes have previously been shown to be differentially regulated in atherosclerosis.

Most prominent among the classes of genes identified were those involved in inflammation. Genes encoding a variety of cytokines were identified. These included the CD4⁺ TH1 pro-inflammatory cytokine interferon γ, the related cytokine interleukin (IL)-18, and IL-1α. Potent chemokines which mediate leukocyte trafficking, such as IL-8 and RANTES, were also found to be upregulated. To determine whether inflammatory genes were more highly represented among the up-regulated probes identified by TNoM score in diseased samples, an overabundance analysis was performed comparing gene ontology (GO) annotation for these probes versus probes found not to be differentially regulated (Ashburner, M., Nat Genet 25, 25-9, 2000). This novel analytical approach allowed a rigorous assessment of differentially regulated signaling pathways. The composite category “inflammation,” which included GO terms immune response, defense response, inflammatory response, chemotaxis, and interferon, was significantly over-represented in the identified group of probes (p<0.005). Other specific terms found to be over-represented in this group included “cytokine” (p<0.001) and “chemokine” (p<0.05).

Table 6 presents results of the gene ontology analyses. Analyses evaluated included diseased vs. non-diseased vessels (Lesion status), diabetic vs. non-diabetic vessels (Diabetes status), diabetes vs. non-diabetes analysis with normal vessels (Diabetes status-normal vessels), statin therapy analysis with all samples (Statin therapy), and statin therapy analysis with diabetic vessels (Statin therapy-diabetic vessels). Upward and downward arrows indicate terms that were significantly overrepresented (p<0.05) or underrepresented (p<0.05), respectively. The composite category “inflammation,” included GO terms immune response, defense response, inflammatory response, chemotaxis, and interferon.

TABLE 5
Characteristics of the patient sample group.
Clinical data

| | | |
|---|---|---|
| Patient characteristics | Age (yrs.) | 52.5 ± 15.0 |
| | Male/Female | (13/4) |
| Risk factors, n (%) | Coronary Artery Disease | 8 (47.1%) |
| | Diabetes (type 2) | 5 (29.4%) |
| | Family History of CAD | 3 (17.6%) |
| | History of Tobacco Use | 11 (64.7%) |
| | Hypercholesterolemia | 7 (41.2%) |
| | Hypertension | 4 (23.5%) |
| Medications, n (%) | ACE Inhibitors/ARBs | 13 (76.5%) |
| | Aspirin | 5 (29.4%) |
| | Beta Blockers | 9 (52.9%) |
| | Diuretics | 14 (82.3%) |
| | Insulin | 2 (11.8%) |
| | Nitrates | 4 (23.5%) |
| | Statins | 7 (41.2%) |

TABLE 6
Statistical analysis of the distribution of terms relating to inflammatory
pathways.

| | | | Diabetes | | |
|---|---|---|---|---|---|
| | | | status- | | Statin therapy- |
| | Lesion | Diabetes | normal | Statin | diabetic |
| GO terms | status | status | vessels | therapy | vessels |
| inflammation | ↑ | | ↑ | ↓ | ↓ |
| cytokine | ↑ | ↑ | | | ↓ |
| interleukin | | ↑ | | ↓ | ↓ |
| chemokine | ↑ | | | | ↓ |
| interferon | ↑ | | | | |

### Example 2

### Identification of Genes that are Differentially Expressed in Atherosclerotic or Normal Blood Vessels in Diabetic Individuals

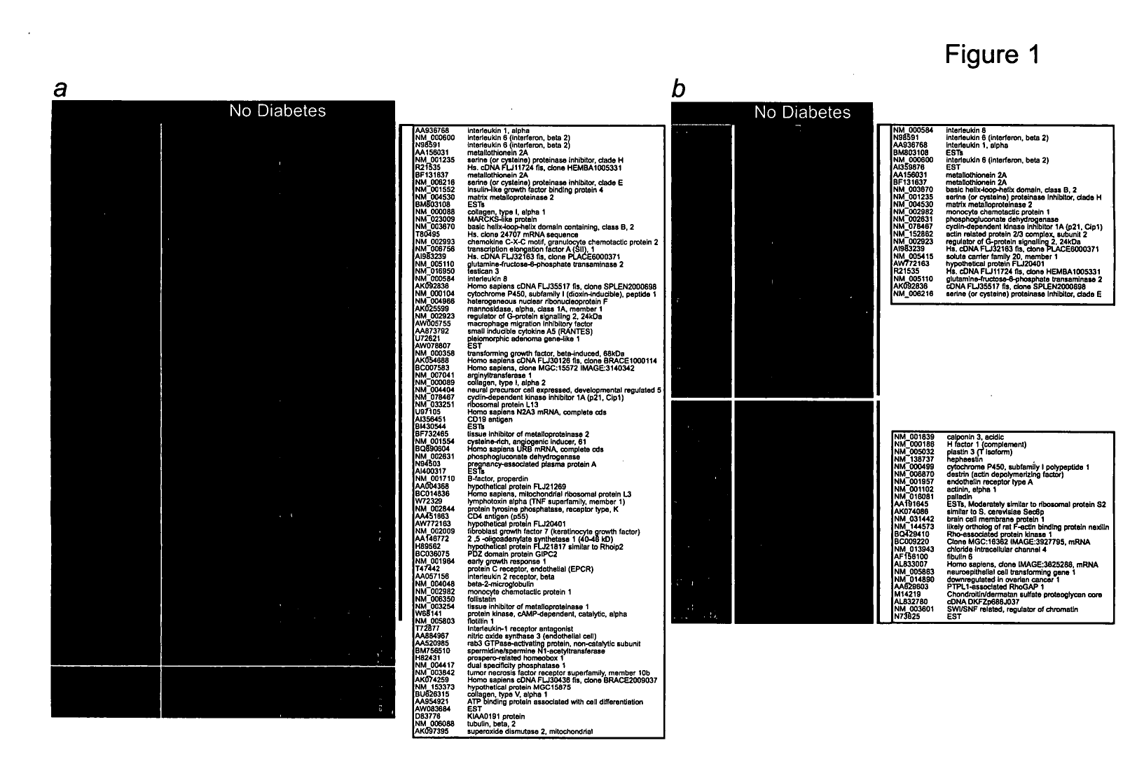

The influence of cardiovascular risk factors on vascular wall gene expression was evaluated with SAM and the TNoM score. When we analyzed gene expression differences for all the major risk factors (see Table 5), the diabetes analysis yielded the most dramatic results. When transcriptional profiles were compared between diabetic (34/103) and non-diabetic samples (69/103), SAM identified 1215 differentially expressed probes (FDR 0.04). A similar group of 1630 differentially regulated probes was found using TNoM score (FDR 0.05). A heatmap and partial gene list representing the 342 most differentially regulated genes identified by SAM (FDR<0.005) are shown (FIG. 1a, Table 2). Genes encoding cell surface receptors, signaling molecules, and matrix components were newly identified as potential vascular disease markers. For example CXCL6 was among the genes identified as a vascular disease marker in this analysis. Cytokine-responsive genes identified included matrix remodeling factor MMP2, tissue inhibitor of metalloproteinase (TIMP-1), and TIMP-1. Higher expression of immune cell genes specific for B cells (CD19, properdin) and T cells (CD4) in diabetic vascular samples suggested increased infiltration of these cell types in this subset of patients. A number of novel cytokine genes and genes encoding immune response factors were more highly expressed in samples from diabetics as shown in Table 2. Granulocyte chemotactic protein 2 (CXCL6), a factor known to mediate granulocyte migration by binding to the IL-8 receptor but not previously associated with CAD, was expressed at much higher levels in diabetic arteries. Differentially expressed inflammatory genes included cytokines IL-6 and IL-1a, chemokines IL-8, RANTES, macrophage chemoattractant protein (MCP-1), and lymphokine macrophage migration inhibitory factor. Statistical analysis using GO annotation identified “interleukin” and “cytokine” as terms that were over-represented in the TNoM group of differentially expressed probes (p<0.05) (Table 6).

To further characterize the inflammatory transcriptional profile observed in diabetic samples, analyses were restricted to diseased or normal tissues. When diseased samples from diabetics were compared to diseased samples from non-diabetics, higher-level expression of cytokine and cytokine-responsive genes was observed in the diabetic group (Table 3). Surprisingly, when the analysis was limited to normal, non-diseased vascular samples, the diabetic group was again found to express higher levels of cytokine and cytokine-responsive genes (FIG. 1b and Table 4). This list included cytokines IL-6 and IL-1α, chemokines IL-8 and MCP-1, and prominent cytokine-responsive genes such as the adhesion molecule ICAM-2 and MMP-2 (FIG. 1b). Analysis of GO nomenclature associated with probes identified by TNoM score at an FDR of 0.05 revealed overrepresentation of the grouping of “inflammatory” terms (p<0.05) (Table 6).

Our results provide the strongest evidence to date linking diabetes, a major clinical risk factor for CAD, to the activation of an inflammatory transcriptional program in the vessel wall. Our studies are of particular significance since they provide direct evidence of the activation of inflammatory signaling pathways in a human study. Our statistical analysis of diabetic coronary vascular samples revealed markedly higher levels of a broad range of cytokines, chemokines, and immune markers reflecting T-cell and B-cell infiltration. This inflammatory pattern was seen even when normal, non-diseased samples were analyzed in the context of diabetes status. While not wishing to be bound by any theory, these results strongly suggest that diabetes activates a transcriptional program of coronary inflammation that is present even in the absence of atherosclerosis and suggest specific targets for diagnosis and therapy, e.g., any of the inflammatory mediators, immune markers, cytokines, and/or chemokines identified herein as being overexpressed

### Example 3

### Identification of Genes Whose Expression is Modulated by Statins and Aspririn

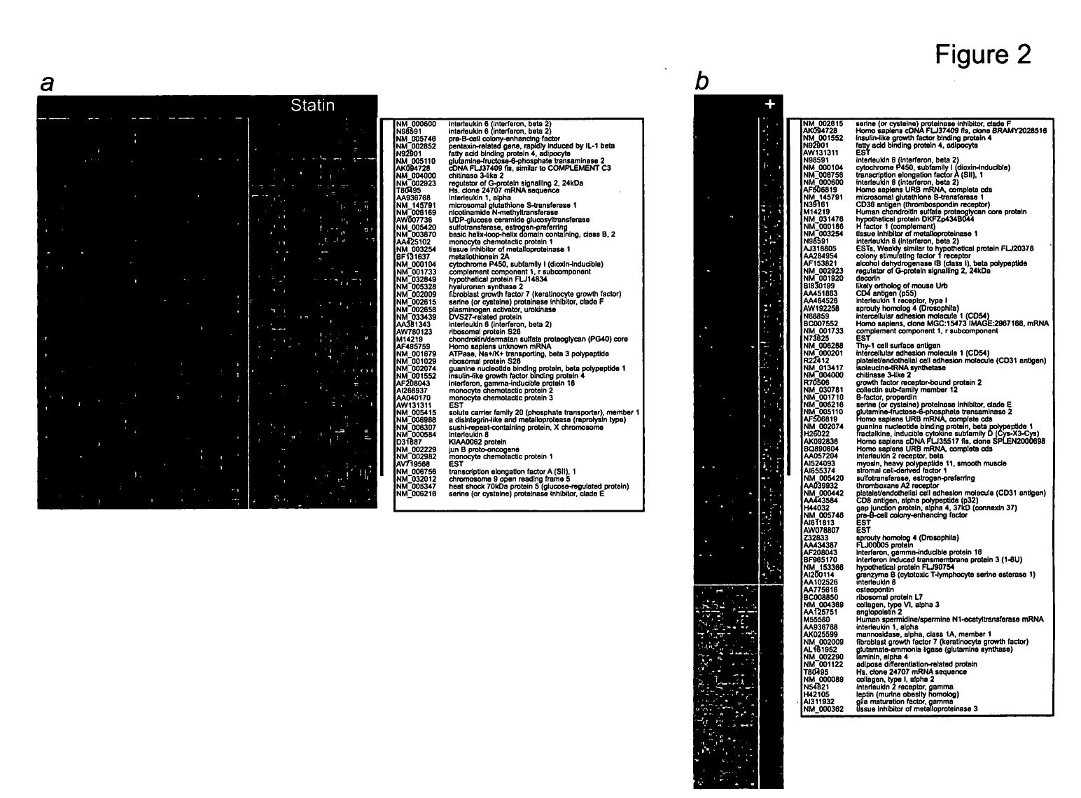

To evaluate how pharmacotherapies modulate vascular wall gene expression and identify additional targets for diagnosis and therapy and additional methods of identifying pharmacological agents useful for treating atherosclerosis, we conducted a comprehensive analysis with all coronary artery samples looking at basic classes of cardiovascular medications (Table 5). The most significant findings were generated from the analysis of statin use (FIG. 2a and Table 8). SAM identified 117 probes (FDR=0.05) and TNoM found a similar group of 82 probes expressed at significantly lower levels in vascular samples obtained from patients treated with statins (39/100) when compared to samples from untreated patients (61/100). A strong association was established between statin use and decreased expression of cytokines IL-1α and IL6, and cytokine-responsive monocyte chemokines MCP-1, MCP-2, and MCP-3. Analysis of GO nomenclature revealed an under-representation of terms “inflammation” (p<0.05) and “interleukin” (p<0.001) (Table 6).

Since coronary arteries from diabetics expressed high levels of inflammatory cytokines, we performed a sub-analysis with these samples alone in the context of statin treatment. Gene expression profiles of statin-treated (9/34) and untreated (25/34) diabetic vascular samples were compared. SAM identified 1830 differentially regulated probes (FDR=0.05) while TNoM found a similar group of 2205 probes. A heat map with a partial gene list representing the 318 most differentially regulated probes identified by SAM (FDR=0.0016) is shown and includes genes in cytokine, chemokine, and immunomodulatory pathways (FIG. 2b). GO analysis confirmed the under-representation of probes with nomenclature “inflammation” (p<0.01), “cytokine” (p<0.05), “interleukin” (p<0.05), and “chemokine” (p<0.05) (Table 6). Matrix protein osteopontin, intercellular adhesion molecule (ICAM1), TIMP-1, and TIMP3 were among the cytokine-responsive genes expressed at lower levels in diabetics exposed to statins (FIG. 2b). Decreased expression of T-cell genes (CD4, CD8, granzyme B, and thy1) in vessels from statin-treated patients suggested that these drugs might decrease the accumulation of this immune cell type in the coronary arteries of diabetics.

We analyzed the effects of aspirin on vascular wall gene expression and found similar results. Expression of cytokines IL-6 and chemokine IL-8 in vascular samples obtained from patients taking aspirin were markedly reduced. Cyclooxygenase-2 (COX-2) and an inflammatory cytokine-responsive metalloproteinase-disintegrin family protein (ADAMTS1) were also expressed at lower levels in these patients.

### Example 4

### Validation of Microarray Data by Quantitative PCR Analysis

Materials and Methods

Quantitative Real-Time PCR.

Expression of five genes was assessed in 30 RNA samples. Total RNA was subjected to reverse transcription and polymerase chain reaction, and amplifications were performed in triplicate. A standard curve was employed for RNA quantification, and RNA quantity expressed relative to the corresponding 18S internal control. Three patient RNA samples were evaluated per clinical condition per gene, and the mean normalized value was calculated.

Results

We performed quantitative real-time polymerase chain reaction (qRT-PCR) for a subset of differentially expressed genes to validate the microarray methodology. IL-8, IL-18, and LOX-1, all expressed at higher levels in lesions versus normal vascular samples by microarray analysis, were found to be similarly differentially expressed using qRT-PCR (Table 7). IL-6 and insulin-like growth factor binding protein 4 (ILGFBP4) transcript levels were substantially higher in diabetic versus non-diabetic vascular samples by both methods. Table 7 provides relative expression values for qRT-PCR as the mean of individual ratios of RNA amounts normalized to 18S RNA for three patients, and microarray data is provided as mean normalized ratios of experimental gene expression compared to reference RNA for the same three patients. Abbreviations: IL, interleukin; LOX-1, low density lipoprotein receptor-1 (LOX-1); ILGFBP4, insulin-like growth factor binding protein 4.

TABLE 7
Comparison of microarray and polymerase chain reaction (qRT-PCR)
expression data.

| Gene: comparison | Taqman | Microarray |
|---|---|---|
| IL-8: Lesion | 22.050 | 20.745 |
| IL-8: No Lesion | 1.338 | 1.332 |
| IL-18: Lesion | 32.229 | 5.968 |
| IL-18: No Lesion | 2.788 | 1.127 |
| LOX-1: Lesion | 15.403 | 14.395 |
| LOX-1: No Lesion | 1.604 | 1.172 |
| IL-6: Diabetes | 702.584 | 30.207 |
| IL-6: No Diabetes | 4.225 | 1.047 |
| ILGFBP4: Diabetes | 26.665 | 7.257 |
| ILGFBP4: No | 3.365 | 1.634 |
| Diabetes | | |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. Furthermore, it is to be understood that the invention encompasses all variations, combinations, and permutations in which one or more limitations, elements, clauses, descriptive terms, etc., from one or more of the listed claims or relevant descriptive material in the specification is introduced into another claim. Any claim that is dependent on another claim can be modified to include one or more limitations found in any other claim that is dependent on the same base claim. In addition, it is to be understood that any particular embodiment of the present invention and/or any element, limitation, feature, or term can be explicitly excluded from any one or more of the claims below or description above. For example, any specific gene, polynucleotide, polypeptide, method of use, etc., can be excluded from any one or more of the claims. For purposes of brevity, all of these various embodiments in which and/or any element, limitation, feature, or term is excluded are not set forth specifically herein. It noted that any embodiment may be deemed to fall within the prior art or be obvious in view of the prior art may be specifically excluded, such embodiments being known to or obvious to one of skill in the art and therefore not explicitly set forth herein.

TABLE 1

| | | | | | | | | |
|---|---|---|---|---|---|---|---|---|
| | | | | | Ratio | | | |
| SystematicName | UnigeneCode | GeneName | GeneSymbol | TNoM p-value | fold change* | t-test score p-value | Change Direction | Comment |

Genes with Known Name/or Functions (Note: Lesion > No lesion, Foldchange positive; No lesion > lesion, negative).

| | | | | | | | | |
|---|---|---|---|---|---|---|---|---|
| AA682386 | Hs.77729 | oxidised low density lipoprotein (lectin-like) receptor 1 | | 1.26E−06 | 2.18 | 2.42E−05 | No Lesion < Lesion | known |
| AA969504 | Hs.856 | interferon, gamma | | 4.84E−04 | 2.09 | 1.12E−03 | No Lesion < Lesion | |
| AA102526 | Hs.624 | interleukin 8 | | 1.60E−05 | 1.68 | 2.89E−05 | No Lesion < Lesion | known |
| AA873792 | Hs.241392 | small inducible cytokine A5 (RANTES) | | 4.84E−04 | 1.65 | 5.17E−03 | No Lesion < Lesion | |
| NM_000930 | Hs.274404 | plasminogen activator, tissue | PLAT | 1.26E−06 | 1.63 | 8.45E−07 | No Lesion < Lesion | |
| NM_058197 | Hs.1174 | cyclin-dependent kinase inhibitor 2A (melanoma, p16, | CDKN2A | 1.60E−05 | 1.60 | 3.88E−03 | No Lesion < Lesion | |
| | | inhibits CDK4) | | | | | | |
| AI129421 | Hs.83077 | interleukin 18 (interferon-gamma-inducing factor) | | 4.84E−04 | 1.58 | 1.92E−04 | No Lesion < Lesion | known |
| NM_002117 | Hs.277477 | major histocompatibility complex, class I, C | HLA-C | 4.62E−06 | 1.58 | 2.32E−06 | No Lesion < Lesion | |
| NM_002510 | Hs.82226 | glycoprotein (transmembrane) nmb | GPNMB | 4.84E−04 | 1.57 | 5.38E−04 | No Lesion < Lesion | |
| AF001893 | Hs.240443 | multiple endocrine neoplasia I | | 1.60E−05 | 1.52 | 5.61E−03 | No Lesion < Lesion | |
| NM_021999 | Data not found | integral membrane protein 2B | ITM2B | 5.24E−05 | 1.51 | 7.90E−04 | No Lesion < Lesion | |
| NM_002356 | Hs.75607 | myristoylated alanine-rich protein kinase C substrate | MARCKS | 1.63E−04 | 1.50 | 3.23E−04 | No Lesion < Lesion | |
| AL133111 | Hs.109150 | SH3-domain binding protein 5 (BTK-associated) | | 1.63E−04 | 1.48 | 1.37E−03 | No Lesion < Lesion | |
| AA621188 | Hs.4996 | putative ankyrin-repeat containing protein | | 4.62E−06 | 1.45 | 1.95E−06 | No Lesion < Lesion | |
| R94661 | Hs.181392 | major histocompatibility complex, class I, E | | 4.84E−04 | 1.44 | 2.22E−04 | No Lesion < Lesion | |
| AI279830 | Hs.45719 | protein phosphatase 1, regulatory (inhibitor) subunit | | 4.62E−06 | 1.43 | 2.59E−04 | No Lesion < Lesion | |
| | | 16B | | | | | | |
| NM_001530 | Hs.197540 | hypoxia-inducible factor 1, alpha subunit (basic helix- | | 4.84E−04 | 1.41 | 1.98E−03 | No Lesion < Lesion | |
| | | loop-helix transcription factor) | | | | | | |
| NM_004183 | Data not found | vitelliform macular dystrophy (Best disease, | VMD2 | 5.24E−05 | 1.39 | 7.50E−05 | No Lesion < Lesion | |
| | | bestrophin) | | | | | | |
| AA057204 | Hs.75596 | interleukin 2 receptor, beta | | 4.84E−04 | 1.38 | 7.79E−03 | No Lesion < Lesion | known |
| BC014989 | Hs.78575 | phospholipid scramblase 3 | | 3.97E−09 | 1.37 | 1.63E−04 | No Lesion < Lesion | |
| N36136 | Hs.41135 | endomucin-2 | | 1.63E−04 | 1.36 | 8.32E−03 | No Lesion < Lesion | |
| AA521362 | Hs.73792 | complement component (3d/Epstein Barr virus) | | 1.63E−04 | 1.35 | 1.51E−03 | No Lesion < Lesion | |
| | | receptor 2 | | | | | | |
| NM_005625 | Hs.8180 | syndecan binding protein (syntenin) | SDCBP | 1.60E−05 | 1.34 | 1.89E−03 | No Lesion < Lesion | |
| NM_004048 | Hs.75415 | beta-2-microglobulin | B2M | 1.60E−05 | 1.33 | 1.86E−05 | No Lesion < Lesion | |
| NM_025197 | Hs.20157 | CDK5 regulatory subunit associated protein 3 | CDK5RAP3 | 5.24E−05 | 1.31 | 8.37E−07 | No Lesion < Lesion | |
| AA418813 | Hs.184167 | splicing factor, arginine/serinE−rich 7 (35 kD) | | 4.84E−04 | 1.31 | 1.18E−04 | No Lesion < Lesion | |
| AA521008 | Hs.394 | adrenomedullin | | 1.84E−08 | 1.31 | 4.58E−09 | No Lesion < Lesion | |
| AA011182 | Hs.243010 | ras homolog gene family, member J | | 1.26E−06 | 1.28 | 7.49E−02 | No Lesion < Lesion | |
| NM_005520 | Hs.245710 | heterogeneous nuclear ribonucleoprotein H1 (H) | HNRPH1 | 1.63E−04 | 1.26 | 3.70E−04 | No Lesion < Lesion | |
| NM_006435 | Hs.174195 | interferon induced transmembrane protein 2 (1-8D) | IFITM2 | 4.84E−04 | 1.26 | 1.12E−02 | No Lesion < Lesion | |
| NM_021034 | Hs.182241 | interferon induced transmembrane protein 3 (1-8U) | IFITM3 | 1.63E−04 | 1.25 | 3.53E−02 | No Lesion < Lesion | |
| NM_006014 | Data not found | DNA segment on chromosome X (unique) 9879 | DXS9879E | 1.63E−04 | 1.25 | 6.63E−02 | No Lesion < Lesion | |
| | | expressed sequence | | | | | | |
| NM_001386 | Hs.173381 | dihydropyrimidinase-like 2 | DPYSL2 | 1.63E−04 | 1.23 | 1.70E−03 | No Lesion < Lesion | |
| AA150505 | Hs.8135 | complement component 1, q subcomponent, receptor 1 | | 1.60E−05 | 1.22 | 1.19E−03 | No Lesion < Lesion | |
| NM_004396 | Hs.76053 | DEAD/H (Asp-Glu-Ala-Asp/His) box polypeptide 5 | DDX5 | 1.63E−04 | 1.22 | 3.89E−04 | No Lesion < Lesion | |
| | | (RNA helicase, 68 kDa) | | | | | | |
| NM_016099 | Hs.7953 | HSPC041 protein | GOLGA7 | 1.63E−04 | 1.22 | 7.86E−05 | No Lesion < Lesion | |
| NM_005063 | Hs.119597 | stearoyl-CoA desaturase (delta-9-desaturase) | SCD | 1.63E−04 | 1.20 | 4.57E−02 | No Lesion < Lesion | |
| N53056 | Hs.100001 | solute carrier family 17 (sodium phosphate), member 1 | | 4.84E−04 | 1.20 | 2.04E−04 | No Lesion < Lesion | |
| AA454176 | Hs.169750 | glutamate-cysteine ligase, modifier subunit | | 1.63E−04 | 1.20 | 1.63E−02 | No Lesion < Lesion | |
| AA449301 | Hs.138671 | fms-related tyrosine kinase 1 (vascular endothelial | | 4.84E−04 | 1.20 | 7.80E−02 | No Lesion < Lesion | |
| | | growth factor/vascular permeability factor receptor) | | | | | | |
| AA450264 | Hs.78996 | proliferating cell nuclear antigen | | 1.60E−05 | 1.18 | 2.06E−02 | No Lesion < Lesion | |
| NM_005015 | Hs.151134 | oxidase (cytochrome c) assembly 1-like | OXA1L | 4.84E−04 | 1.18 | 2.46E−03 | No Lesion < Lesion | |
| AK092006 | Hs.217493 | annexin A2 | | 1.63E−04 | 1.17 | 1.54E−02 | No Lesion < Lesion | |
| NM_001017 | Hs.165590 | ribosomal protein S13 | RPS13 | 4.84E−04 | 1.16 | 1.29E−02 | No Lesion < Lesion | |
| NM_006164 | Hs.155396 | nuclear factor (erythroid-derived 2)-like 2 | NFE2L2 | 5.24E−05 | 1.16 | 4.99E−02 | No Lesion < Lesion | |
| AA425011 | Hs.180799 | C3HC4-type zinc finger protein | | 1.63E−04 | 1.15 | 3.16E−03 | No Lesion < Lesion | |
| NM_001614 | Hs.14376 | actin, gamma 1 | ACTG1 | 1.63E−04 | 1.11 | 2.70E−02 | No Lesion < Lesion | |
| N62629 | Hs.48589 | zinc finger protein 228 | | 4.84E−04 | 1.10 | 2.38E−01 | No Lesion < Lesion | |
| NM_014294 | Hs.4147 | translocating chain-associating membrane protein | TRAM1 | 4.84E−04 | −1.10 | 9.50E−02 | Lesion < No Lesion | |
| AL832675 | Hs.76728 | CD47 antigen (Rh-related antigen, integrin-associated | | 4.84E−04 | −1.13 | 1.55E−01 | Lesion < No Lesion | |
| | | signal transducer) | | | | | | |
| NM_001752 | Hs.76359 | catalase | CAT | 1.63E−04 | −1.15 | 2.60E−02 | Lesion < No Lesion | |
| NM_003999 | Hs.238648 | oncostatin M receptor | OSMR | 4.84E−04 | −1.17 | 9.58E−04 | Lesion < No Lesion | |
| NM_153207 | Hs.285833 | hypothetical protein MGC17922 | AEBP2 | 1.63E−04 | −1.18 | 5.07E−02 | Lesion < No Lesion | |
| X15786 | Hs.241572 | Human ret-II gene | | 5.24E−05 | −1.19 | 1.64E−03 | Lesion < No Lesion | |
| AL832212 | Hs.28505 | ubiquitin-conjugating enzyme E2H (UBC8 homolog, | | 1.63E−04 | −1.20 | 3.18E−03 | Lesion < No Lesion | |
| | | yeast) | | | | | | |
| NM_003299 | Data not found | tumor rejection antigen (gp96) 1 | TRA1 | 1.63E−04 | −1.20 | 1.49E−02 | Lesion < No Lesion | |
| NM_033375 | Hs.286226 | myosin IC | MYO1C | 4.84E−04 | −1.20 | 1.08E−02 | Lesion < No Lesion | |
| X03541 | Hs.85844 | Human mRNA of trk oncogene | | 1.63E−04 | −1.20 | 1.61E−02 | Lesion < No Lesion | |
| BM543083 | Hs.136309 | SH3-domain GRB2-like endophilin B1 | | 5.24E−05 | −1.21 | 4.48E−04 | Lesion < No Lesion | |
| NM_079425 | Data not found | myosin, light polypeptide 6, alkali, smooth muscle and | MYL6 | 1.63E−04 | −1.22 | 1.11E−02 | Lesion < No Lesion | |
| | | non-muscle | | | | | | |
| NM_138799 | Hs.15641 | hypothetical protein BC016005 | OACT2 | 5.24E−05 | −1.22 | 1.12E−03 | Lesion < No Lesion | |
| NM_006206 | Hs.74615 | platelet-derived growth factor receptor, alpha | PDGFRA | 4.84E−04 | −1.22 | 3.16E−03 | Lesion < No Lesion | |
| | | polypeptide | | | | | | |
| AB088120 | Hs.76591 | expressed in T-cells and eosinophils in atopic | | 4.84E−04 | −1.23 | 2.49E−04 | Lesion < No Lesion | |
| | | dermatitis | | | | | | |
| NM_004578 | Hs.119007 | RAB4A, member RAS oncogene family | RAB4A | 4.84E−04 | −1.23 | 1.53E−02 | Lesion < No Lesion | |
| NM_016308 | Hs.11463 | UMP-CMP kinase | UMP-CMPK | 1.63E−04 | −1.23 | 4.19E−03 | Lesion < No Lesion | |
| NM_001177 | Hs.242894 | ADP-ribosylation factor-like 1 | ARL1 | 1.63E−04 | −1.24 | 1.67E−05 | Lesion < No Lesion | |
| NM_006088 | Hs.251653 | tubulin, beta, 2 | TUBB2 | 4.84E−04 | −1.24 | 1.11E−02 | Lesion < No Lesion | |
| NM_004199 | Hs.3622 | procollagen-proline, 2-oxoglutarate 4-dioxygenase | P4HA2 | 1.63E−04 | −1.25 | 3.64E−03 | Lesion < No Lesion | |
| | | (proline 4-hydroxylase), alpha polypeptide II | | | | | | |
| AB051504 | Hs.78521 | SET domain-containing protein 7 | | 4.84E−04 | −1.25 | 8.11E−04 | Lesion < No Lesion | |
| NM_007107 | Hs.28707 | signal sequence receptor, gamma (translocon- | SSR3 | 1.63E−04 | −1.26 | 9.61E−04 | Lesion < No Lesion | |
| | | associated protein gamma) | | | | | | |
| NM_002956 | Hs.31638 | restin (Reed-Steinberg cell-expressed intermediate | RSN | 4.84E−04 | −1.26 | 1.24E−02 | Lesion < No Lesion | |
| | | filament-associated protein | | | | | | |
| NM_015523 | Hs.7527 | small fragment nuclease | DKFZP566E144 | 1.60E−05 | −1.26 | 1.86E−04 | Lesion < No Lesion | |
| NM_003676 | Hs.185973 | degenerative spermatocyte homolog, lipid desaturase | DEGS | 1.63E−04 | −1.26 | 1.13E−05 | Lesion < No Lesion | |
| | | (Drosophila) | | | | | | |
| NM_004236 | Hs.30212 | thyroid receptor interacting protein 15 | TRIP15 | 4.84E−04 | −1.27 | 6.71E−03 | Lesion < No Lesion | |
| NM_018955 | Hs.183842 | ubiquitin B | UBB | 5.24E−05 | −1.28 | 4.45E−05 | Lesion < No Lesion | |
| NM_005347 | Hs.75410 | heat shock 70 kDa protein 5 (glucose-regulated protein, | HSPA5 | 1.63E−04 | −1.28 | 8.70E−04 | Lesion < No Lesion | |
| | | 78 kDa) | | | | | | |
| NM_003295 | Data not found | tumor protein, translationally-controlled 1 | TPT1 | 1.60E−05 | −1.28 | 4.56E−04 | Lesion < No Lesion | |
| NM_002157 | Hs.1197 | heat shock 10 kDa protein 1 (chaperonin 10) | HSPE1 | 1.26E−06 | −1.29 | 2.72E−03 | Lesion < No Lesion | |
| NM_001967 | Hs.182429 | eukaryotic translation initiation factor 4A, isoform 2 | EIF4A2 | 4.84E−04 | −1.29 | 6.68E−03 | Lesion < No Lesion | |
| NM_001219 | Hs.7753 | calumenin | CALU | 4.84E−04 | −1.29 | 8.70E−05 | Lesion < No Lesion | |
| NM_001792 | Hs.161 | cadherin 2, type 1, N-cadherin (neuronal) | CDH2 | 4.84E−04 | −1.30 | 5.86E−03 | Lesion < No Lesion | |
| NM_015994 | Hs.272630 | ATPase, H+ transporting, lysosomal 34 kDa, V1 subunit D | ATP6V1D | 5.24E−05 | −1.31 | 3.98E−05 | Lesion < No Lesion | |
| AA489611 | Hs.2795 | lactate dehydrogenase A | | 1.63E−04 | −1.33 | 2.80E−04 | Lesion < No Lesion | |
| NM_030571 | Hs.9788 | likely ortholog of mouse Nedd4 WW binding protein 5 | NDFIP1 | 4.62E−06 | −1.33 | 6.23E−03 | Lesion < No Lesion | |
| NM_002901 | Hs.167791 | reticulocalbin 1, EF-hand calcium binding domain | RCN1 | 1.63E−04 | −1.33 | 4.34E−04 | Lesion < No Lesion | |
| AL832431 | Hs.8107 | guanine nucleotide binding protein (G protein), gamma | | 4.84E−04 | −1.33 | 4.97E−04 | Lesion < No Lesion | |
| | | 12 | | | | | | |
| NM_013436 | Hs.278411 | NCK-associated protein 1 | NCKAP1 | 1.60E−05 | −1.35 | 2.76E−04 | Lesion < No Lesion | |
| NM_001839 | Hs.194662 | calponin 3, acidic | CNN3 | 4.62E−06 | −1.36 | 5.08E−04 | Lesion < No Lesion | |
| NM_003330 | Hs.13046 | thioredoxin reductase 1 | TXNRD1 | 5.24E−05 | −1.38 | 2.71E−03 | Lesion < No Lesion | |
| NM_004735 | Hs.326159 | leucine rich repeat (in FLII) interacting protein 1 | LRRFIP1 | 5.24E−05 | −1.39 | 1.27E−03 | Lesion < No Lesion | |
| BU542589 | Hs.37196 | putative G protein coupled receptor | | 4.84E−04 | −1.39 | 5.81E−04 | Lesion < No Lesion | |
| U72621 | Hs.75825 | pleiomorphic adenoma gene-like 1 | | 1.63E−04 | −1.41 | 1.04E−03 | Lesion < No Lesion | |
| NM_006826 | Hs.74405 | tyrosine 3-monooxygenase/tryptophan 5- | YWHAQ | 4.84E−04 | −1.42 | 2.41E−03 | Lesion < No Lesion | |
| | | monooxygenase activation protein, theta polypeptide | | | | | | |
| NM_053056 | Hs.82932 | cyclin D1 (PRAD1: parathyroid adenomatosis 1) | CCND1 | 1.63E−04 | −1.43 | 3.73E−02 | Lesion < No Lesion | |
| BE300066 | Data not found | heat shock 90 kDa protein 1, alpha | HSPCA | 5.24E−05 | −1.44 | 1.23E−04 | Lesion < No Lesion | |
| BF976811 | Data not found | leucyl-tRNA synthetase | | 1.60E−05 | −1.46 | 7.87E−05 | Lesion < No Lesion | |
| NM_012286 | Hs.173714 | MORF-related gene X | MORF4L2 | 1.63E−04 | −1.48 | 2.46E−04 | Lesion < No Lesion | |
| NM_053275 | Hs.73742 | ribosomal protein, large, P0 | RPLP0 | 4.84E−04 | −1.48 | 3.61E−03 | Lesion < No Lesion | |
| NM_022152 | Hs.184052 | PP1201 protein | PP1201 | 1.63E−04 | −1.48 | 2.43E−04 | Lesion < No Lesion | |
| NM_000366 | Hs.77899 | tropomyosin 1 (alpha) | TPM1 | 4.84E−04 | −1.49 | 5.79E−04 | Lesion < No Lesion | |
| NM_021069 | Data not found | Arg/Abl-interacting protein ArgBP2 | ARGBP2 | 4.84E−04 | −1.49 | 2.21E−02 | Lesion < No Lesion | |
| AJ420488 | Hs.181165 | eukaryotic translation elongation factor 1 alpha 1 | | 1.63E−04 | −1.50 | 2.58E−03 | Lesion < No Lesion | |
| NM_006644 | Hs.36927 | heat shock 105 kD | HSPH1 | 1.26E−06 | −1.51 | 4.14E−06 | Lesion < No Lesion | |
| NM_012111 | Hs.204041 | chromosome 14 open reading frame 3 | AHSA1 | 4.62E−06 | −1.54 | 8.76E−07 | Lesion < No Lesion | |
| NM_018212 | Data not found | enabled homolog (Drosophila) | ENAH | 5.24E−05 | −1.56 | 2.97E−05 | Lesion < No Lesion | |
| NM_004281 | Hs.15259 | BCL2-associated athanogene 3 | BAG3 | 1.26E−06 | −1.57 | 2.11E−03 | Lesion < No Lesion | |
| NM_013943 | Hs.25035 | chloride intracellular channel 4 | CLIC4 | 5.24E−05 | −1.61 | 2.94E−05 | Lesion < No Lesion | |
| NM_007341 | Hs.47438 | SH3 domain binding glutamic acid-rich protein | SH3BGR | 4.84E−04 | −1.62 | 1.83E−04 | Lesion < No Lesion | |
| D83886 | Hs.42500 | ADP-ribosylation factor-like 5 | | 1.63E−04 | −1.63 | 8.64E−05 | Lesion < No Lesion | |
| NM_015701 | Hs.7100 | hypothetical protein CL25084 | C2orf30 | 1.63E−04 | −1.68 | 4.40E−07 | Lesion < No Lesion | |
| NM_001664 | Hs.179735 | ras homolog gene family, member A | RHOA | 5.24E−05 | −1.70 | 1.84E−03 | Lesion < No Lesion | |
| NM_004613 | Hs.8265 | transglutaminase 2 (C polypeptide, protein-glutamine- | | 1.63E−04 | −1.73 | 2.65E−03 | Lesion < No Lesion | |
| | | gamma-glutamyltransferase) | | | | | | |
| NM_002026 | Data not found | fibronectin 1 | FN1 | 4.84E−04 | −1.75 | 8.71E−04 | Lesion < No Lesion | known |
| NM_002046 | Hs.169476 | glyceraldehyde-3-phosphate dehydrogenase | GAPD | 4.84E−04 | −1.80 | 1.41E−03 | Lesion < No Lesion | |
| NM_002211 | Data not found | integrin, beta 1 (fibronectin receptor, beta polypeptide, | | 4.84E−04 | −1.86 | 2.21E−05 | Lesion < No Lesion | |
| | | antigen CD29 includes MDF2, MSK12) | | | | | | |
| NM_001102 | Hs.119000 | actinin, alpha 1 | ACTN1 | 1.26E−06 | −1.90 | 3.60E−03 | Lesion < No Lesion | |
| NM_006597 | Hs.180414 | heat shock 70 kDa protein 8 | HSPA8 | 1.63E−04 | −1.96 | 4.22E−05 | Lesion < No Lesion | |
| NM_005345 | Hs.8997 | heat shock 70 kDa protein 1A | HSPA1A | 4.62E−06 | −1.97 | 3.33E−05 | Lesion < No Lesion | |
| D89937 | Hs.296267 | Homo sapiens mRNA for follistatin-related protein | | 5.24E−05 | −2.15 | 1.69E−07 | Lesion < No Lesion | |
| | | (FRP), complete cds | | | | | | |

Genes with Unknown Name/Functions (Note: Lesion > No lesion, Foldchange pos.; No lesion > lesion, Fold change neg.).

| | | | | | | | |
|---|---|---|---|---|---|---|---|
| BC015869 | Hs.8136 | Homo sapiens clone 23698 mRNA sequence | | 5.24E−05 | 1.58 | 2.99E−04 | No Lesion < Lesion |
| AA147552 | Hs.71832 | ESTs | | 4.84E−04 | 1.51 | 3.24E−04 | No Lesion < Lesion |
| BE615903 | Data not found | EST | | 4.84E−04 | 1.44 | 1.35E−05 | No Lesion < Lesion |
| AA115259 | Hs.103422 | Hs. mRNA; cDNA DKFZp434F1622 (from clone | | 1.63E−04 | 1.33 | 2.55E−03 | No Lesion < Lesion |
| | | DKFZp434F1622) | | | | | |
| N65985 | Hs.124696 | Hs. cDNA FLJ13261 fis, clone OVARC1000885, | | 3.27E−07 | 1.32 | 5.60E−04 | No Lesion < Lesion |
| | | weakly similar to OXIDOREDUCTASE UCPA (EC 1.—.—.—) | | | | | |
| AW148618 | Data not found | EST, Moderately similar to 810024E cytochrome | | 4.84E−04 | 1.27 | 8.41E−03 | No Lesion < Lesion |
| | | oxidase III [Homo sapiens] [H. sapien] | | | | | |
| AA431193 | Hs.19280 | KIAA0544 protein | | 4.84E−04 | 1.26 | 6.40E−02 | No Lesion < Lesion |
| AK074815 | Hs.7099 | hypothetical protein FLJ20265 | | 4.84E−04 | 1.24 | 4.28E−03 | No Lesion < Lesion |
| AA192691 | Data not found | EST | | 5.24E−05 | 1.24 | 2.89E−02 | No Lesion < Lesion |
| AK027088 | Hs.35140 | Homo sapiens cDNA: FLJ23435 fis, clone HRC12631 | | 4.84E−04 | 1.23 | 2.32E−05 | No Lesion < Lesion |
| AA485428 | Hs.301685 | KIAA0620 protein | | 4.84E−04 | 1.23 | 2.81E−02 | No Lesion < Lesion |
| BC021287 | Hs.184544 | Homo sapiens, clone IMAGE: 3355383, mRNA, partial | | 4.84E−04 | 1.22 | 1.23E−04 | No Lesion < Lesion |
| | | cds | | | | | |
| NM_152531 | Hs.150614 | hypothetical protein FLJ35155 | FLJ35155 | 4.84E−04 | 1.22 | 5.18E−02 | No Lesion < Lesion |
| AA968877 | Hs.172928 | Hs. cDNA: FLJ21464 fis, clone COL04768 | | 5.24E−05 | 1.21 | 1.30E−04 | No Lesion < Lesion |
| AA626000 | Hs.94810 | hypothetical protein FLJ12242 | | 1.63E−04 | 1.19 | 4.60E−03 | No Lesion < Lesion |
| BQ070901 | Hs.288967 | Homo sapiens, similar to RIKEN cDNA 0610010I12, | | 4.84E−04 | 1.17 | 6.08E−05 | No Lesion < Lesion |
| | | clone MGC: 35430 IMAGE: 5189880, mRNA, complete | | | | | |
| | | cds | | | | | |
| NM_015138 | Hs.83419 | KIAA0252 protein | KIAA0252 | 1.63E−04 | 1.13 | 3.26E−02 | No Lesion < Lesion |
| BC008758 | Hs.157850 | ESTs, Highly similar to IDHG_HUMAN Isocitrate | | 1.63E−04 | 1.11 | 5.08E−02 | No Lesion < Lesion |
| | | dehydrogenase [NAD] subunit gamma, mitochondrial | | | | | |
| | | precursor (Isocitric dehydrogenase) (NAD+-specific | | | | | |
| | | ICDH) [H. sapiens] | | | | | |
| AK001701 | Data not found | hypothetical protein FLJ10839 | FLJ10839 | 4.84E−04 | 1.10 | 1.33E−01 | No Lesion < Lesion |
| BC007552 | Hs.111334 | Homo sapiens, clone MGC: 15473 IMAGE: 2967168, | | 1.63E−04 | 1.09 | 6.75E−02 | No Lesion < Lesion |
| | | mRNA, complete cds | | | | | |
| BC034962 | Hs.77608 | Homo sapiens, clone IMAGE: 4822098, mRNA, partial | 15E1.2 | 1.63E−04 | −1.15 | 1.66E−03 | Lesion < No Lesion |
| | | cds | | | | | |
| BC015653 | Hs.285122 | Homo sapiens, clone MGC: 23488 IMAGE: 4810553, | | 3.27E−07 | −1.19 | 9.77E−05 | Lesion < No Lesion |
| | | mRNA, complete cds | | | | | |
| NM_032339 | Hs.333526 | hypothetical protein MGC14832 | C17orf37 | 4.84E−04 | −1.21 | 4.98E−04 | Lesion < No Lesion |
| BM477149 | Hs.74267 | ESTs, Highly similar to RL15_HUMAN 60S ribosomal | | 1.63E−04 | −1.22 | 4.84E−03 | Lesion < No Lesion |
| | | protein L15 [H. sapiens] | | | | | |
| AW166001 | Data not found | EST, Weakly similar to 810024E cytochrome oxidase | | 1.63E−04 | −1.23 | 4.76E−04 | Lesion < No Lesion |
| | | III [Homo sapiens] [H. sapiens] | | | | | |
| BG254709 | Data not found | ESTs, Highly similar to I39382 Y box-binding protein 1 — | | 4.84E−04 | −1.24 | 1.00E−03 | Lesion < No Lesion |
| | | human [H. sapiens] | | | | | |
| NM_022063 | Hs.11859 | hypothetical protein FLJ13188 | C10orf84 | 4.84E−04 | −1.24 | 2.60E−02 | Lesion < No Lesion |
| BM473144 | Data not found | ESTs, Highly similar to RLA0_HUMAN 60S acidic | | 4.84E−04 | −1.26 | 2.77E−03 | Lesion < No Lesion |
| | | ribosomal protein P0 (L10E) [H. sapiens] | | | | | |
| BC013729 | Hs.111126 | Homo sapiens, clone IMAGE: 3859592, mRNA | | 4.84E−04 | −1.27 | 6.00E−03 | Lesion < No Lesion |
| NM_032374 | Hs.265317 | hypothetical protein MGC2562 | C14orf153 | 1.63E−04 | −1.28 | 2.68E−04 | Lesion < No Lesion |
| AA553367 | Hs.257631 | ESTs | | 4.84E−04 | −1.29 | 1.20E−02 | Lesion < No Lesion |
| AL833007 | Hs.121520 | Homo sapiens, clone IMAGE: 3625286, mRNA, partial | | 4.84E−04 | −1.31 | 2.67E−02 | Lesion < No Lesion |
| | | cds | | | | | |
| AL832395 | Hs.28578 | Homo sapiens mRNA; cDNA DKFZp667M1012 (from | | 1.63E−04 | −1.34 | 2.36E−03 | Lesion < No Lesion |
| | | clone DKFZp667M1012) | | | | | |
| AL359062 | Hs.284275 | Homo sapiens mRNA full length insert cDNA clone | | 4.84E−04 | −1.35 | 1.21E−02 | Lesion < No Lesion |
| | | EUROIMAGE 1913076 | | | | | |
| AK055112 | Hs.82503 | Homo sapiens cDNA FLJ30550 fis, clone | | 4.84E−04 | −1.38 | 5.23E−03 | Lesion < No Lesion |
| | | BRAWH2001502 | | | | | |
| AK055197 | Hs.77899 | Homo sapiens cDNA FLJ30635 fis, clone | | 4.84E−04 | −1.41 | 1.73E−02 | Lesion < No Lesion |
| | | CTONG2002520 | | | | | |
| AV719568 | Hs.289088 | EST | | 4.84E−04 | −1.44 | 8.28E−04 | Lesion < No Lesion |
| BC009275 | Data not found | Homo sapiens, actin, beta, clone MGC: 10644 | | 1.63E−04 | −1.51 | 5.05E−03 | Lesion < No Lesion |
| | | IMAGE: 3960255, mRNA, complete cds | | | | | |
| BC000611 | Hs.48375 | Homo sapiens, small nuclear ribonucleoprotein | | 4.84E−04 | −1.52 | 4.62E−06 | Lesion < No Lesion |
| | | polypeptide N, clone MGC: 1613 IMAGE: 3347412, | | | | | |
| | | mRNA, complete cds | | | | | |
| BM804430 | Hs.181165 | ESTs, Highly similar to EFHU1 translation elongation | | 5.24E−05 | −1.70 | 2.68E−04 | Lesion < No Lesion |
| | | factor eEF-1 alpha-1 chain - human [H. sapiens] | | | | | |
| BI430544 | Hs.103042 | ESTs | | 4.84E−04 | −1.71 | 3.47E−04 | Lesion < No Lesion |

TABLE 2

| | | | | | |
|---|---|---|---|---|---|
| Gene Name | | | | Expected | |
| Classification info | Gene Accession | Gene Info | Score | Score | FDR |

These genes are up-regulated in diabetic samples and down-regulated in non-diabetic samples.

| | | | | | |
|---|---|---|---|---|---|
| AA936768 | 14N.7.D1 | interleukin 1, alpha | 2.3603 | 1.2098 | 0 |
| NM_000600 | 7F.7.B6 | interleukin 6 (interferon, beta 2) | 2.2486 | 1.107 | 0 |
| N98591 | 14N.7.C4 | interleukin 6 (interferon, beta 2) | 2.2175 | 1.0453 | 0 |
| AA156031 | 14N.4.G12 | metallothionein 2A | 2.2161 | 1.0039 | 0 |
| NM_001235 | 7R.9.A7 | serine (or cysteine) proteinase inhibitor, clade H (heat shock protein 47), | 2.1753 | 0.9733 | 0 |
| | | member 2 | | | |
| R21535 | 14N.2.A11 | Hs. cDNA FLJ11724 fis, clone HEMBA1005331 | 2.1509 | 0.9516 | 0 |
| NM_001235 | 7F.4.D7 | serine (or cysteine) proteinase inhibitor, clade H (heat shock protein 47), | 2.119 | 0.9335 | 0 |
| | | member 2 | | | |
| NM_001235 | 8R.2.D12 | serine (or cysteine) proteinase inhibitor, clade H (heat shock protein 47), | 2.0633 | 0.9188 | 0 |
| | | member 2 | | | |
| NM_001235 | 7R.2.D3 | serine (or cysteine) proteinase inhibitor, clade H (heat shock protein 47), | 2.049 | 0.9023 | 0 |
| | | member 2 | | | |
| BF131637 | 7F.5.E2 | metallothionein 2A | 2.0338 | 0.888 | 0 |
| NM_001235 | 7R.1.H3 | serine (or cysteine) proteinase inhibitor, clade H (heat shock protein 47), | 2.0331 | 0.8753 | 0 |
| | | member 2 | | | |
| AA936768 | 14N.7.C12 | interleukin 1, alpha | 2.026 | 0.863 | 0 |
| NM_000600 | 9R.10.G7 | interleukin 6 (interferon, beta 2) | 2.0141 | 0.8524 | 0 |
| NM_000600 | 7F.2.F2 | interleukin 6 (interferon, beta 2) | 2.0131 | 0.8437 | 0 |
| NM_001235 | 7F.10.E5 | serine (or cysteine) proteinase inhibitor, clade H (heat shock protein 47), | 2.0122 | 0.8349 | 0 |
| | | member 2 | | | |
| NM_006216 | 12R.3.A3 | serine (or cysteine) proteinase inhibitor, clade E (nexin, plasminogen | 2.0108 | 0.8267 | 0 |
| | | activator inhibitor type 1), member 2 | | | |
| NM_006216 | 9R.7.G9 | serine (or cysteine) proteinase inhibitor, clade E (nexin, plasminogen | 1.9914 | 0.8199 | 0.0128 |
| | | activator inhibitor type 1), member 2 | | | |
| NM_001235 | 7R.10.F12 | serine (or cysteine) proteinase inhibitor, clade H (heat shock protein 47), | 1.9847 | 0.8136 | 0.0128 |
| | | member 2 | | | |
| AA936768 | 14N.5.D1 | interleukin 1, alpha | 1.9827 | 0.8074 | 0.0128 |
| NM_006216 | 8F.4.D3 | serine (or cysteine) proteinase inhibitor, clade E (nexin, plasminogen | 1.9674 | 0.8015 | 0.0128 |
| | | activator inhibitor type 1), member 2 | | | |
| NM_001552 | 7R.6.A2 | insulin-like growth factor binding protein 4 | 1.9182 | 0.7955 | 0.0128 |
| NM_001235 | 8R.5.E2 | serine (or cysteine) proteinase inhibitor, clade H (heat shock protein 47), | 1.9084 | 0.7902 | 0.0128 |
| | | member 2 | | | |
| NM_004530 | 7R.1.H4 | matrix metalloproteinase 2 (gelatinase A, 72 kDa gelatinase, 72 kDa type | 1.9069 | 0.7844 | 0.0128 |
| | | IV collagenase) | | | |
| NM_000600 | 7F.9.D11 | interleukin 6 (interferon, beta 2) | 1.9021 | 0.7792 | 0.0128 |
| NM_001235 | 7R.7.B12 | serine (or cysteine) proteinase inhibitor, clade H (heat shock protein 47), | 1.9 | 0.7742 | 0.0128 |
| | | member 2 | | | |
| NM_004530 | 8R.1.B12 | matrix metalloproteinase 2 (gelatinase A, 72 kDa gelatinase, 72 kDa type | 1.8897 | 0.7694 | 0.0128 |
| | | IV collagenase) | | | |
| BM803108 | 7F.4.E7 | ESTs | 1.8888 | 0.765 | 0.0128 |
| NM_006216 | 12F.3.C4 | serine (or cysteine) proteinase inhibitor, clade E (nexin, plasminogen | 1.8833 | 0.761 | 0.0128 |
| | | activator inhibitor type 1), member 2 | | | |
| NM_004530 | 7R.5.D5 | matrix metalloproteinase 2 (gelatinase A, 72 kDa gelatinase, 72 kDa type | 1.8736 | 0.7566 | 0.0128 |
| | | IV collagenase) | | | |
| NHF | 9R.10.H5 | | 1.8731 | 0.7526 | 0.0128 |
| NHF | 7F.8.B7 | | 1.8678 | 0.7487 | 0.0128 |
| NM_001552 | 7F.2.B6 | insulin-like growth factor binding protein 4 | 1.8626 | 0.7446 | 0.0128 |
| NHF | 9R.3.A11 | | 1.8617 | 0.7408 | 0.0128 |
| NM_006216 | 8F.9.E7 | serine (or cysteine) proteinase inhibitor, clade E (nexin, plasminogen | 1.8602 | 0.7369 | 0.0128 |
| | | activator inhibitor type 1), member 2 | | | |
| NM_004530 | 7R.2.F8 | matrix metalloproteinase 2 (gelatinase A, 72 kDa gelatinase, 72 kDa type | 1.8354 | 0.7332 | 0.0128 |
| | | IV collagenase) | | | |
| NM_006216 | 7F.2.A3 | serine (or cysteine) proteinase inhibitor, clade E (nexin, plasminogen | 1.8293 | 0.7295 | 0.0128 |
| | | activator inhibitor type 1), member 2 | | | |
| N98591 | 14N.5.C4 | interleukin 6 (interferon, beta 2) | 1.8119 | 0.7259 | 0.0128 |
| NM_006216 | 8F.10.A8 | serine (or cysteine) proteinase inhibitor, clade E (nexin, plasminogen | 1.8061 | 0.7222 | 0.0128 |
| | | activator inhibitor type 1), member 2 | | | |
| NM_000600 | 7F.5.D4 | interleukin 6 (interferon, beta 2) | 1.8043 | 0.7193 | 0.0128 |
| NM_004530 | 7R.9.G4 | matrix metalloproteinase 2 (gelatinase A, 72 kDa gelatinase, 72 kDa type | 1.8004 | 0.7161 | 0.0233 |
| | | IV collagenase) | | | |
| AA936768 | 14N.5.C12 | interleukin 1, alpha | 1.7784 | 0.7129 | 0.0233 |
| NM_004530 | 7R.8.E8 | matrix metalloproteinase 2 (gelatinase A, 72 kDa gelatinase, 72 kDa type | 1.7784 | 0.7099 | 0.0233 |
| | | IV collagenase) | | | |
| NM_000088 | 8F.9.G4 | collagen, type I, alpha 1 | 1.777 | 0.707 | 0.0233 |
| NM_004530 | 7R.6.C11 | matrix metalloproteinase 2 (gelatinase A, 72 kDa gelatinase, 72 kDa type | 1.7642 | 0.7043 | 0.0299 |
| | | IV collagenase) | | | |
| NM_023009 | 7F.2.D11 | MARCKS-like protein | 1.7612 | 0.7013 | 0.0299 |
| NM_004530 | 7R.1.H1 | matrix metalloproteinase 2 (gelatinase A, 72 kDa gelatinase, 72 kDa type | 1.7564 | 0.6984 | 0.0299 |
| | | IV collagenase) | | | |
| NM_004530 | 8R.3.C9 | matrix metalloproteinase 2 (gelatinase A, 72 kDa gelatinase, 72 kDa type | 1.7526 | 0.6957 | 0.0299 |
| | | IV collagenase) | | | |
| NM_006216 | 8R.4.B7 | serine (or cysteine) proteinase inhibitor, clade E (nexin, plasminogen | 1.7454 | 0.6934 | 0.0299 |
| | | activator inhibitor type 1), member 2 | | | |
| NM_003670 | 9F.7.C8 | basic helix-loop-helix domain containing, class B, 2 | 1.7382 | 0.6908 | 0.0299 |
| T80495 | 14N.4.H12 | Hs. clone 24707 mRNA sequence | 1.7375 | 0.6882 | 0.0299 |
| NM_002993 | 7F.3.E6 | chemokine (C—X—C motif) ligand 6 (granulocyte chemotactic protein 2) | 1.7348 | 0.6853 | 0.0299 |
| NM_006756 | 9F.10.F12 | transcription elongation factor A (SII), 1 | 1.7321 | 0.6825 | 0.0299 |
| NM_006216 | 8R.10.A1 | serine (or cysteine) proteinase inhibitor, clade E (nexin, plasminogen | 1.7275 | 0.6798 | 0.0299 |
| | | activator inhibitor type 1), member 2 | | | |
| NM_004530 | 1F.1.G7 | matrix metalloproteinase 2 (gelatinase A, 72 kDa gelatinase, 72 kDa type | 1.7264 | 0.6774 | 0.0299 |
| | | IV collagenase) | | | |
| NM_004530 | 8F.3.H4 | matrix metalloproteinase 2 (gelatinase A, 72 kDa gelatinase, 72 kDa type | 1.7255 | 0.6752 | 0.0299 |
| | | IV collagenase) | | | |
| NM_004530 | 7R.2.B9 | matrix metalloproteinase 2 (gelatinase A, 72 kDa gelatinase, 72 kDa type | 1.7202 | 0.673 | 0.0299 |
| | | IV collagenase) | | | |
| NM_004530 | 7R.3.B6 | matrix metalloproteinase 2 (gelatinase A, 72 kDa gelatinase, 72 kDa type | 1.7193 | 0.671 | 0.0299 |
| | | IV collagenase) | | | |
| AI983239 | 14N.4.A9 | Hs. cDNA FLJ32163 fis, clone PLACE6000371 | 1.7182 | 0.6688 | 0.0299 |
| NHF | 7R.1.C8 | | 1.716 | 0.6665 | 0.0299 |
| NM_005110 | 8F.7.G2 | glutamine-fructose-6-phosphate transaminase 2 | 1.7125 | 0.6641 | 0.0299 |
| NM_016950 | 7F.8.G3 | testican 3 | 1.7106 | 0.6619 | 0.0299 |
| NM_004530 | 7R.2.C6 | matrix metalloproteinase 2 (gelatinase A, 72 kDa gelatinase, 72 kDa type | 1.7073 | 0.66 | 0.0299 |
| | | IV collagenase) | | | |
| NM_004530 | 7R.5.E7 | matrix metalloproteinase 2 (gelatinase A, 72 kDa gelatinase, 72 kDa type | 1.7049 | 0.6581 | 0.0299 |
| | | IV collagenase) | | | |
| NM_004530 | 8R.10.D10 | matrix metalloproteinase 2 (gelatinase A, 72 kDa gelatinase, 72 kDa type | 1.7037 | 0.6564 | 0.0299 |
| | | IV collagenase) | | | |
| NM_000584 | 7F.8.G8 | interleukin 8 | 1.7004 | 0.6542 | 0.0299 |
| NM_004530 | 7R.3.G11 | matrix metalloproteinase 2 (gelatinase A, 72 kDa gelatinase, 72 kDa type | 1.6889 | 0.6521 | 0.0362 |
| | | IV collagenase) | | | |
| AK092836 | 1R.1.H3 | Homo sapiens cDNA FLJ35517 fis, clone SPLEN2000698 | 1.6722 | 0.6501 | 0.0362 |
| NM_006216 | 7F.6.A12 | serine (or cysteine) proteinase inhibitor, clade E (nexin, plasminogen | 1.6666 | 0.6482 | 0.0556 |
| | | activator inhibitor type 1), member 2 | | | |
| NHF | 7R.10.E11 | | 1.6639 | 0.6465 | 0.0556 |
| NHF | 8F.9.E4 | | 1.6636 | 0.6448 | 0.0556 |
| NHF | 7F.10.G3 | | 1.6521 | 0.6433 | 0.0822 |
| NM_004530 | 9F.4.F10 | matrix metalloproteinase 2 (gelatinase A, 72 kDa gelatinase, 72 kDa type | 1.6436 | 0.6413 | 0.0823 |
| | | IV collagenase) | | | |
| NM_004530 | 7R.3.F8 | matrix metalloproteinase 2 (gelatinase A, 72 kDa gelatinase, 72 kDa type | 1.6427 | 0.6398 | 0.0823 |
| | | IV collagenase) | | | |
| NM_004530 | 7R.5.C6 | matrix metalloproteinase 2 (gelatinase A, 72 kDa gelatinase, 72 kDa type | 1.6355 | 0.6379 | 0.0823 |
| | | IV collagenase) | | | |
| NM_004530 | 7R.2.A7 | matrix metalloproteinase 2 (gelatinase A, 72 kDa gelatinase, 72 kDa type | 1.6342 | 0.6362 | 0.0823 |
| | | IV collagenase) | | | |
| NM_006216 | 8F.6.B8 | serine (or cysteine) proteinase inhibitor, clade E (nexin, plasminogen | 1.6298 | 0.6346 | 0.0823 |
| | | activator inhibitor type 1), member 2 | | | |
| NM_004530 | 8R.3.A11 | matrix metalloproteinase 2 (gelatinase A, 72 kDa gelatinase, 72 kDa type | 1.6214 | 0.6329 | 0.0823 |
| | | IV collagenase) | | | |
| NM_004530 | 7R.8.H8 | matrix metalloproteinase 2 (gelatinase A, 72 kDa gelatinase, 72 kDa type | 1.6185 | 0.6314 | 0.0824 |
| | | IV collagenase) | | | |
| NM_000104 | 12R.3.E7 | cytochrome P450, subfamily I (dioxin-inducible), polypeptide 1 | 1.6088 | 0.6296 | 0.0824 |
| | | (glaucoma 3, primary infantile) | | | |
| NHF | 9R.5.G4 | | 1.6074 | 0.628 | 0.0824 |
| NM_001235 | 8R.6.B12 | serine (or cysteine) proteinase inhibitor, clade H (heat shock protein 47), | 1.6063 | 0.6264 | 0.0824 |
| | | member 2 | | | |
| NM_004530 | 7R.7.G7 | matrix metalloproteinase 2 (gelatinase A, 72 kDa gelatinase, 72 kDa type | 1.5962 | 0.6249 | 0.0899 |
| | | IV collagenase) | | | |
| NM_006216 | 8R.3.A3 | serine (or cysteine) proteinase inhibitor, clade E (nexin, plasminogen | 1.5928 | 0.6235 | 0.0899 |
| | | activator inhibitor type 1), member 2 | | | |
| NM_004530 | 7R.3.D11 | matrix metalloproteinase 2 (gelatinase A, 72 kDa gelatinase, 72 kDa type | 1.592 | 0.622 | 0.0899 |
| | | IV collagenase) | | | |
| NM_004530 | 7R.7.G8 | matrix metalloproteinase 2 (gelatinase A, 72 kDa gelatinase, 72 kDa type | 1.589 | 0.6203 | 0.0899 |
| | | IV collagenase) | | | |
| NM_004530 | 7R.4.E10 | matrix metalloproteinase 2 (gelatinase A, 72 kDa gelatinase, 72 kDa type | 1.587 | 0.6187 | 0.0934 |
| | | IV collagenase) | | | |
| NHF | 9R.5.A7 | | 1.5857 | 0.6173 | 0.0934 |
| NM_004966 | 7F.8.F5 | heterogeneous nuclear ribonucleoprotein F | 1.5768 | 0.6157 | 0.0968 |
| NM_004530 | 7R.9.D7 | matrix metalloproteinase 2 (gelatinase A, 72 kDa gelatinase, 72 kDa type | 1.5684 | 0.6143 | 0.0968 |
| | | IV collagenase) | | | |
| NHF | 7F.1.H10 | | 1.5594 | 0.6129 | 0.0969 |
| AK025599 | 9F.10.E12 | mannosidase, alpha, class 1A, member 1 | 1.5573 | 0.6116 | 0.0969 |
| NM_004530 | 7R.8.G6 | matrix metalloproteinase 2 (gelatinase A, 72 kDa gelatinase, 72 kDa type | 1.5565 | 0.61 | 0.0969 |
| | | IV collagenase) | | | |
| NHF | 7F.8.E7 | | 1.5511 | 0.6088 | 0.0969 |
| NM_004530 | 7R.7.H8 | matrix metalloproteinase 2 (gelatinase A, 72 kDa gelatinase, 72 kDa type | 1.5494 | 0.6073 | 0.0969 |
| | | IV collagenase) | | | |
| NM_002923 | 7R.9.F12 | regulator of G-protein signalling 2, 24 kDa | 1.5435 | 0.6061 | 0.105 |
| NM_000088 | 7R.2.H9 | collagen, type I, alpha 1 | 1.5432 | 0.6049 | 0.105 |
| NHF | 8F.9.G11 | | 1.5399 | 0.6036 | 0.1106 |
| NM_004530 | 9R.7.H8 | matrix metalloproteinase 2 (gelatinase A, 72 kDa gelatinase, 72 kDa type | 1.5393 | 0.6022 | 0.1106 |
| | | IV collagenase) | | | |
| NM_004530 | 7R.9.E8 | matrix metalloproteinase 2 (gelatinase A, 72 kDa gelatinase, 72 kDa type | 1.5375 | 0.6009 | 0.1106 |
| | | IV collagenase) | | | |
| NHF | 8F.9.D4 | | 1.5373 | 0.5995 | 0.1106 |
| AW005755 | 14N.5.G8 | macrophage migration inhibitory factor (glycosylation-inhibiting factor) | 1.5156 | 0.5982 | 0.1458 |
| NM_006216 | 8F.7.F6 | serine (or cysteine) proteinase inhibitor, clade E (nexin, plasminogen | 1.5148 | 0.5968 | 0.1458 |
| | | activator inhibitor type 1), member 2 | | | |
| NM_005110 | 8F.4.G2 | glutamine-fructose-6-phosphate transaminase 2 | 1.5097 | 0.5955 | 0.157 |
| AA873792 | 14N.8.D11 | small inducible cytokine A5 (RANTES) | 1.5056 | 0.5943 | 0.1585 |
| U72621 | 7F.2.C12 | pleiomorphic adenoma gene-like 1 | 1.5044 | 0.5931 | 0.1585 |
| NM_004530 | 7R.9.E10 | matrix metalloproteinase 2 (gelatinase A, 72 kDa gelatinase, 72 kDa type | 1.4992 | 0.5918 | 0.1613 |
| | | IV collagenase) | | | |
| NHF | 8F.9.A12 | | 1.4954 | 0.5906 | 0.164 |
| AW078807 | 7R.10.H7 | EST | 1.489 | 0.5892 | 0.1705 |
| NM_006216 | 8R.7.B1 | serine (or cysteine) proteinase inhibitor, clade E (nexin, plasminogen | 1.4861 | 0.5879 | 0.1716 |
| | | activator inhibitor type 1), member 2 | | | |
| NM_000358 | 7R.2.H8 | transforming growth factor, beta-induced, 68 kDa | 1.4853 | 0.5868 | 0.1716 |
| AK054688 | 8F.5.F10 | Homo sapiens cDNA FLJ30126 fis, clone BRACE1000114 | 1.4827 | 0.5856 | 0.1752 |
| NM_001235 | 7R.5.G1 | serine (or cysteine) proteinase inhibitor, clade H (heat shock protein 47), | 1.4812 | 0.5845 | 0.1752 |
| | | member 2 | | | |
| BC007583 | 8R.3.B8 | Homo sapiens, clone MGC: 15572 IMAGE: 3140342, mRNA, complete | 1.4812 | 0.5833 | 0.1752 |
| | | cds | | | |
| NM_007041 | 7F.3.E3 | arginyltransferase 1 | 1.4776 | 0.5823 | 0.1821 |
| NM_000088 | 7R.3.B10 | collagen, type I, alpha 1 | 1.4723 | 0.5812 | 0.1821 |
| NM_000089 | 7R.7.H9 | collagen, type I, alpha 2 | 1.4705 | 0.5801 | 0.1821 |
| NM_004404 | 9R.7.F10 | neural precursor cell expressed, developmentally down-regulated 5 | 1.4585 | 0.579 | 0.1901 |
| NM_004530 | 7R.8.A9 | matrix metalloproteinase 2 (gelatinase A, 72 kDa gelatinase, 72 kDa type | 1.4555 | 0.5779 | 0.2072 |
| | | IV collagenase) | | | |
| NM_001235 | 7R.10.C2 | serine (or cysteine) proteinase inhibitor, clade H (heat shock protein 47), | 1.4527 | 0.5767 | 0.2072 |
| | | member 2 | | | |
| NM_004530 | 7R.10.B8 | matrix metalloproteinase 2 (gelatinase A, 72 kDa gelatinase, 72 kDa type | 1.4497 | 0.5755 | 0.2072 |
| | | IV collagenase) | | | |
| NM_006216 | 9R.6.E12 | serine (or cysteine) proteinase inhibitor, clade E (nexin, plasminogen | 1.449 | 0.5743 | 0.2072 |
| | | activator inhibitor type 1), member 2 | | | |
| NM_001235 | 9R.4.H5 | serine (or cysteine) proteinase inhibitor, clade H (heat shock protein 47), | 1.4394 | 0.5731 | 0.2273 |
| | | member 2 | | | |
| NM_078467 | 9F.8.F8 | cyclin-dependent kinase inhibitor 1A (p21, Cip1) | 1.4376 | 0.5721 | 0.2273 |
| NM_005110 | 8F.7.C8 | glutamine-fructose-6-phosphate transaminase 2 | 1.4339 | 0.5711 | 0.229 |
| NM_033251 | 8R.3.B11 | ribosomal protein L13 | 1.4311 | 0.57 | 0.2298 |
| U97105 | 7F.8.G12 | Homo sapiens N2A3 mRNA, complete cds | 1.4251 | 0.5691 | 0.2358 |
| AI356451 | 14N.7.E9 | CD19 antigen | 1.4248 | 0.568 | 0.2358 |
| BI430544 | 11F.1.H5 | ESTs | 1.4231 | 0.567 | 0.2358 |
| BF732465 | 7F.6.G10 | tissue inhibitor of metalloproteinase 2 | 1.4228 | 0.5659 | 0.2358 |
| NM_001554 | 1R.1.H8 | cysteine-rich, angiogenic inducer, 61 | 1.4205 | 0.5648 | 0.2373 |
| NM_004530 | 7R.9.D3 | matrix metalloproteinase 2 (gelatinase A, 72 kDa gelatinase, 72 kDa type | 1.4181 | 0.5637 | 0.2416 |
| | | IV collagenase) | | | |
| NHF | 9R.5.A3 | | 1.4076 | 0.5627 | 0.2527 |
| NM_004530 | 7R.1.A3 | matrix metalloproteinase 2 (gelatinase A, 72 kDa gelatinase, 72 kDa type | 1.4064 | 0.5617 | 0.2527 |
| | | IV collagenase) | | | |
| NM_078467 | 9R.1.B8 | cyclin-dependent kinase inhibitor 1A (p21, Cip1) | 1.4036 | 0.5606 | 0.2527 |
| NM_004530 | 7R.1.G4 | matrix metalloproteinase 2 (gelatinase A, 72 kDa gelatinase, 72 kDa type | 1.402 | 0.5596 | 0.2527 |
| | | IV collagenase) | | | |
| NHF | 9F.3.G4 | | 1.401 | 0.5586 | 0.254 |
| BQ890604 | 9R.6.A8 | Homo sapiens URB mRNA, complete cds | 1.3978 | 0.5577 | 0.2633 |
| NM_002631 | 1F.1.D7 | phosphogluconate dehydrogenase | 1.3966 | 0.5566 | 0.2646 |
| N94503 | 7F.4.G10 | pregnancy-associated plasma protein A | 1.3938 | 0.5555 | 0.267 |
| AI400317 | 14N.3.H6 | ESTs | 1.3938 | 0.5545 | 0.267 |
| NM_078467 | 8R.5.H3 | cyclin-dependent kinase inhibitor 1A (p21, Cip1) | 1.3917 | 0.5536 | 0.2708 |
| NM_004530 | 7R.9.F4 | matrix metalloproteinase 2 (gelatinase A, 72 kDa gelatinase, 72 kDa type | 1.389 | 0.5526 | 0.2769 |
| | | IV collagenase) | | | |
| NM_000089 | 9R.8.F11 | collagen, type I, alpha 2 | 1.3832 | 0.5517 | 0.2904 |
| NM_001710 | 7F.6.G11 | B-factor, properdin | 1.3819 | 0.5508 | 0.2904 |
| NM_004530 | 7R.6.D8 | matrix metalloproteinase 2 (gelatinase A, 72 kDa gelatinase, 72 kDa type | 1.3766 | 0.5498 | 0.291 |
| | | IV collagenase) | | | |
| NHF | 7R.1.C2 | | 1.3755 | 0.5489 | 0.291 |
| AA004368 | 14N.1.G12 | hypothetical protein FLJ21269 | 1.3707 | 0.5481 | 0.298 |
| NHF | 7F.9.B10 | | 1.3652 | 0.5471 | 0.3 |
| BC007583 | 7R.4.E8 | Homo sapiens, clone MGC: 15572 IMAGE: 3140342, mRNA, complete | 1.3624 | 0.5462 | 0.3034 |
| | | cds | | | |
| BI430544 | 12R.1.F10 | ESTs | 1.3591 | 0.5454 | 0.3116 |
| BC014836 | 7F.4.G11 | Homo sapiens, mitochondrial ribosomal protein L3, clone MGC: 9373 | 1.3553 | 0.5445 | 0.3199 |
| | | IMAGE: 3860982, mRNA, complete cds | | | |
| W72329 | 14N.7.B12 | lymphotoxin alpha (TNF superfamily, member 1) | 1.3546 | 0.5435 | 0.3199 |
| NHF | 9R.3.C2 | | 1.3544 | 0.5427 | 0.3199 |
| NM_000584 | 7F.8.E11 | interleukin 8 | 1.3537 | 0.5418 | 0.3199 |
| NM_002993 | 7F.5.G8 | chemokine (C—X—C motif) ligand 6 (granulocyte chemotactic protein 2) | 1.3438 | 0.5409 | 0.3528 |
| NM_002844 | 8F.8.F10 | protein tyrosine phosphatase, receptor type, K | 1.3435 | 0.54 | 0.3528 |
| AA451863 | 14N.7.C11 | CD4 antigen (p55) | 1.3424 | 0.539 | 0.3528 |
| AW772163 | 14N.1.B11 | hypothetical protein FLJ20401 | 1.336 | 0.5381 | 0.3704 |
| NM_000088 | 8F.5.D12 | collagen, type I, alpha 1 | 1.332 | 0.5372 | 0.3784 |
| BI430544 | 11F.1.H7 | ESTs | 1.3292 | 0.5362 | 0.3784 |
| NM_002009 | 9R.7.D9 | fibroblast growth factor 7 (keratinocyte growth factor) | 1.3249 | 0.5352 | 0.3818 |
| AA146772 | 14N.4.G10 | 2,5-oligoadenylate synthetase 1 (40-46 kD) | 1.3231 | 0.5344 | 0.3879 |
| H89562 | 14N.4.F7 | hypothetical protein FLJ21817 similar to Rhoip2 | 1.3223 | 0.5337 | 0.3879 |
| BC036075 | 1R.1.D10 | PDZ domain protein GIPC2 | 1.322 | 0.5329 | 0.3879 |
| NM_001964 | 9F.6.G6 | early growth response 1 | 1.3187 | 0.5322 | 0.3884 |
| NM_006216 | 8R.5.D8 | serine (or cysteine) proteinase inhibitor, clade E (nexin, plasminogen | 1.3176 | 0.5313 | 0.3933 |
| | | activator inhibitor type 1), member 2 | | | |
| NHF | 7F.8.H7 | | 1.316 | 0.5306 | 0.4031 |
| T47442 | 14N.4.D10 | protein C receptor, endothelial (EPCR) | 1.3125 | 0.5296 | 0.4057 |
| AA057156 | 14N.1.E7 | interleukin 2 receptor, beta | 1.3109 | 0.5287 | 0.4061 |
| NM_004048 | 1R.1.G11 | beta-2-microglobulin | 1.3098 | 0.528 | 0.4069 |
| NHF | 8F.4.G9 | | 1.3086 | 0.5271 | 0.4069 |
| NM_004530 | 9R.8.D6 | matrix metalloproteinase 2 (gelatinase A, 72 kDa gelatinase, 72 kDa type | 1.3072 | 0.5263 | 0.4138 |
| | | IV collagenase) | | | |
| NM_002982 | 7F.4.H8 | chemokine (C—C motif) ligand 2 | 1.3045 | 0.5255 | 0.4145 |
| NM_006350 | 7F.3.E10 | follistatin | 1.304 | 0.5247 | 0.4145 |
| NM_000089 | 8R.1.G3 | collagen, type I, alpha 2 | 1.3017 | 0.5239 | 0.4195 |
| NHF | 9R.1.H3 | | 1.3017 | 0.5231 | 0.4195 |
| NHF | 8F.1.G11 | | 1.3003 | 0.5223 | 0.4262 |
| NM_003254 | 7R.4.B12 | tissue inhibitor of metalloproteinase 1 (erythroid potentiating activity, | 1.2967 | 0.5216 | 0.4328 |
| | | collagenase inhibitor) | | | |
| W68141 | 14N.1.C8 | protein kinase, cAMP-dependent, catalytic, alpha | 1.2939 | 0.5208 | 0.4372 |
| NM_004530 | 7R.9.F1 | matrix metalloproteinase 2 (gelatinase A, 72 kDa gelatinase, 72 kDa type | 1.288 | 0.52 | 0.4544 |
| | | IV collagenase) | | | |
| NM_005803 | 7R.10.H8 | flotillin 1 | 1.2856 | 0.5192 | 0.4566 |
| T72877 | 14N.4.D8 | EST | 1.2832 | 0.5184 | 0.4634 |
| NHF | 7F.8.G5 | | 1.2808 | 0.5176 | 0.4634 |
| AA884967 | 14N.8.D7 | nitric oxide synthase 3 (endothelial cell) | 1.2806 | 0.5168 | 0.4634 |
| AA520985 | 14N.2.G8 | rab3 GTPase-activating protein, non-catalytic subunit (150 kD) | 1.2765 | 0.516 | 0.4677 |
| NHF | 7F.4.G12 | | 1.2763 | 0.5153 | 0.4677 |
| NHF | 9R.10.A1 | | 1.2736 | 0.5146 | 0.474 |
| BM756510 | 7F.5.G10 | spermidine/spermine N1-acetyltransferase | 1.2733 | 0.5139 | 0.474 |
| NHF | 9R.3.F7 | | 1.2692 | 0.5132 | 0.4842 |
| H82431 | 14N.2.G7 | prospero-related homeobox 1 | 1.2671 | 0.5124 | 0.4923 |
| NM_004417 | 9R.10.H7 | dual specificity phosphatase 1 | 1.2657 | 0.5118 | 0.4923 |
| NM_003842 | 1R.2.H12 | tumor necrosis factor receptor superfamily, member 10b | 1.2653 | 0.511 | 0.4923 |
| AK074259 | 7F.1.H3 | Homo sapiens cDNA FLJ30436 fis, clone BRACE2009037 | 1.265 | 0.5103 | 0.4923 |
| NM_153373 | 8F.4.H10 | hypothetical protein MGC15875 | 1.2649 | 0.5096 | 0.4923 |
| BU626315 | 8F.9.D2 | collagen, type V, alpha 1 | 1.2611 | 0.5088 | 0.5057 |
| AA954921 | 14N.4.G11 | ATP binding protein associated with cell differentiation | 1.2608 | 0.5081 | 0.5057 |
| NM_002982 | 7F.6.F3 | chemokine (C—C motif) ligand 2 | 1.2581 | 0.5073 | 0.5076 |
| AW083684 | 14N.4.A12 | EST | 1.2574 | 0.5067 | 0.5076 |
| D83776 | 7F.8.D11 | KIAA0191 protein | 1.2561 | 0.506 | 0.5094 |
| NHF | 7R.2.G6 | | 1.2551 | 0.5054 | 0.5094 |
| NM_006216 | 8F.2.G11 | serine (or cysteine) proteinase inhibitor, clade E (nexin, plasminogen | 1.2539 | 0.5048 | 0.515 |
| | | activator inhibitor type 1), member 2 | | | |
| NM_000089 | 8F.5.G9 | collagen, type I, alpha 2 | 1.2501 | 0.504 | 0.5342 |
| NM_000584 | 7F.1.D11 | interleukin 8 | 1.2491 | 0.5033 | 0.5342 |
| NM_006088 | 7F.7.G6 | tubulin, beta, 2 | 1.2485 | 0.5026 | 0.5342 |
| NM_078467 | 8R.1.E10 | cyclin-dependent kinase inhibitor 1A (p21, Cip1) | 1.2476 | 0.502 | 0.5342 |
| AK097395 | 7F.5.A10 | superoxide dismutase 2, mitochondrial | 1.2431 | 0.5012 | 0.5484 |
| NM_004000 | 7F.4.G5 | chitinase 3-like 2 | 1.2419 | 0.5005 | 0.5616 |
| NHF | 7F.9.E7 | | 1.2399 | 0.4999 | 0.5616 |
| BU626315 | 8F.7.D3 | collagen, type V, alpha 1 | 1.2398 | 0.4992 | 0.5616 |
| AJ238214 | 8F.1.B9 | WD repeat domain 9 | 1.2352 | 0.4986 | 0.5664 |
| BC015615 | 7R.6.G6 | Homo sapiens, Similar to peroxisomal biogenesis factor 6, clone | 1.2349 | 0.498 | 0.5664 |
| | | MGC: 23066 IMAGE: 4840674, mRNA, complete cds | | | |
| W02227 | 14N.1.H10 | hypothetical protein MGC5391 | 1.2318 | 0.4973 | 0.569 |
| AJ238214 | 8F.5.C6 | WD repeat domain 9 | 1.231 | 0.4967 | 0.569 |
| H22922 | 14N.4.H9 | manic fringe homolog (Drosophila) | 1.2305 | 0.496 | 0.569 |
| NM_002026 | 7R.8.B8 | fibronectin 1 | 1.2301 | 0.4953 | 0.569 |
| NM_002659 | 9F.1.C8 | plasminogen activator, urokinase receptor | 1.2287 | 0.4948 | 0.5704 |
| AV763779 | 7F.3.G4 | WD-repeat protein | 1.2276 | 0.4941 | 0.5753 |
| BE963194 | 7F.8.H10 | EST | 1.2191 | 0.4935 | 0.6178 |
| NM_000584 | 7F.4.D3 | interleukin 8 | 1.2162 | 0.4928 | 0.6258 |
| NM_002291 | 8R.7.C9 | laminin, beta 1 | 1.2139 | 0.4922 | 0.6287 |
| BI830199 | 8R.9.E11 | likely ortholog of mouse Urb | 1.2134 | 0.4914 | 0.6287 |
| NM_032704 | 7R.6.A3 | tubulin alpha 6 | 1.213 | 0.4908 | 0.6287 |
| NHF | 7F.8.A6 | | 1.2127 | 0.4901 | 0.6287 |
| NM_001997 | 1R.1.H11 | Finkel-Biskis-Reilly murine sarcoma virus (FBR-MuSV) ubiquitously | 1.2125 | 0.4895 | 0.6287 |
| | | expressed (fox derived); ribosomal protein S30 | | | |
| NM_005110 | 8F.5.E12 | glutamine-fructose-6-phosphate transaminase 2 | 1.2113 | 0.4888 | 0.6332 |
| BU626315 | 8F.3.E7 | collagen, type V, alpha 1 | 1.2086 | 0.4883 | 0.6505 |
| AI432366 | 14N.3.H5 | ESTs | 1.2081 | 0.4876 | 0.6505 |
| AK025773 | 12R.1.E3 | Homo sapiens cDNA: FLJ22120 fis, clone HEP18874 | 1.2077 | 0.4869 | 0.6505 |
| AV706813 | 7R.7.G1 | ESTs, Highly similar to IPYR_HUMAN Inorganic pyrophosphatase | 1.2013 | 0.4863 | 0.6752 |
| | | (Pyrophosphate phospho-hydrolase) (PPase) [H. sapiens] | | | |
| NM_000104 | 12F.2.G4 | cytochrome P450, subfamily I (dioxin-inducible), polypeptide 1 | 1.2012 | 0.4857 | 0.6752 |
| | | (glaucoma 3, primary infantile) | | | |
| AF067170 | 7F.4.B2 | endosulfine alpha | 1.1975 | 0.485 | 0.6885 |
| NM_005415 | 9F.2.H7 | solute carrier family 20 (phosphate transporter), member 1 | 1.1968 | 0.4844 | 0.6885 |
| NM_016306 | 7R.5.D11 | DnaJ (Hsp40) homolog, subfamily B, member 11 | 1.1961 | 0.4837 | 0.6885 |
| NM_004199 | 7R.2.H7 | procollagen-proline, 2-oxoglutarate 4-dioxygenase (proline 4- | 1.1959 | 0.4831 | 0.6885 |
| | | hydroxylase), alpha polypeptide II | | | |
| BU626315 | 8F.2.C5 | collagen, type V, alpha 1 | 1.1952 | 0.4825 | 0.6885 |
| NHF | 7F.8.D6 | | 1.1951 | 0.482 | 0.6885 |
| NM_005347 | 8F.7.F2 | heat shock 70 kDa protein 5 (glucose-regulated protein, 78 kDa) | 1.193 | 0.4814 | 0.6941 |
| NM_006307 | 7F.4.A3 | sushi-repeat-containing protein, X chromosome | 1.1894 | 0.4808 | 0.6994 |
| BM690558 | 7R.6.G10 | ESTs, Highly similar to interferon induced transmembrane protein 3 (1- | 1.1873 | 0.4802 | 0.6994 |
| | | 8U); interferon-inducible [Homo sapiens] [H. sapiens] | | | |
| NM_012425 | 8F.6.G8 | Ras suppressor protein 1 | 1.187 | 0.4796 | 0.6994 |
| AI359876 | 12F.2.C11 | EST | 1.1863 | 0.479 | 0.6994 |
| AA448261 | 14N.1.F3 | high-mobility group (nonhistone chromosomal) protein isoforms I and Y | 1.1855 | 0.4784 | 0.6994 |
| N95334 | 14N.3.F3 | activin A receptor type II-like 1 | 1.1849 | 0.4778 | 0.6994 |
| NHF | 8R.4.F4 | | 1.1843 | 0.4773 | 0.6994 |
| AK025773 | 12F.3.H10 | Homo sapiens cDNA: FLJ22120 fis, clone HEP18874 | 1.1842 | 0.4767 | 0.6994 |
| AF506819 | 7R.9.F6 | Homo sapiens URB mRNA, complete cds | 1.1836 | 0.4762 | 0.6994 |
| NM_021034 | 7R.1.C5 | interferon induced transmembrane protein 3 (1-8U) | 1.1812 | 0.4755 | 0.7123 |
| NM_007040 | 7F.3.G1 | E1B-55 kDa-associated protein 5 | 1.18 | 0.475 | 0.7134 |
| NM_001235 | 8F.5.F1 | serine (or cysteine) proteinase inhibitor, clade H (heat shock protein 47), | 1.1796 | 0.4744 | 0.7134 |
| | | member 2 | | | |
| NHF | 9F.6.H10 | | 1.1788 | 0.4738 | 0.7134 |
| A995402 | 14N.5.D12 | colony stimulating factor 2 (granulocyte-macrophage) | 1.1758 | 0.4732 | 0.7202 |
| AA625981 | 14N.2.C5 | FK506 binding protein 1A (12 kD) | 1.1745 | 0.4727 | 0.7411 |
| NHF | 7F.6.F9 | | 1.1711 | 0.472 | 0.7647 |
| NM_001101 | 7R.5.G8 | actin, beta | 1.1677 | 0.4715 | 0.782 |
| NHF | 8F.8.E10 | | 1.1667 | 0.4709 | 0.787 |
| NM_003003 | 1F.1.H1 | SEC14-like 1 (S. cerevisiae) | 1.1632 | 0.4704 | 0.804 |
| AA676848 | 14N.2.G11 | far upstream element (FUSE) binding protein 1 | 1.1622 | 0.4699 | 0.8075 |
| NM_000089 | 7R.7.H2 | collagen, type I, alpha 2 | 1.1572 | 0.4693 | 0.8371 |
| NHF | 8R.1.E8 | | 1.1535 | 0.4689 | 0.8521 |
| NM_000521 | 8R.8.D8 | hexosaminidase B (beta polypeptide) | 1.1531 | 0.4682 | 0.8521 |
| NM_152862 | 7F.6.F1 | actin related protein 2/3 complex, subunit 2, 34 kDa | 1.1503 | 0.4677 | 0.8624 |
| AI273932 | 8F.7.G9 | EST | 1.1473 | 0.4671 | 0.8708 |
| AB051510 | 1R.1.B11 | deleted in liver cancer 1 | 1.1467 | 0.4666 | 0.8708 |
| AJ318805 | 8F.8.F7 | ESTs, Weakly similar to hypothetical protein FLJ20378 [Homo sapiens] | 1.1461 | 0.466 | 0.8708 |
| | | [H. sapiens] | | | |
| NM_006290 | 7F.4.G6 | tumor necrosis factor, alpha-induced protein 3 | 1.1453 | 0.4655 | 0.8708 |
| AW088013 | 14N.4.A10 | EST | 1.1437 | 0.465 | 0.8926 |
| NM_006216 | 8R.4.A3 | serine (or cysteine) proteinase inhibitor, clade E (nexin, plasminogen | 1.1409 | 0.4645 | 0.9027 |
| | | activator inhibitor type 1), member 2 | | | |
| NM_021874 | 9R.2.A8 | cell division cycle 25B | 1.1394 | 0.464 | 0.9027 |
| AK097395 | 8F.3.D7 | superoxide dismutase 2, mitochondrial | 1.1375 | 0.4634 | 0.9087 |
| NHF | 8F.1.H8 | | 1.1369 | 0.4629 | 0.9087 |
| NM_004530 | 7R.6.D11 | matrix metalloproteinase 2 (gelatinase A, 72 kDa gelatinase, 72 kDa type | 1.1347 | 0.4623 | 0.9171 |
| | | IV collagenase) | | | |
| | | serine (or cysteine) proteinase inhibitor, clade E (nexin, plasminogen | | | |
| NM_006216 | 7F.3.E5 | activator inhibitor type 1), member 2 | 1.1293 | 0.4617 | 0.9733 |
| NM_000584 | 7F.6.B4 | interleukin 8 | 1.1264 | 0.4612 | 0.988 |
| NM_001831 | 7R.10.H11 | clusterin (complement lysis inhibitor, SP-40, 40, sulfated glycoprotein 2, | 1.1225 | 0.4607 | 1.004 |
| | | testosterone-repressed prostate message 2, apolipoprotein J) | | | |
| NM_005402 | 8F.7.A6 | v-ral simian leukemia viral oncogene homolog A (ras related) | 1.1214 | 0.4601 | 1.0053 |
| AK000724 | 12F.1.G12 | Homo sapiens cDNA FLJ20717 fis, clone HEP18380 | 1.1199 | 0.4596 | 1.0144 |
| NM_002966 | 7R.6.A11 | S100 calcium binding protein A10 (annexin II ligand, calpactin I, light | 1.1182 | 0.4591 | 1.0295 |
| | | polypeptide (p11)) | | | |
| NM_005746 | 1R.2.H5 | pre-B-cell colony-enhancing factor | 1.1165 | 0.4585 | 1.0295 |
| NHF | 8F.7.F12 | | 1.1164 | 0.4581 | 1.0295 |
| NM_005720 | 8F.7.D12 | actin related protein 2/3 complex, subunit 1B, 41 kDa | 1.1162 | 0.4576 | 1.0295 |
| NM_004530 | 7R.7.H12 | matrix metalloproteinase 2 (gelatinase A, 72 kDa gelatinase, 72 kDa type | 1.1159 | 0.4571 | 1.0295 |
| | | IV collagenase) | | | |
| NM_001444 | 1R.1.H5 | fatty acid binding protein 5 (psoriasis-associated) | 1.1143 | 0.4566 | 1.0344 |
| NHF | 7R.3.G6 | | 1.1136 | 0.4561 | 1.0381 |
| NM_002982 | 9R.4.E6 | chemokine (C—C motif) ligand 2 | 1.1123 | 0.4556 | 1.0418 |
| NM_006216 | 8F.6.B2 | serine (or cysteine) proteinase inhibitor, clade E (nexin, plasminogen | 1.1112 | 0.455 | 1.0432 |
| | | activator inhibitor type 1), member 2 | | | |
| NM_000584 | 7F.1.C5 | interleukin 8 | 1.1103 | 0.4545 | 1.0432 |
| NM_021103 | 7R.3.F5 | thymosin, beta 10 | 1.1099 | 0.454 | 1.0432 |
| NM_003190 | 1F.1.A10 | TAP binding protein (tapasin) | 1.1092 | 0.4535 | 1.0432 |
| NM_138271 | 12R.2.H8 | alpha thalassemia/mental retardation syndrome X-linked (RAD54 | 1.1086 | 0.453 | 1.0432 |
| | | homolog, S. cerevisiae) | | | |
| NM_006307 | 8R.10.D11 | sushi-repeat-containing protein, X chromosome | 1.1081 | 0.4525 | 1.0432 |
| AA857343 | 14N.4.G4 | TAF15 RNA polymerase II, TATA box binding protein (TBP)-associated | 1.1077 | 0.4521 | 1.0432 |
| | | factor, 68 kD | | | |
| BU626315 | 8F.10.F6 | collagen, type V, alpha 1 | 1.1076 | 0.4516 | 1.0432 |
| AV719568 | 1R.1.B6 | EST | 1.1069 | 0.4511 | 1.0432 |
| NM_003816 | 7F.8.G10 | a disintegrin and metalloproteinase domain 9 (meltrin gamma) | 1.1065 | 0.4506 | 1.0432 |
| BC008791 | 7R.4.D8 | Homo sapiens, tubulin, beta 5, clone MGC: 4029 IMAGE: 3617988, | 1.102 | 0.4501 | 1.0628 |
| | | mRNA, complete cds | | | |
| AB033056 | 1R.1.E12 | PTPRF interacting protein, binding protein 1 (liprin beta 1) | 1.0998 | 0.4496 | 1.0725 |
| AK095469 | 7F.6.F12 | Homo sapiens cDNA FLJ38150 fis, clone D9OST2004073 | 1.0978 | 0.449 | 1.0821 |
| NHF | 8F.7.D11 | | 1.0971 | 0.4485 | 1.0831 |
| AA884967 | 14N.6.D7 | nitric oxide synthase 3 (endothelial cell) | 1.0948 | 0.4481 | 1.091 |
| BI430544 | 11F.1.F9 | ESTs | 1.0944 | 0.4476 | 1.091 |
| NM_004434 | 7R.2.H12 | echinoderm microtubule associated protein like 1 | 1.0942 | 0.4472 | 1.091 |
| BQ009646 | 8F.6.E11 | modulator recognition factor 2 | 1.0936 | 0.4467 | 1.0956 |
| NHF | 7R.1.E3 | | 1.0924 | 0.4462 | 1.1 |
| AB046844 | 7F.3.D6 | G protein-coupled receptor 107 | 1.092 | 0.4457 | 1.1 |
| NM_005402 | 8F.4.B8 | v-ral simian leukemia viral oncogene homolog A (ras related) | 1.0899 | 0.4452 | 1.101 |
| NM_002421 | 12R.3.H11 | matrix metalloproteinase 1 (interstitial collagenase) | 1.0889 | 0.4447 | 1.1019 |
| N54794 | 14N.1.C11 | serine (or cysteine) proteinase inhibitor, clade E (nexin, plasminogen | 1.0873 | 0.4443 | 1.1333 |
| | | activator inhibitor type 1), member 1 | | | |
| T86934 | 14N.7.B10 | CD79A antigen (immunoglobulin-associated alpha) | 1.083 | 0.4438 | 1.156 |
| NM_000393 | 7R.7.G11 | collagen, type V, alpha 2 | 1.0813 | 0.4432 | 1.1663 |
| NM_005720 | 8F.10.A2 | actin related protein 2/3 complex, subunit 1B, 41 kDa | 1.0794 | 0.4427 | 1.1756 |
| NM_001878 | 8F.8.C3 | cellular retinoic acid binding protein 2 | 1.0788 | 0.4423 | 1.1756 |
| NM_001235 | 9R.9.F12 | serine (or cysteine) proteinase inhibitor, clade H (heat shock protein 47), | 1.0786 | 0.4418 | 1.1756 |
| | | member 2 | | | |
| NM_005720 | 8F.10.C6 | actin related protein 2/3 complex, subunit 18, 41 kDa | 1.0756 | 0.4412 | 1.197 |
| NM_000584 | 7F.6.H6 | interleukin 8 | 1.0754 | 0.4408 | 1.197 |
| BI870836 | 8F.1.D1 | ESTs, Moderately similar to 810024J URF 4 [Homo sapiens] [H. sapiens] | 1.0724 | 0.4404 | 1.2248 |
| NM_000584 | 7F.1.D1 | interleukin 8 | 1.0722 | 0.44 | 1.2248 |
| NM_006169 | 9R.10.G11 | nicotinamide N-methyltransferase | 1.0716 | 0.4395 | 1.2248 |
| AI813947 | 7F.1.D5 | ESTs, Highly similar to ribosomal protein S2; 40S ribosomal protein S2 | 1.0711 | 0.439 | 1.2248 |
| | | [Homo sapiens] [H. sapiens] | | | |
| M14219 | 7F.5.F12 | Human chondroitin/dermatan sulfate proteoglycan (PG40) core protein | 1.0704 | 0.4385 | 1.2265 |
| | | mRNA, complete cds | | | |
| N68859 | 14N.7.G4 | intercellular adhesion molecule 1 (CD54), human rhinovirus receptor | 1.0689 | 0.438 | 1.2397 |
| AK097395 | 7F.10.E3 | superoxide dismutase 2, mitochondrial | 1.0678 | 0.4375 | 1.2449 |
| NM_021111 | 7F.8.G6 | reversion-inducing-cysteine-rich protein with kazal motifs | 1.0659 | 0.437 | 1.2534 |
| AA102526 | 14N.7.C2 | interleukin 8 | 1.0655 | 0.4365 | 1.2534 |
| NM_006335 | 7R.7.G2 | translocase of inner mitochondrial membrane 17 homolog A (yeast) | 1.0655 | 0.4361 | 1.2534 |
| NM_003029 | 7R.6.A8 | SHC (Src homology 2 domain containing) transforming protein 1 | 1.0642 | 0.4356 | 1.2629 |
| AA454607 | 14N.1.H8 | BRIX | 1.0638 | 0.4351 | 1.2629 |
| NM_000980 | 7R.6.A4 | ribosomal protein L18a | 1.0633 | 0.4347 | 1.2629 |
| AA039932 | 14N.7.A12 | thromboxane A2 receptor | 1.0624 | 0.4343 | 1.2646 |
| NM_005347 | 7F.6.G6 | heat shock 70 kDa protein 5 (glucose-regulated protein, 78 kDa) | 1.0614 | 0.4338 | 1.2665 |
| NM_004369 | 7F.1.E9 | collagen, type VI, alpha 3 | 1.0608 | 0.4334 | 1.2665 |
| NM_002707 | 7F.6.F2 | protein phosphatase 1G (formerly 2C), magnesium-dependent, gamma | 1.0602 | 0.433 | 1.2665 |
| | | isoform | | | |
| N68859 | 14N.5.G4 | intercellular adhesion molecule 1 (CD54), human rhinovirus receptor | 1.0601 | 0.4326 | 1.2665 |
| NM_006307 | 8R.4.B10 | sushi-repeat-containing protein, X chromosome | 1.0591 | 0.4321 | 1.2665 |
| AI620703 | 8F.3.C7 | ESTs, Moderately similar to 0512543A oxidase II, cytochrome [Homo | 1.0586 | 0.4317 | 1.2665 |
| | | sapiens] [H. sapiens] | | | |
| NM_002508 | 8R.3.G11 | nidogen (enactin) | 1.0579 | 0.4312 | 1.2665 |
| AI311932 | 14N.3.B3 | glia maturation factor, gamma | 1.0578 | 0.4308 | 1.2665 |
| BI830199 | 8R.1.E11 | likely ortholog of mouse Urb | 1.0573 | 0.4303 | 1.2692 |
| AW148618 | 9R.7.D1 | EST, Moderately similar to 810024E cytochrome oxidase III [Homo | 1.056 | 0.4299 | 1.2768 |
| | | sapiens] [H. sapiens] | | | |
| NHF | 9F.3.A3 | | 1.0559 | 0.4295 | 1.2768 |
| NHF | 8F.1.G7 | | 1.0535 | 0.4291 | 1.3056 |
| BI830199 | 8R.1.D6 | likely ortholog of mouse Urb | 1.0521 | 0.4287 | 1.3151 |
| NHF | 7F.1.H5 | Homo sapiens, Similar to helicase-like protein NHL, clone MGC: 665 | 1.0516 | 0.4283 | 1.3151 |
| BC000673 | 7R.1.B1 | IMAGE: 3347926, mRNA, complete cds | 1.0509 | 0.4278 | 1.3176 |
| W80688 | 14N.1.H6 | KIAA0852 protein | 1.0497 | 0.4274 | 1.322 |
| NM_006435 | 7R.7.B10 | interferon induced transmembrane protein 2 (1-8D) | 1.0493 | 0.427 | 1.322 |
| NM_015380 | 7F.3.D8 | CGI-51 protein | 1.0491 | 0.4266 | 1.322 |
| AA102526 | 14N.5.C3 | interleukin 8 | 1.0481 | 0.4262 | 1.3312 |
| NM_006307 | 8R.4.H12 | sushi-repeat-containing protein, X chromosome | 1.0474 | 0.4258 | 1.333 |
| AW148618 | 8R.9.A4 | EST, Moderately similar to 810024E cytochrome oxidase III [Homo | 1.0464 | 0.4254 | 1.3354 |
| | | sapiens] [H. sapiens] | | | |
| NHF | 8R.8.H3 | | 1.0406 | 0.425 | 1.3904 |
| BC011620 | 7F.8.F8 | hypothetical protein MGC2668 | 1.0404 | 0.4245 | 1.3904 |
| NM_002290 | 9R.7.H12 | laminin, alpha 4 | 1.04 | 0.4241 | 1.3904 |
| NM_006325 | 8F.10.G7 | RAN, member RAS oncogene family | 1.0399 | 0.4238 | 1.3904 |
| NM_001533 | 1R.1.D11 | heterogeneous nuclear ribonucleoprotein L | 1.0392 | 0.4233 | 1.3926 |
| H02884 | 14N.3.B12 | cadherin 5, type 2, VE-cadherin (vascular epithelium) | 1.0385 | 0.423 | 1.3939 |
| NM_000584 | 7F.6.H9 | interleukin 8 | 1.0378 | 0.4226 | 1.3941 |
| AA626356 | 14N.2.F7 | ubiquitin specific protease 18 | 1.0362 | 0.4221 | 1.4087 |
| AF116718 | 8F.2.B4 | hypothetical protein PRO2900 | 1.0357 | 0.4218 | 1.4109 |
| NM_012242 | 9F.1.G12 | dickkopf homolog 1 (Xenopus laevis) | 1.0346 | 0.4213 | 1.4234 |
| AI334914 | 14N.5.E12 | integrin, alpha 2b (platelet glycoprotein IIb of IIb/IIIa complex, antigen | 1.0328 | 0.4209 | 1.4379 |
| | | CD41B) | | | |
| NM_002290 | 8R.1.G9 | laminin, alpha 4 | 1.0325 | 0.4205 | 1.4379 |
| AJ131244 | 8F.5.G6 | SEC24 related gene family, member A (S. cerevisiae) | 1.0305 | 0.4201 | 1.4423 |
| NM_002982 | 8R.9.A2 | chemokine (C—C motif) ligand 2 | 1.0305 | 0.4197 | 1.4423 |
| NM_000584 | 7F.2.D12 | interleukin 8 | 1.0303 | 0.4193 | 1.4423 |
| NHF | 7R.5.G11 | | 1.0301 | 0.4189 | 1.4423 |
| R16547 | 14N.1.F12 | hypothetical protein BC014339 | 1.028 | 0.4185 | 1.4495 |
| AI652836 | 14N.7.F11 | T cell activation, increased late expression | 1.0278 | 0.4181 | 1.4495 |
| NM_004000 | 7F.7.E10 | chitinase 3-like 2 | 1.0268 | 0.4178 | 1.4607 |
| NM_001122 | 12F.2.G3 | adipose differentiation-related protein | 1.0268 | 0.4174 | 1.4607 |
| NHF | 7R.10.H5 | | 1.0264 | 0.417 | 1.4607 |
| AI961881 | 14N.4.C3 | SEC13-like 1 (S. cerevisiae) | 1.0262 | 0.4166 | 1.4607 |
| NM_002852 | 9R.5.B1 | pentaxin-related gene, rapidly induced by IL-1 beta | 1.0256 | 0.4162 | 1.4608 |
| AK025773 | 7F.8.D8 | Homo sapiens cDNA: FLJ22120 fis, clone HEP18874 | 1.0231 | 0.4158 | 1.4883 |
| NHF | 8F.8.C12 | | 1.0227 | 0.4154 | 1.4883 |
| AL832199 | 1R.1.C7 | hypothetical protein FLJ30829 | 1.0218 | 0.415 | 1.501 |
| NHF | 7R.1.B5 | | 1.0214 | 0.4147 | 1.501 |
| NM_003191 | 12R.1.F12 | threonyl-tRNA synthetase | 1.0207 | 0.4143 | 1.5104 |
| AA629264 | 14N.2.G3 | pleckstrin homology, Sec7 and coiled/coil domains 3 | 1.0194 | 0.4139 | 1.5104 |
| N25262 | 14N.1.G11 | hypothetical protein FLJ10607 similar to glucosamine-phosphate N- | 1.0192 | 0.4136 | 1.5104 |
| | | acetyltransferase | | | |
| AA192691 | 8F.5.A1 | EST | 1.0189 | 0.4132 | 1.5104 |
| NM_015185 | 1R.1.A11 | Cdc42 guanine nucleotide exchange factor (GEF) 9 | 1.0185 | 0.4128 | 1.5104 |
| NM_005803 | 8F.10.F7 | flotillin 1 | 1.0183 | 0.4124 | 1.5104 |
| NHF | 7F.8.G11 | | 1.0181 | 0.4119 | 1.5104 |
| NM_003246 | 1R.2.B8 | thrombospondin 1 | 1.018 | 0.4115 | 1.5104 |
| NM_000584 | 7F.4.E10 | interleukin 8 | 1.0176 | 0.4111 | 1.5104 |
| NHF | 7F.1.B11 | | 1.0172 | 0.4107 | 1.5104 |
| NM_004530 | 7R.9.D4 | matrix metalloproteinase 2 (gelatinase A, 72 kDa gelatinase, 72 kDa type | 1.017 | 0.4103 | 1.5104 |
| | | IV collagenase) | | | |
| NM_005063 | 8F.5.F12 | stearoyl-CoA desaturase (delta-9-desaturase) | 1.0165 | 0.4099 | 1.5113 |
| NM_006745 | 9F.10.G7 | sterol-C4-methyl oxidase-like | 1.0141 | 0.4095 | 1.5254 |
| NM_002160 | 7F.5.G6 | tenascin C (hexabrachion) | 1.0139 | 0.4092 | 1.5254 |
| AK026408 | 7F.6.D8 | Homo sapiens cDNA: FLJ22755 fis, clone KAIA0769 | 1.0123 | 0.4088 | 1.5394 |
| AA423867 | 14N.1.E8 | multimerin | 1.0098 | 0.4084 | 1.5569 |
| NM_005420 | 8R.4.B11 | sulfotransferase, estrogen-preferring | 1.0093 | 0.408 | 1.5569 |
| NHF | 8F.4.G12 | | 1.0093 | 0.4076 | 1.5569 |
| NHF | 7R.8.D7 | | 1.0079 | 0.4072 | 1.579 |
| NM_007178 | 7F.1.H12 | unr-interacting protein | 1.0069 | 0.4068 | 1.5804 |
| NM_001711 | 7R.6.G9 | biglycan | 1.0064 | 0.4065 | 1.5804 |
| NHF | 7F.8.F11 | | 1.0061 | 0.4061 | 1.5804 |
| NHF | 8R.1.D3 | | 1.0048 | 0.4057 | 1.5896 |
| AK092836 | 12R.3.B10 | Homo sapiens cDNA FLJ35517 fis, clone SPLEN2000698 | 1.0037 | 0.4054 | 1.5896 |
| AA666269 | 14N.6.C11 | integrin, beta 3 (platelet glycoprotein IIIa, antigen CD61) | 1.0036 | 0.405 | 1.5896 |
| D86961 | 1F.1.D6 | lipoma HMGIC fusion partner-like 2 | 1.0033 | 0.4046 | 1.5896 |
| AB037793 | 9F.3.F12 | KIAA1372 protein | 1.0027 | 0.4042 | 1.5896 |
| NM_021129 | 12R.3.H7 | pyrophosphatase (inorganic) | 1.0024 | 0.4039 | 1.5903 |
| NM_024583 | 12F.1.H3 | hypothetical protein FLJ23142 | 1.0014 | 0.4035 | 1.5996 |
| AK000847 | 12F.3.G1 | zinc finger protein 236 | 1.001 | 0.4032 | 1.6004 |
| BG036466 | 8R.9.H10 | cyclin fold protein 1 | 0.9999 | 0.4028 | 1.6073 |
| NM_001616 | 12F.3.C12 | activin A receptor, type II | 0.9987 | 0.4025 | 1.6073 |
| BE963194 | 7F.4.B5 | EST | 0.9987 | 0.4021 | 1.6073 |
| NM_003246 | 8R.10.E4 | thrombospondin 1 | 0.9986 | 0.4018 | 1.6073 |
| NM_001780 | 8F.1.G8 | CD63 antigen (melanoma 1 antigen) | 0.9985 | 0.4014 | 1.6073 |
| BC008330 | 9F.2.D3 | Homo sapiens, tubulin alpha 1, clone MGC: 15803 IMAGE: 3505537, | 0.9983 | 0.4011 | 1.6073 |
| | | mRNA, complete cds | | | |
| AK022804 | 7F.5.H9 | Homo sapiens cDNA FLJ12742 fis, clone NT2RP2000644 | 0.9974 | 0.4008 | 1.6098 |
| NHF | 8R.8.H11 | | 0.9966 | 0.4004 | 1.6103 |
| NM_004791 | 8R.9.G7 | integrin, beta-like 1 (with EGF-like repeat domains) | 0.9965 | 0.4001 | 1.6103 |
| NM_000981 | 7R.2.H10 | ribosomal protein L19 | 0.9955 | 0.3998 | 1.6146 |
| AL833600 | 7R.8.E11 | dynein, cytoplasmic, heavy polypeptide 1 | 0.9942 | 0.3994 | 1.6349 |
| NM_000701 | 7F.10.A12 | ATPase, Na+/K+ transporting, alpha 1 polypeptide | 0.9938 | 0.399 | 1.6362 |
| NM_153649 | 12F.2.F6 | tropomyosin 3 | 0.9934 | 0.3987 | 1.6362 |
| NM_000584 | 7F.9.H8 | interleukin 8 | 0.993 | 0.3983 | 1.6412 |
| NM_005420 | 8R.6.G3 | sulfotransferase, estrogen-preferring | 0.9919 | 0.3979 | 1.6425 |
| BF811751 | 8F.8.A8 | Homo sapiens, clone IMAGE: 4074138, mRNA | 0.9916 | 0.3975 | 1.6425 |
| BI830199 | 8R.7.F9 | likely ortholog of mouse Urb | 0.9906 | 0.3972 | 1.6518 |
| NM_005878 | 8R.10.G1 | trinucleotide repeat containing 3 | 0.9894 | 0.3968 | 1.6594 |
| NM_005625 | 7F.8.A11 | syndecan binding protein (syntenin) | 0.9878 | 0.3964 | 1.6765 |
| NM_000584 | 7F.10.F8 | interleukin 8 | 0.986 | 0.3961 | 1.7089 |
| AV700889 | 12R.1.C3 | ESTs | 0.9857 | 0.3958 | 1.7089 |
| NM_003288 | 7F.2.A5 | tumor protein D52-like 2 | 0.9853 | 0.3954 | 1.7089 |
| AI273932 | 8F.8.G9 | EST | 0.9845 | 0.3951 | 1.7108 |
| R19276 | 14N.6.C6 | cholesteryl ester transfer protein, plasma | 0.9842 | 0.3948 | 1.7108 |
| AA487223 | 14N.4.E11 | synovial sarcoma translocation gene | 0.9837 | 0.3944 | 1.7121 |
| M55580 | 7F.9.E12 | Human spermidine/spermine N1-acetyltransferase mRNA, complete cds | 0.9824 | 0.394 | 1.725 |
| AA284954 | 14N.5.C8 | colony stimulating factor 1 receptor, formerly McDonough feline | 0.9816 | 0.3937 | 1.7259 |
| | | sarcoma viral (v-fms) oncogene homolog | | | |
| NHF | 7F.8.E12 | | 0.9815 | 0.3934 | 1.7259 |
| NM_005063 | 8F.8.G8 | stearoyl-CoA desaturase (delta-9-desaturase) | 0.9785 | 0.3931 | 1.7546 |
| AK092774 | 7R.9.B3 | ribosomal protein, large P2 | 0.9781 | 0.3928 | 1.7546 |
| NM_000944 | 11F.1.H3 | protein phosphatase 3 (formerly 2B), catalytic subunit, alpha isoform | 0.9778 | 0.3924 | 1.7546 |
| | | (calcineurin A alpha) | | | |
| NM_006432 | 7R.6.H11 | Niemann-Pick disease, type C2 | 0.9776 | 0.392 | 1.7546 |
| AA181233 | 14N.4.F11 | ESTs | 0.9768 | 0.3917 | 1.7599 |
| BI830199 | 8R.10.A10 | likely ortholog of mouse Urb | 0.975 | 0.3913 | 1.7717 |
| NM_000584 | 7F.8.E3 | interleukin 8 | 0.9749 | 0.391 | 1.7717 |
| NM_003029 | 7R.1.H12 | SHC (Src homology 2 domain containing) transforming protein 1 | 0.9742 | 0.3907 | 1.7737 |
| NM_006009 | 7F.2.G1 | tubulin, alpha 3 | 0.9734 | 0.3904 | 1.7821 |
| NM_003479 | 7F.3.D10 | protein tyrosine phosphatase type IVA, member 2 | 0.9726 | 0.3901 | 1.7821 |
| NM_001530 | 9R.1.C1 | hypoxia-inducible factor 1, alpha subunit (basic helix-loop-helix | 0.9725 | 0.3897 | 1.7821 |
| | | transcription factor) | | | |
| AI620865 | 8F.10.G3 | EST, Moderately similar to 810024J URF 4 [Homo sapiens] [H. sapiens] | 0.97 | 0.3894 | 1.8055 |
| NM_002421 | 8R.6.D12 | matrix metalloproteinase 1 (interstitial collagenase) | 0.97 | 0.389 | 1.8055 |
| AF495759 | 7R.3.D9 | Homo sapiens unknown mRNA | 0.9687 | 0.3887 | 1.8147 |
| NM_003330 | 8R.1.D7 | thioredoxin reductase 1 | 0.9677 | 0.3884 | 1.8392 |
| NM_002982 | 7F.2.E4 | chemokine (C—C motif) ligand 2 | 0.9672 | 0.3881 | 1.8392 |
| NHF | 8F.1.C9 | | 0.9671 | 0.3877 | 1.8392 |
| AA412509 | 14N.2.C8 | EH-domain containing 4 | 0.9642 | 0.3874 | 1.8699 |
| NHF | 12R.1.F8 | | 0.9641 | 0.3871 | 1.8699 |
| AK094541 | 7F.6.D9 | Homo sapiens cDNA FLJ37222 fis, clone BRAMY1000130, highly | 0.9639 | 0.3867 | 1.8699 |
| | | similar to Homo sapiens MAGE-E1b mRNA | | | |
| NM_021874 | 7R.4.F3 | cell division cycle 25B | 0.9638 | 0.3864 | 1.8699 |
| AA457474 | 14N.4.F12 | receptor (calcitonin) activity modifying protein 2 | 0.9635 | 0.3861 | 1.8699 |
| BE966413 | 11R.1.F12 | EST | 0.9631 | 0.3857 | 1.8699 |
| AL832838 | 7R.7.A8 | hypothetical protein FLJ13952 | 0.9631 | 0.3854 | 1.8699 |
| BC007583 | 7R.5.C9 | Homo sapiens, clone MGC: 15572 IMAGE: 3140342, mRNA, complete | 0.9615 | 0.385 | 1.8856 |
| | | cds | | | |
| NM_005720 | 8F.2.B9 | actin related protein 2/3 complex, subunit 1B, 41 kDa | 0.9601 | 0.3847 | 1.8969 |
| NHF | 11F.1.H10 | | 0.9601 | 0.3844 | 1.8969 |
| N94616 | 14N.2.A2 | laminin, alpha 4 | 0.9586 | 0.3841 | 1.9067 |
| AK091661 | 7R.7.H1 | dynactin 3 (p22) | 0.9585 | 0.3837 | 1.9067 |
| NM_006307 | 8R.2.D6 | sushi-repeat-containing protein, X chromosome | 0.9582 | 0.3834 | 1.9067 |
| NHF | 7F.8.G1 | | 0.9574 | 0.383 | 1.9078 |
| AK091360 | 1R.1.D7 | APC11 anaphase promoting complex subunit 11 homolog (yeast) | 0.9573 | 0.3827 | 1.9078 |
| NM_005324 | 12F.3.H6 | H3 histone, family 3B (H3.3B) | 0.9558 | 0.3824 | 1.9214 |
| NM_005110 | 8F.5.C8 | glutamine-fructose-6-phosphate transaminase 2 | 0.9555 | 0.3821 | 1.9214 |
| NHF | 7F.1.E1 | | 0.9554 | 0.3817 | 1.9214 |
| NHF | 9R.3.G6 | | 0.9549 | 0.3814 | 1.9214 |
| NM_000584 | 8R.4.D11 | interleukin 8 | 0.9548 | 0.381 | 1.9214 |
| NM_000308 | 8F.10.B5 | protective protein for beta-galactosidase (galactosialidosis) | 0.9546 | 0.3807 | 1.9214 |
| NM_000088 | 7R.1.D9 | collagen, type I, alpha 1 | 0.9545 | 0.3804 | 1.9214 |
| NHF | 7R.8.A12 | | 0.9543 | 0.3801 | 1.9214 |
| AI754813 | 8F.7.E2 | collagen, type V, alpha 1 | 0.9533 | 0.3797 | 1.925 |
| AA481464 | 14N.2.A9 | peptidylprolyl isomerase B (cyclophilin B) | 0.9533 | 0.3794 | 1.925 |
| NM_000201 | 9R.8.A6 | intercellular adhesion molecule 1 (CD54), human rhinovirus receptor | 0.9523 | 0.379 | 1.925 |
| NM_000584 | 7F.6.C3 | interleukin 8 | 0.9523 | 0.3787 | 1.925 |
| R19276 | 14N.8.C6 | cholesteryl ester transfer protein, plasma | 0.9521 | 0.3784 | 1.925 |
| AW665223 | 14N.1.B6 | adenylate kinase 5 | 0.951 | 0.3781 | 1.9365 |
| NM_006435 | 7F.1.A1 | interferon induced transmembrane protein 2 (1-8D) | 0.9491 | 0.3778 | 1.9556 |
| NM_001022 | 1R.1.G12 | ribosomal protein S19 | 0.9491 | 0.3775 | 1.9556 |
| AJ238214 | 8F.2.G9 | WD repeat domain 9 | 0.9482 | 0.3772 | 1.9625 |
| NHF | 9F.5.H12 | | 0.9475 | 0.3769 | 1.9642 |
| AK000745 | 12R.2.G7 | Homo sapiens mRNA; cDNA DKFZp564C1563 (from clone | 0.9471 | 0.3766 | 1.9642 |
| | | DKFZp564C1563) | | | |
| NM_024835 | 11F.1.H6 | C3HC4-type zinc finger protein | 0.9465 | 0.3763 | 1.9642 |
| BU536672 | 1R.1.E6 | Homo sapiens mRNA; cDNA DKFZp586O1224 (from clone | 0.9464 | 0.376 | 1.9642 |
| | | DKFZp586O1224) | | | |
| BU684939 | 12R.1.C4 | ESTs | 0.9456 | 0.3756 | 1.9702 |
| AW148618 | 8R.2.H2 | EST, Moderately similar to 810024E cytochrome oxidase III [Homo | 0.9442 | 0.3754 | 1.9867 |
| | | sapiens] [H. sapiens] | | | |
| AI700484 | 14N.1.A9 | hypothetical protein FLJ14050 | 0.9436 | 0.3751 | 1.9897 |
| NM_023032 | 7F.6.B10 | methyltransferase-like 1 | 0.9431 | 0.3748 | 1.9897 |
| NM_003254 | 7F.1.H1 | tissue inhibitor of metalloproteinase 1 (erythroid potentiating activity, | 0.943 | 0.3745 | 1.9897 |
| | | collagenase inhibitor) | | | |
| NHF | 12R.1.D1 | | 0.9373 | 0.3742 | 2.0747 |
| NM_006014 | 8F.10.B10 | DNA segment on chromosome X (unique) 9879 expressed sequence | 0.9365 | 0.3738 | 2.0903 |
| BF965170 | 7R.4.D10 | interferon induced transmembrane protein 3 (1-8U) | 0.9363 | 0.3735 | 2.0903 |
| NHF | 9R.3.D5 | | 0.9356 | 0.3732 | 2.0991 |
| AW005755 | 14N.7.G8 | macrophage migration inhibitory factor (glycosylation-inhibiting factor) | 0.9349 | 0.3729 | 2.1004 |
| NM_004419 | 8R.10.D12 | dual specificity phosphatase 5 | 0.9342 | 0.3726 | 2.1031 |
| AI612803 | 8R.9.D12 | EST | 0.9327 | 0.3723 | 2.1186 |
| NM_003029 | 7R.5.H2 | SHC (Src homology 2 domain containing) transforming protein 1 | 0.9326 | 0.372 | 2.1186 |
| NM_004369 | 8F.2.G2 | collagen, type VI, alpha 3 | 0.932 | 0.3717 | 2.1206 |
| AW166001 | 8R.9.B4 | EST, Weakly similar to 810024E cytochrome oxidase III [Homo sapiens] | 0.9317 | 0.3714 | 2.1211 |
| | | [H. sapiens] | | | |
| NM_004530 | 8R.1.C11 | matrix metalloproteinase 2 (gelatinase A, 72 kDa gelatinase, 72 kDa type | 0.9312 | 0.3711 | 2.1224 |
| | | IV collagenase) | | | |
| BE736438 | 7R.8.D10 | ESTs, Highly similar to A29170 phosphopyruvate hydratase (EC | 0.9305 | 0.3709 | 2.1289 |
| | | 4.2.1.11) alpha - human [H. sapiens] | | | |
| NHF | 9F.3.C2 | | 0.9301 | 0.3705 | 2.1289 |
| NM_007361 | 7R.2.G10 | nidogen 2 (osteonidogen) | 0.93 | 0.3701 | 2.1289 |
| NM_002074 | 9F.10.E10 | guanine nucleotide binding protein (G protein), beta polypeptide 1 | 0.9291 | 0.3699 | 2.1457 |
| NM_004238 | 9F.6.D3 | thyroid hormone receptor interactor 12 | 0.9287 | 0.3695 | 2.1469 |
| AA192691 | 8F.9.D9 | EST | 0.9264 | 0.3692 | 2.1729 |
| BC001805 | 12R.1.D3 | Homo sapiens, clone IMAGE: 3543670, mRNA, partial cds | 0.9261 | 0.3689 | 2.1729 |
| NM_000089 | 8F.10.E3 | collagen, type I, alpha 2 | 0.9257 | 0.3686 | 2.1729 |
| BC019019 | 7R.7.F1 | Homo sapiens, WAS protein family, member 1, clone MGC: 20657 | 0.9253 | 0.3683 | 2.1729 |
| | | IMAGE: 3841135, mRNA, complete cds | | | |
| NM_005720 | 7R.1.B4 | actin related protein 2/3 complex, subunit 1B, 41 kDa | 0.9251 | 0.368 | 2.1729 |
| NM_001530 | 12R.3.A11 | hypoxia-inducible factor 1, alpha subunit (basic helix-loop-helix | 0.9247 | 0.3678 | 2.1729 |
| | | transcription factor) | | | |
| BI430544 | 11F.1.C11 | ESTs | 0.9245 | 0.3675 | 2.1729 |
| AA191645 | 8R.10.H6 | ESTs, Moderately similar to ribosomal protein S2; 40S ribosomal protein | 0.9239 | 0.3672 | 2.1765 |
| | | S2 [Homo sapiens] [H. sapiens] | | | |
| AW779971 | 14N.1.B12 | ESTs, Weakly similar to hypothetical protein FLJ20378 [Hs.] [H. sapiens] | 0.9239 | 0.3669 | 2.1765 |
| NM_000358 | 7R.10.H6 | transforming growth factor, beta-induced, 68 kDa | 0.9231 | 0.3666 | 2.1838 |
| NM_005628 | 7R.5.D7 | solute carrier family 1 (neutral amino acid transporter), member 5 | 0.9221 | 0.3663 | 2.1925 |
| AA453774 | 14N.1.D9 | regulator of G-protein signalling 16 | 0.9218 | 0.366 | 2.1936 |
| X69392 | 7R.6.G5 | H. sapiens mRNA for ribosomal protein L26 | 0.9175 | 0.3657 | 2.2527 |
| NM_002356 | 8F.6.D7 | myristoylated alanine-rich protein kinase C substrate | 0.9174 | 0.3654 | 2.2527 |
| BU626315 | 8F.7.D1 | collagen, type V, alpha 1 | 0.9154 | 0.3651 | 2.2828 |
| NM_002993 | 9R.7.H10 | chemokine (C—X—C motif) ligand 6 (granulocyte chemotactic protein 2) | 0.9145 | 0.3648 | 2.2848 |
| NM_000365 | 7R.4.F7 | triosephosphate isomerase 1 | 0.9143 | 0.3645 | 2.2848 |
| AA873792 | 14N.6.D11 | small inducible cytokine A5 (RANTES) | 0.9136 | 0.3642 | 2.2972 |
| NM_020650 | 7R.5.F1 | hypothetical protein LOC57333 | 0.913 | 0.364 | 2.3001 |
| NHF | 8F.4.G3 | | 0.9121 | 0.3637 | 2.3193 |
| BI430544 | 12R.2.G9 | ESTs | 0.9115 | 0.3634 | 2.3277 |
| AA485883 | 14N.4.E2 | von Willebrand factor | 0.9097 | 0.3632 | 2.3407 |
| AA489314 | 14N.4.D12 | gp25L2 protein | 0.9095 | 0.3629 | 2.3407 |
| NHF | 7F.3.E7 | | 0.9092 | 0.3626 | 2.3407 |
| NM_016522 | 9R.1.F11 | neurotrimin | 0.9088 | 0.3623 | 2.3407 |
| NM_002982 | 7F.7.D9 | chemokine (C—C motif) ligand 2 | 0.9087 | 0.362 | 2.3407 |
| AV694354 | 7F.6.H5 | KIAA1671 protein | 0.9081 | 0.3617 | 2.3407 |
| AA406585 | 14N.1.D7 | Lysosomal-associated multispanning membrane protein-5 | 0.9081 | 0.3614 | 2.3407 |
| AK095169 | 8F.6.E8 | Homo sapiens cDNA FLJ37850 fis, clone BRSSN2013733, weakly | 0.9079 | 0.3611 | 2.3407 |
| | | similar to Homo sapiens mRNA for ALEX1 | | | |
| NM_002982 | 7F.10.B10 | chemokine (C—C motif) ligand 2 | 0.9077 | 0.3609 | 2.3407 |
| NM_000584 | 7F.2.G10 | interleukin 8 | 0.907 | 0.3606 | 2.3486 |
| NM_003246 | 8R.1.E9 | thrombospondin 1 | 0.9059 | 0.3603 | 2.3606 |
| AA775616 | 14N.8.C10 | secreted phosphoprotein 1 (osteopontin, bone sialoprotein I, early T- | 0.9059 | 0.36 | 2.3606 |
| | | lymphocyte activation 1) | | | |
| NHF | 7F.9.B1 | | 0.905 | 0.3598 | 2.3719 |
| NM_005878 | 8F.2.D8 | trinucleotide repeat containing 3 | 0.9047 | 0.3595 | 2.3719 |
| NM_003246 | 9R.8.H2 | thrombospondin 1 | 0.9025 | 0.3592 | 2.416 |
| NM_000584 | 7F.8.D12 | interleukin 8 | 0.902 | 0.3589 | 2.4202 |
| NM_002356 | 12F.2.A2 | myristoylated alanine-rich protein kinase C substrate | 0.9019 | 0.3587 | 2.4202 |
| AA156022 | 14N.4.G2 | roundabout homolog 4, magic roundabout (Drosophila) | 0.9017 | 0.3584 | 2.4202 |
| NM_005324 | 12F.1.G6 | H3 histone, family 3B (H3.3B) | 0.901 | 0.3581 | 2.4268 |
| BI830199 | 9R.6.D7 | likely ortholog of mouse Urb | 0.901 | 0.3578 | 2.4268 |
| AJ237724 | 8R.7.B8 | solute carrier family 19 (thiamine transporter), member 2 | 0.9006 | 0.3575 | 2.4282 |
| NM_003842 | 7F.10.B6 | tumor necrosis factor receptor superfamily, member 10b | 0.9003 | 0.3572 | 2.4308 |
| NM_020529 | 7F.6.D1 | nuclear factor of kappa light polypeptide gene enhancer in B-cells | 0.8976 | 0.3569 | 2.4743 |
| | | inhibitor, alpha | | | |
| NM_000358 | 7R.8.F11 | transforming growth factor, beta-induced, 68 kDa | 0.8955 | 0.3567 | 2.4969 |
| BE963194 | 8R.1.A1 | EST | 0.8954 | 0.3564 | 2.4969 |
| NM_053275 | 7F.6.B8 | ribosomal protein, large, P0 | 0.8953 | 0.3562 | 2.4969 |
| NM_000584 | 8R.3.E1 | interleukin 8 | 0.894 | 0.3559 | 2.5106 |
| AW007736 | 9R.10.F6 | UDP-glucose ceramide glucosyltransferase | 0.8934 | 0.3556 | 2.5235 |
| AI334914 | 14N.7.E12 | integrin, alpha 2b (platelet glycoprotein IIb of IIb/IIIa complex, antigen | 0.8931 | 0.3554 | 2.5235 |
| | | CD41B) | | | |
| BC004215 | 8R.10.A2 | Homo sapiens, eukaryotic translation elongation factor 1 gamma, clone | 0.8919 | 0.3551 | 2.5367 |
| | | MGC: 4501 IMAGE: 2964623, mRNA, complete cds | | | |
| NM_006088 | 8R.7.G4 | tubulin, beta, 2 | 0.8918 | 0.3548 | 2.5367 |
| NHF | 8F.1.G4 | | 0.8915 | 0.3546 | 2.5367 |
| AA608531 | 14N.4.G3 | hypothetical protein DJ667H12.2 | 0.8906 | 0.3543 | 2.5367 |
| BF976811 | 12R.1.B6 | leucyl-tRNA synthetase | 0.8905 | 0.354 | 2.5367 |
| NM_006216 | 8F.3.E1 | serine (or cysteine) proteinase inhibitor, clade E (nexin, plasminogen | 0.8904 | 0.3537 | 2.5367 |
| | | activator inhibitor type 1), member 2 | | | |
| BF247987 | 8R.10.B6 | tumor up-regulated CARD-containing antagonist of caspase nine | 0.8903 | 0.3535 | 2.5367 |
| AA284495 | 14N.1.D6 | mesoderm development candidate 2 | 0.8901 | 0.3532 | 2.5367 |
| BC007583 | 8R.8.G9 | Homo sapiens, clone MGC: 15572 IMAGE: 3140342, mRNA, complete | 0.89 | 0.3529 | 2.5367 |
| | | cds | | | |
| NM_002356 | 12R.1.G8 | myristoylated alanine-rich protein kinase C substrate | 0.889 | 0.3527 | 2.5528 |
| NM_005520 | 7F.8.B6 | heterogeneous nuclear ribonucleoprotein H1 (H) | 0.8887 | 0.3524 | 2.5528 |
| NM_005803 | 8F.9.G1 | flotillin 1 | 0.8881 | 0.3521 | 2.5528 |
| NHF | 9R.2.D10 | | 0.8875 | 0.3518 | 2.5568 |
| NM_004530 | 8R.10.D6 | matrix metalloproteinase 2 (gelatinase A, 72 kDa gelatinase, 72 kDa type | 0.8871 | 0.3515 | 2.5568 |
| | | IV collagenase) | | | |
| AA147552 | 14N.2.A1 | ESTs | 0.887 | 0.3512 | 2.5568 |
| AW166001 | 9R.5.G6 | EST, Weakly similar to 810024E cytochrome oxidase III [Homo sapiens] | 0.8859 | 0.3509 | 2.5694 |
| | | [H. sapiens] | | | |
| NM_000365 | 7R.10.G11 | triosephosphate isomerase 1 | 0.8853 | 0.3506 | 2.5742 |
| NM_001122 | 12F.2.F3 | adipose differentiation-related protein | 0.8848 | 0.3503 | 2.5783 |
| AK098378 | 12F.1.B7 | Homo sapiens cDNA FLJ25512 fis, clone CBR06118 | 0.8844 | 0.3501 | 2.5788 |
| NM_000584 | 7F.5.C5 | interleukin 8 | 0.8838 | 0.3498 | 2.5804 |
| NM_012335 | 7R.6.E5 | myosin IF | 0.8836 | 0.3496 | 2.5804 |
| NM_000393 | 9F.9.A3 | collagen, type V, alpha 2 | 0.8829 | 0.3493 | 2.5821 |
| NM_007075 | 7R.9.B10 | JM5 protein | 0.8826 | 0.349 | 2.5838 |
| BI830199 | 8R.3.B7 | likely ortholog of mouse Urb | 0.8819 | 0.3487 | 2.5944 |
| NHF | 8R.4.C7 | | 0.8816 | 0.3484 | 2.5944 |
| NM_000701 | 1F.1.E5 | ATPase, Na+/K+ transporting, alpha 1 polypeptide | 0.8809 | 0.3482 | 2.608 |
| AW166001 | 8R.1.F9 | EST, Weakly similar to 810024E cytochrome oxidase III [Homo sapiens] | 0.8802 | 0.3479 | 2.6375 |
| | | [H. sapiens] | | | |
| AW148618 | 9F.4.E11 | EST, Moderately similar to 810024E cytochrome oxidase III [Homo | 0.8796 | 0.3476 | 2.6387 |
| | | sapiens] [H. sapiens] | | | |
| BM804630 | 7F.4.H7 | Human HepG2 3 region cDNA, clone hmd6c02 | 0.8789 | 0.3474 | 2.6387 |
| NM_005720 | 8F.4.E10 | actin related protein 2/3 complex, subunit 1B, 41 kDa | 0.8788 | 0.3471 | 2.6387 |
| NM_000584 | 7F.10.C7 | interleukin 8 | 0.8787 | 0.3469 | 2.6387 |
| BI430544 | 12R.3.F1 | ESTs | 0.8786 | 0.3466 | 2.6387 |
| NM_002229 | 9R.2.A5 | jun B proto-oncogene | 0.8783 | 0.3464 | 2.6387 |
| AA485428 | 14N.1.G10 | KIAA0620 protein | 0.8783 | 0.3461 | 2.6387 |
| AW192258 | 8R.4.F1 | sprouty homolog 4 (Drosophila) | 0.8778 | 0.3459 | 2.6439 |
| NM_001530 | 9F.5.E6 | hypoxia-inducible factor 1, alpha subunit (basic helix-loop-helix | 0.877 | 0.3456 | 2.6537 |
| | | transcription factor) | | | |
| NM_001530 | 12R.1.G5 | hypoxia-inducible factor 1, alpha subunit (basic helix-loop-helix | 0.8763 | 0.3453 | 2.6594 |
| | | transcription factor) | | | |
| BI830199 | 8R.8.B7 | likely ortholog of mouse Urb | 0.8757 | 0.345 | 2.6637 |
| BF247987 | 8R.4.F2 | tumor up-regulated CARD-containing antagonist of caspase nine | 0.8755 | 0.3448 | 2.6637 |
| NHF | 9R.3.G9 | | 0.8734 | 0.3445 | 2.7033 |
| AK098212 | 8F.1.G10 | hypothetical protein FLJ10359 | 0.8722 | 0.3442 | 2.7242 |
| NHF | 12R.2.F1 | | 0.8721 | 0.344 | 2.7242 |
| BE908954 | 7F.9.D5 | ESTs, Highly similar to FRHUH ferritin heavy chain - human [H. sapiens] | 0.872 | 0.3437 | 2.7242 |
| NM_006009 | 1F.1.F11 | tubulin, alpha 3 | 0.8715 | 0.3435 | 2.7256 |
| NM_002318 | 7R.5.F6 | lysyl oxidase-like 2 | 0.8711 | 0.3432 | 2.7341 |
| AI810848 | 8F.4.G10 | ubiquitin-conjugating enzyme E2I (UBC9 homolog, yeast) | 0.869 | 0.343 | 2.7615 |
| NM_004048 | 7F.6.E7 | beta-2-microglobulin | 0.869 | 0.3427 | 2.7615 |
| AV719568 | 12R.2.H5 | EST | 0.8673 | 0.3424 | 2.7703 |
| NM_002356 | 12F.3.B11 | myristoylated alanine-rich protein kinase C substrate | 0.8672 | 0.3422 | 2.7703 |
| NM_005803 | 8F.10.C10 | flotillin 1 | 0.8671 | 0.3419 | 2.7703 |
| NM_018975 | 1F.1.F7 | telomeric repeat binding factor 2, interacting protein | 0.867 | 0.3417 | 2.7703 |
| NHF | 1R.1.A12 | | 0.867 | 0.3414 | 2.7703 |
| NM_003246 | 9F.1.G10 | thrombospondin 1 | 0.8668 | 0.3412 | 2.7703 |
| AA464163 | 14N.1.E3 | acyl-Coenzyme A dehydrogenase, very long chain | 0.8665 | 0.341 | 2.7703 |
| NM_033301 | 7R.7.A10 | ribosomal protein L8 | 0.8663 | 0.3407 | 2.7703 |
| AW297729 | 14N.1.C5 | uncharacterized hematopoietic stem/progenitor cells protein MDS026 | 0.866 | 0.3405 | 2.7723 |
| BE963194 | 7F.4.F12 | EST | 0.865 | 0.3402 | 2.7904 |
| NHF | 8F.8.G7 | | 0.8635 | 0.34 | 2.8167 |
| N64508 | 14N.2.E7 | podocalyxin-like | 0.863 | 0.3398 | 2.825 |
| NHF | 9R.1.F1 | | 0.8628 | 0.3395 | 2.825 |
| NM_005347 | 12F.1.G4 | heat shock 70 kDa protein 5 (glucose-regulated protein, 78 kDa) | 0.8623 | 0.3393 | 2.8298 |
| AW148618 | 8F.8.A7 | EST, Moderately similar to 810024E cytochrome oxidase III [Homo | 0.8603 | 0.339 | 2.8763 |
| | | sapiens] [H. sapiens] | | | |
| AK095629 | 8F.3.C8 | SEC13-like 1 (S. cerevisiae) | 0.8595 | 0.3388 | 2.884 |
| NM_012207 | 8R.1.E6 | heterogeneous nuclear ribonucleoprotein H3 (2H9) | 0.8591 | 0.3386 | 2.884 |
| BF976811 | 12F.3.D2 | leucyl-tRNA synthetase | 0.8591 | 0.3383 | 2.884 |
| AW772832 | 14N.4.H3 | ribosomal protein S17 | 0.8586 | 0.338 | 2.8858 |
| AI262059 | 1R.1.E10 | ESTs | 0.8567 | 0.3378 | 2.9278 |
| NM_000584 | 7F.4.C9 | interleukin 8 | 0.8566 | 0.3375 | 2.9278 |
| BM011169 | 8F.3.E8 | ESTs, Highly similar to RL23_HUMAN 60S ribosomal protein L23 (L17) | 0.8562 | 0.3373 | 2.9278 |
| | | [H. sapiens] | | | |
| AV719568 | 12R.1.B2 | EST | 0.8554 | 0.337 | 2.9278 |
| AK027663 | 8F.2.C4 | stanniocalcin 2 | 0.8554 | 0.3368 | 2.9278 |
| AA057204 | 14N.7.B3 | interleukin 2 receptor, beta | 0.8553 | 0.3365 | 2.9278 |
| NM_000089 | 8F.4.H9 | collagen, type I, alpha 2 | 0.8552 | 0.3362 | 2.9278 |
| BC018130 | 7F.6.C6 | coagulation factor II (thrombin) receptor-like 1 | 0.8551 | 0.336 | 2.9278 |
| BI430544 | 11R.1.G6 | ESTs | 0.855 | 0.3357 | 2.9278 |
| R70506 | 14N.1.F8 | growth factor receptor-bound protein 2 | 0.8548 | 0.3354 | 2.9278 |
| BG680524 | 7R.9.E3 | RNA, U67 small nucleolar | 0.8527 | 0.3352 | 2.9594 |
| NM_002317 | 12R.1.G3 | lysyl oxidase | 0.8526 | 0.3349 | 2.9594 |
| NM_001628 | 7F.10.A9 | aldo-keto reductase family 1, member B1 (aldose reductase) | 0.8525 | 0.3347 | 2.9594 |
| NM_006307 | 9R.4.E5 | sushi-repeat-containing protein, X chromosome | 0.852 | 0.3344 | 2.9594 |
| NM_000584 | 7F.5.D3 | interleukin 8 | 0.8517 | 0.3342 | 2.9594 |
| NM_004530 | 8R.6.G6 | matrix metalloproteinase 2 (gelatinase A, 72 kDa gelatinase, 72 kDa type | 0.8514 | 0.3339 | 2.9594 |
| | | IV collagenase) | | | |
| NM_003246 | 8R.10.E5 | thrombospondin 1 | 0.8513 | 0.3337 | 2.9594 |
| NM_016357 | 7R.5.G9 | epithelial protein lost in neoplasm beta | 0.8513 | 0.3335 | 2.9594 |
| NM_002727 | 7F.10.E8 | proteoglycan 1, secretory granule | 0.851 | 0.3332 | 2.9594 |
| BC019019 | 7F.10.E11 | Homo sapiens, WAS protein family, member 1, clone MGC: 20657 | 0.8508 | 0.333 | 2.9594 |
| | | IMAGE: 3841135, mRNA, complete cds | | | |
| BM924182 | 8R.4.A8 | ESTs, Weakly similar to retinal short-chain dehydrogenase/reductase | 0.8506 | 0.3327 | 2.9594 |
| | | retSDR2 [Homo sapiens] [H. sapiens] | | | |
| NM_002982 | 7F.9.A2 | chemokine (C—C motif) ligand 2 | 0.8502 | 0.3325 | 2.9638 |
| NM_002982 | 7F.3.C12 | chemokine (C—C motif) ligand 2 | 0.8487 | 0.3322 | 2.9792 |
| NHF | 7F.8.H12 | | 0.8484 | 0.332 | 2.9792 |
| NM_002026 | 7R.3.G7 | fibronectin 1 | 0.8479 | 0.3317 | 2.9792 |
| NM_002818 | 7F.5.E12 | proteasome (prosome, macropain) activator subunit 2 (PA28 beta) | 0.8477 | 0.3315 | 2.9792 |
| NM_001357 | 8R.8.H7 | DEAD/H (Asp-Glu-Ala-Asp/His) box polypeptide 9 (RNA helicase A, | 0.8477 | 0.3312 | 2.9792 |
| | | nuclear DNA helicase II; leukophysin) | | | |
| AI074784 | 14N.5.F4 | colony stimulating factor 3 (granulocyte) | 0.8476 | 0.331 | 2.9792 |
| AI860401 | 7R.4.D11 | EST, Moderately similar to 810024E cytochrome oxidase III [Homo | 0.8474 | 0.3307 | 2.9792 |
| | | sapiens] [H. sapiens] | | | |
| AY092084 | 7F.2.B5 | RNA polymerase III subunit RPC2 | 0.8471 | 0.3305 | 2.9792 |
| NHF | 7F.5.G12 | | 0.847 | 0.3303 | 2.9792 |
| NM_003842 | 1R.1.B1 | tumor necrosis factor receptor superfamily, member 10b | 0.8469 | 0.3301 | 2.9792 |
| NHF | 8F.8.F12 | | 0.846 | 0.3298 | 3.0053 |
| NM_006307 | 8R.5.C8 | sushi-repeat-containing protein, X chromosome | 0.8443 | 0.3296 | 3.0302 |
| BM458752 | 7F.7.G10 | ESTs, Highly similar to histone H2A.F/Z variant, isoform 1; purine-rich | 0.8443 | 0.3293 | 3.0302 |
| | | binding element protein B [Homo sapiens] [H. sapiens] | | | |
| NHF | 12R.1.C2 | | 0.844 | 0.3291 | 3.0302 |
| NHF | 8R.8.G2 | | 0.8439 | 0.3289 | 3.0302 |
| NHF | 7F.4.B3 | | 0.8436 | 0.3287 | 3.0302 |

These genes are down-regulated in diabetic samples and up-regulated in non-diabetic samples.

| | | | | | |
|---|---|---|---|---|---|
| AB007916 | 8F.9.B12 | KIAA0447 gene product | −0.9678 | −0.4529 | 3.0374 |
| AF034176 | 8R.7.D12 | Homo sapiens clone 23872 mRNA sequence | −0.9678 | −0.4534 | 3.0374 |
| AK095036 | 9R.7.B10 | Homo sapiens cDNA FLJ37717 fis, clone BRHIP2018998, weakly | −0.9679 | −0.4539 | 3.0374 |
| | | similar to FLAGELLAR WD-REPEAT PROTEIN PF20 | | | |
| Z24725 | 7F.7.H4 | mitogen inducible 2 | −0.9682 | −0.4544 | 3.0374 |
| AF267856 | 1F.1.H9 | hypothetical protein dJ465N24.2.1 | −0.9683 | −0.4549 | 3.0374 |
| BC015869 | 8F.3.F7 | Homo sapiens clone 23698 mRNA sequence | −0.9687 | −0.4554 | 3.0374 |
| AK055662 | 9F.8.D11 | Homo sapiens cDNA FLJ31100 fis, clone IMR321000242, weakly | −0.9712 | −0.4559 | 3.0112 |
| | | similar to ZINC FINGER PROTEIN 33A | | | |
| NHF | 7F.9.A9 | | −0.9726 | −0.4564 | 2.9947 |
| BQ674142 | 7F.8.B9 | Homo sapiens mRNA; cDNA DKFZp762N156 (from clone | −0.9748 | −0.457 | 2.9616 |
| | | DKFZp762N156) | | | |
| NM_014765 | 8R.4.H2 | translocase of outer mitochondrial membrane 20 (yeast) homolog | −0.9753 | −0.4575 | 2.9616 |
| NM_002026 | 8R.6.C3 | fibronectin 1 | −0.9766 | −0.458 | 2.9408 |
| AK090550 | 9F.1.C1 | Homo sapiens cDNA: FLJ21533 fis, clone COL06072 | −0.9771 | −0.4585 | 2.9408 |
| | | integrin, beta 1 (fibronectin receptor, beta polypeptide, antigen CD29 | | | |
| NM_002211 | 9R.8.G8 | includes MDF2, MSK12) | −0.9775 | −0.459 | 2.9408 |
| BM820221 | 7R.4.A10 | ribosomal protein L5 | −0.9785 | −0.4595 | 2.9395 |
| NM_004939 | 8F.6.D11 | DEAD/H (Asp-Glu-Ala-Asp/His) box polypeptide 1 | −0.9793 | −0.4601 | 2.9323 |
| NM_002211 | 12R.3.F9 | integrin, beta 1 (fibronectin receptor, beta polypeptide, antigen CD29 | −0.9804 | −0.4607 | 2.9025 |
| | | includes MDF2, MSK12) | | | |
| M14219 | 8R.6.H11 | Human chondroitin/dermatan sulfate proteoglycan (PG40) core protein | −0.9812 | −0.4612 | 2.8996 |
| | | mRNA, complete cds | | | |
| NHF | 9R.10.E6 | | −0.9815 | −0.4617 | 2.8996 |
| NM_003932 | 9F.1.D9 | suppression of tumorigenicity 13 (colon carcinoma) (Hsp70 interacting | −0.9822 | −0.4623 | 2.8996 |
| | | protein) | | | |
| BG742464 | 9R.8.D2 | Homo sapiens cDNA FLJ25106 fis, clone CBR01467 | −0.9846 | −0.4628 | 2.848 |
| NM_024408 | 12F.3.C7 | Notch homolog 2 (Drosophila) | −0.9853 | −0.4633 | 2.845 |
| NM_002211 | 1F.1.H12 | integrin, beta 1 (fibronectin receptor, beta polypeptide, antigen CD29 | −0.9861 | −0.4639 | 2.8442 |
| | | includes MDF2, MSK12) | | | |
| NM_001745 | 9R.8.E9 | calcium modulating ligand | −0.9868 | −0.4644 | 2.8384 |
| NM_006329 | 8R.6.F6 | fibulin 5 | −0.9886 | −0.4649 | 2.7972 |
| L27560 | 9R.1.E4 | insulin-like growth factor binding protein 5 | −0.9892 | −0.4655 | 2.7969 |
| NM_002211 | 12R.3.D10 | integrin, beta 1 (fibronectin receptor, beta polypeptide, antigen CD29 | −0.9909 | −0.466 | 2.7777 |
| | | includes MDF2, MSK12) | | | |
| NM_144573 | 8R.2.G10 | likely ortholog of rat F-actin binding protein nexilin | −0.9922 | −0.4665 | 2.7659 |
| NM_001920 | 12R.1.C7 | decorin | −0.9941 | −0.4671 | 2.7425 |
| AL832642 | 9F.9.H4 | CD44 antigen (homing function and Indian blood group system) | −0.9959 | −0.4676 | 2.7139 |
| NM_004071 | 8F.5.B2 | CDC-like kinase 1 | −0.9969 | −0.4682 | 2.7106 |
| AB040951 | 7R.2.E6 | KIAA1518 protein | −0.9986 | −0.4688 | 2.6836 |
| NM_002211 | 8F.10.B9 | integrin, beta 1 (fibronectin receptor, beta polypeptide, antigen CD29 | −0.9998 | −0.4693 | 2.6797 |
| | | includes MDF2, MSK12) | | | |
| NHF | 12R.2.C11 | | −1 | −0.4699 | 2.6797 |
| Z24725 | 9F.3.B3 | mitogen inducible 2 | −1.0029 | −0.4704 | 2.6335 |
| NM_012175 | 9F.7.C2 | F-box only protein 3 | −1.0075 | −0.471 | 2.5469 |
| BI430544 | 1R.2.C10 | ESTs | −1.0076 | −0.4715 | 2.5469 |
| NM_001752 | 7F.3.D11 | catalase | −1.0089 | −0.472 | 2.544 |
| NHF | 9F.9.E3 | | −1.0106 | −0.4726 | 2.5204 |
| NM_018507 | 8R.2.H11 | hypothetical protein PRO1843 | −1.0108 | −0.4732 | 2.5204 |
| BE966143 | 9F.5.B6 | EST | −1.0134 | −0.4739 | 2.485 |
| NHF | 9R.4.H9 | | −1.0143 | −0.4744 | 2.4819 |
| NM_001679 | 9F.1.H5 | ATPase, Na+/K+ transporting, beta 3 polypeptide | −1.0149 | −0.475 | 2.4819 |
| NM_020755 | 7R.2.D9 | likely ortholog of mouse tumor differentially expressed 1, like | −1.0154 | −0.4756 | 2.4819 |
| NM_005100 | 12R.3.E6 | A kinase (PRKA) anchor protein (gravin) 12 | −1.0171 | −0.4762 | 2.4497 |
| NM_014585 | 8R.3.G3 | solute carrier family 11 (proton-coupled divalent metal ion transporters), | −1.0182 | −0.4769 | 2.4428 |
| | | member 3 | | | |
| NM_033305 | 7R.7.F12 | chorea acanthocytosis | −1.0192 | −0.4774 | 2.437 |
| AJ420488 | 7F.5.H10 | eukaryotic translation elongation factor 1 alpha 1 | −1.0201 | −0.478 | 2.4344 |
| AF278532 | 8R.1.D11 | netrin 4 | −1.0211 | −0.4787 | 2.4331 |
| NHF | 8R.8.C5 | | −1.0215 | −0.4793 | 2.4331 |
| NM_030571 | 9F.9.H3 | likely ortholog of mouse Nedd4 WW binding protein 5 | −1.0228 | −0.4799 | 2.4329 |
| AK092475 | 9R.8.H3 | Homo sapiens cDNA FLJ35156 fis, clone PLACE6011057 | −1.023 | −0.4805 | 2.4329 |
| NM_002211 | 7R.3.C8 | integrin, beta 1 (fibronectin receptor, beta polypeptide, antigen CD29 | −1.0243 | −0.481 | 2.4247 |
| | | includes MDF2, MSK12) | | | |
| NM_001967 | 9R.9.D1 | eukaryotic translation initiation factor 4A, isoform 2 | −1.0261 | −0.4817 | 2.3902 |
| NHF | 9R.10.D1 | | −1.027 | −0.4823 | 2.3869 |
| NM_001102 | 8F.6.A8 | actinin, alpha 1 | −1.0313 | −0.483 | 2.3215 |
| NM_018507 | 8R.7.A5 | hypothetical protein PRO1843 | −1.0326 | −0.4836 | 2.3125 |
| NHF | 7R.9.H12 | | −1.0327 | −0.4842 | 2.3125 |
| NM_000454 | 9R.10.A9 | superoxide dismutase 1, soluble (amyotrophic lateral sclerosis 1 (adult)) | −1.0331 | −0.4849 | 2.3125 |
| NM_000919 | 8R.1.B7 | peptidylglycine alpha-amidating monooxygenase | −1.0335 | −0.4856 | 2.3125 |
| NM_015904 | 12F.3.C2 | translation initiation factor IF2 | −1.0344 | −0.4862 | 2.3125 |
| NM_144617 | 8R.10.H2 | hypothetical protein FLJ32389 | −1.0354 | −0.4869 | 2.3125 |
| NM_004939 | 9F.4.C12 | DEAD/H (Asp-Glu-Ala-Asp/His) box polypeptide 1 | −1.0364 | −0.4875 | 2.3125 |
| NM_016081 | 12R.3.F2 | palladin | −1.0379 | −0.4881 | 2.2949 |
| NHF | 8R.2.G7 | | −1.0384 | −0.4888 | 2.2949 |
| NM_001753 | 9R.2.B9 | caveolin 1, caveolae protein, 22 kDa | −1.0391 | −0.4894 | 2.2949 |
| NM_031885 | 9F.6.F4 | Bardet-Biedl syndrome 2 | −1.041 | −0.4901 | 2.255 |
| U23841 | 9R.10.C8 | ESTs | −1.0412 | −0.4908 | 2.255 |
| NM_000274 | 9R.8.H7 | ornithine aminotransferase (gyrate atrophy) | −1.0419 | −0.4914 | 2.255 |
| AA001757 | 9R.2.A6 | ubiquitin-conjugating enzyme E2I (UBC9 homolog, yeast) | −1.0443 | −0.4921 | 2.2169 |
| NM_002948 | 8R.5.A1 | ribosomal protein L15 | −1.0465 | −0.4927 | 2.2124 |
| NM_001012 | 8R.9.A7 | ribosomal protein S8 | −1.0468 | −0.4934 | 2.2124 |
| NHF | 9R.7.F1 | | −1.0475 | −0.494 | 2.2124 |
| NHF | 8R.2.E6 | | −1.0477 | −0.4948 | 2.2124 |
| NM_032926 | 8F.3.B10 | hypothetical protein MGC15737 | −1.0477 | −0.4954 | 2.2124 |
| NM_003405 | 7F.4.G7 | tyrosine 3-monooxygenase/tryptophan 5-monooxygenase activation | −1.0513 | −0.496 | 2.2098 |
| | | protein, eta polypeptide | | | |
| NM_020182 | 7R.7.G12 | transmembrane, prostate androgen induced RNA | −1.0516 | −0.4967 | 2.2098 |
| NM_004071 | 8R.10.C4 | CDC-like kinase 1 | −1.0522 | −0.4974 | 2.2098 |
| NHF | 9F.8.C4 | | −1.0563 | −0.498 | 2.1516 |
| NM_001753 | 12R.3.F7 | caveolin 1, caveolae protein, 22 kDa | −1.0583 | −0.4988 | 2.139 |
| NHF | 9R.6.B1 | | −1.059 | −0.4995 | 2.1357 |
| NM_001102 | 9R.6.E10 | actinin, alpha 1 | −1.0615 | −0.5002 | 2.1041 |
| NM_018181 | 7F.10.C9 | zinc finger protein | −1.0616 | −0.5009 | 2.1041 |
| NM_001901 | 7R.1.B12 | connective tissue growth factor | −1.063 | −0.5015 | 2.1041 |
| AF267856 | 8R.7.A7 | hypothetical protein dJ465N24.2.1 | −1.0649 | −0.5023 | 2.0957 |
| NM_002026 | 8R.6.E5 | fibronectin 1 | −1.0652 | −0.503 | 2.0957 |
| AK074073 | 7F.10.G9 | hypothetical protein MGC3222 | −1.0657 | −0.5037 | 2.0957 |
| NM_006835 | 7R.8.C12 | cyclin I | −1.0662 | −0.5045 | 2.0957 |
| U16850 | 9F.3.F8 | Homo sapiens calmodulin-I (CALM1) mRNA, 3 UTR, partial sequence | −1.0665 | −0.5052 | 2.0957 |
| NM_004282 | 9R.8.A4 | BCL2-associated athanogene 2 | −1.0673 | −0.5059 | 2.0957 |
| NM_001387 | 7F.6.C7 | dihydropyrimidinase-like 3 | −1.0685 | −0.5066 | 2.0957 |
| NM_002430 | 9R.2.C10 | meningioma (disrupted in balanced translocation) 1 | −1.0689 | −0.5074 | 2.0957 |
| NHF | 9F.8.H12 | | −1.0713 | −0.5081 | 2.0877 |
| H26022 | 14N.8.D2 | small inducible cytokine subfamily D (Cys-X3-Cys), member 1 | −1.074 | −0.5089 | 2.0182 |
| | | (fractalkine, neurotactin) | | | |
| BU542589 | 9F.7.D10 | putative G protein coupled receptor | −1.0762 | −0.5096 | 2.0102 |
| NHF | 9F.2.A3 | | −1.0767 | −0.5102 | 2.0102 |
| NM_001957 | 9F.3.G7 | endothelin receptor type A | −1.078 | −0.511 | 2.0066 |
| NHF | 9F.4.H8 | | −1.0792 | −0.5118 | 2.0044 |
| NM_001102 | 8F.2.H10 | actinin, alpha 1 | −1.0795 | −0.5125 | 2.0044 |
| AK090952 | 9R.6.H11 | Homo sapiens cDNA FLJ33633 fis, clone BRAMY2022786, highly | −1.0813 | −0.5132 | 1.9956 |
| | | similar to Homo sapiens dickkopf-3 (DKK-3) mRNA | | | |
| NM_001102 | 8F.3.B1 | actinin, alpha 1 | −1.0814 | −0.5139 | 1.9956 |
| NM_002948 | 8F.5.E10 | ribosomal protein L15 | −1.084 | −0.5147 | 1.9786 |
| NM_004487 | 12F.2.A8 | golgi autoantigen, golgin subfamily b, macrogolgin (with transmembrane | −1.0852 | −0.5154 | 1.9778 |
| | | signal), 1 | | | |
| AI381513 | 14N.3.H2 | xylosylprotein beta1,4-galactosyltransferase, polypeptide 7 | −1.0854 | −0.5162 | 1.9778 |
| | | (galactosyltransferase I) | | | |
| NHF | 9R.6.F4 | | −1.0862 | −0.517 | 1.9778 |
| N72289 | 7F.3.A3 | Homo sapiens cDNA FLJ12052 fis, clone HEMBB1002042, moderately | −1.0874 | −0.5177 | 1.9778 |
| | | similar to CYTOCHROME P450 4C1 (EC 1.14.14.1) | | | |
| AL360199 | 9F.3.H6 | Homo sapiens mRNA full length insert cDNA clone EUROIMAGE | −1.0902 | −0.5186 | 1.9434 |
| | | 179942 | | | |
| BI762020 | 9F.2.A4 | ESTs, Weakly similar to JC5963 stable tubule only polypeptide - mouse | −1.0905 | −0.5192 | 1.9434 |
| | | [M. musculus] | | | |
| H26022 | 14N.6.D2 | small inducible cytokine subfamily D (Cys-X3-Cys), member 1 | −1.0945 | −0.5201 | 1.9134 |
| | | (fractalkine, neurotactin) | | | |
| NM_013943 | 1R.2.E9 | chloride intracellular channel 4 | −1.0951 | −0.5209 | 1.9134 |
| AK091994 | 8F.10.C5 | Homo sapiens cDNA FLJ34675 fis, clone LIVER2001608 | −1.0981 | −0.5217 | 1.8736 |
| NM_030968 | 7F.8.B10 | C1q and tumor necrosis factor related protein 1 | −1.0983 | −0.5224 | 1.8736 |
| NM_001102 | 9R.8.C9 | actinin, alpha 1 | −1.0992 | −0.5233 | 1.8736 |
| AA081507 | 8F.3.H2 | ESTs | −1.1029 | −0.5242 | 1.8461 |
| BC038508 | 8F.6.F1 | transposon-derived Buster1 transposase-like protein | −1.1035 | −0.525 | 1.8461 |
| AK057652 | 7F.3.A2 | Homo sapiens cDNA FLJ33090 fis, clone TRACH2000559 | −1.1039 | −0.5258 | 1.8461 |
| NM_001920 | 8R.2.G8 | decorin | −1.1047 | −0.5266 | 1.8461 |
| NM_018507 | 12R.3.D2 | hypothetical protein PRO1843 | −1.1053 | −0.5275 | 1.8461 |
| AF267856 | 8R.7.B9 | hypothetical protein dJ465N24.2.1 | −1.1078 | −0.5283 | 1.8328 |
| AL080234 | 12F.2.G9 | Homo sapiens clone FBD3 Cri-du-chat critical region mRNA | −1.1136 | −0.5291 | 1.7611 |
| NM_001568 | 9F.8.B4 | eukaryotic translation initiation factor 3, subunit 6 48 kDa | −1.1156 | −0.53 | 1.7578 |
| BC017189 | 7R.2.H1 | Homo sapiens, myo-inositol 1-phosphate synthase A1, clone MGC: 726 | −1.1159 | −0.5309 | 1.7578 |
| | | IMAGE: 3140452, mRNA, complete cds | | | |
| NHF | 9F.8.H2 | | −1.1162 | −0.5317 | 1.7578 |
| AA348414 | 9R.2.B3 | ESTs, Weakly similar to JC5314 CDC28/cdc2-like kinase associating | −1.1167 | −0.5326 | 1.7578 |
| | | arginine-serine cyclophilin - human [H. sapiens] | | | |
| AK091994 | 8F.10.A1 | Homo sapiens cDNA FLJ34675 fis, clone LIVER2001608 | −1.1196 | −0.5335 | 1.7295 |
| NM_002026 | 7F.5.H7 | fibronectin 1 | −1.1199 | −0.5344 | 1.7295 |
| BI430544 | 12F.2.C6 | ESTs | −1.1205 | −0.5351 | 1.7295 |
| NM_024071 | 7R.8.F12 | hypothetical protein MGC2550 | −1.1216 | −0.536 | 1.7295 |
| BM473685 | 7F.2.C4 | UDP-glucose pyrophosphorylase 2 | −1.1218 | −0.537 | 1.7295 |
| NM_002948 | 7F.4.E5 | ribosomal protein L15 | −1.122 | −0.5378 | 1.7295 |
| NM_004939 | 8F.10.E12 | DEAD/H (Asp-Glu-Ala-Asp/His) box polypeptide 1 | −1.1237 | −0.5385 | 1.7295 |
| NHF | 9F.5.D3 | | −1.1266 | −0.5394 | 1.7264 |
| NM_024071 | 7R.7.B6 | hypothetical protein MGC2550 | −1.1288 | −0.5403 | 1.7168 |
| NHF | 9R.3.G3 | | −1.1313 | −0.5413 | 1.6945 |
| BC011987 | 8F.2.C10 | Homo sapiens, clone IMAGE: 3857153, mRNA | −1.1314 | −0.5422 | 1.6945 |
| NM_005032 | 8R.4.H6 | plastin 3 (T isoform) | −1.1326 | −0.5431 | 1.6945 |
| NM_005063 | 8F.8.B5 | stearoyl-CoA desaturase (delta-9-desaturase) | −1.1338 | −0.544 | 1.6945 |
| NM_005359 | 7F.10.C2 | MAD, mothers against decapentaplegic homolog 4 (Drosophila) | −1.1349 | −0.5451 | 1.6945 |
| NHF | 8F.8.E2 | | −1.1364 | −0.5462 | 1.6901 |
| NM_004130 | 1F.1.F3 | glycogenin | −1.1423 | −0.5472 | 1.6263 |
| NHF | 9F.9.D7 | | −1.1433 | −0.5481 | 1.6257 |
| NM_001901 | 9R.5.G12 | connective tissue growth factor | −1.1445 | −0.5491 | 1.625 |
| NM_030915 | 9R.9.G3 | likely ortholog of mouse limb-bud and heart gene | −1.1484 | −0.5502 | 1.598 |
| NM_018212 | 9F.7.F11 | enabled homolog (Drosophila) | −1.1485 | −0.5512 | 1.598 |
| AV719568 | 11F.1.H4 | EST | −1.1489 | −0.5522 | 1.598 |
| NM_001102 | 8F.3.F3 | actinin, alpha 1 | −1.1531 | −0.5533 | 1.5839 |
| NM_032476 | 9R.7.F12 | mitochondrial ribosomal protein S6 | −1.155 | −0.5543 | 1.5661 |
| BG483036 | 9R.8.C4 | integrin, alpha 1 | −1.1594 | −0.5551 | 1.5301 |
| NM_001102 | 8F.10.G9 | actinin, alpha 1 | −1.1629 | −0.5561 | 1.4981 |
| NM_003715 | 9R.5.A5 | vesicle docking protein p115 | −1.1664 | −0.5571 | 1.4736 |
| NM_000499 | 1F.1.B7 | cytochrome P450, subfamily I (aromatic compound-inducible), | −1.1672 | −0.5581 | 1.4736 |
| | | polypeptide 1 | | | |
| NM_001102 | 8F.1.F9 | actinin, alpha 1 | −1.172 | −0.5591 | 1.4245 |
| AF278532 | 1R.1.G4 | netrin 4 | −1.1739 | −0.5603 | 1.4162 |
| BG420559 | 9F.2.E5 | ESTs, Weakly similar to hypothetical protein FLJ22184 [Homo sapiens] | −1.1758 | −0.5613 | 1.4045 |
| | | [H. sapiens] | | | |
| NHF | 9R.3.G10 | | −1.1795 | −0.5624 | 1.3512 |
| BC020516 | 1R.2.A10 | Homo sapiens cDNA FLJ32368 fis, clone PUAEN1000275 | −1.1798 | −0.5635 | 1.3512 |
| NM_033014 | 9R.7.H3 | osteoglycin (osteoinductive factor, mimecan) | −1.1804 | −0.5645 | 1.3512 |
| AV719568 | 12R.3.H2 | EST | −1.1814 | −0.5655 | 1.3512 |
| NM_000627 | 8R.1.A8 | latent transforming growth factor beta binding protein 1 | −1.1849 | −0.5666 | 1.3469 |
| NM_014765 | 9F.1.C11 | translocase of outer mitochondrial membrane 20 (yeast) homolog | −1.1857 | −0.5679 | 1.3469 |
| U23841 | 9R.9.E4 | ESTs | −1.1861 | −0.569 | 1.3469 |
| AK096204 | 9F.3.D7 | Homo sapiens cDNA FLJ38885 fis, clone MESAN2017417, moderately | −1.189 | −0.5701 | 1.3438 |
| | | similar to REGULATOR OF G-PROTEIN SIGNALING 4 | | | |
| BI430544 | 12R.2.B5 | ESTs | −1.189 | −0.5712 | 1.3438 |
| NHF | 9R.10.A10 | | −1.1892 | −0.5723 | 1.3438 |
| NM_004130 | 1F.1.D12 | glycogenin | −1.1951 | −0.5734 | 1.3316 |
| BC026873 | 9R.9.D10 | Homo sapiens, similar to RIKEN cDNA 1110018M03, clone MGC: 24932 | −1.1969 | −0.5746 | 1.3277 |
| | | IMAGE: 4938507, mRNA, complete cds | | | |
| NM_053056 | 9F.5.A5 | cyclin D1 (PRAD1: parathyroid adenomatosis 1) | −1.1995 | −0.5756 | 1.3056 |
| NM_005863 | 8R.4.B9 | neuroepithelial cell transforming gene 1 | −1.1996 | −0.5767 | 1.3056 |
| AK093643 | 9F.4.C9 | esterase D/formylglutathione hydrolase | −1.2001 | −0.5778 | 1.3056 |
| AK055112 | 12F.3.A11 | Homo sapiens cDNA FLJ30550 fis, clone BRAWH2001502 | −1.2019 | −0.579 | 1.3056 |
| BI430544 | 12R.2.A3 | ESTs | −1.2051 | −0.5801 | 1.2893 |
| H64346 | 14N.1.E4 | syndecan 2 (heparan sulfate proteoglycan 1, cell surface-associated, | −1.2137 | −0.5815 | 1.2118 |
| | | fibroglycan) | | | |
| NM_138737 | 9R.5.H6 | hephaestin | −1.2147 | −0.5827 | 1.2118 |
| NM_003932 | 12F.1.F8 | suppression of tumorigenicity 13 (colon carcinoma) (Hsp70 interacting | −1.2182 | −0.5839 | 1.2081 |
| | | protein) | | | |
| NM_152535 | 8R.4.C5 | hypothetical protein FLJ31131 | −1.2238 | −0.5851 | 1.156 |
| AL832012 | 7F.5.G4 | transmembrane trafficking protein | −1.228 | −0.5864 | 1.1401 |
| AL833550 | 12F.3.G3 | exportin 1 (CRM1 homolog, yeast) | −1.23 | −0.5875 | 1.1333 |
| NM_002948 | 9F.4.B12 | ribosomal protein L15 | −1.232 | −0.5888 | 1.1292 |
| NM_005032 | 8R.8.F3 | plastin 3 (T isoform) | −1.2484 | −0.5902 | 1.0306 |
| BE621121 | 7R.5.D8 | EST | −1.2487 | −0.5915 | 1.0306 |
| NM_002306 | 8R.5.F8 | lectin, galactoside-binding, soluble, 3 (galectin 3) | −1.2519 | −0.5926 | 1.0195 |
| BF976811 | 12R.3.D11 | leucyl-tRNA synthetase | −1.2525 | −0.5938 | 1.0195 |
| AL359062 | 7F.2.D6 | Homo sapiens mRNA full length insert cDNA clone EUROIMAGE | −1.2537 | −0.5951 | 1.0195 |
| | | 1913076 | | | |
| AF231512 | 12R.3.G11 | Homo sapiens RNA binding motif protein 8B (RBM8B) mRNA, complete | −1.2538 | −0.5964 | 1.0195 |
| | | cds | | | |
| BQ223934 | 1R.1.A1 | UDP-glucose pyrophosphorylase 2 | −1.2605 | −0.5976 | 0.9907 |
| NM_005345 | 7F.2.G9 | heat shock 70 kDa protein 1A | −1.2626 | −0.599 | 0.9907 |
| NM_000366 | 8R.7.H12 | tropomyosin 1 (alpha) | −1.2667 | −0.6006 | 0.976 |
| NM_014765 | 9F.1.B6 | translocase of outer mitochondrial membrane 20 (yeast) homolog | −1.2689 | −0.6021 | 0.9733 |
| BG118720 | 7F.8.C11 | putative G protein coupled receptor | −1.2696 | −0.6033 | 0.9733 |
| AI707775 | 8F.9.H4 | EST | −1.2727 | −0.6046 | 0.938 |
| NM_000366 | 7R.7.H5 | tropomyosin 1 (alpha) | −1.2771 | −0.6061 | 0.9311 |
| AF156100 | 8R.5.A9 | fibulin 6 | −1.2782 | −0.6073 | 0.9311 |
| NM_033014 | 9R.9.D9 | osteoglycin (osteoinductive factor, mimecan) | −1.288 | −0.6086 | 0.8854 |
| NHF | 9R.10.E8 | | −1.2889 | −0.6102 | 0.8854 |
| BG028195 | 7F.9.F8 | Homo sapiens cDNA FLJ38755 fis, clone KIDNE2012775, weakly | −1.3011 | −0.6117 | 0.8111 |
| | | similar to Homo sapiens mRNA for transport-secretion protein 2.1 | | | |
| BM543221 | 9R.8.E5 | ESTs | −1.3024 | −0.6131 | 0.8111 |
| AK090550 | 7R.4.B9 | Homo sapiens cDNA: FLJ21533 fis, clone COL06072 | −1.3051 | −0.6144 | 0.8111 |
| AL359052 | 9F.8.D6 | Homo sapiens mRNA full length insert cDNA clone EUROIMAGE | −1.306 | −0.616 | 0.8111 |
| | | 1968422 | | | |
| AB014527 | 8F.5.E4 | cytoplasmic linker associated protein 2 | −1.3108 | −0.6178 | 0.7934 |
| AL833137 | 9F.8.A7 | Homo sapiens, clone IMAGE: 3915000, mRNA | −1.3178 | −0.6194 | 0.7668 |
| NHF | 9R.3.A10 | | −1.3196 | −0.621 | 0.7668 |
| NM_014000 | 12F.3.D8 | vinculin | −1.3216 | −0.6226 | 0.7668 |
| BI430544 | 12R.1.A5 | ESTs | −1.3234 | −0.6242 | 0.7463 |
| BQ879275 | 7F.10.A11 | Homo sapiens, clone IMAGE: 4296901, mRNA | −1.3278 | −0.6258 | 0.7404 |
| N27086 | 14N.2.F2 | Hs. cDNA FLJ11363 fis, clone HEMBA1000251 | −1.3427 | −0.6275 | 0.6794 |
| NM_002884 | 1R.2.D11 | RAP1A, member of RAS oncogene family | −1.3473 | −0.629 | 0.6571 |
| NM_000627 | 9F.2.B6 | latent transforming growth factor beta binding protein 1 | −1.3482 | −0.6306 | 0.6571 |
| NM_005730 | 8R.1.H1 | conserved gene amplified in osteosarcoma | −1.3488 | −0.6323 | 0.6571 |
| NHF | 9F.2.A1 | | −1.3555 | −0.634 | 0.6373 |
| NM_024071 | 7R.1.B11 | hypothetical protein MGC2550 | −1.3556 | −0.636 | 0.6373 |
| AK055197 | 9F.1.E1 | Homo sapiens cDNA FLJ30635 fis, clone CTONG2002520 | −1.3692 | −0.6376 | 0.5845 |
| NM_005032 | 8R.9.F1 | plastin 3 (T isoform) | −1.3727 | −0.6395 | 0.5845 |
| AK096260 | 9F.4.C2 | hypothetical protein FLJ14399 | −1.3728 | −0.6412 | 0.5845 |
| AL833007 | 7R.2.E4 | Homo sapiens, clone IMAGE: 3625286, mRNA, partial cds | −1.3729 | −0.6431 | 0.5845 |
| NM_000362 | 9F.4.F5 | tissue inhibitor of metalloproteinase 3 (Sorsby fundus dystrophy, | −1.3866 | −0.645 | 0.5569 |
| | | pseudoinflammatory) | | | |
| BU075881 | 11R.1.H10 | small proline-rich protein 2A | −1.3874 | −0.647 | 0.5569 |
| NHF | 7F.7.H7 | | −1.3934 | −0.6489 | 0.5342 |
| BI430544 | 11R.1.D10 | ESTs | −1.3946 | −0.6508 | 0.5342 |
| NM_007341 | 7F.1.D7 | SH3 domain binding glutamic acid-rich protein | −1.3963 | −0.6527 | 0.5342 |
| NHF | 9R.3.D12 | | −1.3981 | −0.6545 | 0.5342 |
| AK055197 | 7R.7.F7 | Homo sapiens cDNA FLJ30635 fis, clone CTONG2002520 | −1.3994 | −0.6566 | 0.5342 |
| NM_014851 | 9R.3.B4 | KIAA0469 gene product | −1.4052 | −0.6583 | 0.5342 |
| AK096403 | 9R.8.H10 | Homo sapiens cDNA FLJ39084 fis, clone NT2RP7018871 | −1.4085 | −0.6605 | 0.528 |
| NHF | 7F.7.A10 | | −1.4106 | −0.6625 | 0.5243 |
| NM_021069 | 1R.2.B3 | Arg/Abl-interacting protein ArgBP2 | −1.4204 | −0.6649 | 0.4882 |
| NHF | 9R.3.E6 | | −1.425 | −0.667 | 0.4802 |
| NM_144573 | 8R.8.G7 | likely ortholog of rat F-actin binding protein nexilin | −1.4258 | −0.6691 | 0.4802 |
| NM_002026 | 8R.5.G8 | fibronectin 1 | −1.4367 | −0.6713 | 0.4609 |
| AF231512 | 7R.4.C9 | Homo sapiens RNA binding motif protein 8B (RBM8B) mRNA, complete | −1.4463 | −0.6737 | 0.4438 |
| | | cds | | | |
| NM_005032 | 8R.1.A9 | plastin 3 (T isoform) | −1.4611 | −0.6756 | 0.4004 |
| NHF | 9R.5.F6 | | −1.4701 | −0.6778 | 0.3813 |
| NHF | 7F.7.H1 | | −1.4791 | −0.6801 | 0.3651 |
| NM_016081 | 12F.3.G2 | palladin | −1.5035 | −0.6826 | 0.3 |
| NHF | 9R.6.C7 | | −1.5115 | −0.6852 | 0.2946 |
| NM_033138 | 7R.4.E12 | caldesmon 1 | −1.5188 | −0.6878 | 0.291 |
| AK027088 | 12F.1.F12 | Homo sapiens cDNA: FLJ23435 fis, clone HRC12631 | −1.522 | −0.6903 | 0.2832 |
| NHF | 9R.3.E3 | | −1.5294 | −0.6928 | 0.2758 |
| AL833007 | 9R.9.F8 | Homo sapiens, clone IMAGE: 3625286, mRNA, partial cds | −1.5299 | −0.6951 | 0.2758 |
| NM_005730 | 8R.7.F11 | conserved gene amplified in osteosarcoma | −1.5452 | −0.6978 | 0.2473 |
| BE966567 | 8R.1.H7 | EST | −1.5509 | −0.7006 | 0.2459 |
| AF153821 | 8R.5.E12 | alcohol dehydrogenase IB (class I), beta polypeptide | −1.5512 | −0.7033 | 0.2459 |
| NM_000627 | 8F.10.D7 | latent transforming growth factor beta binding protein 1 | −1.5568 | −0.7062 | 0.2458 |
| NM_138737 | 9F.2.A7 | hephaestin | −1.5658 | −0.7088 | 0.2326 |
| NM_000627 | 8F.5.G2 | latent transforming growth factor beta binding protein 1 | −1.5689 | −0.7117 | 0.231 |
| NM_000627 | 8F.4.G8 | latent transforming growth factor beta binding protein 1 | −1.5702 | −0.7152 | 0.231 |
| NM_006870 | 8F.10.F2 | destrin (actin depolymerizing factor) | −1.5723 | −0.7184 | 0.231 |
| NM_005032 | 8R.6.A11 | plastin 3 (T isoform) | −1.5764 | −0.7214 | 0.231 |
| NM_000627 | 8F.5.D2 | latent transforming growth factor beta binding protein 1 | −1.578 | −0.7243 | 0.231 |
| NM_006014 | 8F.7.H5 | DNA segment on chromosome X (unique) 9879 expressed sequence | −1.5805 | −0.7278 | 0.231 |
| NM_005032 | 8R.2.H8 | plastin 3 (T isoform) | −1.5823 | −0.7314 | 0.231 |
| AB029018 | 9R.8.B10 | likely ortholog of mouse semaF cytoplasmic domain associated protein 3 | −1.5831 | −0.735 | 0.231 |
| NHF | 9R.3.C9 | | −1.5876 | −0.7385 | 0.231 |
| BI430544 | 11R.1.D6 | ESTs | −1.5956 | −0.7421 | 0.231 |
| NM_014819 | 12R.2.A7 | KIAA0438 gene product | −1.6048 | −0.7458 | 0.2298 |
| NM_014890 | 12F.2.H3 | downregulated in ovarian cancer 1 | −1.6064 | −0.7502 | 0.2298 |
| BI430544 | 12F.2.B6 | ESTs | −1.6119 | −0.7541 | 0.2298 |
| AK055197 | 7R.9.F8 | Homo sapiens cDNA FLJ30635 fis, clone CTONG2002520 | −1.6158 | −0.7578 | 0.2298 |
| NM_017813 | 8R.4.G8 | hypothetical protein FLJ20421 | −1.6196 | −0.762 | 0.2298 |
| AL833007 | 8F.9.D1 | Homo sapiens, clone IMAGE: 3625286, mRNA, partial cds | −1.6351 | −0.7663 | 0.2072 |
| BC009220 | 7R.3.A9 | Homo sapiens, clone MGC: 16362 IMAGE: 3927795, mRNA, complete | −1.6395 | −0.7708 | 0.2072 |
| | | cds | | | |
| BM982785 | 9R.8.E1 | Rho-associated, coiled-coil containing protein kinase 1 | −1.6468 | −0.7748 | 0.2072 |
| NM_000627 | 8F.5.B4 | latent transforming growth factor beta binding protein 1 | −1.6584 | −0.7795 | 0.2069 |
| AF156100 | 9R.8.F9 | fibulin 6 | −1.6597 | −0.7845 | 0.2069 |
| NM_016081 | 12R.1.C11 | palladin | −1.6684 | −0.7901 | 0.2069 |
| NM_003601 | 7F.7.H8 | SWI/SNF related, matrix associated, actin dependent regulator of | −1.6823 | −0.795 | 0.1879 |
| | | chromatin, subfamily a, member 5 | | | |
| NHF | 9F.4.H11 | | −1.7004 | −0.8009 | 0.1705 |
| NHF | 7F.7.G12 | | −1.7048 | −0.807 | 0.1705 |
| NM_016081 | 12R.3.F8 | palladin | −1.7055 | −0.8131 | 0.1705 |
| AB040951 | 9R.9.F2 | KIAA1518 protein | −1.7171 | −0.8194 | 0.1705 |
| AA629603 | 14N.4.E6 | PTPL1-associated RhoGAP 1 | −1.7233 | −0.8256 | 0.1705 |
| AF156100 | 8R.7.B10 | fibulin 6 | −1.7374 | −0.8328 | 0.1667 |
| NHF | 7F.8.G4 | | −1.7599 | −0.8407 | 0.1144 |
| BU584993 | 7F.7.B5 | ESTs, Highly similar to potassium voltage-gated channel, lsk-related | −1.7638 | −0.8475 | 0.1144 |
| | | subfamily, gene 4; potassium voltage-gated channel-like protein, lsk- | | | |
| | | related subfamily [Homo sapiens] [H. sapiens] | | | |
| NM_016081 | 12R.2.G2 | palladin | −1.7686 | −0.8557 | 0.1144 |
| AA046932 | 9F.8.E3 | My015 | −17686 | −0.8652 | 0.1144 |
| NHF | 7F.7.A6 | | −1.777 | −0.8756 | 0.1144 |
| NM_016081 | 12R.3.A6 | palladin | −1.7834 | −0.8862 | 0.1144 |
| BQ429410 | 9R.2.A7 | Rho-associated, coiled-coil containing protein kinase 1 | −1.7843 | −0.8969 | 0.1144 |
| NM_031442 | 12F.1.A1 | brain cell membrane protein 1 | −1.7917 | −0.9081 | 0.1144 |
| AL833007 | 9R.2.C1 | Homo sapiens, clone IMAGE: 3625286, mRNA, partial cds | −1.7936 | −0.9201 | 0.1144 |
| NM_016081 | 12R.1.E8 | palladin | −1.8093 | −0.9334 | 0.1144 |
| AL833007 | 9R.9.B6 | Homo sapiens, clone IMAGE: 3625286, mRNA, partial cds | −1.82 | −0.9492 | 0.1144 |
| NM_016081 | 12R.3.E9 | palladin | −1.8265 | −0.9661 | 0.1144 |
| BC009220 | 9R.4.G4 | Homo sapiens, clone MGC: 16362 IMAGE: 3927795, mRNA, complete | −1.8368 | −0.9876 | 0.1144 |
| | | cds | | | |
| N73625 | 14N.2.B2 | EST | −1.8724 | −1.0113 | 0.1144 |
| NHF | 9R.8.H12 | | −2.0128 | −1.038 | 0.0824 |
| M69181 | 1F.1.D2 | myosin, heavy polypeptide 10, non-muscle | −2.0278 | −1.075 | 0.0824 |
| NHF | 7F.7.E4 | | −2.2048 | −1.1383 | 0.0299 |
| AL832780 | 7F.7.D4 | Homo sapiens mRNA; cDNA DKFZp686J037 (from clone | −2.5085 | −1.2589 | 0.0299 |
| | | DKFZp686J037) | | | |

TABLE 3

| | | | | | | | |
|---|---|---|---|---|---|---|---|
| | | | | | | t-test score | |
| SystematicName | UnigeneCode | GeneName | GeneSymbol | TNoM p-value | Ratio fold change | p-value | Change Directions |

Genes with Known Name/or Functions (Note: Lesion > No lesion, Foldchange positive; No lesion > lesion, fold change negative).

| | | | | | | | |
|---|---|---|---|---|---|---|---|
| N98591 | Hs.93913 | interleukin 6 (interferon, beta 2) | | 9.42E−04 | 2.29 | 6.82E−03 | Lesion and no DM < Lesion and DM |
| NM_004530 | HS.111301 | matrix metalloproteinase 2 (gelatinase A, 72 kDa | MMP2 | 3.03E−06 | 2.21 | 1.93E−08 | Lesion and no DM < Lesion and DM |
| | | gelatinase, 72 kDa type IV collagenase) | | | | | |
| NM_001552 | Hs.1516 | insulin-like growth factor binding protein 4 | IGFBP4 | 1.79E−09 | 2.21 | 1.50E−04 | Lesion and no DM < Lesion and DM |
| NM_006216 | Data not found | serine (or cysteine) proteinase inhibitor, clade E | SERPINE2 | 5.65E−07 | 2.12 | 3.32E−04 | Lesion and no DM < Lesion and DM |
| | | (nexin, plasminogen activator inhibitor type 1), | | | | | |
| | | member 2 | | | | | |
| NM_000104 | Data not found | cytochrome P450, subfamily I (dioxin-inducible), | CYP1B1 | 1.39E−08 | 2.09 | 3.14E−04 | Lesion and no DM < Lesion and DM |
| | | polypeptide 1 (glaucoma 3, primary infantile) | | | | | |
| AA936768 | Hs.1722 | interleukin 1, alpha | | 1.47E−05 | 2.08 | 1.23E−09 | Lesion and no DM < Lesion and DM |
| NM_000088 | Data not found | collagen, type I, alpha 1 | COL1A1 | 6.43E−05 | 2.03 | 5.44E−05 | Lesion and no DM < Lesion and DM |
| NM_001235 | Hs.9930 | serine (or cysteine) proteinase inhibitor, clade H | SERPINH1 | 1.44E−12 | 2.01 | 7.42E−09 | Lesion and no DM < Lesion and DM |
| | | (heat shock protein 47), member 2 | | | | | |
| AA156031 | Hs.118786 | metallothionein 2A | | 5.65E−07 | 1.91 | 4.72E−07 | Lesion and no DM < Lesion and DM |
| AF506819 | Hs.343483 | Homo sapiens URB mRNA, complete cds | URB | 1.47E−05 | 1.86 | 6.68E−03 | Lesion and no DM < Lesion and DM |
| NM_003254 | Hs.5831 | tissue inhibitor of metalloproteinase 1 (erythroid | TIMP1 | 1.47E−05 | 1.85 | 1.14E−03 | Lesion and no DM < Lesion and DM |
| | | potentiating activity, collagenase inhibitor) | | | | | |
| NM_006756 | Hs.78869 | transcription elongation factor A (SII), 1 | TCEA1 | 1.39E−08 | 1.80 | 2.73E−03 | Lesion and no DM < Lesion and DM |
| BI830199 | Data not found | likely ortholog of mouse Urb | | 6.43E−05 | 1.79 | 2.15E−03 | Lesion and no DM < Lesion and DM |
| NM_000089 | Data not found | collagen, type I, alpha 2 | COL1A2 | 9.42E−04 | 1.76 | 2.75E−03 | Lesion and no DM < Lesion and DM |
| AK025599 | Hs.25253 | mannosidase, alpha, class 1A, member 1 | | 1.87E−11 | 1.76 | 1.15E−06 | Lesion and no DM < Lesion and DM |
| M14219 | Hs.76152 | Human chondroitin/dermatan sulfate | | 9.42E−04 | 1.72 | 1.47E−03 | Lesion and no DM < Lesion and DM |
| | | proteoglycan (PG40) core protein mRNA, | | | | | |
| | | complete cds | | | | | |
| U72621 | Hs.75825 | pleiomorphic adenoma gene-like 1 | | 1.39E−08 | 1.68 | 2.47E−08 | Lesion and no DM < Lesion and DM |
| NM_000358 | Hs.118787 | transforming growth factor, beta-induced, 68 kDa | TGFBI | 3.03E−06 | 1.66 | 6.58E−06 | Lesion and no DM < Lesion and DM |
| NM_006307 | Data not found | sushi-repeat-containing protein, X chromosome | SRPX | 1.47E−05 | 1.65 | 9.94E−06 | Lesion and no DM < Lesion and DM |
| NM_002923 | Hs.78944 | regulator of G-protein signalling 2, 24 kDa | RGS2 | 2.57E−04 | 1.64 | 1.62E−02 | Lesion and no DM < Lesion and DM |
| NM_000090 | Data not found | collagen, type III, alpha 1 (Ehlers-Danlos | COL3A1 | 1.47E−05 | 1.64 | 1.79E−02 | Lesion and no DM < Lesion and DM |
| | | syndrome type IV, autosomal dominant) | | | | | |
| NM_005110 | Data not found | glutamine-fructose-6-phosphate transaminase 2 | GFPT2 | 9.42E−04 | 1.64 | 2.40E−03 | Lesion and no DM < Lesion and DM |
| NM_004404 | Hs.155595 | neural precursor cell expressed, | NEDD5 | 3.03E−06 | 1.64 | 3.97E−05 | Lesion and no DM < Lesion and DM |
| | | developmentally down-regulated 5 | | | | | |
| NM_004369 | Hs.80988 | collagen, type VI, alpha 3 | COL6A3 | 2.57E−04 | 1.63 | 4.02E−03 | Lesion and no DM < Lesion and DM |
| AA146772 | Hs.82396 | 2′,5′-oligoadenylate synthetase 1 (40-46 kD) | | 9.42E−04 | 1.62 | 8.27E−04 | Lesion and no DM < Lesion and DM |
| NM_023009 | Hs.75061 | MARCKS-like protein | MLP | 5.65E−07 | 1.59 | 1.48E−06 | Lesion and no DM < Lesion and DM |
| NM_006435 | Hs.174195 | interferon induced transmembrane protein 2 (1-8D) | IFITM2 | 3.03E−06 | 1.58 | 4.66E−06 | Lesion and no DM < Lesion and DM |
| BC014836 | Hs.79086 | Homo sapiens, mitochondrial ribosomal protein | | 2.57E−04 | 1.57 | 7.88E−03 | Lesion and no DM < Lesion and DM |
| | | L3, clone MGC: 9373 IMAGE: 3860982, mRNA, | | | | | |
| | | complete cds | | | | | |
| NM_001022 | Hs.298262 | ribosomal protein S19 | RPS19 | 5.65E−07 | 1.55 | 7.37E−08 | Lesion and no DM < Lesion and DM |
| BU626315 | Data not found | collagen, type V, alpha 1 | | 6.43E−05 | 1.54 | 1.30E−02 | Lesion and no DM < Lesion and DM |
| NM_001710 | Hs.69771 | B-factor, properdin | BF | 6.43E−05 | 1.53 | 6.98E−04 | Lesion and no DM < Lesion and DM |
| NM_006745 | Hs.239926 | sterol-C4-methyl oxidase-like | SC4MOL | 2.57E−04 | 1.53 | 3.62E−03 | Lesion and no DM < Lesion and DM |
| NM_003029 | Hs.81972 | SHC (Src homology 2 domain containing) | SHC1 | 1.47E−05 | 1.51 | 2.51E−07 | Lesion and no DM < Lesion and DM |
| | | transforming protein 1 | | | | | |
| NM_002009 | Hs.164568 | fibroblast growth factor 7 (keratinocyte growth | FGF7 | 9.42E−04 | 1.51 | 1.19E−04 | Lesion and no DM < Lesion and DM |
| | | factor) | | | | | |
| NM_053275 | Hs.73742 | ribosomal protein, large, P0 | RPLP0 | 1.99E−10 | 1.47 | 1.07E−03 | Lesion and no DM < Lesion and DM |
| NM_000365 | Data not found | triosephosphate isomerase 1 | TPI1 | 9.42E−04 | 1.47 | 1.75E−05 | Lesion and no DM < Lesion and DM |
| BC008791 | Hs.179661 | Homo sapiens, tubulin, beta 5, clone MGC: 4029 | | 2.57E−04 | 1.46 | 4.53E−05 | Lesion and no DM < Lesion and DM |
| | | IMAGE: 3617988, mRNA, complete cds | | | | | |
| AB051510 | Hs.8700 | deleted in liver cancer 1 | | 2.57E−04 | 1.46 | 1.17E−04 | Lesion and no DM < Lesion and DM |
| NM_002707 | Hs.17883 | protein phosphatase 1G (formerly 2C), | | 9.42E−04 | 1.46 | 2.92E−02 | Lesion and no DM < Lesion and DM |
| | | magnesium-dependent, gamma isoform | | | | | |
| NM_000201 | Hs.168383 | intercellular adhesion molecule 1 (CD54), | ICAM1 | 6.43E−05 | 1.46 | 1.89E−02 | Lesion and no DM < Lesion and DM |
| | | human rhinovirus receptor | | | | | |
| NM_002291 | Hs.82124 | laminin, beta 1 | LAMB1 | 9.42E−08 | 1.46 | 2.58E−03 | Lesion and no DM < Lesion and DM |
| NM_021034 | Hs.182241 | interferon induced transmembrane protein 3 (1-8U) | IFITM3 | 9.42E−08 | 1.45 | 6.39E−05 | Lesion and no DM < Lesion and DM |
| NM_015933 | Hs.171774 | hypothetical protein HSPC016 | HSPC016 | 9.42E−04 | 1.44 | 3.23E−05 | Lesion and no DM < Lesion and DM |
| AW005755 | Hs.73798 | macrophage migration inhibitory factor | | 1.47E−05 | 1.43 | 2.81E−06 | Lesion and no DM < Lesion and DM |
| | | (glycosylation-inhibiting factor) | | | | | |
| NM_005803 | Hs.179986 | flotillin 1 | FLOT1 | 9.42E−04 | 1.41 | 3.36E−05 | Lesion and no DM < Lesion and DM |
| NM_001734 | Hs.169756 | complement component 1, s subcomponent | C1S | 3.03E−06 | 1.41 | 6.35E−02 | Lesion and no DM < Lesion and DM |
| NM_004199 | Hs.3622 | procollagen-proline, 2-oxoglutarate 4- | P4HA2 | 9.42E−04 | 1.41 | 5.94E−03 | Lesion and no DM < Lesion and DM |
| | | dioxygenase (proline 4-hydroxylase), alpha | | | | | |
| | | polypeptide II | | | | | |
| NM_021103 | Hs.76293 | thymosin, beta 10 | TMSB10 | 2.57E−04 | 1.41 | 1.91E−04 | Lesion and no DM < Lesion and DM |
| AK092774 | Data not found | ribosomal protein, large P2 | RPLP2 | 9.42E−04 | 1.40 | 6.21E−03 | Lesion and no DM < Lesion and DM |
| AK091661 | Hs.15961 | dynactin 3 (p22) | | 9.42E−04 | 1.39 | 3.68E−03 | Lesion and no DM < Lesion and DM |
| AK091360 | Hs.183180 | APC11 anaphase promoting complex subunit | | 1.47E−05 | 1.39 | 5.13E−05 | Lesion and no DM < Lesion and DM |
| | | 11 homolog (yeast) | | | | | |
| NM_005347 | Hs.75410 | heat shock 70 kDa protein 5 (glucose-regulated | HSPA5 | 1.47E−05 | 1.39 | 1.52E−02 | Lesion and no DM < Lesion and DM |
| | | protein, 78 kDa) | | | | | |
| NM_000935 | Hs.41270 | procollagen-lysine, 2-oxoglutarate 5- | PLOD2 | 3.03E−06 | 1.39 | 3.78E−04 | Lesion and no DM < Lesion and DM |
| | | dioxygenase (lysine hydroxylase) 2 | | | | | |
| NM_032704 | Data not found | tubulin alpha 6 | TUBA6 | 2.57E−04 | 1.38 | 3.43E−07 | Lesion and no DM < Lesion and DM |
| AA625981 | Hs.752 | FK506 binding protein 1A (12 kD) | | 6.43E−05 | 1.38 | 1.73E−06 | Lesion and no DM < Lesion and DM |
| NM_020650 | Hs.39619 | hypothetical protein LOC57333 | RCN3 | 2.57E−04 | 1.38 | 9.13E−04 | Lesion and no DM < Lesion and DM |
| NM_033301 | Hs.178551 | ribosomal protein L8 | RPL8 | 6.43E−05 | 1.38 | 2.48E−06 | Lesion and no DM < Lesion and DM |
| NM_006432 | Hs.119529 | Niemann-Pick disease, type C2 | NPC2 | 9.42E−04 | 1.38 | 2.20E−05 | Lesion and no DM < Lesion and DM |
| BF976811 | Data not found | leucyl-tRNA synthetase | | 2.57E−04 | 1.37 | 1.37E−02 | Lesion and no DM < Lesion and DM |
| NM_005625 | Data not found | syndecan binding protein (syntenin) | SDCBP | 6.43E−05 | 1.37 | 1.82E−03 | Lesion and no DM < Lesion and DM |
| AI088089 | Hs.164568 | fibroblast growth factor 7 (keratinocyte growth | | 6.43E−05 | 1.37 | 1.06E−02 | Lesion and no DM < Lesion and DM |
| | | factor) | | | | | |
| NM_002631 | Hs.75888 | phosphogluconate dehydrogenase | PGD | 2.57E−04 | 1.36 | 1.30E−03 | Lesion and no DM < Lesion and DM |
| NM_003479 | Hs.82911 | protein tyrosine phosphatase type IVA, member 2 | PTP4A2 | 2.57E−04 | 1.36 | 7.18E−03 | Lesion and no DM < Lesion and DM |
| NM_080388 | Data not found | hypothetical protein MGC17528 | S100A16 | 9.42E−04 | 1.36 | 2.58E−03 | Lesion and no DM < Lesion and DM |
| AA481464 | Hs.699 | peptidylprolyl isomerase B (cyclophilin B) | | 1.39E−08 | 1.36 | 1.07E−04 | Lesion and no DM < Lesion and DM |
| NM_015414 | Data not found | ribosomal protein L36 | RPL36 | 9.42E−04 | 1.35 | 3.69E−03 | Lesion and no DM < Lesion and DM |
| BG680524 | Hs.129673 | RNA, U67 small nucleolar | | 2.57E−04 | 1.35 | 6.60E−06 | Lesion and no DM < Lesion and DM |
| NM_006335 | Hs.20716 | translocase of inner mitochondrial membrane | TIMM17A | 6.43E−05 | 1.33 | 3.41E−03 | Lesion and no DM < Lesion and DM |
| | | 17 homolog A (yeast) | | | | | |
| NM_002074 | Hs.215595 | guanine nucleotide binding protein (G protein), | GNB1 | 2.57E−04 | 1.33 | 5.48E−02 | Lesion and no DM < Lesion and DM |
| | | beta polypeptide 1 | | | | | |
| NM_000393 | Hs.82985 | collagen, type V, alpha 2 | COL5A2 | 2.57E−04 | 1.32 | 2.06E−06 | Lesion and no DM < Lesion and DM |
| BC004215 | Data not found | Homo sapiens, eukaryotic translation elongation | | 9.42E−04 | 1.31 | 2.68E−02 | Lesion and no DM < Lesion and DM |
| | | factor 1 gamma, clone MGC: 4501 | | | | | |
| | | IMAGE: 2964623, mRNA, complete cds | | | | | |
| NM_007075 | Data not found | JM5 protein | WDRX1 | 3.03E−06 | 1.30 | 2.15E−03 | Lesion and no DM < Lesion and DM |
| NM_002615 | Hs.173594 | serine (or cysteine) proteinase inhibitor, clade F | SERPINF1 | 3.03E−06 | 1.30 | 2.41E−01 | Lesion and no DM < Lesion and DM |
| | | (alpha-2 antiplasmin, pigment epithelium | | | | | |
| | | derived factor), member 1 | | | | | |
| NM_001780 | Hs.76294 | CD63 antigen (melanoma 1 antigen) | CD63 | 1.47E−05 | 1.30 | 2.39E−07 | Lesion and no DM < Lesion and DM |
| NM_003299 | Hs.82689 | tumor rejection antigen (gp96) 1 | TRA1 | 9.42E−04 | 1.30 | 3.65E−02 | Lesion and no DM < Lesion and DM |
| AF208043 | Hs.155530 | interferon, gamma-inducible protein 16 | | 6.43E−05 | 1.30 | 2.25E−02 | Lesion and no DM < Lesion and DM |
| NM_000138 | Hs.750 | fibrillin 1 (Marfan syndrome) | FBN1 | 2.57E−04 | 1.29 | 3.82E−02 | Lesion and no DM < Lesion and DM |
| NM_000944 | Hs.272458 | protein phosphatase 3 (formerly 2B), catalytic | PPP3CA | 9.42E−04 | 1.29 | 1.49E−03 | Lesion and no DM < Lesion and DM |
| | | subunit, alpha isoform (calcineurin A alpha) | | | | | |
| H02884 | Hs.76206 | cadherin 5, type 2, VE-cadherin (vascular | | 9.42E−04 | 1.29 | 5.53E−03 | Lesion and no DM < Lesion and DM |
| | | epithelium) | | | | | |
| BU838358 | Hs.102267 | lysyl oxidase | | 9.42E−04 | 1.27 | 1.48E−02 | Lesion and no DM < Lesion and DM |
| NM_005324 | Hs.180877 | H3 histone, family 3B (H3.3B) | H3F3B | 2.57E−04 | 1.27 | 1.51E−05 | Lesion and no DM < Lesion and DM |
| NM_021109 | Hs.75968 | thymosin, beta 4, X chromosome | | 9.42E−04 | 1.27 | 1.37E−02 | Lesion and no DM < Lesion and DM |
| NM_032682 | Data not found | forkhead box P1 | FOXP1 | 9.42E−04 | 1.27 | 7.68E−02 | Lesion and no DM < Lesion and DM |
| BE047418 | Hs.119122 | ribosomal protein L13a | | 2.57E−04 | 1.27 | 2.84E−03 | Lesion and no DM < Lesion and DM |
| AJ238214 | Data not found | WD repeat domain 9 | | 2.57E−04 | 1.27 | 2.27E−03 | Lesion and no DM < Lesion and DM |
| NM_153649 | Hs.85844 | tropomyosin 3 | TPM3 | 9.42E−04 | 1.26 | 2.47E−03 | Lesion and no DM < Lesion and DM |
| NM_000990 | Hs.76064 | ribosomal protein L27a | RPL27A | 9.42E−04 | 1.26 | 9.61E−03 | Lesion and no DM < Lesion and DM |
| NM_001012 | Hs.151604 | ribosomal protein S8 | RPS8 | 9.42E−04 | 1.25 | 4.77E−03 | Lesion and no DM < Lesion and DM |
| NM_002818 | Hs.179774 | proteasome (prosome, macropain) activator | PSME2 | 9.42E−04 | 1.25 | 7.06E−04 | Lesion and no DM < Lesion and DM |
| | | subunit 2 (PA28 beta) | | | | | |
| NM_005878 | Hs.21858 | trinucleotide repeat containing 3 | | 2.57E−04 | 1.25 | 5.94E−02 | Lesion and no DM < Lesion and DM |
| R22412 | Hs.78146 | platelet/endothelial cell adhesion molecule | | 2.57E−04 | 1.23 | 1.46E−01 | Lesion and no DM < Lesion and DM |
| | | (CD31 antigen) | | | | | |
| NM_004099 | Data not found | stomatin | | 9.42E−04 | 1.23 | 1.35E−02 | Lesion and no DM < Lesion and DM |
| AY092084 | Hs.197642 | RNA polymerase III subunit RPC2 | | 2.57E−04 | 1.21 | 9.52E−03 | Lesion and no DM < Lesion and DM |
| BC015601 | Hs.73722 | APEX nuclease (multifunctional DNA repair | | 2.57E−04 | 1.21 | 3.40E−03 | Lesion and no DM < Lesion and DM |
| | | enzyme) 1 | | | | | |
| NM_003463 | Hs.227777 | protein tyrosine phosphatase type IVA, member 1 | PTP4A1 | 2.57E−04 | 1.19 | 2.45E−02 | Lesion and no DM < Lesion and DM |
| AJ318805 | Data not found | ESTs, Weakly similar to hypothetical protein | B2M | 1.47E−05 | 1.19 | 5.82E−03 | Lesion and no DM < Lesion and DM |
| | | FLJ20378 [Homo sapiens] [H. sapiens] | | | | | |
| NM_001614 | Hs.14376 | actin, gamma 1 | ACTG1 | 3.03E−06 | 1.19 | 4.20E−03 | Lesion and no DM < Lesion and DM |
| NM_005040 | Data not found | prolylacarboxypeptidase (angiotensinase C) | PRCP | 9.42E−04 | 1.18 | 4.58E−02 | Lesion and no DM < Lesion and DM |
| AA292025 | Hs.75545 | interleukin 4 receptor | | 9.42E−04 | 1.18 | 1.14E−02 | Lesion and no DM < Lesion and DM |
| W02761 | Hs.159 | tumor necrosis factor receptor superfamily, | | 9.42E−04 | 1.16 | 4.67E−02 | Lesion and no DM < Lesion and DM |
| | | member 1A | | | | | |
| NM_016019 | Hs.7194 | CGI-74 protein | LUC7L2 | 9.42E−04 | 1.14 | 1.20E−02 | Lesion and no DM < Lesion and DM |
| NM_005566 | Hs.2795 | lactate dehydrogenase A | LDHA | 9.42E−04 | 1.12 | 1.70E−01 | Lesion and no DM < Lesion and DM |
| AA464526 | Hs.82112 | interleukin 1 receptor, type I | | 9.42E−04 | 1.07 | 6.63E−01 | Lesion and no DM < Lesion and DM |
| AB033075 | Hs.10669 | development and differentiation enhancing | | 9.42E−04 | 1.06 | 3.66E−01 | Lesion and no DM < Lesion and DM |
| | | factor 1 | | | | | |
| NM_016406 | Hs.177507 | hypothetical protein HSPC155 | Ufc1 | 2.57E−04 | −1.06 | 3.73E−01 | Lesion and DM < Lesion and no DM |
| AA136125 | Hs.89718 | spermine synthase | | 2.57E−04 | −1.11 | 1.80E−01 | Lesion and DM < Lesion and no DM |
| BC007259 | Hs.286 | Homo sapiens, ribosomal protein L4, clone | | 2.57E−04 | −1.11 | 1.87E−02 | Lesion and DM < Lesion and no DM |
| | | MGC: 15542 IMAGE: 3050317, mRNA, complete cds | | | | | |
| NM_001769 | Hs.1244 | CD9 antigen (p24) | CD9 | 9.42E−04 | −1.11 | 2.07E−01 | Lesion and DM < Lesion and no DM |
| AK097914 | Hs.108124 | ribosomal protein S4, X-linked | | 9.42E−04 | −1.12 | 1.61E−03 | Lesion and DM < Lesion and no DM |
| AK094555 | Data not found | DEAD/H (Asp-Glu-Ala-Asp/His) box polypeptide 24 | | 3.03E−06 | −1.12 | 1.84E−01 | Lesion and DM < Lesion and no DM |
| NM_006937 | Hs.180139 | SMT3 suppressor of mif two 3 homolog 2 | SUMO2 | 9.42E−04 | −1.12 | 7.66E−03 | Lesion and DM < Lesion and no DM |
| | | (yeast) | | | | | |
| AL527635 | Hs.180789 | S164 protein | S164 | 2.57E−04 | −1.13 | 4.39E−02 | Lesion and DM < Lesion and no DM |
| NM_002192 | Data not found | inhibin, beta A (activin A, activin AB alpha | INHBA | 9.42E−04 | −1.13 | 1.66E−01 | Lesion and DM < Lesion and no DM |
| | | polypeptide | | | | | |
| NM_024408 | Hs.8121 | Notch homolog 2 (Drosophila) | NOTCH2 | 2.57E−04 | −1.13 | 9.99E−02 | Lesion and DM < Lesion and no DM |
| AI872254 | Hs.159955 | immunity associated protein 1 | | 6.43E−05 | −1.14 | 1.06E−02 | Lesion and DM < Lesion and no DM |
| AL832349 | Hs.279607 | calpastatin | CAST | 9.42E−04 | −1.14 | 4.59E−03 | Lesion and DM < Lesion and no DM |
| NM_004546 | Hs.198272 | NADH dehydrogenase (ubiquinone) 1 beta | NDUFB2 | 6.43E−05 | −1.15 | 1.78E−02 | Lesion and DM < Lesion and no DM |
| | | subcomplex, 2, 8 kDa | | | | | |
| NM_003756 | Data not found | eukaryotic translation initiation factor 3, subunit | EIF3S3 | 6.43E−05 | −1.15 | 1.66E−02 | Lesion and DM < Lesion and no DM |
| | | 3 gamma, 40 kDa | | | | | |
| NM_002793 | Data not found | proteasome (prosome, macropain) subunit, | PSMB1 | 2.57E−04 | −1.15 | 3.72E−04 | Lesion and DM < Lesion and no DM |
| | | beta type, 1 | | | | | |
| BG698758 | Hs.100293 | O-linked N-acetylglucosamine (GlcNAc) | | 2.57E−04 | −1.16 | 4.52E−02 | Lesion and DM < Lesion and no DM |
| | | transferase (UDP-N- | | | | | |
| | | acetylglucosamine:polypeptide-N- | | | | | |
| | | acetylglucosaminyl transferase) | | | | | |
| NM_057169 | Data not found | G protein-coupled receptor kinase-interactor 2 | GIT2 | 9.42E−04 | −1.16 | 9.93E−03 | Lesion and DM < Lesion and no DM |
| U38894 | Hs.252831 | Human protein tyrosine kinase t-Ror1 (Ror1) | | 2.57E−04 | −1.16 | 3.66E−03 | Lesion and DM < Lesion and no DM |
| | mRNA, complete cds | | | | | | |
| BM977503 | Hs.129872 | sperm associated antigen 9 | | 9.42E−04 | −1.16 | 2.16E−02 | Lesion and DM < Lesion and no DM |
| BF569545 | Hs.146428 | collagen, type V, alpha 1 | | 2.57E−04 | −1.16 | 7.65E−02 | Lesion and DM < Lesion and no DM |
| NM_000983 | Hs.326249 | ribosomal protein L22 | RPL22 | 6.43E−05 | −1.17 | 1.33E−05 | Lesion and DM < Lesion and no DM |
| NM_014372 | Hs.96334 | ring finger protein 11 | RNF11 | 9.42E−04 | −1.17 | 5.34E−02 | Lesion and DM < Lesion and no DM |
| NM_014624 | Hs.275243 | S100 calcium binding protein A6 (calcyclin) | S100A6 | 9.42E−04 | −1.17 | 1.57E−02 | Lesion and DM < Lesion and no DM |
| NM_004064 | Hs.238990 | cyclin-dependent kinase inhibitor 1B (p27, Kip1) | CDKN1B | 9.42E−04 | −1.18 | 1.54E−02 | Lesion and DM < Lesion and no DM |
| NM_004788 | Hs.75275 | ubiquitination factor E4A (UFD2 homolog, | UBE4A | 9.42E−04 | −1.18 | 1.13E−03 | Lesion and DM < Lesion and no DM |
| | | yeast) | | | | | |
| NM_004337 | Hs.40539 | chromosome 8 open reading frame 1 | C8orf1 | 9.42E−04 | −1.18 | 2.66E−01 | Lesion and DM < Lesion and no DM |
| NM_025238 | Hs.21332 | BTB (POZ) domain containing 1 | BTBD1 | 2.57E−04 | −1.18 | 1.20E−02 | Lesion and DM < Lesion and no DM |
| NM_003380 | Hs.297753 | vimentin | VIM | 6.43E−05 | −1.18 | 3.59E−02 | Lesion and DM < Lesion and no DM |
| AA425011 | Hs.180799 | C3HC4-type zinc finger protein | | 3.03E−06 | −1.18 | 1.25E−06 | Lesion and DM < Lesion and no DM |
| AK097395 | Hs.119 | superoxide dismutase 2, mitochondrial | | 2.57E−04 | −1.18 | 1.59E−02 | Lesion and DM < Lesion and no DM |
| NM_017830 | Hs.132071 | ovarian carcinoma immunoreactive antigen | OCIA | 5.65E−07 | −1.18 | 3.41E−03 | Lesion and DM < Lesion and no DM |
| NM_001664 | Hs.77273 | ras homolog gene family, member A | RHOA | 5.65E−07 | −1.18 | 2.62E−04 | Lesion and DM < Lesion and no DM |
| NM_003143 | Hs.923 | single-stranded DNA binding protein | SSBP1 | 6.43E−05 | −1.18 | 9.93E−06 | Lesion and DM < Lesion and no DM |
| NM_000611 | Hs.119663 | CD59 antigen p18-20 (antigen identified by | CD59 | 9.42E−04 | −1.18 | 2.54E−03 | Lesion and DM < Lesion and no DM |
| | | monoclonal antibodies 16.3A5, EJ16, EJ30, | | | | | |
| | | EL32 and G344) | | | | | |
| AB046844 | Hs.6639 | G protein-coupled receptor 107 | | 2.57E−04 | −1.18 | 7.79E−04 | Lesion and DM < Lesion and no DM |
| NM_006597 | Hs.180414 | heat shock 70 kDa protein 8 | HSPA8 | 9.42E−04 | −1.19 | 2.90E−02 | Lesion and DM < Lesion and no DM |
| NM_016055 | Hs.82389 | mitochondrial ribosomal protein L48 | | 9.42E−04 | −1.19 | 1.60E−05 | Lesion and DM < Lesion and no DM |
| NM_138799 | Hs.15641 | hypothetical protein BC016005 | OACT2 | 9.42E−04 | −1.19 | 1.90E−02 | Lesion and DM < Lesion and no DM |
| NM_001642 | Hs.279518 | amyloid beta (A4) precursor-like protein 2 | APLP2 | 6.43E−05 | −1.19 | 9.38E−04 | Lesion and DM < Lesion and no DM |
| NM_144778 | Hs.283609 | muscleblind-like protein MBLL39 | | 6.43E−05 | −1.19 | 5.70E−03 | Lesion and DM < Lesion and no DM |
| NM_016252 | Hs.250646 | baculoviral IAP repeat-containing 6 (apollon) | BIRC6 | 9.42E−04 | −1.20 | 9.74E−05 | Lesion and DM < Lesion and no DM |
| AK074898 | Hs.260622 | butyrate-induced transcript 1 | | 3.03E−06 | −1.20 | 2.50E−04 | Lesion and DM < Lesion and no DM |
| NM_003977 | Data not found | aryl hydrocarbon receptor interacting protein | AIP | 9.42E−04 | −1.20 | 6.67E−02 | Lesion and DM < Lesion and no DM |
| NM_001969 | Data not found | eukaryotic translation initiation factor 5 | EIF5 | 9.42E−04 | −1.20 | 3.01E−03 | Lesion and DM < Lesion and no DM |
| NM_006753 | Hs.274430 | surfeit 6 | SURF6 | 9.42E−04 | −1.21 | 3.32E−02 | Lesion and DM < Lesion and no DM |
| NM_001068 | Hs.75248 | topoisomerase (DNA) II beta 180 kDa | TOP2B | 2.57E−04 | −1.21 | 4.43E−04 | Lesion and DM < Lesion and no DM |
| NM_033546 | Hs.336916 | myosin regulatory light chain | MRLC2 | 9.42E−04 | −1.21 | 9.41E−04 | Lesion and DM < Lesion and no DM |
| NM_053023 | Data not found | zinc finger protein 91 homolog (mouse) | ZFP91 | 9.42E−04 | −1.21 | 1.86E−03 | Lesion and DM < Lesion and no DM |
| NM_006048 | Data not found | ubiquitination factor E4B (UFD2 homolog, | UBE4B | 6.43E−05 | −1.21 | 3.09E−05 | Lesion and DM < Lesion and no DM |
| | | yeast) | | | | | |
| NM_001028 | Hs.113029 | ribosomal protein S25 | RPS25 | 2.57E−04 | −1.21 | 6.43E−04 | Lesion and DM < Lesion and no DM |
| NM_032412 | Hs.323512 | putative nuclear protein ORF1-FL49 | ORF1-FL49 | 9.42E−04 | −1.21 | 1.67E−05 | Lesion and DM < Lesion and no DM |
| NM_005496 | Hs.50758 | SMC4 structural maintenance of chromosomes | SMC4L1 | 9.42E−04 | −1.22 | 1.07E−03 | Lesion and DM < Lesion and no DM |
| | | 4-like 1 (yeast) | | | | | |
| NM_006371 | Data not found | cartilage associated protein | CRTAP | 6.43E−05 | −1.22 | 1.42E−04 | Lesion and DM < Lesion and no DM |
| NM_004156 | Hs.80350 | protein phosphatase 2 (formerly 2A), catalytic | PPP2CB | 9.42E−04 | −1.22 | 2.00E−03 | Lesion and DM < Lesion and no DM |
| | | subunit, beta isoform | | | | | |
| NM_006888 | Hs.177656 | calmodulin 1 (phosphorylase kinase, delta) | CALM1 | 9.42E−08 | −1.22 | 1.84E−04 | Lesion and DM < Lesion and no DM |
| NM_002958 | Hs.79350 | RYK receptor-like tyrosine kinase | RYK | 9.42E−04 | −1.22 | 3.63E−05 | Lesion and DM < Lesion and no DM |
| NM_015270 | Hs.12373 | adenylate cyclase 6 | ADCY6 | 9.42E−04 | −1.22 | 2.68E−03 | Lesion and DM < Lesion and no DM |
| NM_139207 | Hs.179662 | nucleosome assembly protein 1-like 1 | NAP1L1 | 3.03E−06 | −1.23 | 3.05E−06 | Lesion and DM < Lesion and no DM |
| AA919115 | Hs.5807 | RAB14, member RAS oncogene family | | 3.03E−06 | −1.23 | 4.66E−07 | Lesion and DM < Lesion and no DM |
| L10717 | Data not found | IL2-inducible T-cell kinase | | 2.57E−04 | −1.23 | 9.85E−04 | Lesion and DM < Lesion and no DM |
| NM_016091 | Hs.119503 | eukaryotic translation inititation factor 3, subunit | EIF3S6IP | 2.57E−04 | −1.23 | 2.82E−04 | Lesion and DM < Lesion and no DM |
| | | 6 interacting protein | | | | | |
| AK054993 | Hs.173737 | ras-related C3 botulinum toxin substrate 1 (rho | | 3.03E−06 | −1.23 | 6.06E−06 | Lesion and DM < Lesion and no DM |
| | | family, small GTP binding protein Rac1) | | | | | |
| NM_003330 | Hs.13046 | thioredoxin reductase 1 | TXNRD1 | 9.42E−04 | −1.23 | 1.90E−02 | Lesion and DM < Lesion and no DM |
| NM_022151 | Hs.24719 | modulator of apoptosis 1 | MOAP1 | 2.57E−04 | −1.23 | 3.89E−04 | Lesion and DM < Lesion and no DM |
| NM_004578 | Hs.119007 | RAB4A, member RAS oncogene family | RAB4A | 6.43E−05 | −1.23 | 5.44E−04 | Lesion and DM < Lesion and no DM |
| NM_001568 | Hs.106673 | eukaryotic translation initiation factor 3, subunit | EIF3S6 | 6.43E−05 | −1.24 | 1.99E−04 | Lesion and DM < Lesion and no DM |
| | | 6 48 kDa | | | | | |
| NM_134442 | Hs.79194 | cAMP responsive element binding protein 1 | CREB1 | 9.42E−04 | −1.24 | 1.15E−02 | Lesion and DM < Lesion and no DM |
| NM_001001 | Hs.336628 | ribosomal protein L36a-like | RPL36AL | 2.57E−04 | −1.24 | 1.34E−06 | Lesion and DM < Lesion and no DM |
| NM_020191 | Hs.107127 | mitochondrial ribosomal protein S22 | MRPS22 | 9.42E−04 | −1.24 | 8.50E−05 | Lesion and DM < Lesion and no DM |
| NM_003128 | Hs.324648 | spectrin, beta, non-erythrocytic 1 | SPTBN1 | 6.43E−05 | −1.24 | 7.16E−03 | Lesion and DM < Lesion and no DM |
| NM_001967 | Hs.182429 | eukaryotic translation initiation factor 4A, | EIF4A2 | 2.57E−04 | −1.24 | 4.93E−03 | Lesion and DM < Lesion and no DM |
| | | isoform 2 | | | | | |
| D16920 | Hs.184592 | Human HepG2 3′region cDNA, clone hmd3e07 | | 6.43E−05 | −1.24 | 4.32E−05 | Lesion and DM < Lesion and no DM |
| AK055130 | Hs.182278 | calmodulin 2 (phosphorylase kinase, delta) | | 3.03E−06 | −1.24 | 2.64E−04 | Lesion and DM < Lesion and no DM |
| NM_001745 | Hs.13572 | calcium modulating ligand | CAMLG | 2.57E−04 | −1.25 | 3.33E−03 | Lesion and DM < Lesion and no DM |
| NM_018178 | Hs.29379 | hypothetical protein FLJ10687 | GPP34R | 2.57E−04 | −1.25 | 8.63E−02 | Lesion and DM < Lesion and no DM |
| NM_003472 | Hs.110713 | DEK oncogene (DNA binding) | DEK | 9.42E−04 | −1.25 | 1.01E−05 | Lesion and DM < Lesion and no DM |
| NM_023005 | Hs.194688 | bromodomain adjacent to zinc finger domain,1B | BAZ1B | 2.57E−04 | −1.25 | 1.37E−04 | Lesion and DM < Lesion and no DM |
| NM_003405 | Hs.75544 | tyrosine 3-monooxygenase/tryptophan 5- | YWHAH | 6.43E−05 | −1.25 | 1.87E−02 | Lesion and DM < Lesion and no DM |
| | | monooxygenase activation protein, eta | | | | | |
| | | polypeptide | | | | | |
| NM_005100 | Data not found | A kinase (PRKA) anchor protein (gravin) 12 | | 9.42E−04 | −1.25 | 2.02E−04 | Lesion and DM < Lesion and no DM |
| NM_012425 | Data not found | Ras suppressor protein 1 | RSU1 | 9.42E−04 | −1.25 | 3.30E−03 | Lesion and DM < Lesion and no DM |
| NM_005054 | Hs.334733 | RAN binding protein 2-like 1 | RANBP2L1 | 2.57E−04 | −1.26 | 8.11E−04 | Lesion and DM < Lesion and no DM |
| NM_003337 | Hs.811 | ubiquitin-conjugating enzyme E2B (RAD6 | UBE2B | 6.43E−05 | −1.26 | 4.93E−05 | Lesion and DM < Lesion and no DM |
| | | homolog) | | | | | |
| NM_004622 | Hs.75066 | translin | TSN | 9.42E−04 | −1.26 | 6.30E−03 | Lesion and DM < Lesion and no DM |
| AW021657 | Hs.70333 | WW domain-containing adapter with a coiled- | | 5.65E−07 | −1.26 | 4.10E−06 | Lesion and DM < Lesion and no DM |
| | | coil region | | | | | |
| NM_017446 | Hs.167130 | mitochondrial ribosomal protein L39 | | 9.42E−04 | −1.26 | 1.87E−02 | Lesion and DM < Lesion and no DM |
| BC033161 | Hs.75859 | mitochondrial ribosomal protein L49 | | 1.47E−05 | −1.26 | 2.63E−06 | Lesion and DM < Lesion and no DM |
| NM_006471 | Hs.233936 | myosin, light polypeptide, regulatory, non- | MRCL3 | 9.42E−04 | −1.27 | 6.72E−04 | Lesion and DM < Lesion and no DM |
| | | sarcomeric (20 kD) | | | | | |
| BC030594 | Hs.83532 | membrane cofactor protein (CD46, trophoblast- | | 2.57E−04 | −1.27 | 6.08E−08 | Lesion and DM < Lesion and no DM |
| | | lymphocyte cross-reactive antigen) | | | | | |
| NM_153207 | Hs.285833 | hypothetical protein MGC17922 | AEBP2 | 2.57E−04 | −1.27 | 2.91E−03 | Lesion and DM < Lesion and no DM |
| NM_001685 | Hs.73851 | ATP synthase, H + transporting, mitochondrial | ATP5J | 1.47E−05 | −1.27 | 2.93E−05 | Lesion and DM < Lesion and no DM |
| | | F0 complex, subunit F6 | | | | | |
| NM_004236 | Hs.30212 | thyroid receptor interacting protein 15 | TRIP 15 | 6.43E−05 | −1.27 | 8.24E−04 | Lesion and DM < Lesion and no DM |
| AB011182 | Hs.118087 | trans-activated by hepatitis C virus core protein 1 | | 1.47E−05 | −1.27 | 5.50E−06 | Lesion and DM < Lesion and no DM |
| NM_004130 | Hs.174071 | glycogenin | | 1.47E−05 | −1.27 | 1.57E−02 | Lesion and DM < Lesion and no DM |
| NM_001792 | Hs.161 | cadherin 2, type 1, N-cadherin (neuronal) | CDH2 | 9.42E−04 | −1.27 | 5.21E−03 | Lesion and DM < Lesion and no DM |
| BM988640 | Hs.6441 | tissue inhibitor of metalloproteinase 2 | | 1.47E−05 | −1.28 | 2.31E−04 | Lesion and DM < Lesion and no DM |
| AI004325 | Hs.181022 | CGI-07 protein | | 1.47E−05 | −1.28 | 6.64E−04 | Lesion and DM < Lesion and no DM |
| NM_016038 | Hs.110445 | CGI-97 protein | SBDS | 1.47E−05 | −1.28 | 5.15E−03 | Lesion and DM < Lesion and no DM |
| BM462724 | Hs.288971 | myeloid/lymphoid or mixed-lineage leukemia3 | | 2.57E−04 | −1.28 | 2.18E−05 | Lesion and DM < Lesion and no DM |
| NM_032205 | Hs.44159 | hypothetical protein FLJ21615 | CGI-72 | 2.57E−04 | −1.28 | 7.53E−04 | Lesion and DM < Lesion and no DM |
| NM_145693 | Data not found | lipin 1 | LPIN1 | 9.42E−04 | −1.28 | 4.49E−06 | Lesion and DM < Lesion and no DM |
| AW383166 | Hs.110950 | Rag C protein | | 3.03E−06 | −1.29 | 1.66E−06 | Lesion and DM < Lesion and no DM |
| AA974960 | Hs.12865 | likely ortholog of rat p47 | | 1.47E−05 | −1.29 | 2.01E−07 | Lesion and DM < Lesion and no DM |
| NM_007342 | Hs.168352 | nucleoporin-like protein 1 | NUPL2 | 1.47E−05 | −1.29 | 1.56E−07 | Lesion and DM < Lesion and no DM |
| U16850 | Hs.279009 | Homo sapiens calmodulin-1 (CALM1) mRNA, | | 6.43E−05 | −1.29 | 4.78E−03 | Lesion and DM < Lesion and no DM |
| | | 3′UTR, partial sequence | | | | | |
| NM_002567 | Hs.80423 | prostatic binding protein | PBP | 5.65E−07 | −1.29 | 2.42E−09 | Lesion and DM < Lesion and no DM |
| BC036469 | In multiple cluste | chromosome 20 open reading frame 99 | | 2.57E−04 | −1.29 | 4.77E−06 | Lesion and DM < Lesion and no DM |
| NM_003678 | Hs.75361 | chromosome 22 open reading frame 19 | C22orf19 | 6.43E−05 | −1.29 | 2.92E−03 | Lesion and DM < Lesion and no DM |
| AK093643 | Hs.82193 | esterase D/formylglutathione hydrolase | | 6.43E−05 | −1.29 | 4.67E−05 | Lesion and DM < Lesion and no DM |
| NM_020235 | Hs.35380 | bobby sox homolog (Drosophila) | BBX | 6.43E−05 | −1.29 | 1.15E−03 | Lesion and DM < Lesion and no DM |
| NM_005875 | Hs.21756 | translation factor sui1 homolog | GC20 | 9.42E−04 | −1.30 | 2.96E−04 | Lesion and DM < Lesion and no DM |
| BC005850 | Hs.31551 | Homo sapiens, core-binding factor, runt | | 9.42E−04 | −1.30 | 4.00E−04 | Lesion and DM < Lesion and no DM |
| | | domain, alpha subunit 2; translocated to, 1; | | | | | |
| | | cyclin D-related, clone MGC: 2796 | | | | | |
| | | IMAGE: 2961112, mRNA, complete cds | | | | | |
| NM_002633 | Data not found | phosphoglucomutase 1 | PGM1 | 9.42E−04 | −1.30 | 2.38E−04 | Lesion and DM < Lesion and no DM |
| NM_138271 | Hs.96264 | alpha thalassemia/mental retardation syndrome | ATRX | 1.47E−05 | −1.30 | 6.80E−07 | Lesion and DM < Lesion and no DM |
| | | X-linked (RAD54 homolog, S. cerevisiae) | | | | | |
| NM_000274 | Hs.75485 | ornithine aminotransferase (gyrate atrophy) | OAT | 9.42E−04 | −1.30 | 2.22E−05 | Lesion and DM < Lesion and no DM |
| NM_022730 | Hs.114432 | COP9 constitutive photomorphogenic homolog | COPS7B | 6.43E−05 | −1.30 | 1.61E−04 | Lesion and DM < Lesion and no DM |
| | | subunit 7B (Arabidopsis) | | | | | |
| NM_001483 | Hs.152707 | glioblastoma amplified sequence | GBAS | 9.42E−04 | −1.31 | 1.34E−05 | Lesion and DM < Lesion and no DM |
| NM_004939 | Hs.78580 | DEAD/H (Asp-Glu-Ala-Asp/His) box polypeptide 1 | DDX1 | 3.03E−06 | −1.31 | 2.94E−02 | Lesion and DM < Lesion and no DM |
| NM_006835 | Hs.79933 | cyclin I | CCNI | 5.65E−07 | −1.31 | 4.07E−07 | Lesion and DM < Lesion and no DM |
| AK074962 | Data not found | CGI-109 protein | | 2.57E−04 | −1.31 | 1.02E−04 | Lesion and DM < Lesion and no DM |
| NM_001378 | Hs.66881 | dynein, cytoplasmic, intermediate polypeptide 2 | DNCI2 | 3.03E−06 | −1.31 | 7.59E−10 | Lesion and DM < Lesion and no DM |
| AB014511 | Hs.70604 | ATPase, Class II, type 9A | ATP9A | 9.42E−04 | −1.31 | 1.02E−02 | Lesion and DM < Lesion and no DM |
| AA486556 | Hs.54457 | CD81 antigen (target of antiproliferative | | 5.65E−07 | −1.31 | 7.09E−04 | Lesion and DM < Lesion and no DM |
| | | antibody 1) | | | | | |
| AB058688 | Hs.47367 | fem-1 homolog c (C. elegans) | | 6.43E−05 | −1.31 | 7.44E−05 | Lesion and DM < Lesion and no DM |
| NM_004282 | Hs.55220 | BCL2-associated athanogene 2 | BAG2 | 1.47E−05 | −1.32 | 1.95E−02 | Lesion and DM < Lesion and no DM |
| BC025673 | Hs.171626 | S-phase kinase-associated protein 1A (p19A) | | 2.57E−04 | −1.32 | 4.25E−05 | Lesion and DM < Lesion and no DM |
| NM_002489 | Data not found | NADH dehydrogenase (ubiquinone) 1 alpha | NDUFA4 | 2.57E−04 | −1.32 | 4.75E−04 | Lesion and DM < Lesion and no DM |
| | | subcomplex, 4, 9 kDa | | | | | |
| NM_000127 | Hs.184161 | exostoses (multiple) 1 | EXT1 | 2.57E−04 | −1.32 | 1.23E−03 | Lesion and DM < Lesion and no DM |
| AF001893 | Data not found | multiple endocrine neoplasia I | | 9.42E−04 | −1.32 | 2.71E−03 | Lesion and DM < Lesion and no DM |
| NM_014633 | Hs.173288 | likely ortholog of mouse TPR-containing, SH2- | SH2BP1 | 9.42E−04 | −1.32 | 8.29E−03 | Lesion and DM < Lesion and no DM |
| | | binding phosphoprotein | | | | | |
| NM_018981 | Hs.1098 | DKFZp434J1813 protein | DNAJC10 | 6.43E−05 | −1.32 | 1.27E−03 | Lesion and DM < Lesion and no DM |
| BM783345 | Hs.24119 | NIMA (never in mitosis gene a)-related kinase 7 | | 9.42E−04 | −1.33 | 6.18E−05 | Lesion and DM < Lesion and no DM |
| NM_014043 | Hs.11449 | DKFZP564O123 protein | DKFZP564O123 | 9.42E−04 | −1.33 | 9.22E−05 | Lesion and DM < Lesion and no DM |
| NM_021943 | Hs.6120 | testis expressed sequence 27 | TEX27 | 9.42E−04 | −1.34 | 4.50E−04 | Lesion and DM < Lesion and no DM |
| NM_002211 | Data not found | integrin, beta 1 (fibronectin receptor, beta | | 9.42E−04 | −1.34 | 7.16E−05 | Lesion and DM < Lesion and no DM |
| | | polypeptide, antigen CD29 includes MDF2, | | | | | |
| | | MSK12) | | | | | |
| NM_020524 | Hs.8068 | hematopoietic PBX-interacting protein | PBXIP1 | 3.03E−06 | −1.34 | 4.21E−06 | Lesion and DM < Lesion and no DM |
| NM_031885 | Hs.332633 | Bardet-Biedl syndrome 2 | BBS2 | 2.57E−04 | −1.35 | 3.82E−03 | Lesion and DM < Lesion and no DM |
| NM_014000 | Hs.75350 | vinculin | VCL | 6.43E−05 | −1.35 | 1.59E−02 | Lesion and DM < Lesion and no DM |
| NM_032476 | Hs.6945 | mitochondrial ribosomal protein S6 | MRPS6 | 6.43E−05 | −1.35 | 2.53E−03 | Lesion and DM < Lesion and no DM |
| NM_005257 | Hs.50924 | GATA binding protein 6 | GATA6 | 2.57E−04 | −1.36 | 8.88E−04 | Lesion and DM < Lesion and no DM |
| AF278532 | Hs.102541 | netrin 4 | | 2.57E−04 | −1.37 | 2.42E−03 | Lesion and DM < Lesion and no DM |
| NM_033305 | Hs.53542 | chorea acanthocytosis | VPS13A | 2.57E−04 | −1.39 | 2.07E−03 | Lesion and DM < Lesion and no DM |
| NM_002884 | Hs.865 | RAP1A, member of RAS oncogene family | RAP1A | 3.03E−06 | −1.39 | 5.53E−07 | Lesion and DM < Lesion and no DM |
| NM_003715 | Hs.325948 | vesicle docking protein p115 | VDP | 6.43E−05 | −1.40 | 2.50E−08 | Lesion and DM < Lesion and no DM |
| NM_003932 | Hs.119222 | suppression of tumorigenicity 13 (colon | ST13 | 9.42E−08 | −1.40 | 6.26E−09 | Lesion and DM < Lesion and no DM |
| | | carcinoma) (Hsp70 interacting protein) | | | | | |
| NM_015904 | Hs.158688 | translation initiation factor IF2 | EIF5B | 9.42E−08 | −1.40 | 2.09E−06 | Lesion and DM < Lesion and no DM |
| NM_006135 | Hs.184270 | capping protein (actin filament) muscle Z-line, | CAPZA1 | 9.42E−04 | −1.40 | 7.20E−04 | Lesion and DM < Lesion and no DM |
| | | alpha 1 | | | | | |
| AK027166 | Hs.12929 | hypothetical protein FLJ20721 | HLC-8 | 1.47E−05 | −1.40 | 8.61E−05 | Lesion and DM < Lesion and no DM |
| NM_133494 | Data not found | NIMA (never in mitosis gene a)-related kinase 7 | NEK7 | 9.42E−04 | −1.41 | 8.35E−03 | Lesion and DM < Lesion and no DM |
| NM_015906 | Hs.287414 | tripartite motif-containing 33 | | 2.57E−04 | −1.41 | 1.44E−04 | Lesion and DM < Lesion and no DM |
| NM_001102 | Hs.119000 | actinin, alpha 1 | ACTN1 | 3.03E−06 | −1.42 | 6.54E−04 | Lesion and DM < Lesion and no DM |
| NM_002948 | Hs.74267 | ribosomal protein L15 | RPL15 | 3.03E−06 | −1.43 | 9.37E−08 | Lesion and DM < Lesion and no DM |
| BC038508 | Hs.25726 | transposon-derived Buster1 transposase-like | | 3.03E−06 | −1.43 | 5.53E−06 | Lesion and DM < Lesion and no DM |
| | | protein | | | | | |
| BG537456 | Data not found | osteonectin | SPARC | 6.43E−05 | −1.44 | 9.94E−05 | Lesion and DM < Lesion and no DM |
| NM_033138 | Hs.325474 | caldesmon 1 | | 6.43E−05 | −1.44 | 1.21E−02 | Lesion and DM < Lesion and no DM |
| NM_000919 | Data not found | peptidylglycine alpha-amidating | PAM | 6.43E−05 | −1.46 | 6.42E−08 | Lesion and DM < Lesion and no DM |
| | | monooxygenase | | | | | |
| NM_024071 | Data not found | hypothetical protein MGC2550 | ZFYVE21 | 2.57E−04 | −1.47 | 3.06E−04 | Lesion and DM < Lesion and no DM |
| BU075881 | Hs.11261 | small proline-rich protein 2A | HSMPP8 | 6.43E−05 | −1.47 | 1.38E−06 | Lesion and DM < Lesion and no DM |
| NM_005359 | Hs.75862 | MAD, mothers against decapentaplegic | SMAD4 | 1.47E−05 | −1.47 | 5.09E−06 | Lesion and DM < Lesion and no DM |
| | | homolog 4 (Drosophila) | | | | | |
| NM_014819 | Hs.279849 | KIAA0438 gene product | PJA2 | 6.43E−05 | −1.48 | 3.22E−06 | Lesion and DM < Lesion and no DM |
| BQ223934 | Hs.77837 | UDP-glucose pyrophosphorylase 2 | | 3.03E−06 | −1.49 | 2.21E−08 | Lesion and DM < Lesion and no DM |
| BM982785 | Hs.17820 | Rho-associated, coiled-coil containing protein | | 6.43E−05 | −1.49 | 4.40E−06 | Lesion and DM < Lesion and no DM |
| | | kinase 1 | | | | | |
| NM_004071 | Hs.2083 | CDC-like kinase 1 | CLK1 | 2.57E−04 | −1.51 | 5.33E−05 | Lesion and DM < Lesion and no DM |
| NM_000627 | Hs.241257 | latent transforming growth factor beta binding | | 1.47E−05 | −1.53 | 8.45E−05 | Lesion and DM < Lesion and no DM |
| | | protein 1 | | | | | |
| NM_000366 | Hs.77899 | tropomyosin 1 (alpha) | TPM1 | 6.43E−05 | −1.53 | 2.97E−03 | Lesion and DM < Lesion and no DM |
| BQ429410 | Hs.17820 | Rho-associated, coiled-coil containing protein | | 1.47E−05 | −1.55 | 2.50E−06 | Lesion and DM < Lesion and no DM |
| | | kinase 1 | | | | | |
| NM_016081 | Hs.194431 | palladin | KIAA0992 | 1.47E−05 | −1.62 | 2.07E−03 | Lesion and DM < Lesion and no DM |
| NM_014765 | Hs.75187 | translocase of outer mitochondrial membrane | TOMM20 | 1.39E−08 | −1.62 | 3.92E−07 | Lesion and DM < Lesion and no DM |
| | | 20 (yeast) homolog | | | | | |
| NM_005032 | Data not found | plastin 3 (T isoform) | PLS3 | 9.42E−08 | −1.63 | 9.47E−06 | Lesion and DM < Lesion and no DM |
| AA046932 | Hs.228598 | My015 | | 1.39E−08 | −1.68 | 2.56E−11 | Lesion and DM < Lesion and no DM |
| NM_021069 | Data not found | Arg/Abl-interacting protein ArgBP2 | ARGBP2 | 6.43E−05 | −1.73 | 4.82E−04 | Lesion and DM < Lesion and no DM |
| M69181 | Hs.296842 | myosin, heavy polypeptide 10, non-muscle | | 3.03E−06 | −1.87 | 3.34E−06 | Lesion and DM < Lesion and no DM |

Genes with UnKnown Name/Functions (Note: Lesion > No lesion, Foldchange positive; No lesion > lesion, negative).

| | | | | | | | |
|---|---|---|---|---|---|---|---|
| AI983239 | Hs.8881 | Hs.cDNA FLJ32163 fis, clone PLACE6000371 | | 6.43E−05 | 2.05 | 1.98E−03 | Lesion and no DM < Lesion and DM |
| R21535 | Hs.83733 | Hs.cDNA FLJ11724 fis, clone HEMBA1005331 | | 5.65E−07 | 1.89 | 3.55E−07 | Lesion and no DM < Lesion and DM |
| AW078807 | Hs.10029 | EST | | 1.47E−05 | 1.74 | 1.13E−04 | Lesion and no DM < Lesion and DM |
| T80495 | Hs. 124969 | Hs. clone 24707 mRNA sequence | | 9.42E−04 | 1.71 | 1.02E−03 | Lesion and no DM < Lesion and DM |
| AK092836 | Data not found | Homo sapiens cDNA FLJ35517 fis, clone | | 5.65E−07 | 1.61 | 8.88E−05 | Lesion and no DM < Lesion and DM |
| | | SPLEN2000698 | | | | | |
| BM690558 | Data not found | ESTs, Highly similar to interferon induced | | 3.03E−06 | 1.58 | 2.33E−05 | Lesion and no DM < Lesion and DM |
| | | transmembrane protein 3 (1-8U); interferon- | | | | | |
| | | inducible [Homo sapiens] [H. sapiens] | | | | | |
| AV706813 | Hs. 184011 | ESTs, Highly similar to IPYR_HUMAN Inorganic | | 3.03E−06 | 1.54 | 8.42E−06 | Lesion and no DM < Lesion and DM |
| | | pyrophosphatase (Pyrophosphate phospho- | | | | | |
| | | hydrolase) (PPase) [H. sapiens] | | | | | |
| AK094728 | Hs.284394 | Homo sapiens cDNA FLJ37409 fis, clone | | 6.43E−05 | 1.53 | 1.49E−01 | Lesion and no DM < Lesion and DM |
| | | BRAMY2028516, highly similar to | | | | | |
| | | COMPLEMENT C3 PRECURSOR | | | | | |
| AK025773 | Hs.5822 | Homo sapiens cDNA: FLJ22120 fis, clone | | 2.57E−04 | 1.52 | 1.11E−03 | Lesion and no DM < Lesion and DM |
| | | HEP18874 | | | | | |
| BC007583 | Hs.182426 | Homo sapiens, clone MGC: 15572 | | 9.42E−04 | 1.50 | 2.48E−04 | Lesion and no DM < Lesion and DM |
| | | IMAGE: 3140342, mRNA, complete cds | | | | | |
| AW088013 | Hs.118633 | EST | | 6.43E−05 | 1.47 | 3.00E−03 | Lesion and no DM < Lesion and DM |
| AI273932 | Data not found | EST | | 1.47E−05 | 1.46 | 2.95E−06 | Lesion and no DM < Lesion and DM |
| AL832838 | Hs.699 | hypothetical protein FLJ13952 | | 1.79E−09 | 1.46 | 4.14E−05 | Lesion and no DM < Lesion and DM |
| AI612803 | Hs.119122 | EST | | 6.43E−05 | 1.45 | 1.71E−03 | Lesion and no DM < Lesion and DM |
| AA486085 | Hs.76293 | EST | | 2.57E−04 | 1.42 | 8.41E−02 | Lesion and no DM < Lesion and DM |
| AI813947 | Hs.182426 | ESTs, Highly similar to ribosomal protein S2; | | 2.57E−04 | 1.41 | 5.17E−05 | Lesion and no DM < Lesion and DM |
| | | 40S ribosomal protein S2 [Homo sapiens] | | | | | |
| | | [H. sapiens] | | | | | |
| AF495759 | Hs.74170 | Homo sapiens unknown mRNA | | 6.43E−05 | 1.39 | 3.04E−03 | Lesion and no DM < Lesion and DM |
| AK026926 | Hs.182429 | Homo sapiens cDNA: FLJ23273 fis, clone | | 6.43E−05 | 1.38 | 6.41E−04 | Lesion and no DM < Lesion and DM |
| | | HEP02611, highly similar to HSU79278 Human | | | | | |
| | | protein disulfide isomerase-related protein P5 | | | | | |
| | | mRNA | | | | | |
| BU536672 | Data not found | Homo sapiens mRNA; cDNA DKFZp586O1224 | | 1.39E−08 | 1.38 | 1.24E−09 | Lesion and no DM < Lesion and DM |
| | | (from clone DKFZp586O1224) | | | | | |
| BG254478 | Data not found | ESTs, Highly similar to PC7084 GTP-binding | | 2.57E−04 | 1.36 | 5.26E−03 | Lesion and no DM < Lesion and DM |
| | | protein 2 - human (fragment) [H. sapiens] | | | | | |
| BM473144 | Hs.73742 | ESTs, Highly similar to RLAO_HUMAN 60S | | 1.47E−05 | 1.35 | 1.29E−03 | Lesion and no DM < Lesion and DM |
| | | acidic ribosomal protein p0 (L10E) [H. sapiens] | | | | | |
| BM801809 | Hs.119122 | ESTs, Highly similar to S29539 ribosomal | | 2.57E−04 | 1.34 | 1.70E−03 | Lesion and no DM < Lesion and DM |
| | | protein L13a, cytosolic - human [H. sapiens] | | | | | |
| AF116718 | Hs.177516 | hypothetical protein PRO2900 | | 2.57E−04 | 1.34 | 4.55E−05 | Lesion and no DM < Lesion and DM |
| BC011860 | Hs.119598 | Homo sapiens, clone MGC: 20593 | | 9.42E−04 | 1.34 | 3.59E−03 | Lesion and no DM < Lesion and DM |
| | | IMAGE: 4310440, mRNA, complete cds | | | | | |
| BC000673 | Hs.73742 | Homo sapiens, Similar to helicase-like protein | | 9.42E−08 | 1.31 | 2.93E−04 | Lesion and no DM < Lesion and DM |
| | | NHL, clone MGC: 665 IMAGE: 3347926, mRNA, | | | | | |
| | | complete cds | | | | | |
| NM_152452 | Hs.104679 | hypothetical protein MGC18216 | | 9.42E−04 | 1.30 | 5.34E−03 | Lesion and no DM < and DM |
| AK055474 | Hs.7949 | Homo sapiens cDNA: FLJ21721 fis, clone | | 2.57E−04 | 1.29 | 9.73E−04 | Lesion and no DM < Lesion and DM |
| | | COLF0381 | | | | | |
| AW304232 | Data not found | ESTs, Highly similar to RSP4_HUMAN 40S | | 2.57E−04 | 1.25 | 2.28E−04 | Lesion and no DM < Lesion and DM |
| | | ribosomal protein SA (P40) (34/67 kDa laminin | | | | | |
| | | receptor) (Colon carcinoma laminin-binding | | | | | |
| | | protein) (NEM/1CHD4) [H. sapiens] | | | | | |
| BC001805 | Hs.278242 | Homo sapiens, clone IMAGE: 3543670, mRNA, | | 2.57E−04 | 1.25 | 7.35E−03 | Lesion and no DM < Lesion and DM |
| | | partial cds | | | | | |
| AA192691 | Data not found | EST | | 9.42E−04 | 1.24 | 1.87E−02 | Lesion and no DM < Lesion and DM |
| AI359876 | Data not found | EST | | 2.57E−04 | 1.23 | 3.41E−02 | Lesion and no DM < Lesion and DM |
| BF683903 | Hs.76230 | ESTs, Highly similar to S55918 ribosomal | | 9.42E−04 | 1.23 | 9.78E−04 | Lesion and no DM < Lesion and DM |
| | | protein S10, cytosolic - human [H. sapiens] | | | | | |
| BM551542 | Data not found | ESTs, Moderately similar to trinucleotide repeat | | 1.47E−05 | 1.22 | 3.48E−02 | Lesion and no DM < Lesion and DM |
| | | containing 3; CAG repeat containing (glia- | | | | | |
| | | derived nexin I alpha); expanded repeat | | | | | |
| | | domain, CAG/CTG 3; CAG repeat domain | | | | | |
| | | [Homo sapiens] [H. sapiens] | | | | | |
| AI620703 | Data not found | ESTs, Moderately similar to 0512543A oxidase | | 9.42E−04 | 1.22 | 9.91E−03 | Lesion and no DM < Lesion and DM |
| | | II, cytochrome [Homo sapiens] [H. sapiens] | | | | | |
| BE873458 | Hs.55168 | ESTs, Weakly similar to neuronal thread protein | KIAA1337 | 9.42E−04 | 1.22 | 3.12E−03 | Lesion and no DM < Lesion and DM |
| | | [Homo sapiens] [H. sapiens] | | | | | |
| AY044167 | Hs.76064 | Homo sapiens clone IMAGE: BE741130 mRNA | | 9.42E−04 | 1.21 | 8.53E−03 | Lesion and no DM < Lesion and DM |
| | | sequence | | | | | |
| BM011169 | Data not found | ESTs, Highly similar to RL23_HUMAN 60S | | 9.42E−04 | 1.19 | 3.21E−02 | Lesion and no DM < Lesion and DM |
| | | ribosomal protein L23 (L17) [H. sapiens] | | | | | |
| AA158540 | Hs.72242 | EST | | 2.57E−04 | 1.17 | 5.89E−02 | Lesion and no DM < Lesion and DM |
| NM_014679 | Hs.151791 | KIAA0092 gene product | | 2.57E−04 | 1.17 | 3.55E−03 | Lesion and no DM < Lesion and DM |
| BC008758 | Data not found | ESTs, Highly similar to IDHG_HUMAN | | 9.42E−04 | −1.05 | 4.65E−01 | Lesion and DM < Lesion and no DM |
| | | Isocitrate dehydrogenase [NAD] subunit | | | | | |
| | | gamma, mitochondrial precursor (Isocitric | | | | | |
| | | dehydrogenase) (NAD +− specific ICDH) | | | | | |
| | | [H. sapiens] | | | | | |
| NM_052897 | Data not found | KIAA1887 protein | | 9.42E−04 | −1.11 | 8.27E−02 | Lesion and DM < Lesion and no DM |
| BQ016356 | Hs.293287 | Homo sapiens cDNA FLJ39255 fis, clone | | 9.42E−04 | −1.12 | 2.97E−01 | Lesion and DM < Lesion and no DM |
| | | OCBBF2008814 | | | | | |
| BM462590 | Hs.182278 | ESTs, Highly similar to D Chain D, Crystal | | 6.43E−05 | −1.12 | 2.03E−01 | Lesion and DM < Lesion and no DM |
| | | Structure Of The Edema Factor With | | | | | |
| | | Calmodulin And 3′-Datp [H. sapiens] | | | | | |
| AL833549 | Hs.279949 | KIAA1007 protein | | 9.42E−04 | −1.12 | 1.53E−02 | Lesion and DM < Lesion and no DM |
| NM_020462 | Hs.180428 | KIAA1181 protein | | 9.42E−04 | −1.14 | 1.56E−04 | Lesion and DM < Lesion and no DM |
| BM913262 | Hs.181165 | ESTs, Highly similar to EFHU1 translation | | 1.47E−05 | −1.14 | 2.93E−04 | Lesion and DM < Lesion and no DM |
| | | elongation factor eEF-1 alpha-1 chain - human | | | | | |
| | | [H. sapiens] | | | | | |
| AK098136 | Hs.6236 | Homo sapiens cDNA: FLJ21487 fis, clone | | 9.42E−04 | −1.14 | 2.57E−02 | Lesion and DM < Lesion and no DM |
| | | COLO5419 | | | | | |
| BC007568 | Hs.306117 | Homo sapiens, clone IMAGE: 3028427, mRNA, | | 9.42E−04 | −1.16 | 9.14E−02 | Lesion and DM < Lesion and no DM |
| | | partial cds | | | | | |
| NM_032039 | Data not found | hypothetical protein DKFZp761D0211 | DKFZP761D0211 | 9.42E−04 | −1.16 | 1.21E−02 | Lesion and DM < Lesion and no DM |
| BC007607 | Hs.155101 | Homo sapiens, clone MGC: 15690 | | 2.57E−04 | −1.16 | 6.15E−03 | Lesion and DM < Lesion and no DM |
| | | IMAGE: 3351222, mRNA, complete cds | | | | | |
| AL832015 | Hs.59838 | hypothetical protein FLJ10808 | | 9.42E−04 | −1.17 | 1.07E−03 | Lesion and DM < Lesion and no DM |
| AI675728 | Hs.277122 | EST | | 9.42E−04 | −1.19 | 1.79E−02 | Lesion and DM < Lesion and no DM |
| AL832747 | Hs.296261 | Homo sapiens mRNA; cDNA DKFZp686D0521 | | 2.57E−04 | −1.19 | 4.93E−04 | Lesion and DM < Lesion and no DM |
| | | (from clone DKFZp686D0521) | | | | | |
| NM_138357 | Hs.4896 | hypothetical protein BC010682 | C10orf42 | 9.42E−04 | −1.19 | 2.41E−02 | Lesion and DM < Lesion and no DM |
| AW575695 | Hs.157149 | KIAA1627 protein | | 6.43E−05 | −1.20 | 4.82E−05 | Lesion and DM < Lesion and no DM |
| BQ025173 | Hs.110950 | ESTs | | 6.43E−05 | −1.20 | 7.75E−06 | Lesion and DM < Lesion and no DM |
| AK025703 | Hs.173705 | Homo sapiens cDNA: FLJ22050 fis, clone | | 1.47E−05 | −1.20 | 4.07E−05 | Lesion and DM < Lesion and no DM |
| | | HEP09454 | | | | | |
| AW976721 | Hs.293327 | ESTs | | 9.42E−04 | −1.20 | 1.46E−02 | Lesion and DM < Lesion and no DM |
| BC015615 | Hs.104125 | Homo sapiens, Similar to peroxisomal | | 3.03E−06 | −1.21 | 5.64E−04 | Lesion and DM < Lesion and no DM |
| | | biogenesis factor 6, clone MGC: 23066 | | | | | |
| | | IMAGE: 4840674, mRNA, complete cds | | | | | |
| BC031936 | Hs.30174 | Homo sapiens, clone IMAGE: 4819348, mRNA, | | 2.57E−04 | −1.21 | 4.61E−03 | Lesion and DM < Lesion and no DM |
| | | partial cds | | | | | |
| AK000745 | Hs.243901 | Homo sapiens mRNA; cDNA DKFZp564C1563 | AK000745 | 9.42E−04 | −1.22 | 2.72E−04 | Lesion and DM < Lesion and no DM |
| | | (from clone DKFZp564C1563) | | | | | |
| AK026784 | Hs.301296 | Homo sapiens cDNA: FLJ23131 fis, clone | | 9.42E−04 | −1.22 | 2.42E−02 | Lesion and DM < Lesion and no DM |
| | | LNG08502 | | | | | |
| AA643327 | Hs.180946 | ESTs, Highly similar to 2113200A ribosomal | | 9.42E−08 | −1.22 | 1.40E−05 | Lesion and DM < Lesion and no DM |
| | | protein L5 [Homo sapiens] [H. sapiens] | | | | | |
| AK027539 | Hs.112318 | Homo sapiens cDNA FLJ14633 fis, clone | | 6.43E−05 | −1.22 | 1.99E−05 | Lesion and DM < Lesion and no DM |
| | | NT2RP2000938 | | | | | |
| BE276038 | Data not found | ESTs, Highly similar to A32915 nucleophosmin - | | 6.43E−05 | −1.23 | 5.49E−05 | Lesion and DM < Lesion and no DM |
| | | human [H. sapiens] | | | | | |
| AK074073 | Hs.323193 | hypothetical protein MGC3222 | | 9.42E−04 | −1.23 | 6.90E−05 | Lesion and DM < Lesion and no DM |
| AW264180 | Hs.6441 | EST | | 6.43E−05 | −1.23 | 2.73E−06 | Lesion and DM < Lesion and no DM |
| AB046824 | Hs.209464 | KIAA1604 protein | KIAA1604 | 9.42E−04 | −1.24 | 4.66E−04 | Lesion and DM < Lesion and no DM |
| AB014578 | Hs.12707 | KIAA0678 protein | | 9.42E−04 | −1.25 | 3.81E−04 | Lesion and DM < Lesion and no DM |
| BM806103 | Data not found | hypothetical protein FLJ14600 | | 9.42E−04 | −1.25 | 5.67E−04 | Lesion and DM < Lesion and no DM |
| NM_014659 | Hs.156814 | KIAA0377 gene product | KIAA0377 | 9.42E−04 | −1.26 | 1.59E−02 | Lesion and DM < Lesion and no DM |
| BC011987 | Data not found | Homo sapiens, clone IMAGE: 3857153, mRNA | | 2.57E−04 | −1.27 | 4.34E−02 | Lesion and DM < Lesion and no DM |
| NM_014967 | Hs.5400 | KIAA1018 protein | | 9.42E−04 | −1.27 | 1.52E−03 | Lesion and DM < Lesion and no DM |
| AK093924 | Hs.297753 | Homo sapiens cDNA FLJ36605 fis, clone | | 1.47E−05 | −1.28 | 4.69E−03 | Lesion and DM < Lesion and no DM |
| | | TRACH2015316, highly similar to VIMENTIN | | | | | |
| AK022030 | In multiple cluste | Homo sapiens cDNA FLJ11968 fis, clone | | 1.47E−05 | −1.28 | 8.43E−06 | Lesion and DM < Lesion and no DM |
| | | HEMBB1001133 | | | | | |
| AV719568 | Data not found | EST | | 2.57E−04 | −1.28 | 2.01E−04 | Lesion and DM < Lesion and no DM |
| AB011142 | Hs.180948 | KIAA0570 gene product | | 1.47E−05 | −1.29 | 1.97E−07 | Lesion and DM < Lesion and no DM |
| AL553394 | Hs.323164 | hypothetical protein MGC2217 | | 6.43E−05 | −1.30 | 2.57E−04 | Lesion and DM < Lesion and no DM |
| BC021287 | Hs.184544 | Homo sapiens, clone IMAGE: 3355383, mRNA, | | 1.39E−08 | −1.30 | 1.45E−06 | Lesion and DM < Lesion and no DM |
| | | partial cds | | | | | |
| AF034176 | Data not found | Homo sapiens clone 23872 mRNA sequence | | 2.57E−04 | −1.30 | 1.18E−04 | Lesion and DM < Lesion and no DM |
| AK091343 | Hs.106330 | Homo sapiens clone IMAGE: 49795, mRNA | | 2.57E−04 | −1.31 | 1.02E−04 | Lesion and DM < Lesion and no DM |
| | | sequence | | | | | |
| BC015869 | Hs.8136 | Homo sapiens clone 23698 mRNA sequence | | 9.42E−04 | −1.31 | 6.04E−04 | Lesion and DM < Lesion and no DM |
| NM_014969 | Hs.3830 | KIAA0893 protein | KIAA0893 | 2.57E−04 | −1.31 | 1.56E−07 | Lesion and DM < Lesion and no DM |
| BC037313 | Hs.137260 | hypothetical protein FLJ23151 | | 2.57E−04 | −1.32 | 3.79E−03 | Lesion and DM < Lesion and no DM |
| AK091994 | Data not found | Homo sapiens cDNA FLJ34675 fis, clone | | 9.42E−04 | −1.32 | 2.22E−04 | Lesion and DM < Lesion and no DM |
| | | LIVER2001608 | | | | | |
| AL080234 | Hs.8078 | Homo sapiens clone FBD3 Cri-du-chat critical | | 3.03E−06 | −1.32 | 8.62E−10 | Lesion and DM < Lesion and no DM |
| | | region mRNA | | | | | |
| AK055112 | Hs.82503 | Homo sapiens cDNA FLJ30550 fis, clone | | 2.57E−04 | −1.33 | 1.18E−02 | Lesion and DM < Lesion and no DM |
| | | BRAWH2001502 | | | | | |
| AB007916 | Hs.214646 | KIAA0447 gene product | KIAA0447 | 1.79E−09 | −1.33 | 1.23E−08 | Lesion and DM < Lesion and no DM |
| N67474 | Hs.43157 | ESTs | | 6.43E−05 | −1.34 | 3.61E−07 | Lesion and DM < Lesion and no DM |
| AK092475 | Hs.294110 | Homo sapiens cDNA FLJ35156 fis, clone | | 6.43E−05 | −1.34 | 2.61E−05 | Lesion and DM < Lesion and no DM |
| | | PLACE6011057 | | | | | |
| W69378 | Hs.62669 | Hs. mRNA; cDNA DKFZp586D0923 (from clone | | 2.57E−04 | −1.34 | 1.08E−05 | Lesion and DM < Lesion and no DM |
| | | DKFZp586D0923) | | | | | |
| NM_018507 | Hs.93379 | hypothetical protein PRO1843 | | 3.03E−06 | −1.35 | 3.27E−06 | Lesion and DM < Lesion and no DM |
| AK055662 | Hs.5699 | Homo sapiens cDNA FLJ31100 fis, clone | | 1.47E−05 | −1.36 | 1.69E−05 | Lesion and DM < Lesion and no DM |
| | | IMR321000242, weakly similar to ZINC | | | | | |
| | | FINGER PROTEIN 33A | | | | | |
| AF267856 | Hs.8084 | hypothetical protein dJ465N24.2.1 | | 9.42E−04 | −1.37 | 1.01E−04 | Lesion and DM < Lesion and no DM |
| BM543221 | Hs.343472 | ESTs | | 9.42E−04 | −1.39 | 1.81E−05 | Lesion and DM < Lesion and no DM |
| AK027166 | Hs.12929 | hypothetical protein FLJ20721 | FLJ20721 | 1.47E−05 | −1.40 | 8.61E−05 | Lesion and DM < Lesion and no DM |
| AK096403 | Hs.111334 | Homo sapiens cDNA FLJ39084 fis, clone | | 3.03E−06 | −1.41 | 2.97E−05 | Lesion and DM < Lesion and no DM |
| | | NT2RP7018871 | | | | | |
| NM_152535 | Data not found | hypothetical protein FLJ31131 | | 1.47E−05 | −1.41 | 1.90E−07 | Lesion and DM < Lesion and no DM |
| BQ879275 | Hs.182183 | Homo sapiens, clone IMAGE: 4296901, mRNA | | 9.42E−04 | −1.43 | 1.41E−02 | Lesion and DM < Lesion and no DM |
| BG028195 | Hs.302746 | Homo sapiens cDNA FLJ38755 fis, clone | | 3.03E−06 | −1.43 | 5.68E−05 | Lesion and DM < Lesion and no DM |
| | | KIDNE2012775, weakly similar to Homo | | | | | |
| | | sapiens mRNA for transport-secretion protein | | | | | |
| | | 2.1 | | | | | |
| U23841 | Hs.343465 | ESTs | | 3.03E−06 | −1.44 | 2.04E−08 | Lesion and DM < Lesion and no DM |
| AK075484 | Data not found | Homo sapiens cDNA PSEC0178 fis, clone | | 6.43E−05 | −1.45 | 1.94E−04 | Lesion and DM < Lesion and no DM |
| | | OVARC1000636 | | | | | |
| AL833137 | Hs.290259 | Homo sapiens, clone IMAGE: 3915000, mRNA | | 9.42E−08 | −1.47 | 9.74E−08 | Lesion and DM < Lesion and no DM |
| NM_014851 | Hs.7764 | KIAA0469 gene product | | 6.43E−05 | −1.47 | 1.91E−06 | Lesion and DM < Lesion and no DM |
| AK096260 | Hs.12921 | hypothetical protein FLJ14399 | FLJ14399 | 3.03E−06 | −1.48 | 6.64E−05 | Lesion and DM < Lesion and no DM |
| AL833007 | Hs.121520 | Homo sapiens, clone IMAGE: 3625286, mRNA, | | 1.47E−05 | −1.49 | 3.64E−04 | Lesion and DM < Lesion and no DM |
| | | partial cds | | | | | |
| AK055197 | Hs.77899 | Homo sapiens cDNA FLJ30635 fis, clone | | 1.47E−05 | −1.50 | 6.70E−03 | Lesion and DM < Lesion and no DM |
| | | CTONG2002520 | | | | | |
| AW104810 | Hs.244257 | ESTs | | 1.47E−05 | −1.50 | 7.82E−06 | Lesion and DM < Lesion and no DM |
| AK096204 | Hs.227571 | Homo sapiens cDNA FLJ38885 fis, clone | | 1.47E−05 | −1.56 | 2.57E−03 | Lesion and DM < Lesion and no DM |
| | | MESAN2017417, moderately similar to | | | | | |
| | | REGULATOR OF G-PROTEIN SIGNALING 4 | | | | | |
| AF231512 | Hs.10283 | Homo sapiens RNA binding motif protein 8B | | 3.03E−06 | −1.58 | 3.54E−06 | Lesion and DM < Lesion and no DM |
| | | (RBM8B) mRNA, complete cds | | | | | |
| AB040951 | Hs.17311 | KIAA1518 protein | | 2.57E−04 | −1.63 | 7.06E−05 | Lesion and DM < Lesion and no DM |
| BI430544 | Hs.216381 | ESTs | | 9.42E−04 | −1.64 | 4.80E−04 | Lesion and DM < Lesion and no DM |
| BC009220 | Hs.292457 | Homo sapiens, clone MGC: 16362 | | 3.03E−06 | −1.65 | 5.26E−06 | Lesion and DM < Lesion and no DM |
| | | IMAGE: 3927795, mRNA, complete cds | | | | | |

TABLE 4

| | | |
|---|---|---|
| | Gene | |
| Gene Name | Accession | Expected |

| | | | | |
|---|---|---|---|---|
| Classification info | Gene Info | Score | Score | FDR |

These genes are up-regulated in diabetic and down-regulated in non-diabetic samples. Analysis done on samples w/o lesions.

| | | | | | |
|---|---|---|---|---|---|
| NM_000584 | 7F.8.G8 | interleukin 8 | 2.1501 | 1.5569 | 2.8621 |
| N98591 | 14N.7.C4 | interleukin 6 (interferon, beta 2) | 2.1291 | 1.4441 | 2.8621 |
| AA936768 | 14N.7.D1 | interleukin 1, alpha | 2.008 | 1.3737 | 2.8621 |
| BM803108 | 7F.4.E7 | ESTs | 1.9733 | 1.3366 | 2.8621 |
| NM_000600 | 7F.2.F2 | interleukin 6 (interferon, beta 2) | 1.9366 | 1.3001 | 2.8621 |
| NM_000600 | 9R.10.G7 | interleukin 6 (interferon, beta 2) | 1.9077 | 1.2747 | 2.8621 |
| NM_000600 | 7F.7.B6 | interleukin 6 (interferon, beta 2) | 1.8876 | 1.2523 | 2.8621 |
| N98591 | 14N.5.C4 | interleukin 6 (interferon, beta 2) | 1.8809 | 1.2341 | 2.8621 |
| AI359876 | 12F.2.C11 | EST | 1.8679 | 1.2165 | 2.8621 |
| AA156031 | 14N.4.G12 | metallothionein 2A | 1.8589 | 1.2003 | 2.8621 |
| NHF | 9R.3.A11 | | 1.8443 | 1.1861 | 2.8621 |
| NHF | 7F.10.G3 | | 1.8421 | 1.1743 | 2.8621 |
| BF131637 | 7F.5.E2 | metallothionein 2A | 1.8311 | 1.1604 | 2.8621 |
| NM_003670 | 9F.7.C8 | basic helix-loop-helix domain containing, class B, 2 | 1.8277 | 1.1484 | 2.8621 |
| NM_000600 | 7F.9.D11 | interleukin 6 (interferon, beta 2) | 1.7953 | 1.1372 | 3.05 |
| NM_001235 | 7R.9.A7 | serine (or cysteine) proteinase inhibitor, clade H (heat shock protein 47), | 1.7634 | 1.1278 | 3.5781 |
| | | member 2 | | | |
| NHF | 7F.8.A6 | | 1.7405 | 1.118 | 3.8571 |
| NM_004530 | 8R.1.B12 | matrix metalloproteinase 2 (gelatinase A, 72 kDa gelatinase, 72 kDa type IV | 1.7321 | 1.1097 | 3.8571 |
| | | collagenase) | | | |
| NM_001235 | 7F.4.D7 | serine (or cysteine) proteinase inhibitor, clade H (heat shock protein 47), | 1.7154 | 1.1017 | 3.9861 |
| | | member 2 | | | |
| NM_002982 | 7F.4.H8 | chemokine (C—C motif) ligand 2 | 1.6959 | 1.0934 | 4.0625 |
| NM_001235 | 7R.10.F12 | serine (or cysteine) proteinase inhibitor, clade H (heat shock protein 47), | 1.684 | 1.0849 | 4.0625 |
| | | member 2 | | | |
| NM_004530 | 8F.3.H4 | matrix metalloproteinase 2 (gelatinase A, 72 kDa gelatinase, 72 kDa type IV | 1.6822 | 1.0778 | 4.0625 |
| | | collagenase) | | | |
| NM_002631 | 1F.1.D7 | phosphogluconate dehydrogenase | 1.6687 | 1.0709 | 4.125 |
| NM_078467 | 9F.8.F8 | cyclin-dependent kinase inhibitor 1A (p21, Cip1) | 1.6614 | 1.0638 | 4.125 |
| NM_001235 | 7R.2.D3 | serine (or cysteine) proteinase inhibitor, clade H (heat shock protein 47), | 1.6556 | 1.0576 | 4.125 |
| | | member 2 | | | |
| AA936768 | 14N.7.C12 | interleukin 1, alpha | 1.6348 | 1.0512 | 4.3913 |
| NM_152862 | 7F.6.F1 | actin related protein 2/3 complex, subunit 2, 34 kDa | 1.6157 | 1.0457 | 4.5 |
| NM_002923 | 7R.9.F12 | regulator of G-protein signalling 2, 24 kDa | 1.6129 | 1.0401 | 4.5 |
| AI983239 | 14N.4.A9 | Hs. cDNA FLJ32163 fis, clone PLACE6000371 | 1.6013 | 1.0343 | 4.5385 |
| NM_001235 | 7R.1.H3 | serine (or cysteine) proteinase inhibitor, clade H (heat shock protein 47), | 1.5979 | 1.0289 | 4.5385 |
| | | member 2 | | | |
| NM_005415 | 9F.2.H7 | solute carrier family 20 (phosphate transporter), member 1 | 1.5961 | 1.0237 | 4.5385 |
| AA936768 | 14N.5.D1 | interleukin 1, alpha | 1.5911 | 1.0184 | 4.5385 |
| AW772163 | 14N.1.B11 | hypothetical protein FLJ20401 | 1.5695 | 1.0129 | 4.5536 |
| NM_001235 | 8R.2.D12 | serine (or cysteine) proteinase inhibitor, clade H (heat shock protein 47), | 1.5676 | 1.0087 | 4.5536 |
| | | member 2 | | | |
| R21535 | 14N.2.A11 | Hs. cDNA FLJ11724 fis, clone HEMBA1005331 | 1.5674 | 1.004 | 4.5536 |
| NHF | 12R.1.F8 | | 1.565 | 0.999 | 4.5536 |

Genes below are down-regulated in diabetic and up-regulated in non-diabetic samples. Analysis done on samples w/o lesions.

| | | | | | |
|---|---|---|---|---|---|
| NHF | 7F.7.H2 | | −1.6926 | −1.1314 | 4.9237 |
| NM_144573 | 8R.8.G7 | likely ortholog of rat F-actin binding protein nexilin | −1.694 | −1.1384 | 4.9237 |
| BQ429410 | 9R.2.A7 | Rho-associated, coiled-coil containing protein kinase 1 | −1.6977 | −1.1459 | 4.9237 |
| BC009220 | 9R.4.G4 | Homo sapiens, clone MGC: 16362 IMAGE: 3927795, mRNA, complete cds | −1.7433 | −1.1537 | 4.2111 |
| NM_013943 | 1R.2.E9 | chloride intracellular channel 4 | −1.7631 | −1.1626 | 4.125 |
| AF156100 | 8R.7.B10 | fibulin 6 | −1.7833 | −1.1717 | 4.0405 |
| NHF | 7F.7.E4 | | −1.805 | −1.1809 | 3.7576 |
| AL833007 | 9R.9.F8 | Homo sapiens, clone IMAGE: 3625286, mRNA, partial cds | −1.8444 | −1.1891 | 3.3387 |
| NHF | 9R.6.C7 | | −1.8884 | −1.1988 | 2.4 |
| NM_005863 | 8R.4.B9 | neuroepithelial cell transforming gene 1 | −1.8915 | −1.2102 | 2.4 |
| NHF | 9R.3.E3 | | −1.9077 | −1.224 | 2.4 |
| NHF | 9R.5.F6 | | −1.9291 | −1.2365 | 2.4 |
| AL833007 | 7R.2.E4 | Homo sapiens, clone IMAGE: 3625286, mRNA, partial cds | −1.9363 | −1.2512 | 2.4 |
| AL833007 | 9R.2.C1 | Homo sapiens, clone IMAGE: 3625286, mRNA, partial cds | −1.9564 | −1.2656 | 2.4 |
| NM_014890 | 12F.2.H3 | downregulated in ovarian cancer 1 | −2.001 | −1.2832 | 2.1111 |
| AA629603 | 14N.4.E6 | PTPL1-associated RhoGAP 1 | −2.0461 | −1.3011 | 1.6875 |
| M14219 | 9R.9.C4 | Human chondroitin/dermatan sulfate proteoglycan (PG40) core protein | −2.141 | −1.3242 | 1 |
| | | mRNA, complete cds | | | |
| AL833007 | 8F.9.D1 | Homo sapiens, clone IMAGE: 3625286, mRNA, partial cds | −2.1458 | −1.3504 | 1 |
| AL833007 | 9R.9.B6 | Homo sapiens, clone IMAGE: 3625286, mRNA, partial cds | −2.1808 | −1.3778 | 1 |
| NHF | 9R.8.H12 | | −2.215 | −1.4119 | 1 |
| AL832780 | 7F.7.D4 | Homo sapiens mRNA; cDNA DKFZp686J037 (from clone DKFZp686J037) | −2.2441 | −1.4563 | 1 |
| NM_003601 | 7F.7.H8 | SWI/SNF related, matrix associated, actin dependent regulator of chromatin, | −2.2863 | −1.5301 | 1 |
| | | subfamily a, member 5 | | | |
| N73625 | 14N.2.B2 | EST | −2.4132 | −1.6857 | 1 |

TABLE 8

| | | | | | | | | |
|---|---|---|---|---|---|---|---|---|
| | | | | TNoM | Ratio fold | t-test score | Change | |
| SystematicName | UnigeneCode | GeneName | GeneSymbol | p-value | change | p-value | Directions | Comments |

Genes with Known Name/or Functions (Note: Statin > No statin, Foldchange pos.; No statin > statin, Foldchange neg.).

| | | | | | | | | |
|---|---|---|---|---|---|---|---|---|
| R09728 | Hs.26530 | serum deprivation response (phosphatidylserine binding protein) | | 5.83E−06 | 1.49 | 1.08E−05 | No statin < Statin | |
| NM_004772 | Hs.142827 | P311 protein | C5orf13 | 1.75E−06 | 1.40 | 1.16E−08 | No statin < Statin | |
| NM_006925 | Hs.166975 | splicing factor, arginine/serine-rich 5 | SFRS5 | 5.83E−06 | 1.40 | 8.04E−06 | No statin < Statin | |
| NM_006925 | Hs.166975 | splicing factor, arginine/serine-rich 5 | SFRS5 | 5.83E−06 | 1.40 | 8.04E−06 | No statin < Statin | |
| AF001893 | Hs.240443 | multiple endocrine neoplasia I | | 5.53E−05 | 1.39 | 2.58E−04 | No statin < Statin | |
| AI02885 | Hs.2351 | protein C (inactivator of coagulation factors Va and VIIIa) | | 1.84E−05 | 1.36 | 1.47E−03 | No statin < Statin | |
| AA115076 | Hs.82071 | Cbp/p300-interacting transactivator, with Glu/Asp-rich carboxy-terminal | | 1.75E−06 | 1.33 | 6.23E−04 | No statin < Statin | |
| | | domain, 2 | | | | | | |
| AA039932 | Hs.89887 | thromboxane A2 receptor | | 1.58E−04 | 1.31 | 1.56E−03 | No statin < Statin | |
| NM_025197 | Hs.20157 | CDK5 regulatory subunit associated protein 3 | CDK5RAP3 | 1.58E−04 | 1.28 | 5.53E−04 | No statin < Statin | |
| R36467 | Hs.1103 | transforming growth factor, beta 1 | | 1.58E−04 | 1.28 | 5.05E−03 | No statin < Statin | Known |
| NM_000930 | Hs.274404 | plasminogen activator, tissue | PLAT | 5.53E−05 | 1.27 | 2.93E−03 | No statin < Statin | |
| N53447 | Hs.17109 | integral membrane protein 2A | | 4.28E−04 | 1.26 | 1.96E−01 | No statin < Statin | |
| W72329 | Hs.36 | lymphotoxin alpha (TNF superfamily, member 1) | | 1.58E−04 | 1.25 | 1.06E−02 | No statin < Statin | |
| NM_002414 | Hs.177543 | antigen identified by monoclonal antibodies 12E7, F21 and O13 | CD99 | 1.84E−05 | 1.24 | 1.39E−04 | No statin < Statin | |
| NM_032870 | Hs.18368 | SR rich protein | C6orf111 | 1.11E−03 | 1.23 | 3.63E−03 | No statin < Statin | |
| NM_006985 | Hs.251928 | nuclear pore complex interacting protein | NPIP | 1.58E−04 | 1.22 | 2.26E−03 | No statin < Statin | |
| NM_006283 | Hs.173159 | transforming, acidic coiled-coil containing protein 1 | TACC1 | 1.58E−04 | 1.21 | 1.39E−04 | No statin < Statin | |
| NM_021109 | Hs.75968 | thymosin, beta 4, X chromosome | | 1.11E−03 | 1.20 | 2.53E−04 | No statin < Statin | |
| NM_001642 | Hs.279518 | amyloid beta (A4) precursor-like protein 2 | APLP2 | 1.11E−03 | 1.20 | 1.73E−04 | No statin < Statin | |
| AI341605 | Hs.133207 | PTPRF interacting protein, binding protein 1 (liprin beta 1) | | 4.28E−04 | 1.17 | 6.93E−03 | No statin < Statin | |
| NM_005015 | Hs.151134 | oxidase (cytochrome c) assembly 1-like | OXA1L | 1.84E−05 | 1.14 | 5.77E−03 | No statin < Statin | |
| NM_016237 | Hs.7101 | anaphase promoting complex subunit 5 | ANAPC5 | 4.28E−04 | 1.13 | 3.50E−04 | No statin < Statin | |
| H66617 | Hs.78979 | Golgi apparatus protein 1 | | 1.11E−03 | 1.11 | 2.63E−02 | No statin < Statin | |
| NM_005324 | Hs.180877 | H3 histone, family 3B (H3.3B) | H3F3B | 4.28E−04 | −1.13 | 3.52E−03 | Statin < No statin | |
| NM_004508 | Hs.76038 | isopentenyl-diphosphate delta isomerase | IDI1 | 1.11E−03 | −1.16 | 3.03E−03 | Statin < No statin | |
| NM_000611 | Hs.119663 | CD59 antigen p18-20 (antigen identified by monoclonal antibodies 16.3A5, | CD59 | 4.28E−04 | −1.27 | 2.52E−06 | Statin < No statin | |
| | | EJ16, EJ30, EL32 and G344) | | | | | | |
| NM_005347 | Hs.75410 | heat shock 70 kDa protein 5 (glucose-regulated protein, 78 kDa) | HSPAS | 5.53E−05 | −1.40 | 2.44E−04 | Statin < No statin | Known |
| NM_005415 | Hs.78452 | solute carrier family 20 (phosphate transporter), member 1 | SLC20A1 | 1.58E−04 | −1.45 | 3.06E−06 | Statin < No statin | |
| NM_001679 | Hs.76941 | ATPase, Na+/K+ transporting, beta 3 polypeptide | ATP1B3 | 1.84E−05 | −1.47 | 1.34E−06 | Statin < No statin | |
| NM_006216 | Data not found | serine (or cysteine) proteinase inhibitor, clade E (nexin, plasminogen | SERPINE2 | 1.11E−03 | −1.53 | 5.58E−04 | Statin < No statin | |
| | | activator inhibitor type 1), member 2 | | | | | | |
| AW780123 | Hs.299465 | ribosomal protein S26 | | 1.11E−03 | −1.53 | 1.49E−05 | Statin < No statin | |
| NM_002658 | Hs.77274 | plasminogen activator, urokinase | PLAU | 5.53E−05 | −1.54 | 3.80E−07 | Statin < No statin | |
| AW007736 | Hs.23703 | UDP-glucose ceramide glucosyltransferase | | 5.53E−05 | −1.56 | 6.58E−07 | Statin < No statin | |
| NM_003670 | Hs.171825 | basic helix-loop-helix domain containing, class B, 2 | | 5.83E−06 | −1.58 | 7.42E−05 | Statin < No statin | |
| BF131637 | Hs.118786 | metallothionein 2A | | 1.11E−03 | −1.60 | 2.27E−05 | Statin < statin | |
| AA936768 | Hs.1722 | interleukin 1, alpha | | 1.11E−03 | −1.68 | 1.55E−04 | Statin < No statin | Known |
| NM_005110 | Data not found | glutamine-fructose-6-phosphate transaminase 2 | GFPT2 | 5.53E−05 | −1.93 | 4.67E−09 | Statin < No statin | |
| NM_005746 | Hs.239138 | pre-B-cell colony-enhancing factor | PBEF1 | 4.97E−07 | −1.95 | 6.50E−10 | Statin < No statin | |
| NM_002852 | Hs.2050 | pentaxin-related gene, rapidly induced by IL-1 beta | PTX3 | 5.53E−05 | −1.96 | 2.36E−07 | Statin < No statin | |
| N92901 | Hs.83213 | fatty acid binding protein 4, adipocyte | | 4.28E−04 | −2.34 | 1.27E−03 | Statin < No statin | |
| NM_000600 | Hs.93913 | interleukin 6 (interferon, beta 2) | IL6 | 1.75E−06 | −3.27 | 4.86E−10 | Statin < No statin | Known |

Genes with UnKnown Name/Functions (Note: Statin > No statin, Foldchange pos.; No statin > statin, Foldchange neg.).

| | | | | | | | |
|---|---|---|---|---|---|---|---|
| AF267856 | Hs.8084 | hypothetical protein dJ465N24.2.1 | | 5.83E−06 | 1.30 | 3.41E−05 | No statin < Statin |
| BC015869 | Hs.8136 | Homo sapiens clone 23698 mRNA sequence | | 4.28E−04 | 1.24 | 5.95E−03 | No statin < Statin |
| BF204294 | In multiple clusters | EST, Moderately similar to neuronal thread protein [Homo sapiens] | | 5.83E−06 | 1.24 | 1.65E−06 | No statin < Statin |
| | | [H. sapiens] | | | | | |
| BC028718 | Hs.337757 | Homo sapiens, hypothetical protein LOC51233, clone MGC: 33025 | | 4.28E−04 | 1.22 | 2.94E−04 | No statin < Statin |
| | | IMAGE: 5265935, mRNA, complete cds | | | | | |
| AK091047 | Hs.178485 | Homo sapiens cDNA FLJ13919 fis, clone Y79AA1000410 | | 1.11E−03 | 1.21 | 3.90E−03 | No statin < Statin |
| AA115054 | Hs.109438 | hypothetical protein BC013764 | | 1.11E−03 | 1.21 | 8.69E−03 | No statin < Statin |
| NM_015383 | Hs.41569 | hypothetical protein DJ328E19.C1.1 | DJ328E19.C1.1 | 1.11E−03 | 1.16 | 2.56E−01 | No statin < Statin |
| AL833316 | Hs.288156 | hypothetical protein MGC26766 | | 1.58E−04 | 1.16 | 1.38E−02 | No statin < Statin |
| BC030834 | Hs.118893 | ESTs, Weakly similar to T17346 hypothetical protein DKFZp586O1624.1 - | | 1.11E−03 | −1.14 | 7.56E−02 | Statin < No statin |
| | | human (fragment) [H. sapiens] | | | | | |
| BC014568 | Data not found | Homo sapiens, Similar to KDEL (Lys-Asp-Glu-Leu) endoplasmic reticulum | | 1.58E−04 | −1.21 | 5.38E−05 | Statin < No statin |
| | | protein retention receptor 2, clone MGC: 22272 IMAGE: 4063584, mRNA, | | | | | |
| | | complete cds | | | | | |
| AK026926 | Hs.182429 | Homo sapiens cDNA: FLJ23273 fis, clone HEP02611, highly similar to | | 1.58E−04 | −1.25 | 2.60E−05 | Statin < No statin |
| | | HSU79278 Human protein disulfide isomerase-related protein P5 mRNA | | | | | |
| AF495759 | Hs.74170 | Homo sapiens unknown mRNA | | 1.75E−06 | −1.42 | 4.25E−05 | Statin < No statin |
| T80495 | Hs.124969 | Hs. clone 24707 mRNA sequence | | 1.84E−05 | −1.65 | 1.53E−05 | Statin < No statin |

We claim:

1. A composition comprising:
a targeting agent conjugated to a functional moiety, wherein the targeting agent selectively binds to a polypeptide encoded by a DEA gene.

2. The composition of claim 1, wherein the DEA gene is overexpressed between atherosclerotic lesions and normal blood vessel tissue.

3. The composition of claim 1, wherein the DEA gene is differentially expressed between blood vessels of diabetic subjects and blood vessels of nondiabetic subjects.

4. The composition of claim 1, wherein the DEA gene is differentially expressed between non-lesion blood vessel tissue of diabetic subjects and non-lesion blood vessel tissue of nondiabetic subjects.

5. The composition of claim 1, wherein the DEA gene encodes a polypeptide selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

6. The composition of claim 1, wherein the targeting agent comprises an antibody or an antibody fragment that specifically binds to the polypeptide.

7. The composition of claim 1, wherein the functional moiety comprises a therapeutic agent.

8. The composition of claim 1, wherein the functional moiety comprises an imaging agent.

9. The composition of claim 1, wherein the agent is a paramagnetic, radioactive or fluorogenic ion.

10. A method of imaging vascular tissue in a subject comprising steps of:
(i) administering a targeting agent that specifically binds to a DEA polypeptide to the subject, wherein the targeting agent is linked to a functional moiety that enhances detectability of the DEA polypeptide; and
(ii) subjecting the subject to an imaging procedure that detects the functional moiety.

11. The method of claim 10, wherein the targeting agent is an antibody or antibody fragment.

12. The method of claim 10, wherein the targeting agent specifically binds to a polypeptide encoded by a gene selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

13. A method of targeting an agent to an atherosclerotic lesion comprising the step of: administering a conjugate or delivery vehicle comprising the molecule and a targeting agent that specifically binds to a polypeptide encoded by a DEA gene to the subject, wherein the DEA gene is overexpressed in atherosclerotic lesions relative to normal blood vessel tissue.

14. The method of claim 13, wherein the gene is selected from the group consisting of: CXCL6, MARCKS, osteopontin, MMP-10, oxidised low density lipoprotein (lectin-like) receptor 1, integral membrane protein 2A, integral membrane protein 2B, IL-18, IL-1α, IL-8, RANTES, MCP-1, MCP-2, MCP-3, lymphokine macrophage migration inhibitory factor, IL-6, ICAM-2, MMP-2, ICAM1, TIMP-1, TIMP3, CD4, CD8, granzyme B, thy1, COX-2, and ADAMTS1.

15. The method of claim 13, wherein the agent is a diagnostic or therapeutic agent.

16. A method of providing diagnostic or prognostic information related to atherosclerosis comprising steps of:
(i) providing a subject in need of diagnostic or prognostic information related to atherosclerosis;
(ii) determining the level of expression or activity of a DEA polynucleotide or polypeptide, or the level of a ligand for a DEA polypeptide, in the subject or in a biological sample obtained from the subject; and
(iii) utilizing the information to provide diagnostic or prognostic information.

17. The method of claim 16, wherein the step of utilizing comprises comparing the expression level or activity of the DEA polynucleotide or polypeptide, or the level of the ligand, with predetermined ranges of values for the expression level or activity of the DEA polynucleotide or polypeptide, or predetermined ranges of values for the level of the ligand, wherein the ranges are associated with levels of risk that a subject suffers from atherosclerosis, levels of disease severity, degree of response to treatment, or another type of diagnostic or prognostic information, thereby obtaining an indication of the risk, disease severity, or degree of response to treatment.

18. The method of claim 16, wherein the sample is a blood, plasma, or serum sample.

19. A method for identifying an agent that modulates expression or activity of a DEA polynucleotide or polypeptide comprising steps of: (i) providing a sample comprising a DEA polynucleotide or polypeptide; (ii) contacting the sample with a candidate compound; (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the expression or activity of the DEA polynucleotide or polypeptide if the level of expression or activity of the DEA polynucleotide or polypeptide is higher or lower in the presence of the compound relative to its level of expression or activity in the absence of the compound.

20. A method of treating or preventing atherosclerosis or a disease or condition associated with atherosclerosis comprising steps of:
(i) providing a subject at risk of or suffering from atherosclerosis or a disease or condition associated with atherosclerosis; and
(ii) administering a composition that modulates a DEA gene or expression product thereof to the subject.
